SEQUENCE LISTING (208 sequences)

SEQ ID NO: 1 - protein, 430 aa, Artificial Sequence
  fusion protein comprising: a fragment of TRAIL protein, boguanin domain A, sequences of steric linkers, fragment recognized by furin and pegylation linker sequence.
YNTVSFNLGEAYEYPTFIQDLRNELAKGTPVCQLPVTLQTIADDKRFVLVDITTTSKKTVKVAIDVTDVYVVGYQDKWDGKDRAVFLDKVPTVATSKLFPGVTNRVTLTFDGSYQKLVNAAKVDRKDLELGVYKLEFSIEAIHGKTINGQEIAKFFLIVIQMVSEAARFKYIETEVVDRGLYGSFKPNFKVLNLENNWGDISDAIHKSSPQCTTINPALQLISPSNDPWVVNKVSQISPDMGILKFKSGGGGSRKKRASGCGPEGGGGSRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 2 - protein, 453 aa, Artificial Sequence
  synthesized, fusion protein comprising: a fragment of TRAIL protein, domain of ricin A, sequences of steric linkers, fragment recognized by furin, pegylation linker sequence and transporting sequence
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSASGCGPERKKRGGGGSEDNNIFPKQYPIINFTTAGATVQSYTNFIRAVRGRLTTGADVRHEIPVLPNRVGLPINQRFILVELSNHAELSVTLALDVTNAYVVGYRAGNSAYFFHPDNQEDAEAITHLFTDVQNRYTFAFGGNYDRLEQLAGSLRENIELGNGPLEEAISALYYYSTGGTQLPTLARSFIVCIQMISEAARFQYIEGEMRTRIRYNRRSAPDPSVITLENSWGRLSTAIQESNQGAFASPIQLQRRNGSKFSVYDVSILIPIIALMVYRCAPPPKDEL

SEQ ID NO: 3 - protein, 378 aa, Artificial Sequence
  synthesized, fusion protein comprising: a fragment of TRAIL protein, domain of ricin A, sequences of steric linkers, fragment recognized by furin, pegylation linker sequence and transporting sequence.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSASGCGPERKKRGGGGSGPGPKQYPIINFTTAGATVQSYTNFIRAVRGRLTTGADVRHEIPVLPNRVGLPINQRFILVELSNHAELSVTLALDVTNAYVVGYRAGNSAYFFHPDNQEDAEAITHLFTDVQNRYTFAFGGNYDRLEQLAGNLRENIELGNGPLEEAISALYYYSTGGTQLPTLARSFIICIQMISEAARFQYIEGEMRVPKDEL

SEQ ID NO: 4 - protein, 473 aa, Artificial Sequence [sequence omitted]

SEQ ID NO: 5 - protein, 430 aa, Artificial Sequence
  synthesized, fusion protein comprising: a fragment of TRAIL protein, fragment of saporin, sequences of steric linkers and pegylation linker sequence.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSASGCGPEGGGGSVTSITLDLVNPTAGQYSSFVDKIRNNVKDPNLKYGGTDIAVIGPPSKDKFLRINFQSSRGTVSLGLKRDNLYVVAYLAMDNTNVNRAYYFKSEITSAELTALFPEATTANQKALEYTEDYQSIEKNAQITQGDKSRKELGLGIDLLLTFMEAVNKKARVVKNEARFLLIAIQMTAEVARFRYIQNLVTKNFPNKFDSDNKVIQFEVSWRKISTAIYGDAKNGVFNKDYDFGFGKVRQVKDLQMGLLMYLGKP

SEQ ID NO: 6 - protein, 442 aa, Artificial Sequence
  synthesized, fusion protein comprising: a fragment of TRAIL protein, fragment of saporin, sequences of steric linkers, fragment recognized by furin and pegylation linker sequence.
VRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGASGCGPEGGGGGSGGGGSRKKRVTSITLDLVNPTAGQYSSFVDKIRNNVKDPNLKYGGTDIAVIGPPSKDKFLRINFQSSRGTVSLGLKRDNLYVVAYLAMDNTNVNRAYYFKSEITSAELTALFPEATTANQKALEYTEDYQSIEKNAQITQGDKSRKELGLGIDLLLTFMEAVNKKARVVKNEARFLLIAIQMTAEVARFRYIQNLVTKNFPNKFDSDNKVIQFEVSWRKISTAIYGDAKNGVFNKDYDFGFGKVRQVKDLQMGLLMYLGKP

SEQ ID NO: 7 - protein, 429 aa, Artificial Sequence
  synthesized, fusion protein comprising: a fragment of TRAIL protein, trichosantin peptide, sequences of steric linkers, fragment recognized by furin and pegylation linker sequence.
DVSFRLSGATSSSYGVFISNLRKALPNERKLYDIPLLRSSLPGSQRYALIHLTNYADETISVAIDVTNVYIMGYRAGDTSYFFNEASATEAAKYVFKDAMRKVTLPYSGNYERLQTAAGKIRENIPLGLPALDSAITTLFYYNANSAASALMVLIQSTSEAARYKFIEQQIGKRVDKTFLPSLAIISLENSWSALSKQIQIASTNNGQFESPVVLINAQNQRVTITNVDAGVVTSNIALLLNRNNMAGGGGSRKKRASGCGPEGGGGSRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 8 - protein, 427 aa, Artificial Sequence
  synthesized, fusion protein comprising: a fragment of TRAIL protein, trichoanguin peptide, sequences of steric linkers, fragment recognized by furin and pegylation linker sequence.
DVSFDLSTATKKSYSSFITQLRDALPTQGTVCGIPLLPSTASGSQWFRFFNLTNYNDETVTVAVNVTNVYIVAYRADAVSYFFEDTPAEAFKLIFAGTKTVKLPYSGNYDKLQSVVGKQRDMIELGIPALSSAITNMVYYDYQSTAAALLVLIQCTAEAARYKYIEQQVSSHISSNFYPNQAVISLENKWGALSKQIQIANRTGHGQFENPVELYNPDGTRFSVTNTSAGVVKGNIKLLLYYKASGGGGSRKKRASGCGPEGGGGSRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 9 - protein, 427 aa, Artificial Sequence [sequence omitted]

SEQ ID NO: 10 - protein, 462 aa, Artificial Sequence
  synthesized, fusion protein comprising: a fragment of TRAIL protein, a subunit A of ebulin, sequences of steric linkers, sequence cleaved by furin and pegylation linker sequence.
IDYPSVSFNLAGAKSTTYRDFLKNLRDRVATGTYEVNGLPVLRRESEVQVKNRFVLVRLTNYNGDTVTSAVDVTNLYLVAFSANGNSYFFKDATELQKSNLFLGTTQHTLSFTGNYDNLETAAGTRRESIELGPNPLDGAITSLWYDGGVARSLLVLIQMVPEAARFRYIEQEVRRSLQQLTSFTPNALMLSMENNWSSMSLEVQLSGDNVSPFSGTVQLQNYDHTPRLVDNFEELYKITGIAILLFRCVATKTTHNAIRMPHVLVGEDNKFNGGGGSASGCGPERKKRGGGGVRERGPQRRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 11 - protein, 454 aa, Artificial Sequence
  synthesized, fusion protein comprising: a fragment of TRAIL protein, a subunit A of nigrin, sequences of steric linkers,sequence cleaved by furin and pegylation linker sequence.
IDYPSVSFNLDGAKSATYRDFLSNLRKTVATGTYEVNGLPVLRRESEVQVKSRFVLVPLTNYNGNTVTLAVDVTNLYVVAFSGNANSYFFKDATEVQKSNLFVGTKQNTLSFTGNYDNLETAANTRRESIELGPSPLDGAITSLYHGDSVARSLLVVIQMVSEAARFRYIEQEVRRSLQQATSFTPNALMLSMENNWSSMSLEIQQAGNNVSPFFGTVQLLNYDHTHRLVDNFEELYKITGIAILLFRCSSPSNDNAIRMPLDLAGEDNKYNGGGGSRKKRASGCGPEGGGGSRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 12 - protein, 221 aa, Artificial Sequence
  synthesized, fusion protein comprising: a fragment of TRAIL protein, a luffin P1 peptide, a sequence of steric linker and a sequence cleaved by furin.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSRKKRPRGSPRTEYEACRVRCQVAEHGVERQRRCQQVCEKRLREREGRREVDKDEL

SEQ ID NO: 13 - protein, 227 aa, Artificial Sequence
  synthesized, fusion protein comprising: a fragment of TRAIL protein, a luffin P1 peptide, sequences of steric linkers, pegylation linker sequence and a sequence cleaved by furin.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGSASGCGPERKKRASGGPRGSPRTEYEACRVRCQVAEHGVERQRRCQQVCEKRLREREGRREVD

SEQ ID NO: 14 - protein, 254 aa, Artificial Sequence
  synthesized, fusion protein comprising: a fragment of TRAIL protein, a luffin P1 peptide, a sequence of steric linker,pegylation linker sequence, a sequence cleaved by furin and transporting sequence.
TSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSASGCGPERKKRPRGSPRTEYEACRVRCQVAEHGVERQRRCQQVCEKRLREREGRREVDKDEL

SEQ ID NO: 15 - protein, 438 aa, Artificial Sequence
  synthesized, fusion protein comprising: a fragment of TRAIL protein, subunit A of volkensin, sequences of steric linkers, pegylation linker sequence and a sequence cleaved by furin.
VFPKVPFDVPKATVESYTRFIRVLRDELAGGVSPQGIRRLRNPAEIQPSQGFILIQLTGYVGSVTLIMDVRNAYLLGYLSHNVLYHFNDVSASSIASVFPDAQRRQLPFGGGYPSMRNYAPERDQIDHGIVELAYAVDRLYYSQNNNQIALGLVICAGMVAEASRFRYIEGLVRQSIVGPGDYRTFRPDALMYSIVTQWQTLSERIQGSFNGAFQPVQLGYASDPFYWDNVAQAITRLSLMLFVSRSTDGGGGSRVKRASGCGPEGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 16 - protein, 431 aa, Artificial Sequence
  synthesized, fusion protein comprising: a fragment of TRAIL protein, subunit A of volkensin, sequences of steric linkers, pegylation linker sequence, a sequence cleaved by furin and transporting sequence.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSASGCGPEGGGGSVFPKVPFDVPKATVESYTRFIRVLRDELAGGVSPQGIRRLRNPAEIQPSQGFILIQLTGYVGSVTLIMDVRNAYLLGYLSHNVLYHFNDVSASSIASVFPDAQRRQLPFGGGYPSMRNYAPERDQIDHGIVELAYAVDRLYYSQNNNQIALGLVICAGMVAEASRFRYIEGLVRQSIVGPGDYRTFRPDALMYSIVTQWQTLSERIQGSFNGAFQPVQLGYASDPFYWDNVAQAITRLSLMLFVSRSTDKDEL

SEQ ID NO: 17 - protein, 428 aa, Artificial Sequence
  synthesized, fusion protein comprising: a fragment of TRAIL protein, subunit A of momorcharin, sequences of steric linkers, pegylation linker sequence and a sequence cleaved by furin.
DVSFRLSGADPRSYGMFIKDLRNALPFREKVYNIPLLLPSVSGAGRYLLMHLFNYDGKTITVALDVTNVYIMGYLADTTSYFFNEPAAELASQYVFRDARRKITLPYSGNYERLQIAAGKPREKIPIGLPALDSAISTLLHYDSTAAAGALLVLIQTTAEAARFKYIEQQIQERAYRDEVPSLATISLENSWSGLSKQIQLAQGNNGIFRTPIVLVDNKGNRVQITNVTSKVVTSNIQLLLNTRNIGGGGSRKKRASGCGPEGGGGSRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 18 - protein, 515 aa, Artificial Sequence
  synthesized, fusion protein comprising: a fragment of TRAIL protein, fragment of modified sequence of Pseudomonas aeruginosa exotoxin, sequences of steric linkers and a transporting sequence directing the effector peptide to the reticulum.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGSASGGPEGGSLAALTAHQACHLPLETFTRHRQPRGWEQLEQCGYPVQRLVALYLAARLSWNQVDQVIANALASPGSGGDLGEAIRESPEQARLALTLAAAESERFVRQGTGNDEAGAANGPADSGDALLERNYPTGAEFLGDGGDVSFSTRGTQNWTVERLLQAHRQLEEAGYVFVGYHGTFLEAAQSIVFGGVRARSQDLDAIWAGFYIAGDPALAYGYAQDQEPDAAGRIRNGALLRVYVPRSSLPGFYATSLTLAAPEAAGEVERLIGHPLPLRLDAITGPEESGGRLETILGWPLAERTVVIPSAIPTDPRNVGGDLDPSSIPDSEQAISALPDYASQPGKPPKDEL

SEQ ID NO: 19 - protein, 528 aa, Artificial Sequence
  synthesized, fusion protein comprising: a fragment of TRAIL protein,modified sequence of P.aeruginosa exotoxin,sequences of steric linkers,pegylation linker, a sequence cleaved by furin and a transporting sequence.
PQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGSASGCGPERKKRASGGPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDVSFSTRGTQNWTVERLLQAHRQLEEAGYVFVGYHGTFLEAAQSIVFGGVRARSQDLDAIWAGFYIAGDPALAYGYAQDQEPDAAGRIRNGALLRVYVPRSSLPGFYATSLTLAAPEAAGEVERLIGHPLPLRLDAITGPEESGGRLETILGWPLAERTVVIPSAIPTDPRNVGGDLDPSSIPDSEQAISALPDYASQPGKPPKDEL

SEQ ID NO: 20 - protein, 526 aa, Artificial Sequence
  synthesized, fusion protein comprising: a fragment of TRAIL protein, fragment of modified sequence of Pseudomonas aeruginosa exotoxin, sequences of steric linkers, pegylation linker sequence, a sequence cleaved by furin and a transporting sequence.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGSASGCGPERKKRASGGPEGGSLAALTAHQACHLPLETFTRHRQPRGWEQLEQCGYPVQRLVALYLAARLSWNQVDQVIANALASPGSGGDLGEAIRESPEQARLALTLAAAESERFVRQGTGNDEAGAANGPADSGDALLERNYPTGAEFLGDGGDVSFSTRGTQNWTVERLLQAHRQLEEAGYVFVGYHGTFLEAAQSIVFGGVRARSQDLDAIWAGFYIAGDPALAYGYAQDQEPDAAGRIRNGALLRVYVPRSSLPGFYATSLTLAAPEAAGEVERLIGHPLPLRLDAITGPEESGGRLETILGWPLAERTVVIPSAIPTDPRNVGGDLDPSSIPDSEQAISALPDYASQPGKPPKDEL

SEQ ID NO: 21 - protein, 534 aa, Artificial Sequence [sequence omitted]

SEQ ID NO: 22 - protein, 515 aa, Artificial Sequence
  synthesized, fusion protein comprising: a fragment of TRAIL protein, fragment of modified sequence of Pseudomonas aeruginosa exotoxin and a sequence of steric linker.
RVAAHITGTRGRSNTLSSPNSKNEKALGRKINSWESSRSGHSFLSNLHLRNGELVIHEKGFYYIYSQTNFKFREEIKENTKNDKQMVQYIYKYTSYPDPILLMKSARNSCWSKDAEYGLYSIYQGGIFELKENDRIFVSVTNERLRDMHHEASFFGAFLVGGGGSASGGPEGGSLAALTAHQACHLPLETFTRHRQPRGWEQLEQCGYPVQRLVALYLAARLSWNQVDQVIANALASPGSGGDLGEAIRESPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWAGFYIAGDPALAYGYAQDQEPDAAGRIRNGALLRVYVPRSSLPGFYATSLTLAAPEAAGEVERLIGHPLPLRLDAITGPEESGGRLETILGWPLAERTVVIPSAIPTDPRNVGGDLDPSSIPDSEQAISALPDYASQPGKPPKDEL

SEQ ID NO: 23 - protein, 528 aa, Artificial Sequence
  synthesized, fusion protein comprising: a fragment of TRAIL protein, fragment of modified sequence of Pseudomonas aeruginosa exotoxin, a pegylation linker sequence, a sequence cleaved by furin,steric linkers sequences and a transporting sequence.
PQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGSASGCGPERKKRASGGPEGGSLAALTAHQACHLPLETFTRHRQPRGWEQLEQCGYPVQRLVALYLAARLSWNQVDQVIANALASPGSGGDLGEAIRESPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWAGFYIAGDPALAYGYAQDQEPDAAGRIRNGALLRVYVPRSSLPGFYATSLTLAAPEAAGEVERLIGHPLPLRLDAITGPEESGGRLETILGWPLAERTVVIPSAIPTDPRNVGGDLDPSSIPDSEQAISALPDYASQPGKPPKDEL

SEQ ID NO: 24 - protein, 526 aa, Artificial Sequence
  synthesized, fusion protein comprising: a fragment of TRAIL protein, fragment of modified sequence of Pseudomonas aeruginosa exotoxin, a pegylation linker sequence, a sequence cleaved by furin,steric linkers sequences and a transporting sequence.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGSASGCGPERKKRASGGPEGGSLAALTAHQACHLPLETFTRHRQPRGWEQLEQCGYPVQRLVALYLAARLSWNQVDQVIANALASPGSGGDLGEAIRESPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWAGFYIAGDPALAYGYAQDQEPDAAGRIRNGALLRVYVPRSSLPGFYATSLTLAAPEAAGEVERLIGHPLPLRLDAITGPEESGGRLETILGWPLAERTVVIPSAIPTDPRNVGGDLDPSSIPDSEQAISALPDYASQPGKPPKDEL

SEQ ID NO: 25 - protein, 423 aa, Artificial Sequence
  synthesized, fusion protein comprising: a fragment of TRAIL protein, variant of Shiga toxin stx, a sequence cleaved by furin and sequences of steric linkers.
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHASGGGGSRVKRVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 26 - protein, 432 aa, Artificial Sequence
  synthesized, fusion protein comprising: a fragment of TRAIL protein, variant of Shiga toxin stx, a pegylation linker sequence, a sequence cleaved by furin, sequences of steric linkers and a transporting sequence.
QRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGSASGCGPERKKRGGGGSKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHASKDEL

SEQ ID NO: 27 - protein, 338 aa, Artificial Sequence
  synthesized, fusion protein comprising: a fragment of TRAIL protein, restrictocin peptide, a pegylation linker sequence, a sequence cleaved by furin and sequences of steric linkers.
ATWTCINQQLNPKTNKWEDKRLLYSQAKAESNSHHAPLSDGKTGSSYPHWFTNGYDGNGKLIKGRTPIKFGKADCDRPPKHSQNGMGKDDHYLLEFPTFPDGHDYKFDSKKPKEDPGPARVIYTYPNKVFCGIVAHQRGNQGDLRLCSHGGGGSGGGGSRKKRASGCGPEVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 28 - protein, 335 aa, Artificial Sequence
  synthesized, fusion protein comprising: a fragment of TRAIL protein, restrictocin peptide, a pegylation linker sequence, a sequence cleaved by furin, sequences of steric linkers and a transporting sequence.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSASGCGPERKKRGGGGSATWTCINQQLNPKTNKWEDKRLLYSQAKAESNSHHAPLSDGKTGSSYPHWFTNGYDGNGKLIKGRTPIKFGKADCDRPPKHSQNGMGKDDHYLLEFPTFPDGHDYKFDSKKPKEDPGPARVIYTYPNKVFCGIVAHQRGNQGDLRLCSHKDEL

SEQ ID NO: 29 - protein, 319 aa, Artificial Sequence
  synthesized, fusion protein comprising: a fragment of TRAIL protein, hirsutellin peptide, a pegylation linker sequence, a sequence cleaved by furin and sequences of steric linkers.
APIVTCRPKLDGREKPFKVDVATAQAQARKAGLTTGKSGDPHRYFAGDHIRWGVNNCDKADAILWEYPIYWVGKNAEWAKDVKTSQQKGGPTPIRVVYANSRGAVQYCGVMTHSKVDKNNQGKEFFEKCDGGGGSGGGGSRKKRASGCGPEVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 30 - protein, 290 aa, Artificial Sequence [sequence omitted]

SEQ ID NO: 31 - protein, 277 aa, Artificial Sequence [sequence omitted]

SEQ ID NO: 32 - protein, 228 aa, Artificial Sequence
  synthesized, fusion protein comprising: a fragment of TRAIL protein, CcdB protein, a pegylation linker sequence, a sequence cleaved by furin, a sequence of steric linker and transporting sequence.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSASGCGPERKKRQFKVYTYKGGSGGRLLSDKVSRELGGSGGSHRENDIKNAINLMFWGIKDEL

SEQ ID NO: 33 - protein, 275 aa, Artificial Sequence
  synthesized, fusion protein comprising: a fragment of TRAIL protein, RelE protein, a pegylation linker sequence, a sequence cleaved by furin, a sequence of steric linker and transporting sequence.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSASGCGPERKKRAYFLDFDERALKEWRKLGSTVREQLKKKLVEVLESPRIEANKLRGMPDCYKIKLRSSGYRLVYQVIDEKVVVFVISVGKRERSEVYSEAVKRILKDEL

SEQ ID NO: 34 - protein, 271 aa, Artificial Sequence [sequence omitted]

SEQ ID NO: 35 - protein, 429 aa, Artificial Sequence
  synthesized, fusion protein comprising: a fragment of TRAIL protein, gelonin peptide, and sequences of steric linkers.
GLDTVSFSTKGATYITYVNFLNELRVKLKPEGNSHGIPLLRKKADDPGKAFVLVALSNDNGQLAEIAIDVTSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPKGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 36 - protein, 434 aa, Artificial Sequence [sequence omitted]

SEQ ID NO: 37 - protein, 427 aa, Artificial Sequence
  synthesized, fusion protein comprising: a fragment of TRAIL protein, gelonin peptide, a sequence of steric linker, a pegylation linker sequence and transporting sequence directing the effector peptide to the endoplasmic reticulum.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGASGCGPEGGGGSGLDTVSFSTKGATYITYVNFLNELRVKLKPEGNSHGIPLLRKKADDPGKAFVLVALSNDNGQLAEIAIDVTSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPKDEL

SEQ ID NO: 38 - protein, 433 aa, Artificial Sequence
  synthesized, fusion protein comprising: a fragment of TRAIL protein, gelonin peptide, sequences of steric linkers, a pegylation linker sequence and a sequence cleaved by furin.
GLDTVSFSTKGATYITYVNFLNELRVKLKPEGNSHGIPLLRKKADDPGKAFVLVALSNDNGQLAEIAIDVTSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPKGGGGSRKKRASGCGPEGGGGSRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 39 - protein, 558 aa, Artificial Sequence
  synthesized, fusion protein comprising: a fragment of TRAIL protein, subunit A of diphteria toxin and sequences of steric linkers.
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHGGGGSGGGGSRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 40 - protein, 481 aa, Artificial Sequence [sequence omitted]

SEQ ID NO: 41 - protein, 481 aa, Artificial Sequence
  synthesized, fusion protein comprising: a fragment of TRAIL protein, catalytic domain of diphtheria toxin, sequences of steric linkers, sequences cleaved by furin and a sequence of transporting domain.
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRKKRGGGGSPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNGRKKRGGGGSGGGGSRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 42 - protein, 433 aa, Artificial Sequence
  synthesized, fusion protein comprising: a fragment of TRAIL protein, domain A of abrin, and sequences of steric linkers.
ARHMEDRPIKFSTEGATSQSYKQFIEALRERLRGGLIHDIPVLPDPTTLQERNRYITVELSNSDTESIEVGIDVTNAYVVAYRAGTQSYFLRDAPSSASDYLFTGTDQHSLPFYGTYGDLERWAHQSRQQIPLGLQALTHGISFFRSGGNDNEEKARTLIVIIQMVAEAARFRYISNRVRVSIQTGTAFQPDAAMISLENNWDNLSRGVQESVQDTFPNQVTLTNIRNEPVIVDSLSHPTVAVLALMLFVCNPPNGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 43 - protein, 447 aa, Artificial Sequence
  synthesized, fusion protein comprising: a fragment of TRAIL protein, domain A of abrin, sequences of steric linkers, a sequence of integrin ligand and a sequence cleaved by urokinase.
ARHMEDRPIKFSTEGATSQSYKQFIEALRERLRGGLIHDIPVLPDPTTLQERNRYITVELSNSDTESIEVGIDVTNAYVVAYRAGTQSYFLRDAPSSASDYLFTGTDQHSLPFYGTYGDLERWAHQSRQQIPLGLQALTHGISFFRSGGNDNEEKARTLIVIIQMVAEAARFRYISNRVRVSIQTGTAFQPDAAMISLENNWDNLSRGVQESVQDTFPNQVTLTNIRNEPVIVDSLSHPTVAVLALMLFVCNPPNGGGGSCFCDGRCDCARVVRGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 44 - protein, 433 aa, Artificial Sequence
  synthesized, fusion protein comprising: a fragment of TRAIL protein, domain A of abrin, sequences of steric linkers and a sequence cleaved by urokinase.
EDRPIKFSTEGATSQSYKQFIEALRERLRGGLIHDIPVLPDPTTLQERNRYITVELSNSDTESIEVGIDVTNAYVVAYRAGTQSYFLRDAPSSASDYLFTGTDQHSLPFYGTYGDLERWAHQSRQQIPLGLQALTHGISFFRSGGNDNEEKARTLIVIIQMVAEAARFRYISNRVRVSIQTGTAFQPDAAMISLENNWDNLSRGVQESVQDTFPNQVTLTNIRNEPVIVDSLSHPTVAVLALMLFVCNPPNGGGGSGGGGSRVVRVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 45 - protein, 441 aa, Artificial Sequence
  synthesized, fusion protein comprising: a fragment of TRAIL protein, domain A of abrin, sequences of steric linkers, a sequence cleaved by urokinase and arginine transporting sequence.
EDRPIKFSTEGATSQSYKQFIEALRERLRGGLIHDIPVLPDPTTLQERNRYITVELSNSDTESIEVGIDVTNAYVVAYRAGTQSYFLRDAPSSASDYLFTGTDQHSLPFYGTYGDLERWAHQSRQQIPLGLQALTHGISFFRSGGNDNEEKARTLIVIIQMVAEAARFRYISNRVRVSIQTGTAFQPDAAMISLENNWDNLSRGVQESVQDTFPNQVTLTNIRNEPVIVDSLSHPTVAVLALMLFVCNPPNRRRRRRRRRVVRGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 46 - protein, 550 aa, Artificial Sequence [sequence omitted]

SEQ ID NO: 47 - protein, 459 aa, Artificial Sequence [sequence omitted]

SEQ ID NO: 48 - protein, 443 aa, Artificial Sequence
  synthesized, fusion protein comprising: a fragment of TRAIL protein, domain A of abrin, sequences of steric linkers, a sequence cleaved by urokinase and pegylation linker sequence.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSASGCGPERKKRGGGGSARHMEDRPIKFSTEGATSQSYKQFIEALRERLRGGLIHDIPVLPDPTTLQERNRYITVELSNSDTESIEVGIDVTNAYVVAYRAGTQSYFLRDAPSSASDYLFTGTDQHSLPFYGTYGDLERWAHQSRQQIPLGLQALTHGISFFRSGGNDNEEKARTLIVIIQMVAEAARFRYISNRVRVSIQTGTAFQPDAAMISLENNWDNLSRGVQESVQDTFPNQVTLTNIRNEPVIVDSLSHPTVAVLALMLFVCNPPNVRERGP

SEQ ID NO: 49 - protein, 447 aa, Artificial Sequence
  synthesized, fusion protein comprising: a fragment of TRAIL protein, domain A of abrin, sequences of steric linkers, a sequence cleaved by urokinase, a pegylation linker sequence and a transporting sequence.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSASGCGPERKKRGGGGSARHMEDRPIKFSTEGATSQSYKQFIEALRERLRGGLIHDIPVLPDPTTLQERNRYITVELSNSDTESIEVGIDVTNAYVVAYRAGTQSYFLRDAPSSASDYLFTGTDQHSLPFYGTYGDLERWAHQSRQQIPLGLQALTHGISFFRSGGNDNEEKARTLIVIIQMVAEAARFRYISNRVRVSIQTGTAFQPDAAMISLENNWDNLSRGVQESVQDTFPNQVTLTNIRNEPVIVDSLSHPTVAVLALMLFVCNPPNVRERGPKDEL

SEQ ID NO: 50 - protein, 441 aa, Artificial Sequence
  synthesized, fusion protein comprising: a fragment of TRAIL protein, domain A of abrin, sequences of steric linkers, a sequence cleaved by urokinase and a pegylation linker sequence.
EDRPIKFSTEGATSQSYKQFIEALRERLRGGLIHDIPVLPDPTTLQERNRYITVELSNSDTESIEVGIDVTNAYVVAYRAGTQSYFLRDAPSSASDYLFTGTDQHSLPFYGTYGDLERWAHQSRQQIPLGLQALTHGISFFRSGGNDNEEKARTLIVIIQMVAEAARFRYISNRVRVSIQTGTAFQPDAAMISLENNWDNLSRGVQESVQDTFPNQVTLTNIRNEPVIVDSLSHPTVAVLALMLFVCNPPNGGGGSGGGGSRKKRASGCGPECVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 51 - protein, 515 aa, Artificial Sequence
  synthesized, fusion protein comprising: a fragment of mutated TRAIL protein, modified Pseudomonas aeruginosa exotoxin sequence,sequences of steric linkers and transporting sequence directing the effector peptide to endoplasmic reticulum.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPHPILLMKSARNSCWSKDAEYGLYSIYQGGIFELKENDRIFVSVTNEHLIDMDHEASFFGAFLVGGGGSASGGPEGGSLAALTAHQACHLPLETFTRHRQPRGWEQLEQCGYPVQRLVALYLAARLSWNQVDQVIANALASPGSGGDLGEAIRESPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWAGFYIAGDPALAYGYAQDQEPDAAGRIRNGALLRVYVPRSSLPGFYATSLTLAAPEAAGEVERLIGHPLPLRLDAITGPEESGGRLETILGWPLAERTVVIPSAIPTDPRNVGGDLDPSSIPDSEQAISALPDYASQPGKPPKDEL

SEQ ID NO: 52 - protein, 515 aa, Artificial Sequence; identical to SEQ ID NO: 22
  synthesized, fusion protein comprising: a fragment of mutated TRAIL protein, modified Pseudomonas aeruginosa exotoxin sequence, sequences of steric linkers and transporting sequence directing the effector peptide to endoplasmic reticulum.

SEQ ID NO: 53 - protein, 519 aa, Artificial Sequence
  synthesized, fusion protein comprising: a fragment of mutated TRAIL protein, modified Pseudomonas aeruginosa exotoxin sequence, a sequence of steric linker and a sequence of pegylation linker and transporting sequence.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPHPILLMKSARNSCWSKDAEYGLYSIYQGGIFELKENDRIFVSVTNEHLIDMDHEASFFGAFLVGGGGSGASGCGPEPEGGSLAALTAHQACHLPLETFTRHRQPRGWEQLEQCGYPVQRLVALYLAARLSWNQVDQVIANALASPGSGGDLGEAIRESPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWAGFYIAGDPALAYGYAQDQEPDAAGRIRNGALLRVYVPRSSLPGFYATSLTLAAPEAAGEVERLIGHPLPLRLDAITGPEESGGRLETILGWPLAERTVVIPSAIPTDPRNVGGDLDPSSIPDSEQAISALPDYASQPGKPPKDEL

SEQ ID NO: 54 - protein, 515 aa, Artificial Sequence; identical to SEQ ID NO: 22
  synthesized, fusion protein comprising: a fragment of mutated TRAIL protein, modified Pseudomonas aeruginosa exotoxin sequence, sequences of steric linkers and transporting sequence directing the effector peptide to endoplasmic reticulum.

SEQ ID NO: 55 - protein, 248 aa, Bougainvillea spectabilis
  ref <308>: DATABASE ACCESSION NUMBER: GenBank/AAL35962.1
  ref <309>: DATABASE ENTRY DATE: 2001-11-29
  ref <313>: RELEVANT RESIDUES IN SEQ ID NO: (27)..(247)
YNTVSFNLGEAYEYPTFIQDLRNELAKGTPVCQLPVTLQTIADDKRFVLVDITTTSKKTVKVAIDVTDVYVVGYQDKWDGKDRAVFLDKVPTVATSKLFPGVTNRVTLTFDGSYQKLVNAAKVDRKDLELGVYKLEFSIEAIHGKTINGQEIAKFFLIVIQMVSEAARFKYIETEVVDRGLYGSFKPNFKVLNLENNWGDISDAIHKSSPQCTTINPALQLISPSNDPWVVNKVSQISPDMGILKFKS

SEQ ID NO: 56 - protein, 267 aa, Ricinus communis
  ref <308>: DATABASE ACCESSION NUMBER: GenBank/ABG65738.1
  ref <309>: DATABASE ENTRY DATE: 2006-07-16
  ref <313>: RELEVANT RESIDUES IN SEQ ID NO: (1)..(267)
EDNNIFPKQYPIINFTTAGATVQSYTNFIRAVRGRLTTGADVRHEIPVLPNRVGLPINQRFILVELSNHAELSVTLALDVTNAYVVGYRAGNSAYFFHPDNQEDAEAITHLFTDVQNRYTFAFGGNYDRLEQLAGSLRENIELGNGPLEEAISALYYYSTGGTQLPTLARSFIVCIQMISEAARFQYIEGEMRTRIRYNRRSAPDPSVITLENSWGRLSTAIQESNQGAFASPIQLQRRNGSKFSVYDVSILIPIIALMVYRCAPPP

SEQ ID NO: 57 - protein, 192 aa, Ricinus communis
  ref <308>: DATABASE ACCESSION NUMBER: GenBank/ACZ56254.1
  ref <309>: DATABASE ENTRY DATE: 2009-12-05
  ref <313>: RELEVANT RESIDUES IN SEQ ID NO: (1)..(192)
GPGPKQYPIINFTTAGATVQSYTNFIRAVRGRLTTGADVRHEIPVLPNRVGLPINQRFILVELSNHAELSVTLALDVTNAYVVGYRAGNSAYFFHPDNQEDAEAITHLFTDVQNRYTFAFGGNYDRLEQLAGNLRENIELGNGPLEEAISALYYYSTGGTQLPTLARSFIICIQMISEAARFQYIEGEMRVP

SEQ ID NO: 58 - protein, 290 aa, Phytolacca americana
  ref <308>: DATABASE ACCESSION NUMBER: GenBank/CAA66702.1
  ref <309>: DATABASE ENTRY DATE: 2005-04-18
  ref <313>: RELEVANT RESIDUES IN SEQ ID NO: (25)..(314)
INTITFDAGNATINKYATFMESLRNQAKDPKLKCYGIPMLPDTNSTPKYLLVKLQGANLKTITLMLRRNNLYVMGYSDPFNGNKCRYHIFNDITSTERTDVENTLCSSSSSRVAMSINYNSLYPTMEKKAEVNSRNQVQLGIQILSSDIGKISGVDSFPVKTEAFFLLVAIQMVSEAARFKYIENQVKTNFNRAFYPDPKVINLEEKWGKISEAIHNAKNGALPKPLELVDAKGTKWIVLRVDEINRDVALLKYVNGTCQTTYQNAMFSQVIISTYYNYMSNLGDLFEGF

SEQ ID NO: 59 - protein, 252 aa, Saponaria officinalis
  ref <308>: DATABASE ACCESSION NUMBER: GenBank/CAA48886.1
  ref <309>: DATABASE ENTRY DATE: 2005-04-18
  ref <313>: RELEVANT RESIDUES IN SEQ ID NO: (1)..(251)
VTSITLDLVNPTAGQYSSFVDKIRNNVKDPNLKYGGTDIAVIGPPSKDKFLRINFQSSRGTVSLGLKRDNLYVVAYLAMDNTNVNRAYYFKSEITSAELTALFPEATTANQKALEYTEDYQSIEKNAQITQGDKSRKELGLGIDLLLTFMEAVNKKARVVKNEARFLLIAIQMTAEVARFRYIQNLVTKNFPNKFDSDNKVIQFEVSWRKISTAIYGDAKNGVFNKDYDFGFGKVRQVKDLQMGLLMYLGKP

SEQ ID NO: 60 - protein, 247 aa, Trichosanthes kirilowii
  ref <308>: DATABASE ACCESSION NUMBER: GenBank/AAB22585.1
  ref <309>: DATABASE ENTRY DATE: 1993-05-08
  ref <313>: RELEVANT RESIDUES IN SEQ ID NO: (1)..(247)
DVSFRLSGATSSSYGVFISNLRKALPNERKLYDIPLLRSSLPGSQRYALIHLTNYADETISVAIDVTNVYIMGYRAGDTSYFFNEASATEAAKYVFKDAMRKVTLPYSGNYERLQTAAGKIRENIPLGLPALDSAITTLFYYNANSAASALMVLIQSTSEAARYKFIEQQIGKRVDKTFLPSLAIISLENSWSALSKQIQIASTNNGQFESPVVLINAQNQRVTITNVDAGVVTSNIALLLNRNNMA

SEQ ID NO: 61 - protein, 245 aa, Trichosanthes anguina
  ref <308>: DATABASE ACCESSION NUMBER: GenBank/AAD02686.1
  ref <309>: DATABASE ENTRY DATE: 1999-03-03
  ref <313>: RELEVANT RESIDUES IN SEQ ID NO: (20)..(264)
DVSFDLSTATKKSYSSFITQLRDALPTQGTVCGIPLLPSTASGSQWFRFFNLTNYNDETVTVAVNVTNVYIVAYRADAVSYFFEDTPAEAFKLIFAGTKTVKLPYSGNYDKLQSVVGKQRDMIELGIPALSSAITNMVYYDYQSTAAALLVLIQCTAEAARYKYIEQQVSSHISSNFYPNQAVISLENKWGALSKQIQIANRTGHGQFENPVELYNPDGTRFSVTNTSAGVVKGNIKLLLYYKAS

SEQ ID NO: 62 - protein, 249 aa, Viscum album
  ref <308>: DATABASE ACCESSION NUMBER: GenBank/AAL87006.1
  ref <309>: DATABASE ENTRY DATE: 2002-03-17
  ref <313>: RELEVANT RESIDUES IN SEQ ID NO: (1)..(249)
YERLRLRVTHQTTGEEYFRFITLLRDYVSSGSFSNEIPLLRQSTIPVSDAQRFVLVELTNEGGDSITAAIDVTNLYVVAYQAGDQSYFLRDAPRGAETHLFTGTTRSSLPFNGSYPDLERYAGHRDQIPLGIDQLIQSVTALRFPGGSTRTQARSILILIQMISEAARFNPILWRARQYINSGASFLPDVYMLELETSWGQQSTQVQQSTDGVFNNPIRLAIPPGNFVTLTNVRDVIASLAIMLFVCGE

SEQ ID NO: 63 - protein, 273 aa, Sambucus ebulus L
  ref <308>: DATABASE ACCESSION NUMBER: GenBank/CAC33178.1
  ref <309>: DATABASE ENTRY DATE: 2001-02-17
  ref <313>: RELEVANT RESIDUES IN SEQ ID NO: (26)..(298)
IDYPSVSFNLAGAKSTTYRDFLKNLRDRVATGTYEVNGLPVLRRESEVQVKNRFVLVRLTNYNGDTVTSAVDVTNLYLVAFSANGNSYFFKDATELQKSNLFLGTTQHTLSFTGNYDNLETAAGTRRESIELGPNPLDGAITSLWYDGGVARSLLVLIQMVPEAARFRYIEQEVRRSLQQLTSFTPNALMLSMENNWSSMSLEVQLSGDNVSPFSGTVQLQNYDHTPRLVDNFEELYKITGIAILLFRCVATKTTHNAIRMPHVLVGEDNKFN

SEQ ID NO: 64 - protein, 272 aa, Sambucus nigra
  ref <308>: DATABASE ACCESSION NUMBER: GenBank/AAB39475.1
  ref <309>: DATABASE ENTRY DATE: 1996-12-13
  ref <313>: RELEVANT RESIDUES IN SEQ ID NO: (26)..(297)
IDYPSVSFNLDGAKSATYRDFLSNLRKTVATGTYEVNGLPVLRRESEVQVKSRFVLVPLTNYNGNTVTLAVDVTNLYVVAFSGNANSYFFKDATEVQKSNLFVGTKQNTLSFTGNYDNLETAANTRRESIELGPSPLDGAITSLYHGDSVARSLLVVIQMVSEAARFRYIEQEVRRSLQQATSFTPNALMLSMENNWSSMSLEIQQAGNNVSPFFGTVQLLNYDHTHRLVDNFEELYKITGIAILLFRCSSPSNDNAIRMPLDLAGEDNKYN

SEQ ID NO: 65 - protein, 47 aa, Luffa cylindrica
  ref <308>: DATABASE ACCESSION NUMBER: Swiss-Prot/P56568.1
  ref <309>: DATABASE ENTRY DATE: 2011-06-28
  ref <313>: RELEVANT RESIDUES IN SEQ ID NO: (1)..(47)
PRGSPRTEYEACRVRCQVAEHGVERQRRCQQVCEKRLREREGRREVD

SEQ ID NO: 66 - protein, 249 aa, Adenia volkensii
  ref <308>: DATABASE ACCESSION NUMBER: GenBank/CAD61022.1
  ref <309>: DATABASE ENTRY DATE: 2005-04-15
  ref <313>: RELEVANT RESIDUES IN SEQ ID NO: (1)..(244)
VFPKVPFDVPKATVESYTRFIRVLRDELAGGVSPQGIRRLRNPAEIQPSQGFILIQLTGYVGSVTLIMDVRNAYLLGYLSHNVLYHFNDVSASSIASVFPDAQRRQLPFGGGYPSMRNYAPERDQIDHGIVELAYAVDRLYYSQNNNQIALGLVICAGMVAEASRFRYIEGLVRQSIVGPGDYRTFRPDALMYSIVTQWQTLSERIQGSFNGAFQPVQLGYASDPFYWDNVAQAITRLSLMLFVSRSTD

SEQ ID NO: 67 - protein, 246 aa, Momordica charantia
  ref <308>: DATABASE ACCESSION NUMBER: GenBank/AAB22586.1
  ref <309>: DATABASE ENTRY DATE: 1993-05-08
  ref <313>: RELEVANT RESIDUES IN SEQ ID NO: (1)..(246)
DVSFRLSGADPRSYGMFIKDLRNALPFREKVYNIPLLLPSVSGAGRYLLMHLFNYDGKTITVALDVTNVYIMGYLADTTSYFFNEPAAELASQYVFRDARRKITLPYSGNYERLQIAAGKPREKIPIGLPALDSAISTLLHYDSTAAAGALLVLIQTTAEAARFKYIEQQIQERAYRDEVPSLATISLENSWSGLSKQIQLAQGNNGIFRTPIVLVDNKGNRVQITNVTSKVVTSNIQLLLNTRNI

SEQ ID NO: 68 - protein, 342 aa, Pseudomonas aeruginosa
  ref <302>: TITLE: MUTATED PSEUDOMONAS EXOTOXINS WITH REDUCED ANTIGENICITY
  ref <310>: PATENT DOCUMENT NUMBER: WO/2007/016150
  ref <311>: PATENT FILING DATE: 2006-07-25
  ref <312>: PUBLICATION DATE: 2007-02-08
PEGGSLAALTAHQACHLPLETFTRHRQPRGWEQLEQCGYPVQRLVALYLAARLSWNQVDQVIANALASPGSGGDLGEAIRESPEQARLALTLAAAESERFVRQGTGNDEAGAANGPADSGDALLERNYPTGAEFLGDGGDVSFSTRGTQNWTVERLLQAHRQLEEAGYVFVGYHGTFLEAAQSIVFGGVRARSQDLDAIWAGFYIAGDPALAYGYAQDQEPDAAGRIRNGALLRVYVPRSSLPGFYATSLTLAAPEAAGEVERLIGHPLPLRLDAITGPEESGGRLETILGWPLAERTVVIPSAIPTDPRNVGGDLDPSSIPDSEQAISALPDYASQPGKPP

SEQ ID NO: 69 - protein, 354 aa, Pseudomonas aeruginosa
  ref <302>: TITLE: MUTATED PSEUDOMONAS EXOTOXINS WITH REDUCED ANTIGENICITY
  ref <310>: PATENT DOCUMENT NUMBER: WO/2007/016150
  ref <311>: PATENT FILING DATE: 2006-07-25
  ref <312>: PUBLICATION DATE: 2007-02-08
PEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 70 - protein, 241 aa, Shigella dysentarie
  ref <308>: DATABASE ACCESSION NUMBER: GenBank/ABR09970.1
  ref <309>: DATABASE ENTRY DATE: 2007-12-21
  ref <313>: RELEVANT RESIDUES IN SEQ ID NO: (6)..(244)
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILN

SEQ ID NO: 71 - protein, 149 aa, Aspergillus restrictus
  ref <308>: DATABASE ACCESSION NUMBER: GenBank/AAA32707.1
  ref <309>: DATABASE ENTRY DATE: 1993-04-27
  ref <313>: RELEVANT RESIDUES IN SEQ ID NO: (28)..(176)
ATWTCINQQLNPKTNKWEDKRLLYSQAKAESNSHHAPLSDGKTGSSYPHWFTNGYDGNGKLIKGRTPIKFGKADCDRPPKHSQNGMGKDDHYLLEFPTFPDGHDYKFDSKKPKEDPGPARVIYTYPNKVFCGIVAHQRGNQGDLRLCSH

SEQ ID NO: 72 - protein, 130 aa, Hirsutella thompsonii var thompsonii
  ref <308>: DATABASE ACCESSION NUMBER: GenBank/AAB47280.1
  ref <309>: DATABASE ENTRY DATE: 2003-01-15
  ref <313>: RELEVANT RESIDUES IN SEQ ID NO: (35)..(164)
APIVTCRPKLDGREKPFKVDVATAQAQARKAGLTTGKSGDPHRYFAGDHIRWGVNNCDKADAILWEYPIYWVGKNAEWAKDVKTSQQKGGPTPIRVVYANSRGAVQYCGVMTHSKVDKNNQGKEFFEKCD

SEQ ID NO: 73 - protein, 109 aa, Salmonella typhi
  ref <308>: DATABASE ACCESSION NUMBER: GenBank/ACJ63596.1
  ref <309>: DATABASE ENTRY DATE: 2010-04-22
  ref <313>: RELEVANT RESIDUES IN SEQ ID NO: (28)..(136)
ERGEIWLVSLDPTAGHEQQGTRPVLIVTPAAFNRVTRLPVVVPVTSGGNFARTAGFAVSLDGVGIRTTGVVRCDQPRTIDMKARGGKRLERVPETIMNEVLGRLSTILT

SEQ ID NO: 74 - protein, 100 aa, Escherichia coli
  ref <308>: DATABASE ACCESSION NUMBER: GenBank/AAL77580.1
  ref <309>: DATABASE ENTRY DATE: 2005-06-29
  ref <313>: RELEVANT RESIDUES IN SEQ ID NO: (27)..(126)
QFKVYTYKRESRYRLFVDVQSDIIDTPGRRMVIPLASARLLSDKVSRELYPVVHIGDESWRMMTTDMASVPVSVIGEEVADLSHRENDIKNAINLMFWGI

SEQ ID NO: 75 - protein, 47 aa, Escherichia coli
  ref <301>: AUTHORS: Trovatti E, Cotrim CA, Garrido SS, Barros RS, Marchetto R.
  ref <302>: TITLE: Peptides based on CcdB protein as novel inhibitors of bacterial topoisomerases
  ref <303>: JOURNAL: Bioorg Med Chem Lett.
  ref <304>: VOLUME: 18
  ref <305>: ISSUE: 23
  ref <306>: PAGES: 6161-6164
  ref <307>: DATE: 2008-10-07
QFKVYTYKGGSGGRLLSDKVSRELGGSGGSHRENDIKNAINLMFWGI

SEQ ID NO: 76 - protein, 94 aa, Escherichia coli
  ref <308>: DATABASE ACCESSION NUMBER: NCBI Reference Sequence/NP_416081.1
  ref <309>: DATABASE ENTRY DATE: 2011-02-13
  ref <313>: RELEVANT RESIDUES IN SEQ ID NO: (2)..(95)
AYFLDFDERALKEWRKLGSTVREQLKKKLVEVLESPRIEANKLRGMPDCYKIKLRSSGYRLVYQVIDEKVVVFVISVGKRERSEVYSEAVKRIL

SEQ ID NO: 77 - protein, 90 aa, Paracoccus methylutens
  ref <308>: DATABASE ACCESSION NUMBER: NCBI Reference Sequence/ YP_001429553.1
  ref <309>: DATABASE ENTRY DATE: 2010-11-03
  ref <313>: RELEVANT RESIDUES IN SEQ ID NO: (2)..(91)
PELEWKAAAVADLLAIVDYISDDNPDAAFALMEEIQDKVAQLPAHPKRCRPGRVEGTRELVVRPNYLVVYAETPAVVTILRVLHAAQMWP

SEQ ID NO: 78 - protein, 251 aa, Gelonium multoflorum
  ref <301>: AUTHORS: Veenendaal LM, Jin H, Ran S, Cheung L, Navone N, Marks JW, Waltenberger J,
  ref <302>: TITLE: In vitro and in vivo studies of a VEGF121/rGelonin
  ref <303>: JOURNAL: Proc Natl Acad Sci U S A.
  ref <304>: VOLUME: 99
  ref <305>: ISSUE: 12
  ref <306>: PAGES: 7866-71
  ref <307>: DATE: 2001-06-11
GLDTVSFSTKGATYITYVNFLNELRVKLKPEGNSHGIPLLRKKADDPGKAFVLVALSNDNGQLAEIAIDVTSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPK

SEQ ID NO: 79 - protein, 387 aa, Corynobacterium diptheriae
  ref <308>: DATABASE ACCESSION NUMBER: GenBank/AAA72359.1
  ref <309>: DATABASE ENTRY DATE: 1993-02-23
  ref <313>: RELEVANT RESIDUES IN SEQ ID NO: (2)..(388)
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTH

SEQ ID NO: 80 - protein, 300 aa, Corynobacterium diptheriae
  ref <308>: DATABASE ACCESSION NUMBER: GenBank/AAA72359.1
  ref <309>: DATABASE ENTRY DATE: 1993-07-23
  ref <313>: RELEVANT RESIDUES IN SEQ ID NO: (2)..(194)
ADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNG

SEQ ID NO: 81 - protein, 297 aa, Corynobacterium diptheriae
  ref <308>: DATABASE ACCESSION NUMBER: GenBank/AAA72359.1
  ref <309>: DATABASE ENTRY DATE: 1993-07-23
  ref <313>: RELEVANT RESIDUES IN SEQ ID NO: (2)..(190)
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNG

SEQ ID NO: 82 - protein, 251 aa, Abrus precatorius
  ref <308>: DATABASE ACCESSION NUMBER: GenBank/CAA38655.1
  ref <309>: DATABASE ENTRY DATE: 2005-04-18
  ref <313>: RELEVANT RESIDUES IN SEQ ID NO: (2)..(252)
EDRPIKFSTEGATSQSYKQFIEALRERLRGGLIHDIPVLPDPTTLQERNRYITVELSNSDTESIEVGIDVTNAYVVAYRAGTQSYFLRDAPSSASDYLFTGTDQHSLPFYGTYGDLERWAHQSRQQIPLGLQALTHGISFFRSGGNDNEEKARTLIVIIQMVAEAARFRYISNRVRVSIQTGTAFQPDAAMISLENNWDNLSRGVQESVQDTFPNQVTLTNIRNEPVIVDSLSHPTVAVLALMLFVCNPPN

SEQ ID NO: 83 - protein, 342 aa, Pseudomonas aeruginosa
  ref <302>: TITLE: MUTATED PSEUDOMONAS EXOTOXINS WITH REDUCED ANTIGENICITY
  ref <310>: PATENT DOCUMENT NUMBER: WO/2007/016150
  ref <311>: PATENT FILING DATE: 2006-07-25
  ref <312>: PUBLICATION DATE: 2007-02-08
PEGGSLAALTAHQACHLPLETFTRHRQPRGWEQLEQCGYPVQRLVALYLAARLSWNQVDQVIANALASPGSGGDLGEAIRESPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWAGFYIAGDPALAYGYAQDQEPDAAGRIRNGALLRVYVPRSSLPGFYATSLTLAAPEAAGEVERLIGHPLPLRLDAITGPEESGGRLETILGWPLAERTVVIPSAIPTDPRNVGGDLDPSSIPDSEQAISALPDYASQPGKPP

SEQ ID NO: 84 - protein, 342 aa, Pseudomonas aeruginosa; identical to SEQ ID NO: 68
  ref <302>: TITLE: WO/2007/016150
  ref <310>: PATENT DOCUMENT NUMBER: WO/2007/016150
  ref <311>: PATENT FILING DATE: 2006-07-25
  ref <312>: PUBLICATION DATE: 2007-02-08

SEQ ID NO: 85 - DNA, 1290 bp, Artificial Sequence
  synthesized, encoding the fusion protein comprising: a fragment of TRAIL protein, boguanin domain A, sequences of steric linkers, fragment recognized by furin and pegylation linker sequence.
tataacaccgtgtcatttaatctgggcgaagcctatgaatatccgacctttattcaggatctgcgtaatgaactggcaaaaggtacaccggtttgtcagctgccggttaccctgcagaccattgcagatgataaacgttttgttctggtggatatcaccaccaccagtaaaaaaaccgttaaagttgccatcgatgtgaccgatgtttatgttgttggctatcaggataaatgggatggtaaagatcgtgccgtttttctggataaagttccgaccgttgcaaccagcaaactgtttccgggtgttaccaatcgtgttaccctgacctttgatggtagctatcagaaactggttaatgcagccaaagtggatcgtaaagatctggaactgggcgtttataaactggaattcagcattgaagccatccatggtaaaaccattaacggccaagaaatcgccaaatttttcctgattgtgattcagatggttagcgaagccgcacgctttaaatacattgaaaccgaagttgtggatcgcggtctgtatggtagctttaaaccgaactttaaagtgctgaacctggaaaataactggggtgatattagcgacgcaattcataaaagcagtccgcagtgtaccaccattaatccggcactgcagctgattagcccgagcaatgatccgtgggttgttaataaagttagccagattagtccggacatgggcattctgaaattcaaaagcggtggtggtggtagccgtaaaaaacgtgcaagcggttgtggtccggaaggtggtggcggtagtcgtgttgcagcacatattaccggcacccgtggtcgtagcaataccctgagcagcccgaatagcaaaaatgaaaaagccctgggtcgcaaaattaacagctgggaaagcagccgtagcggtcatagctttctgagcaatctgcatctgcgcaatggtgaactggtgattcacgaaaaaggcttctattatatctacagccagacctatttccgcttccaagaagagatcaaagagaacaccaaaaacgacaaacaaatggtgcagtacatctataaatacaccagctatccggatccgattctgctgatgaaaagcgcacgtaatagctgttggagcaaagatgcagaatatggcctgtatagcatctatcagggtggcatttttgaactgaaagaaaacgatcgcatctttgtgagcgtgaccaatgaacatctgattgatatggatcacgaagccagcttttttggtgcctttctggttggt

SEQ ID NO: 86 - DNA, 1359 bp, Artificial Sequence
  synthesized, encoding fusion protein comprising: a fragment of TRAIL protein, domain of ricin A, sequences of steric linkers, fragment recognized by furin, pegylation linker sequence and transporting sequence.
cgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgataaacaaatggtgcagtacatctataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggtggtggtggcggtagcgcaagcggttgtggtccggaacgtaaaaaacgcggtggtggtggtagtgaagataataatatttttccgaaacagtacccgattattaattttaccaccgcaggcgcaaccgttcagagctataccaattttattcgtgcagttcgtggtcgtctgacaaccggtgcggatgttcgtcatgaaattccggttctgccgaatcgtgttggtctgccgattaatcagcgttttattctggttgaactgagcaatcatgcagaactgagcgttaccctggcactggatgttaccaatgcctatgttgttggttatcgtgcaggtaatagcgcctatttttttcatccggataatcaggaagatgcagaagcaattacccacctgtttaccgatgtgcagaatcgttatacctttgcatttggtggcaattatgatcgtctggaacagctggcaggtagcctgcgtgaaaatattgaactgggtaatggtccgctggaagaagcaattagcgcactgtattattatagcaccggtggcacccagctgccgaccctggcacgtagctttattgtttgtattcagatgattagcgaagcagcccgttttcagtatattgaaggtgaaatgcgtacccgcattcgttataatcgtcgtagcgcaccggatccgagcgttattaccctggaaaatagctggggtcgtctgagtaccgcaattcaggaatcaaatcagggagcatttgcaagcccgattcagctgcagcgtcgtaatggtagcaaatttagcgtttatgatgtgagcattctgattccgattattgccctgatggtttatcgttgtgcaccgcctccgaaagaagatctg

SEQ ID NO: 87 - DNA, 1134 bp, Artificial Sequence
  synthesized, encoding the fusion protein comprising: a fragment of TRAIL protein, domain of ricin A, sequences of steric linkers, fragment recognized by furin, pegylation linker sequence and transporting sequence.
cgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgataaacaaatggtgcagtacatctataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggtggtggtggcggtagcgcaagcggttgtggtccggaacgtaaaaaacgcggtggtggtggtagtggtccgggtccgaaacagtacccgattattaattttaccaccgcaggcgcaaccgttcagagctataccaattttattcgtgcagttcgtggtcgtctgacaaccggtgcggatgttcgtcatgaaattccggttctgccgaatcgtgttggtctgccgattaatcagcgttttattctggttgaactgagcaatcatgcagaactgagcgttaccctggcactggatgttaccaatgcctatgttgttggttatcgtgcaggtaatagcgcctatttttttcatccggataatcaggaagatgcagaagcaattacccacctgtttaccgatgtgcagaatcgttatacctttgcatttggtggcaattatgatcgtctggaacagctggcaggtaatctgcgtgaaaatattgaactgggtaatggtccgctggaagaagcaattagcgcactgtattattatagcaccggtggcacccagctgccgaccctggcacgtagctttattatttgtattcagatgattagcgaagccgcacgctttcagtatattgaaggtgaaatgcgtgtgccgaaagaagatctg

SEQ ID NO: 88 - DNA, 1419 bp, Artificial Sequence
  synthesized, encoding the fusion protein comprising: a fragment of TRAIL protein, homolog of PAP toxin, sequences of steric linkers,  pegylation linker sequence and transporting sequence.
cgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaagaagaaattaaagaaaacaccaaaaatgataaacaaatggtgcagtacatttataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggtggtggtggcggtagcgcaagcggttgtggtccggaaggtggtggtggtagtgcaattaataccattacctttgatgcaggcaatgccaccattaataaatatgccacctttatggaaagcctgcgtaatcaggcaaaagatccgaaactgaaatgctatggtattccgatgctgccggataccaatagcaccccgaaatatctgctggttaaactgcagggtgcaaatctgaaaaccattaccctgatgctgcgtcgtaataatctgtatgttatgggttatagcgatccgtttaatggtaataaatgccgctatcatatttttaatgatattaccagcaccgaacgcaccgatgttgaaaataccctgtgtagcagcagcagcagtcgtgttgcaatgagcattaattataatagcctgtatccgaccatggaaaaaaaagcagaagtgaatagccgtaatcaggttcagctgggtattcagattctgagcagcgatattggtaaaatcagcggtgttgatagctttccggttaaaaccgaagcattttttctgctggttgccattcagatggttagcgaagcagcacgctttaaatatattgaaaatcaggtgaaaaccaattttaatcgtgccttttatccggatccgaaagtgattaatctggaagaaaaatggggcaaaattagcgaagccattcataatgcaaaaaatggtgcactgccgaaaccgctggaactggttgatgcaaaaggcaccaaatggattgttctgcgtgtggatgaaattaatcgtgatgttgccctgctgaaatatgttaatggcacctgtcagaccacctatcagaatgcaatgtttagccaggtgattattagcacctattataattatatgagcaatctgggcgacctgtttgaaggctttaaagatgaactg

SEQ ID NO: 89 - DNA, 1290 bp, Artificial Sequence
  synthesized, encoding the fusion protein comprising: a fragment of TRAIL protein, fragment of saporin, sequences of steric linkers and pegylation linker sequence.
cgtgttgcagcacatattaccggcacccgtggtcgtagcaataccctgagcagcccgaatagcaaaaatgaaaaagcactgggtcgcaaaatcaatagctgggaaagcagccgtagcggtcatagctttctgagcaatctgcatctgcgtaatggtgaactggtgattcatgaaaaaggcttttattatatttatagccagacctattttcgctttcaagaagagattaaagaaaataccaaaaatgataaacaaatggtgcagtacatttataaatataccagctatccggacccgattctgctgatgaaaagcgcacgtaatagctgttggagcaaagatgcagaatatggtctgtatagcatttatcagggtggcatctttgagctgaaagaaaatgatcgcatctttgttagcgtgaccaacgaacatctgatcgatatggatcatgaagccagcttttttggtgcatttctggttggtggtggtggcggtagcgcaagcggttgtggtccggaaggtggtggtggtagtgttaccagcattaccctggatctggttaatccgaccgcaggtcagtatagcagctttgttgataaaattcgcaataacgtgaaagatccgaatctgaaatatggtggcaccgatattgcagttattggtccgcctagcaaagataaatttctgcgcattaactttcagagcagccgtggcaccgttagcctgggtctgaaacgtgataatctgtatgttgttgcatatctggccatggataataccaatgttaaccgtgcctattattttaaaagcgaaatcaccagcgcagaactgaccgcactgtttccggaagcaaccaccgcaaatcagaaagcactggaatataccgaagattatcagagcatcgaaaaaaatgcccagattacccagggtgataaaagccgtaaagaactgggtctgggtattgatctgctgctgacctttatggaagccgttaataaaaaagcccgtgtggttaaaaacgaagcacgttttctgctgattgcaattcagatgaccgcagaagttgcacgttttcgttatattcagaacctggtgaccaaaaactttccgaacaaattcgacagcgataacaaagtgatccagtttgaagttagctggcgtaaaatttccaccgcaatttatggtgatgccaaaaatggcgtgtttaacaaagattatgacttcggttttggcaaagtgcgtcaggttaaagatctgcagatgggtctgctgatgtatctgggtaaaccg

SEQ ID NO: 90 - DNA, 1326 bp, Artificial Sequence
  synthesized, encoding the fusion protein comprising: a fragment of TRAIL protein, fragment of saporin, sequences of steric linkers, fragment recognized by furin and pegylation linker sequence.
gttcgtgaacgtggtccgcagcgtgttgcagcacatattaccggcacccgtggtcgtagcaataccctgagcagcccgaatagcaaaaatgaaaaagcactgggtcgcaaaatcaatagctgggaaagcagccgtagcggtcatagctttctgagcaatctgcatctgcgtaatggtgaactggtgattcatgaaaaaggcttttattatatctatagccagacctattttcgctttcaagaagagattaaagaaaataccaaaaatgataaacaaatggtgcagtatatctataaatataccagctatccggacccgattctgctgatgaaaagcgcacgtaatagctgttggagcaaagatgcagaatatggtctgtatagcatttatcagggtggcatctttgagctgaaagaaaatgatcgcatctttgttagcgtgaccaacgaacatctgatcgatatggatcatgaagccagcttttttggtgcatttctggttggtgcaagcggttgtggtccggaaggtggtggtggtggcagcggtggtggcggtagccgtaaaaaacgtgttaccagcattaccctggatctggttaatccgaccgcaggtcagtatagcagctttgttgataaaattcgcaataacgtgaaagatccgaatctgaaatatggtggcaccgatattgcagttattggtccgcctagcaaagataaatttctgcgcattaactttcagagcagccgtggcaccgttagcctgggtctgaaacgtgataatctgtatgttgttgcatatctggccatggataataccaatgttaaccgtgcctattattttaaaagcgaaatcaccagcgcagaactgaccgcactgtttccggaagcaaccaccgcaaatcagaaagcactggaatataccgaagattatcagagcatcgaaaaaaatgcccagattacccagggtgataaaagccgtaaagaactgggtctgggtattgatctgctgctgacctttatggaagccgttaataaaaaagcccgtgtggttaaaaacgaagcacgttttctgctgattgcaattcagatgaccgcagaagttgcacgttttcgttatattcagaacctggtgaccaaaaactttccgaacaaattcgacagcgataacaaagtgatccagtttgaagttagctggcgtaaaatttccaccgcaatttatggtgatgccaaaaatggcgtgtttaacaaagattatgacttcggttttggcaaagtgcgtcaggttaaagatctgcagatgggtctgctgatgtatctgggtaaaccg

SEQ ID NO: 91 - DNA, 1287 bp, Artificial Sequence
  synthesized, encoding the fusion protein comprising: a fragment of TRAIL protein, trichosantin peptide, sequences of steric linkers, fragment recognized by furin and pegylation linker sequence.
gatgttagctttcgtctgagcggtgcaaccagcagcagctatggtgtttttattagcaatctgcgtaaagcactgccgaatgaacgtaaactgtatgatattccgctgctgcgtagcagcctgcctggtagccagcgttatgcactgattcatctgaccaattatgccgatgaaaccattagcgttgccattgatgtgaccaatgtgtatattatgggttatcgtgcaggcgataccagctatttttttaatgaagcaagcgcaaccgaagcagccaaatatgtttttaaagatgccatgcgtaaagtgaccctgccgtatagcggtaattatgaacgtctgcagaccgcagcaggtaaaattcgtgaaaatattccgctgggtctgcctgcactggatagcgcaattaccaccctgttttattataatgcaaatagcgcagcaagcgcactgatggttctgattcagagcaccagcgaagcagcacgttataaatttattgaacagcagattggcaaacgcgtggataaaacctttctgccgagcctggcaattattagcctggaaaatagctggtcagcactgagcaaacaaattcagattgcaagcaccaataatggccagtttgaaagtccggttgttctgattaatgcacagaatcagcgtgtgaccattaccaatgttgatgccggtgttgttaccagcaatattgcactgctgctgaatcgtaataatatggcaggcggtggtggtagccgtaaaaaacgtgcaagcggttgtggtccggaaggtggtggtggtagtcgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaacgataaacaaatggtgcagtatatctataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggtgggt

SEQ ID NO: 92 - DNA, 1281 bp, Artificial Sequence
  synthesized, encoding the fusion protein comprising: a fragment of TRAIL protein, trichoanguin peptide, sequences of steric linkers, fragment recognized by furin and pegylation linker sequence.
gatgtgtcatttgatctgagcaccgcaaccaaaaaaagctatagcagctttattacccagctgcgtgatgcactgccgacccagggcaccgtttgtggtattccgctgctgccgagcaccgcaagcggtagccagtggtttcgtttttttaatctgaccaattataatgatgaaaccgttaccgtggccgttaatgttaccaatgtttatattgttgcctatcgtgcagatgccgtgagctatttttttgaagatacaccggcagaagcctttaaactgatttttgcaggcaccaaaaccgttaaactgccgtatagcggcaattatgataaactgcagagcgttgttggtaaacagcgtgatatgattgaactgggtattccggcactgagcagcgcaattaccaatatggtgtattatgattatcagagcaccgcagcagcactgctggttctgattcagtgtaccgcagaagcagcacgctataaatatattgaacagcaggttagcagccatattagcagcaatttttatccgaatcaggccgttattagcctggaaaataaatggggtgcactgagcaaacaaattcagattgcaaatcgtaccggtcatggccagtttgaaaatccggttgaactgtataatccggatggcacccgttttagcgttaccaataccagtgccggtgttgttaaaggcaatattaaactgctgctgtattataaagccagcggtggtggtggtagccgtaaaaaacgtgcaagcggttgtggtccggaaggtggtggtggcagtcgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaacgataaacaaatggtgcagtatatctataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggtgggt

SEQ ID NO: 93 - DNA, 1281 bp, Artificial Sequence
  synthesized, encoding fusion protein comprising: a fragment of TRAIL protein, a chain of mistletoe lectin A, sequences of steric linkers and pegylation linker sequence.
tatgaacgtctgcgtctgcgtgttacccatcagaccaccggtgaagaatattttcgttttattaccctgctgcgcgattatgttagcagcggtagctttagcaatgaaattccgctgctgcgtcagagcaccattccggttagtgatgcacagcgttttgttctggttgaactgaccaatgaaggtggtgatagcattaccgcagcaattgatgttaccaatctgtatgttgttgcatatcaggcaggcgatcagagctattttctgcgtgatgcaccgcgtggtgcagaaacccacctgtttaccggcaccacccgtagcagcctgccgtttaatggtagctatccggatctggaacgttatgcaggtcatcgtgatcagattccgctgggtattgatcagctgattcagagcgttaccgcactgcgttttccgggtggtagcacccgtacccaggcacgtagcattctgattctgattcagatgattagcgaagcagcacgttttaatccgattctgtggcgtgcacgtcagtatattaatagcggtgccagctttctgccggatgtttatatgctggaactggaaaccagctggggtcagcagagcacccaggttcagcagagtaccgatggtgtttttaataatccgattcgtctggcaattccgcctggtaattttgttaccctgaccaatgttcgtgatgttattgcaagcctggccattatgctgtttgtttgtggtgaaggtggtggtggtggcagcgcaagcggttgtggtccggaaggtggtggcggtagccgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaatcaaagaaaataccaaaaatgataaacaaatggtgcagtatatctataaatataccagttatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggtt

SEQ ID NO: 94 - DNA, 1386 bp, Artificial Sequence
  synthesized, encoding fusion protein comprising: a fragment of TRAIL protein, a subunit A of ebulin, sequences of steric linkers, sequence cleaved by furin and pegylation linker sequence.
attgattatccgagcgtgtcctttaatctggcaggcgcaaaaagcaccacctatcgtgattttctgaaaaatctgcgtgatcgtgttgcaaccggcacctatgaagttaatggtctgccggttctgcgtcgtgaaagcgaagttcaggttaaaaatcgttttgttctggtgcgcctgaccaattataatggtgataccgttaccagcgcagttgatgttaccaatctgtatctggttgcatttagcgcaaatggcaatagctatttttttaaagatgccaccgaactgcagaaaagcaacctgtttctgggcaccacccagcataccctgagctttaccggtaattatgataatctggaaaccgcagcaggcacccgtcgtgaaagtattgaactgggtccgaatccgctggatggtgcaattaccagcctgtggtatgatggtggtgttgcacgtagcctgctggttctgattcagatggttccggaagcagcacgttttcgttatattgaacaggaagttcgtcgtagcctgcagcagctgaccagctttaccccgaatgcactgatgctgagcatggaaaataattggagcagcatgagcctggaagttcagctgagcggtgataatgttagcccgtttagcggcaccgttcagctgcagaattatgatcatacaccgcgtctggtggataattttgaagaactgtataaaattaccggcattgccattctgctgtttcgttgtgttgcaaccaaaaccacccataatgcaattcgtatgccgcatgttctggttggtgaagataataaatttaatggtggtggtggtagcgcaagcggttgtggtccggaacgtaaaaaacgtggtggtggcggtgttcgtgaacgtggtccgcagcgtcgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgataaacaaatggtgcagtatatctataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcctttctggttggt

SEQ ID NO: 95 - DNA, 1362 bp, Artificial Sequence
  synthesized, encoding the fusion protein comprising: a fragment of TRAIL protein, a subunit A of nigrin, sequences of steric linkers, sequence cleaved by furin and pegylation linker sequence.
attgattatccgagcgtgtcttttaatctggatggtgcaaaaagcgcaacctatcgtgattttctgagcaatctgcgtaaaaccgttgcaaccggcacctatgaagttaatggtctgccggttctgcgtcgtgaaagcgaagttcaggttaaaagccgttttgttctggttccgctgaccaattataatggtaataccgttaccctggccgttgatgttaccaatctgtatgttgttgcctttagcggtaatgccaatagctatttttttaaagatgccaccgaagtgcagaaaagcaacctgtttgttggcaccaaacagaataccctgagctttaccggcaattatgataatctggaaaccgcagcaaatacccgtcgtgaaagtattgaactgggtccgagtccgctggatggtgccattaccagcctgtatcatggtgatagcgttgcacgtagcctgctggttgttattcagatggttagcgaagcagcacgttttcgttatattgaacaggaagttcgtcgtagcctgcagcaggcaaccagctttaccccgaatgcactgatgctgagcatggaaaataattggagcagcatgagcctggaaattcagcaggcaggtaataatgttagcccgttttttggcaccgttcagctgctgaattatgatcatacccatcgtctggtggataattttgaagaactgtataaaattaccggcattgccattctgctgtttcgttgtagcagcccgagcaatgataatgcaattcgtatgccgctggatctggcaggcgaagataataaatataatggtggtggtggcagccgcaaaaaacgtgcaagcggttgtggtccggaaggtggtggtggtagtcgtgttgcagcacatattaccggcacccgtggtcgtagcaataccctgagtagcccgaatagcaaaaatgaaaaagcactgggtcgcaaaattaatagctgggaaagcagccgtagcggtcatagctttctgtcaaatctgcatctgcgtaatggtgaactggtgattcatgaaaaaggcttttattatatttatagccagacctattttcgctttcaggaagaaattaaagaaaataccaaaaatgataaacaaatggtgcagtatatctataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggt

SEQ ID NO: 96 - DNA, 663 bp, Artificial Sequence
  synthesized, encoding fusion protein comprising: a fragment of TRAIL protein, a luffin P1 peptide, a sequence of steric linker and a sequence cleaved by furin.
cgtgttgcagcacatattaccggcacccgtggtcgtagcaataccctgagcagcccgaatagcaaaaatgaaaaagcactgggtcgcaaaatcaatagctgggaaagcagccgtagcggtcatagctttctgagcaatctgcatctgcgtaatggtgaactggtgattcatgaaaaaggcttttattatatttatagccagacctattttcgctttcaagaagagattaaagaaaataccaaaaatgataaacaaatggtgcagtacatttataaatacaccagctatccggacccgattctgctgatgaaaagcgcacgtaatagctgttggagcaaagatgcagaatatggtctgtatagcatttatcagggtggcatctttgagctgaaagaaaatgatcgcatctttgttagcgtgaccaacgaacatctgatcgatatggatcatgaagccagcttttttggtgcatttctggttggtggtggtggcggtagccgtaaaaaacgtccgcgtggtagtccgcgtaccgaatatgaagcatgtcgtgttcgttgtcaggttgcagaacatggtgttgaacgtcagcgtcgttgtcagcaggtttgtgaaaaacgtctgcgtgaacgtgaaggtcgtcgtgaagttgataaagatgaactg

SEQ ID NO: 97 - DNA, 681 bp, Artificial Sequence
  synthesized, encoding the fusion protein comprising: a fragment of TRAIL protein, a luffin P1 peptide, sequences of steric linkers, pegylation linker sequence and a sequence cleaved by furin.
cgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaagaagaaattaaagaaaacaccaaaaatgataaacaaatggtgcagtatatctataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggtggtggtggtagcgcaagcggttgtggtccggaacgtaaaaaacgtgcaagcggtggtccgcgtggtagtccgcgtaccgaatatgaagcatgtcgtgttcgttgtcaggttgcagaacatggtgttgaacgtcagcgtcgttgtcagcaggtttgtgaaaaacgtctgcgtgaacgtgaaggtcgtcgtgaagttgat

SEQ ID NO: 98 - DNA, 762 bp, Artificial Sequence
  synthesized, encoding the fusion protein comprising: a fragment of TRAIL protein, a luffin P1 peptide, a sequence of steric linker, pegylation linker sequence, a sequence cleaved by furin and transporting sequence.
accagcgaagaaaccattagcaccgttcaagaaaaacagcagaatattagtccgctggttcgtgaacgtggtccgcagcgtgttgcagcacatattaccggcacccgtggtcgtagcaataccctgagcagcccgaatagcaaaaatgaaaaagcactgggtcgcaaaatcaatagctgggaaagcagccgtagcggtcatagctttctgagcaatctgcatctgcgtaatggtgaactggtgattcatgaaaaaggcttctactatatctacagccagacctattttcgcttccaagaagaaatcaaagagaacaccaaaaacgacaaacaaatggtgcagtacatctacaaatataccagctatccggatccgattctgctgatgaaaagcgcacgtaatagctgttggagcaaagatgcagaatatggtctgtatagcatttatcagggtggcatctttgagctgaaagaaaatgatcgcatctttgttagcgtgaccaacgaacatctgatcgatatggatcatgaagccagcttttttggtgcatttctggttggtggtggtggcggtagcgcaagcggttgtggtccggaacgtaaaaaacgtccgcgtggtagtccgcgtaccgaatatgaagcatgtcgtgttcgttgtcaggttgcagaacatggtgttgaacgtcagcgtcgttgtcagcaggtttgtgaaaaacgtctgcgtgaacgtgaaggtcgtcgtgaagttgataaagatgaactg

SEQ ID NO: 99 - DNA, 1314 bp, Artificial Sequence
  synthesized, encoding the fusion protein comprising: a fragment of TRAIL protein, subunit A of volkensin, sequences of steric linkers, pegylation linker sequence and a sequence cleaved by furin.
gtttttccgaaagtgccgtttgatgttccgaaagcaaccgttgaaagctatacccgttttattcgtgttctgcgtgatgaactggcaggcggtgtttctccgcagggtattcgtcgtctgcgtaatccggcagaaattcagccgagccagggttttattctgattcagctgaccggttatgttggtagcgttaccctgattatggatgtgcgtaatgcatatctgctgggttatctgagccataatgtgctgtatcattttaatgatgttagcgcaagcagcattgcaagcgtttttccggatgcacagcgtcgtcagctgccgtttggtggtggttatccgagcatgcgtaattatgcaccggaacgcgatcagattgatcatggtattgtggaactggcctatgcagttgatcgtctgtattatagccagaataacaatcagattgccctgggtctggttatttgtgcaggtatggttgcagaagcaagccgttttcgttatattgaaggtctggtgcgtcagagcattgttggtccgggtgattatcgtacctttcgtcctgatgcactgatgtatagcattgttacccagtggcagaccctgagcgaacgtattcagggtagctttaatggtgcatttcagccggttcagctgggttatgcaagcgatccgttttattgggataatgttgcacaggcaattacccgtctgagcctgatgctgtttgttagccgtagcaccgatggtggtggtggtagccgtgttaaacgtgcgtctggttgcggcccggaaggcggcggtggcagcgttcgtgaacgtggtccgcagcgtgttgcagcacatattaccggcacccgtggtcgtagcaataccctgagcagcccgaatagcaaaaatgaaaaagccctgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgataaacaaatggtgcagtatatctataaatataccagctatccggatccgattctgctgatgaaaagcgcacgtaatagctgttggagcaaagatgcagaatatggcctgtatagcatttatcagggtggcatttttgaactgaaagaaaatgatcgcatttttgtgagcgtgaccaatgaacatctgattgatatggatcatgaagccagcttttttggtgcatttctggtgggc

SEQ ID NO: 100 - DNA, 1293 bp, Artificial Sequence
  synthesized, encoding the fusion protein comprising: a fragment of TRAIL protein, subunit A of volkensin, sequences of steric linkers, pegylation linker sequence, a sequence cleaved by furin and transporting sequence.
cgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaagaagaaattaaagaaaataccaaaaatgataagcagatggtgcagtatatctataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggtggtggtggcggtagcgcaagcggttgtggtccggaaggtggtggtggtagtgtttttccgaaagttccgtttgatgttccgaaagcaaccgttgaaagctatacccgttttattcgtgttctgcgtgatgaactggcaggcggtgttagtccgcagggtattcgtcgtctgcgtaatccggcagaaattcagccgagccagggttttattctgattcagctgaccggttatgttggtagcgttaccctgattatggatgttcgtaatgcatatctgctgggttatctgagccataatgtgctgtatcattttaatgatgttagcgcaagcagcattgcaagcgtttttccggatgcacagcgtcgtcagctgccgtttggtggtggttatccgagcatgcgtaattatgcaccggaacgtgatcagattgatcatggtattgttgaactggcctatgcagttgatcgtctgtattatagccagaataacaatcagattgccctgggtctggttatttgtgcaggtatggttgcagaagcaagccgttttcgttatattgaaggtctggttcgtcagagcattgttggtccgggtgattatcgtacctttcgtccggatgcactgatgtatagcattgttacccagtggcagaccctgagcgaacgtattcagggtagctttaatggtgcatttcagccggttcagctgggttatgcaagcgatccgttttattgggataatgttgcacaggcaattacccgtctgagcctgatgctgtttgttagccgtagcaccgataaagaagatctg

SEQ ID NO: 101 - DNA, 1284 bp, Artificial Sequence
  synthesized, encoding fusion protein comprising: a fragment of TRAIL protein, subunit A of momorcharin, sequences of steric linkers, pegylation linker sequence and a sequence cleaved by furin.
gatgttagctttcgtctgagcggtgcagatccgcgtagctatggtatgtttattaaagatctgcgtaacgcactgccgtttcgtgaaaaagtttacaatattccgctgctgctgccgagcgttagcggtgcaggtcgttatctgctgatgcacctgtttaattatgacggtaaaaccattaccgttgcactggatgttaccaacgtgtatattatgggttatctggcagataccacgagctatttttttaatgaaccggcagcagaactggcaagccagtatgtttttcgtgatgcacgtcgtaaaatcaccctgccgtatagcggtaattatgaacgtctgcagattgcagcaggtaaaccgcgtgaaaaaattccgattggtctgcctgcactggatagcgcaattagcaccctgctgcattatgatagcaccgcagcagccggtgcactgctggttctgattcagaccaccgcagaagcagcacgttttaaatatattgagcagcagattcaagagcgtgcatatcgtgatgaagttccgagcctggcaaccattagcctggaaaatagctggtcaggtctgagcaaacaaattcagctggcacagggtaataatggtatttttcgtaccccgattgtgctggttgataataaaggtaatcgcgtgcagattaccaatgttaccagcaaagttgtgaccagcaatatccagctgctgctgaatacccgtaatattggtggtggtggtagccgtaaaaaacgtgcaagcggttgtggtccggaaggtggtggtggcagtcgtgttgcagcacatattaccggcacccgtggtcgtagcaataccctgagcagcccgaatagcaaaaatgaaaaagcactgggtcgcaaaatcaatagctgggaaagcagccgtagcggtcatagctttctgagcaatctgcatctgcgtaatggtgaactggtgattcatgaaaaaggcttttattatatttatagccagacctattttcgctttcaagaagagattaaagaaaataccaaaaatgataaacaaatggtgcagtatatctataaatataccagctatccggacccgattctgctgatgaaaagcgcacgtaatagctgttggagcaaagatgcagaatatggtctgtatagcatttatcagggtggcatctttgagctgaaagaaaatgatcgcatctttgttagcgtgaccaacgaacatctgatcgatatggatcatgaagccagcttttttggtgcatttctggtgggt

SEQ ID NO: 102 - DNA, 1545 bp, Artificial Sequence
  synthesized, encoding the fusion protein comprising: a fragment of TRAIL protein, fragment of modified sequence of P. aeruginosa exotoxin,sequences of steric linkers and a transporting sequence.
cgtgttgcagcacatattaccggcacccgtggtcgtagcaataccctgagcagcccgaatagcaaaaatgaaaaagccctgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaacaccaaaaatgataaacaaatggtgcagtatatctataaatataccagctatccggatccgattctgctgatgaaaagcgcacgtaatagctgttggagcaaagatgcagaatatggcctgtatagcatttatcagggtggcatttttgaactgaaagaaaatgatcgcatttttgtgagcgtgaccaatgaacatctgattgatatggatcatgaagccagcttttttggtgcatttctggttggtggtggtggtagcgcaagcggtggtccggaaggtggtagcctggcagcactgaccgcacatcaggcatgtcatctgccgctggaaacctttacccgtcatcgtcagcctcgtggttgggaacagctggaacagtgtggttatccggttcagcgtctggttgcactgtatctggcagcacgtctgagctggaatcaggttgatcaggttattgcaaatgcactggcatctccgggtagcggtggtgatctgggtgaagcaattcgtgaatctccggaacaggcacgtctggcactgaccctggcagcagcagaaagcgaacgttttgttcgtcagggcaccggtaatgatgaagccggtgcagcaaatggtccggcagatagcggtgatgcactgctggaacgtaattatccgaccggtgcagaatttctgggtgatggcggtgatgttagctttagcacccgtggcacccagaattggaccgttgaacgtctgctgcaggcacatcgtcagctggaagaagccggttacgtttttgtgggttatcatggcacctttctggaagcagcacagagcattgtttttggtggtgttcgtgcacgtagccaggatctggatgcaatttgggcaggcttttatattgccggtgatccggcactggcatatggttatgcacaggatcaggaaccggacgcagccggtcgtattcgtaatggtgcactgctgcgtgtttatgttccgcgtagcagcctgcctggtttttatgcaaccagcctgaccctggctgcaccggaagcagcgggtgaagtggaacgtctgattggtcatccgctgccgctgcgtctggatgccattaccggtccggaagaatctggtggtcgtctggaaaccattctgggttggcctctggcagaacgcaccgttgttattccgagcgcaattccgaccgatccgcgtaatgttggtggcgatctggatccgagcagcattccggatagcgaacaggcaattagcgcactgccggattatgccagccagcctggtaaacctccgaaagatgaactg

SEQ ID NO: 103 - DNA, 1578 bp, Artificial Sequence
  synthesized, encoding fusion protein comprising: a fragment of TRAIL protein,modified sequence of P.aeruginosa exotoxin,sequences of steric linkers,pegylation linker, a sequence cleaved by furin and a transporting sequence.
cgtgtggcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgataaacaaatggtgcagtatatctataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggtggtggtggtagcgcaagcggttgtggtccggaacgtaaaaaacgtgcaagcggtggtccggaaggtggtagcctggcagcactgaccgcacatcaggcatgtcatctgccgctggaaacctttacccgtcatcgtcagcctcgtggttgggaacagctggaacagtgtggttatccggttcagcgtctggttgcactgtatctggcagcacgtctgagctggaatcaggttgatcaggttattgcaaatgcactggcaagtccgggtagcggtggtgatctgggtgaagcaattcgtgaaagtccggaacaggcacgtctggcactgaccctggcagcagcagaaagcgaacgttttgttcgtcagggcaccggtaatgatgaagccggtgcagcaaatggtccggcagatagcggtgatgcactgctggaacgtaattatccgaccggtgcagaatttctgggtgatggcggtgatgttagctttagcacccgtggcacccagaattggaccgttgaacgtctgctgcaggcacatcgtcagctggaagaagcaggttacgtttttgttggttatcatggcacctttctggaagcagcacagagcattgtttttggtggtgttcgtgcacgtagccaggatctggatgcaatttgggcaggtttttatattgccggtgatccggcactggcttatggttatgcacaggatcaggaaccggacgcagcaggtcgtattcgtaatggtgcactgctgcgtgtttatgttccgcgtagcagcctgcctggtttttatgcaaccagcctgaccctggctgcaccggaagcagccggtgaagtggaacgtctgattggtcatccgctgccgctgcgtctggatgccattaccggtccggaagaaagcggtggtcgtctggaaaccattctgggttggcctctggcagaacgtaccgttgttattccgagcgcaattccgaccgatccgcgtaatgttggtggcgatctggatccgagcagcattccggatagcgaacaggcaattagcgcactgccggattatgcaagccagcctggtaaacctccgaaagatgaactg

SEQ ID NO: 104 - DNA, 1578 bp, Artificial Sequence
  synthesized, encoding the fusion protein comprising: a fragment of TRAIL protein, fragment of modified sequence of P.aeruginosa exotoxin, sequences of steric linkers, pegylation linker sequence, a sequence cleaved by furin and a transporting
cgtgtggcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgataaacaaatggtgcagtatatctataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggtggtggtggtagcgcaagcggttgtggtccggaacgtaaaaaacgtgcaagcggtggtccggaaggtggtagtctggcagcactgaccgcacatcaggcatgtcatctgccgctggaaacctttacccgtcatcgtcagcctcgtggttgggaacagctggaacagtgtggttatccggttcagcgtctggttgcactgtatctggcagctcgtctgagctggaatcaggttgatcaggttattcgtaatgcactggcaagtccgggtagcggtggcgatctgggtgaagcaattcgtgaacagccggaacaggcacgtctggcactgaccctggcagcagcagaaagcgaacgttttgttcgtcagggcaccggtaatgatgaagccggtgcagcaaatggtccggcagatagcggtgatgcactgctggaacgtaattatccgaccggtgcagaatttctgggtgatggcggtgatgttagctttagcacccgtggcacccagaattggaccgttgaacgtctgctgcaggcacatcgtcagctggaagaagccggttacgtttttgtgggttatcatggcacctttctggaagcagcacagagcattgtttttggtggtgttcgtgcacgtagccaggatctggatgcaatttgggcaggcttttatattgccggtgatccggcactggcatacggttatgcacaggatcaggaaccggacgcagccggtcgtattcgtaatggtgcactgctgcgtgtttatgttccgcgtagcagcctgcctggtttttatgcaaccagcctgaccctggctgcaccggaagcagcgggtgaagtggaacgtctgattggtcatccgctgccgctgcgtctggatgccattaccggtccggaagaatctggtggtcgtctggaaaccattctgggttggcctctggcagaacgcaccgttgttattccgagcgcaattccgaccgatccgcgtaatgttggtggcgatctggatccgagcagcattccggatagcgaacaggcaattagcgcactgccggattatgccagccagcctggtaaacctccgaaagatgaactg

SEQ ID NO: 105 - DNA, 1602 bp, Artificial Sequence
  synthesized, encoding the fusion protein comprising: a fragment of TRAIL protein, fragment of modified sequence of Pseudomonas aeruginosa exotoxin, sequences of steric linkers, pegylation linker sequence and a sequence cleaved by furin.
cgtgttgcagcacatattaccggcacccgtggtcgtagcaataccctgagcagcccgaatagcaaaaatgaaaaagcactgggtcgcaaaattaacagctgggaaagcagccgtagcggtcatagctttctgagcaatctgcatctgcgtaatggtgaactggtgattcatgaaaaaggcttctactatatctacagccagacctattttcgcttccaagaagagattaaagaaaacaccaaaaacgataaacaaatggtgcagtacatctataaatacaccagctatccggatccgattctgctgatgaaaagcgcacgtaatagctgttggagcaaagatgcagaatatggcctgtatagcatttatcagggtggcatctttgaactgaaagaaaacgatcgtattttcgtgagcgtgaccaatgaacatctgatcgatatggatcatgaagccagcttttttggtgcatttctggtgggtggtggtggtagcgcaagcggttgtggtccggaacgtaaaaaacgtgcaagtggcggtccggaaggtggtagcctggcagcactgaccgcacatcaggcatgtcatctgccgctggaaacctttacccgtcatcgtcagcctcgtggttgggaacagctggaacagtgtggttatccggttcagcgtctggttgcactgtatctggcagcccgtctgagctggaatcaggttgatcaggttattcgtaatgcactggcaagtccgggtagcggtggtgatctgggtgaagcaattcgtgaacagcctgaacaggcacgtctggcactgaccctggcagccgcagaaagcgaacgttttgttcgtcagggcaccggtaatgttgttagcctgacctgtccggttgcagccggtgaatgtgcaggtccggcagatagcggtgatgcactgctggaacgtaattatccgaccggtgcagaatttctgggtgatggcggtgatgttagctttagtacccgtggcacccagaattggaccgttgaacgtctgctgcaggcacaccgtcagctggaagaacgtggttatgtttttgttggttatcatggcacctttctggaagcagcacagagcattgtgtttggtggtgttcgtgcacgtagccaggatctggatgcaatttggcgtggtttctatattgccggtgatccggcactggcctatggttatgcacaggatcaagaaccggatgcacgtggtcgcattcgcaatggtgccctgctgcgtgtttatgttccgcgtagcagcctgcctggtttttatcgtaccagcctgacactggctgcaccggaagcagcgggtgaagtggaacgtctgattggtcatccgctgccgctgcgtctggatgcgattaccggtcctgaagaagaaggcggtcgtctggaaaccattctgggttggcctctggcagaacgtaccgttgttattccgagcgcaattccgaccgatccgcgtaatgttggtggcgatctggatccgagcagcattccggataaagaacaggcaattagcgcactgccggattatgcaagccagcctggtaaaccgcctcgtgaagatctgaaa

SEQ ID NO: 106 - DNA, 1545 bp, Artificial Sequence
  synthesized, encoding the fusion protein comprising: a fragment of TRAIL protein, fragment of modified sequence of Pseudomonas aeruginosa exotoxin and a sequence of steric linker.
cgtgtggcggcgcatattaccggcacccgtggccgtagcaacaccctgagcagcccgaacagcaaaaacgaaaaagcgctgggccgtaaaattaacagctgggaaagcagccgtagcggccatagctttctgagcaacctgcatctgcgtaacggcgaactggtgattcatgaaaaaggcttttattatatttatagccagaccaactttaaatttcgtgaagaaattaaagaaaacaccaaaaacgataaacagatggtgcagtatatttataaatataccagctatccggatccgattctgctgatgaaaagcgcgcgtaacagctgctggagcaaagatgcggaatatggcctgtatagcatttatcagggcggcatttttgaactgaaagaaaacgatcgtatttttgtgagcgtgaccaacgaacgtctgcgtgatatgcatcatgaagcgagcttttttggcgcgtttctggtgggcggcggcggcagcgcgagcggcggcccggaaggcggcagcctggcggcgctgaccgcgcatcaggcgtgccatctgccgctggaaacctttacccgtcatcgtcagccgcgtggctgggaacagctggaacagtgcggctatccggtgcagcgtctggtggcgctgtatctggcggcgcgtctgagctggaaccaggtggatcaggtgattgcgaacgcgctggcgagcccgggcagcggcggcgatctgggcgaagcgattcgtgaaagcccggaacaggcgcgtctggcgctgaccctggcggcggcggaaagcgaacgttttgtgcgtcagggcaccggcaacgatgaagcgggcgcggcgaacggcccggcggatagcggcgatgcgctgctggaacgtaactatccgaccggcgcggaatttctgggcgatggcggcgatgtgagctttagcacccgtggcacccagaactggaccgtggaacgtctgctgcaggcgcatcgtcagctggaagaacgtggctatgtgtttgtgggctatcatggcacctttctggaagcggcgcagagcattgtgtttggcggcgtgcgtgcgcgtagccaggatctggatgcgatttgggcgggcttttatattgcgggcgatccggcgctggcgtatggctatgcgcaggatcaggaaccggatgcggcgggccgtattcgtaacggcgcgctgctgcgtgtgtatgtgccgcgtagcagcctgccgggcttttatgcgaccagcctgaccctggcggcgccggaagcggcgggcgaagtggaacgtctgattggccatccgctgccgctgcgtctggatgcgattaccggcccggaagaaagcggcggccgtctggaaaccattctgggctggccgctggcggaacgtaccgtggtgattccgagcgcgattccgaccgatccgcgtaacgtgggcggcgatctggatccgagcagcattccggatagcgaacaggcgattagcgcgctgccggattatgcgagccagccgggcaaaccgccgaaagatgaactg

SEQ ID NO: 107 - DNA, 1578 bp, Artificial Sequence
  synthesized, encoding the fusion protein comprising: a fragment of TRAIL protein, fragment of modified sequence of P.aeruginosa exotoxin, a pegylation linker sequence, a sequence cleaved by furin,steric linkers sequences and a transporting sequence.
agagtggcagcacatataacaggaacaagaggaagatcaaatacattatcatcaccaaattcaaagaatgaaaaggcattaggaagaaagataaattcatgggaatcatcaagatcaggacattcatttttatcaaatttacatttaagaaatggagaattagtgatacatgaaaagggattttattatatatattcacaaacatattttagatttcaagaagaaataaaggaaaatacaaagaatgataagcaaatggtgcaatatatatataagtatacatcatatccagatccaatattattaatgaagtcagcaagaaattcatgttggtcaaaggatgcagaatatggattatattcaatatatcaaggaggaatatttgaattaaaggaaaatgatagaatatttgtgtcagtgacaaatgaacatttaatagatatggatcatgaagcatcattttttggagcatttttagtgggaggaggaggatcagcatcaggatgtggaccagaaagaaagaagagagcatcaggaggaccagaaggaggatcattagcagcattaacagcacatcaagcatgtcatttaccattagaaacatttacaagacatagacaaccaagaggatgggaacaattagaacaatgtggatatccagtgcaaagattagtggcattatatttagcagcaagattatcatggaatcaagtggatcaagtgatagcaaatgcattagcatcaccaggatcaggaggagatttaggagaagcaataagagaatcaccagaacaagcaagattagcattaacattagcagcagcagaatcagaaagatttgtgagacaaggaacaggaaatgatgaagcaggagcagcaaatggaccagcagattcaggagatgcattattagaaagaaattatccaacaggagcagaatttttaggagatggaggagatgtgtcattttcaacaagaggaacacaaaattggacagtggaaagattattacaagcacatagacaattagaagaaagaggatatgtgtttgtgggatatcatggaacatttttagaagcagcacaatcaatagtgtttggaggagtgagagcaagatcacaagatttagatgcaatatgggcaggattttatatagcaggagatccagcattagcatatggatatgcacaagatcaagaaccagatgcagcaggaagaataagaaatggagcattattaagagtgtatgtgccaagatcatcattaccaggattttatgcaacatcattaacattagcagcaccagaagcagcaggagaagtggaaagattaataggacatccattaccattaagattagatgcaataacaggaccagaagaatcaggaggaagattagaaacaatattaggatggccattagcagaaagaacagtggtgataccatcagcaataccaacagatccaagaaatgtgggaggagatttagatccatcatcaataccagattcagaacaagcaatatcagcattaccagattatgcatcacaaccaggaaagccaccaaaggatgaatta

SEQ ID NO: 108 - DNA, 1578 bp, Artificial Sequence
  synthesized, encoding the fusion protein comprising: a fragment of TRAIL protein, fragment of modified sequence of P.aeruginosa exotoxin, a pegylation linker sequence, a sequence cleaved by furin,steric linkers sequences and a transporting sequence.
cgtgtggcggcgcatattaccggcacccgtggccgtagcaacaccctgagcagcccgaacagcaaaaacgaaaaagcgctgggccgtaaaattaacagctgggaaagcagccgtagcggccatagctttctgagcaacctgcatctgcgtaacggcgaactggtgattcatgaaaaaggcttttattatatttatagccagacctattttcgttttcaggaagaaattaaagaaaacaccaaaaacgataaacagatggtgcagtatatttataaatataccagctatccggatccgattctgctgatgaaaagcgcgcgtaacagctgctggagcaaagatgcggaatatggcctgtatagcatttatcagggcggcatttttgaactgaaagaaaacgatcgtatttttgtgagcgtgaccaacgaacatctgattgatatggatcatgaagcgagcttttttggcgcgtttctggtgggcggcggcggcagcgcgagcggctgcggcccggaacgtaaaaaacgtgcgagcggcggcccggaaggcggcagcctggcggcgctgaccgcgcatcaggcgtgccatctgccgctggaaacctttacccgtcatcgtcagccgcgtggctgggaacagctggaacagtgcggctatccggtgcagcgtctggtggcgctgtatctggcggcgcgtctgagctggaaccaggtggatcaggtgattgcgaacgcgctggcgagcccgggcagcggcggcgatctgggcgaagcgattcgtgaaagcccggaacaggcgcgtctggcgctgaccctggcggcggcggaaagcgaacgttttgtgcgtcagggcaccggcaacgatgaagcgggcgcggcgaacggcccggcggatagcggcgatgcgctgctggaacgtaactatccgaccggcgcggaatttctgggcgatggcggcgatgtgagctttagcacccgtggcacccagaactggaccgtggaacgtctgctgcaggcgcatcgtcagctggaagaacgtggctatgtgtttgtgggctatcatggcacctttctggaagcggcgcagagcattgtgtttggcggcgtgcgtgcgcgtagccaggatctggatgcgatttgggcgggcttttatattgcgggcgatccggcgctggcgtatggctatgcgcaggatcaggaaccggatgcggcgggccgtattcgtaacggcgcgctgctgcgtgtgtatgtgccgcgtagcagcctgccgggcttttatgcgaccagcctgaccctggcggcgccggaagcggcgggcgaagtggaacgtctgattggccatccgctgccgctgcgtctggatgcgattaccggcccggaagaaagcggcggccgtctggaaaccattctgggctggccgctggcggaacgtaccgtggtgattccgagcgcgattccgaccgatccgcgtaacgtgggcggcgatctggatccgagcagcattccggatagcgaacaggcgattagcgcgctgccggattatgcgagccagccgggcaaaccgccgaaagatgaactg

SEQ ID NO: 109 - DNA, 1269 bp, Artificial Sequence
  synthesized, encoding the fusion protein comprising:a fragment of TRAIL protein, variant of Shiga toxin stx, a sequence cleaved by furin and sequences of steric linkers.
aaagaatttaccctggattttagcaccgcaaaaacctatgttgatagcctgaatgttattcgtagcgcaattggtacaccgctgcagaccattagcagcggtggcaccagcctgctgatgattgatagcggcaccggtgataacctgtttgcagttgatgtgcgtggtattgatccggaagaaggtcgctttaataatctgcgcctgattgtggaacgtaataatctgtatgtgaccggttttgtgaatcgtaccaataatgtgttttatcgctttgccgattttagccatgttacctttccgggtacaaccgcagttaccctgagcggtgatagcagctataccaccctgcagcgtgttgcaggtattagccgtaccggtatgcagattaatcgtcatagcctgaccacctcttatctggatctgatgagccatagcggtacaagcctgacccagagcgttgcacgtgcaatgctgcgttttgttaccgttaccgcagaagcactgcgttttcgtcagattcagcgtggttttcgtaccaccctggatgatctgagcggtcgtagctatgttatgaccgcagaagatgttgatctgaccctgaattggggtcgtctgagcagcgttctgccggattatcatggtcaggatagcgttcgtgttggtcgtattagctttggtagcattaatgcaattctgggtagcgttgcactgattctgaatagccatcatgcaagcggtggtggtggtagccgtgttaaacgtgttcgtgaacgtggtccgcagcgtgtggcagcacatattaccggcacccgtggtcgtagcaataccctgagcagcccgaatagcaaaaatgaaaaagccctgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgataaacaaatggtgcagtatatttataaatacaccagctatccggatccgattctgctgatgaaaagcgcacgtaatagctgttggagcaaagatgcagaatatggcctgtatagcatttatcagggtggcatttttgaactgaaagaaaatgatcgcatttttgtgagcgtgaccaatgaacatctgattgatatggatcatgaagccagcttttttggtgcatttctggtgggc

SEQ ID NO: 110 - DNA, 1296 bp, Artificial Sequence
  synthesized, encoding the fusion protein comprising: a fragment of TRAIL protein, variant of Shiga toxin stx, a pegylation linker sequence, a sequence cleaved by furin, sequences of steric linkers and a transporting sequence.
cagcgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgataaacaaatggtgcagtacatctataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggtggtggtggtagcgcaagcggttgtggtccggaacgtaaaaaacgtggtggtggcggtagtaaagaatttaccctggattttagcaccgccaaaacctatgttgatagcctgaatgttattcgtagcgcaattggtacaccgctgcagaccattagcagcggtggcaccagcctgctgatgattgatagcggcaccggtgataacctgtttgcagttgatgttcgtggtattgatccggaagaaggtcgttttaataatctgcgtctgattgtggaacgcaataatctgtatgttaccggttttgtgaatcgcaccaataatgtgttttatcgctttgccgattttagccatgttacctttccgggtacaaccgcagttaccctgagcggtgatagcagctataccaccctgcagcgtgtggcaggtattagccgtaccggtatgcagattaatcgtcatagcctgaccaccagttatctggatctgatgagccatagcggtacaagcctgacccagagcgttgcacgcgctatgctgcgttttgttaccgttaccgcagaagcactgcgttttcgtcagattcagcgtggttttcgtaccaccctggatgatctgagcggtcgtagctatgttatgaccgcagaagatgttgatctgaccctgaattggggtcgtctgagcagcgttctgccggattatcatggtcaggatagcgttcgtgttggtcgtattagctttggtagcattaatgcaattctgggtagcgttgcactgattctgaatagccatcatgcaagcaaagaagatctg

SEQ ID NO: 111 - DNA, 1076 bp, Artificial Sequence
  synthesized, encoding the fusion protein comprising: synthesized, fusion protein comprising: a fragment of TRAIL protein, restrictocin peptide, a pegylation linker sequence, a sequence cleaved by furin and sequences of steric linkers.
gcaacctggacctgtattaatcagcagctgaatccgaaaaccaacaaatgggaagataaacgtctgctgtatagccaggcaaaagcagaaagcaatagccatcatgcaccgctgagtgatggtaaaaccggtagcagctatccgcattggtttaccaatggttatgatggtaacggcaaactgattaaaggtcgtaccccgattaaatttggtaaagcagattgtgatcgccctccgaaacattcacagaatggtatgggtaaagatgatcactatctgctggaatttccgacctttccggatggtcacgattataaatttgatagcaaaaaaccgaaagaggatccgggtccggcacgtgttatttatacctatccgaataaagtgttctgcggtattgttgcacatcagcgtggtaatcagggtgatctgcgtctgtgtagccatggtggtggtggtagcggtggtggtggcagccgtaaaaaacgtgcaagcggttgtggtccggaagttcgtgaacgtggtccgcagcgtgttgcagcacatattaccggcacccgtggtcgtagcaataccctgagcagcccgaatagcaaaaatgaaaaagcactgggtcgcaaaatcaatagctgggaaagcagccgtagcggtcatagctttctgagcaatctgcatctgcgtaatggtgaactggtgattcatgaaaaaggcttttattatatttatagccagacctattttcgctttcaagaagagattaaagaaaataccaaaaatgataaacaaatggtgcagtacatctataaatataccagctatccggacccgattctgctgatgaaaagcgcacgtaatagctgttggagcaaagatgcagaatatggtctgtatagcatttatcagggtggcatctttgagctgaaagaaaatgatcgcatctttgttagcgtgaccaacgaacatctgatcgatatggatcatgaagccagcttttttggtgcatttctggttggtaagctttagtaactcgagattatgagctctggagcacaagactggcctcatgggccttccgc

SEQ ID NO: 112 - DNA, 1005 bp, Artificial Sequence
  synthesized, encoding the fusion protein comprising: a fragment of TRAIL protein, restrictocin peptide, a pegylation linker sequence, a sequence cleaved by furin, sequences of steric linkers and a transporting sequence.
cgtgttgcagcacatattaccggcacccgtggtcgtagcaataccctgagcagcccgaatagcaaaaatgaaaaagcactgggtcgcaaaatcaatagctgggaaagcagccgtagcggtcatagctttctgagcaatctgcatctgcgtaatggtgaactggtgattcatgaaaaaggcttttattatatttatagccagacctattttcgctttcaagaagagattaaagaaaataccaaaaatgataaacaaatggtgcagtacatctataaatataccagctatccggacccgattctgctgatgaaaagcgcacgtaatagctgttggagcaaagatgcagaatatggtctgtatagcatttatcagggtggcatctttgagctgaaagaaaatgatcgcatctttgttagcgtgaccaacgaacatctgatcgatatggatcatgaagccagcttttttggtgcatttctggttggtggtggtggcggtagcgcaagcggttgtggtccggaacgtaaaaaacgcggtggtggtggtagtgcaacctggacctgtattaatcagcagctgaatccgaaaaccaacaaatgggaagataaacgtctgctgtatagccaggcaaaagcagaaagcaatagccatcatgcaccgctgagtgatggtaaaaccggtagcagctatccgcattggtttaccaatggttatgatggtaacggcaaactgattaaaggtcgtaccccgattaaatttggtaaagcagattgtgatcgccctccgaaacattcacagaatggtatgggtaaagatgatcactatctgctggaatttccgacctttccggatggtcacgattataaatttgatagcaaaaaaccgaaagaggatccgggtccggcacgtgttatttatacctatccgaataaagtgttctgcggtattgttgcacatcagcgtggtaatcagggtgatctgcgtctgtgtagccataaagaagatctg

SEQ ID NO: 113 - DNA, 957 bp, Artificial Sequence
  synthesized, encoding the fusion protein comprising: a fragment of TRAIL protein, hirsutellin peptide, a pegylation linker sequence, a sequence cleaved by furin and sequences of steric linkers.
gcaccgattgttacctgtcgtccgaaactggatggtcgtgaaaaaccgtttaaagttgatgttgcaaccgcacaggcacaggcacgtaaagcaggtctgaccaccggtaaaagcggtgatccgcatcgttattttgccggtgatcatattcgttggggtgttaataattgcgataaagcagatgccatcctgtgggaatatccgatttattgggttggtaaaaatgccgaatgggccaaagatgttaaaaccagccagcagaaaggtggtccgaccccgattcgtgttgtttatgcaaatagccgtggtgcagttcagtattgtggtgttatgacccatagcaaagtggataaaaacaaccagggcaaagaattttttgaaaaatgtgatggtggtggtggtagcggtggtggtggcagccgtaaaaaacgtgcaagcggttgtggtccggaagttcgtgaacgtggtccgcagcgtgttgcagcacatattaccggcacccgtggtcgtagcaataccctgagcagcccgaatagcaaaaatgaaaaagcactgggtcgcaaaatcaatagctgggaaagcagccgtagcggtcatagctttctgagcaatctgcatctgcgtaatggtgaactggtgattcatgaaaaaggcttttattatatttatagccagacctattttcgctttcaagaagagattaaagaaaataccaaaaatgataaacaaatggtgcagtacatctataaatataccagctatccggacccgattctgctgatgaaaagcgcacgtaatagctgttggagcaaagatgcagaatatggtctgtatagcatttatcagggtggcatctttgagctgaaagaaaatgatcgcatctttgttagcgtgaccaacgaacatctgatcgatatggatcatgaagccagcttttttggtgcatttctggtgggt

SEQ ID NO: 114 - DNA, 870 bp, Artificial Sequence
  synthesized, encoding fusion protein comprising: a fragment of TRAIL protein, Kid protein, a pegylation linker sequence, a sequence cleaved by furin, a sequence of steric linker and transporting sequence.
cgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaagaagaaatcaaagaaaataccaaaaatgataaacaaatggtgcagtatatttacaaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggtggtggtggcggtagcgcaagcggttgtggtccggaacgtaaaaaacgtgaacgtggtgaaatttggctggttagcctggatccgaccgcaggtcatgaacagcagggcacccgtccggttctgattgttacaccggcagcatttaatcgtgttacccgtctgccggttgttgttccggttaccagcggtggtaattttgcacgtaccgcaggttttgcagtgagcctggatggtgttggtattcgtaccaccggtgttgttcgttgtgatcagcctcgtaccattgatatgaaagcacgtggtggtaaacgtctggaacgtgttccggaaaccattatgaatgaagttctgggtcgtctgagcaccattctgaccaaagaagatctg

SEQ ID NO: 115 - DNA, 831 bp, Artificial Sequence
  synthesized, encoding the fusion protein comprising: a fragment of TRAIL protein, CcdB protein, a pegylation linker sequence, sequence cleaved by furin and a sequence of steric linker.
cgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaagaagaaatcaaagaaaataccaaaaatgataaacaaatggtgcagtatatttacaaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggtggtggtggcggtagcgcaagcggttgtggtccggaacgtaaaaaacgtcagtttaaagtgtatacctataaacgcgaaagccgttatcgtctgtttgttgatgttcagagcgatattattgatacaccgggtcgtcgtatggttattccgctggcaagcgcacgtctgctgagcgataaagttagccgtgaactgtatccggttgttcatattggtgatgaaagctggcgtatgatgaccaccgatatggcaagcgttccggttagcgttattggtgaagaagttgcagatctgagccatcgtgaaaatgatattaaaaatgccattaatctgatgttttggggcatt

SEQ ID NO: 116 - DNA, 684 bp, Artificial Sequence
  synthesized, encoding the fusion protein comprising: a fragment of TRAIL protein, CcdB protein, a pegylation linker sequence, a sequence cleaved by furin, a sequence of steric linker and transporting sequence.
cgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaagaagaaatcaaagaaaataccaaaaatgataaacaaatggtgcagtatatttacaaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggtggtggtggcggtagcgcaagcggttgtggtccggaacgtaaaaaacgtcagtttaaagtgtatacctataaaggtggtagcggtggtcgtctgctgagcgataaagttagccgtgaactgggtggtagtggaggtagccatcgtgaaaatgatattaaaaatgccattaatctgatgttttggggcattaaagaagatctg

SEQ ID NO: 117 - DNA, 825 bp, Artificial Sequence
  synthesized, encoding the fusion protein comprising: a fragment of TRAIL protein, RelE protein, a pegylation linker sequence, a sequence cleaved by furin, a sequence of steric linker and transporting sequence.
cgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaagaagaaatcaaagaaaataccaaaaatgataaacaaatggtgcagtatatctataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggtggtggtggcggtagcgcaagcggttgtggtccggaacgtaaaaaacgtgcatattttctggattttgatgaacgtgccctgaaagaatggcgtaaactgggtagcaccgttcgtgaacagctgaaaaaaaaactggttgaagttctggaaagtccgcgtattgaagcaaataaactgcgtggtatgccggattgctataaaattaaactgcgtagcagcggctatcgtctggtttatcaggttattgatgaaaaagtggtggtgtttgtgattagcgttggtaaacgtgaacgtagcgaagtttatagcgaagcagttaaacgcattctgaaagaagatctg

SEQ ID NO: 118 - DNA, 813 bp, Artificial Sequence
  synthesized, encoding fusion protein comprising: a fragment of TRAIL protein, StaB protein, a pegylation linker sequence, a sequence cleaved by furin, a sequence of steric linker and transporting sequence.
cgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaagaagaaatcaaagaaaataccaaaaatgataaacaaatggtgcagtatatttacaaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggtggtggtggcggtagcgcaagcggttgtggtccggaacgtaaaaaacgtccggaactggaatggaaagcagcagcagttgcagatctgctggcaattgttgattatattagtgatgataatccggatgcagcatttgcactgatggaagaaattcaggataaagttgcacagctgcctgcacatccgaaacgttgtcgtccgggtcgtgttgaaggcacccgtgaactggttgttcgtccgaattatctggttgtttatgcagaaacaccggcagttgttaccattctgcgtgttctgcatgcagcacagatgtggccgaaagaagatctg

SEQ ID NO: 119 - DNA, 1287 bp, Artificial Sequence
  synthesized, encoding the fusion protein comprising: a fragment of TRAIL protein, gelonin peptide and sequences of steric linkers.
ggcctggataccgttagctttagcaccaaaggtgcaacctatattacctatgtgaattttctgaatgaactgcgcgttaaactgaaaccggaaggtaatagccatggtattccgctgctgcgtaaaaaagcagatgatccgggtaaagcatttgttctggttgcactgagcaatgataatggtcagctggccgaaattgcaattgatgtgaccagcgtttatgtggttggttatcaggttcgtaatcgcagctatttttttaaagatgcaccggatgcagcctatgaaggcctgtttaaaaataccattaaaacccgtctgcattttggtggtagctatccgagcctggaaggtgaaaaagcatatcgtgaaaccaccgatctgggtattgaaccgctgcgtattggcattaaaaaactggatgaaaatgccattgataattataaaccgaccgaaattgcctctagcctgctggttgttattcagatggttagcgaagcagcacgttttacctttattgaaaatcagattcgcaataattttcagcagcgtattcgtccggcaaataataccattagcctggaaaataaatggggcaaactgagctttcagattcgtaccagcggtgcaaatggtatgtttagcgaagccgttgaactggaacgtgccaatggcaaaaaatactatgtgaccgcagtggatcaggttaaaccgaaaattgccctgctgaaatttgttgataaagatccgaaaggtggtggtggtagcggtggcggtggctctgttcgtgaacgtggtccgcagcgtgttgcagcacatattaccggcacccgtggtcgtagcaataccctgagcagcccgaatagcaaaaatgaaaaagccctgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaacaccaaaaatgataaacaaatggtgcagtatatctataaatataccagctatccggatccgattctgctgatgaaaagcgcacgtaatagctgttggagcaaagatgcagaatatggcctgtatagcatttatcagggtggcatttttgaactgaaagaaaatgatcgcatttttgtgagcgtgaccaatgaacatctgattgatatggatcatgaagccagcttttttggtgcatttctggtgggc

SEQ ID NO: 120 - DNA, 1302 bp, Artificial Sequence
  synthesized, encoding the fusion protein comprising: a fragment of TRAIL protein, gelonin peptide, sequences of steric linkers, a sequence cleaved by furin and pegylation linker sequence.
ggcctggataccgttagctttagcaccaaaggtgcaacctatattacctatgtgaattttctgaatgaactgcgcgttaaactgaaaccggaaggtaatagccatggtattccgctgctgcgtaaaaaagcagatgatccgggtaaagcatttgttctggttgcactgagcaatgataatggtcagctggccgaaattgcaattgatgtgaccagcgtttatgtggttggttatcaggttcgtaatcgcagctatttttttaaagatgcaccggatgcagcctatgaaggcctgtttaaaaataccattaaaacccgtctgcattttggtggtagctatccgagcctggaaggtgaaaaagcatatcgtgaaaccaccgatctgggtattgaaccgctgcgtattggcattaaaaaactggatgaaaatgccattgataattataaaccgaccgaaattgcctctagcctgctggttgttattcagatggttagcgaagcagcacgttttacctttattgaaaatcagattcgcaataattttcagcagcgtattcgtccggcaaataataccattagcctggaaaataaatggggcaaactgagctttcagattcgtaccagcggtgcaaatggtatgtttagcgaagccgttgaactggaacgtgccaatggcaaaaaatactatgtgaccgcagtggatcaggttaaaccgaaaattgccctgctgaaatttgttgataaagatccgaaaggtggtggtggcagccgtaaaaaacgtgcaagcggttgtggtccggaaggtggtggtggtagccagcgtgttgcagcacatattaccggcacccgtggtcgtagcaataccctgagcagcccgaatagcaaaaatgaaaaagccctgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaacaccaaaaatgataaacaaatggtgcagtatatctataaatataccagctatccggatccgattctgctgatgaaaagcgcacgtaatagctgttggagcaaagatgcagaatatggcctgtatagcatttatcagggtggcatttttgaactgaaagaaaatgatcgcatttttgtgagcgtgaccaatgaacatctgattgatatggatcatgaagccagcttttttggtgcatttctggtgggc

SEQ ID NO: 121 - DNA, 1281 bp, Artificial Sequence
  synthesized, encoding the fusion protein comprising a fragment of TRAIL protein, gelonin peptide, a sequence of steric linker, a pegylation linker sequence and transporting sequence.
cgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaagaagaaattaaagaaaacaccaaaaatgataaacaaatggtgcagtacatttataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggtgcaagcggttgtggtccggaaggtggtggtggtagcggtctggataccgttagctttagcaccaaaggtgcaacctatattacctatgtgaattttctgaatgaactgcgcgtgaaactgaaaccggaaggtaatagccatggtattccgctgctgcgtaaaaaagcagatgatccgggtaaagcatttgttctggttgcactgagcaatgataatggtcagctggcagaaattgccattgatgttaccagcgtttatgttgttggttatcaggttcgtaatcgcagctatttttttaaagatgcaccggatgcagcctatgaaggtctgtttaaaaataccattaaaacccgtctgcattttggtggtagctatccgagcctggaaggtgaaaaagcatatcgtgaaaccaccgatctgggtattgaaccgctgcgtattggtattaaaaaactggatgaaaatgccattgataattataaaccgaccgaaattgcaagcagcctgctggttgttattcagatggttagcgaagcagcacgctttacctttattgaaaatcagattcgcaataattttcagcagcgtattcgtccggcaaataataccattagcctggaaaataaatggggcaaactgagctttcagattcgtaccagcggtgcaaatggtatgtttagcgaagccgttgaactggaacgtgccaatggcaaaaaatactatgttaccgcagtggatcaggtgaaaccgaaaattgcactgctgaaatttgtggataaagatccgaaagatgaactg

SEQ ID NO: 122 - DNA, 1299 bp, Artificial Sequence
  synthesized, encoding the fusion protein comprising: a fragment of TRAIL protein, gelonin peptide, sequences of steric linkers, a pegylation linker sequence and a sequence cleaved by furin.
ggtctggataccgtgtcctttagcaccaaaggtgcaacctatattacctatgtgaattttctgaatgaactgcgcgtgaaactgaaaccggaaggtaatagccatggtattccgctgctgcgtaaaaaagcagatgatccgggtaaagcatttgttctggttgcactgagcaatgataatggtcagctggcagaaattgccattgatgttaccagcgtttatgttgttggttatcaggttcgtaatcgcagctatttttttaaagatgcaccggatgcagcctatgaaggtctgtttaaaaataccattaaaacccgtctgcattttggtggtagctatccgagcctggaaggtgaaaaagcatatcgtgaaaccaccgatctgggtattgaaccgctgcgtattggtattaaaaaactggatgaaaatgccattgataattataaaccgaccgaaattgcaagcagcctgctggttgttattcagatggttagcgaagcagcacgctttacctttattgaaaatcagattcgcaataattttcagcagcgtattcgtccggcaaataataccattagcctggaaaataaatggggcaaactgagctttcagattcgtaccagcggtgcaaatggtatgtttagcgaagccgttgaactggaacgtgccaatggcaaaaagtattatgttaccgcagtggatcaggtaaaaccgaaaattgcactgctgaaatttgtggataaagatccgaaaggtggtggtggtagccgtaaaaaacgtgcaagcggttgtggtccggaaggtggtggcggttcacgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgataaacaaatggtgcagtatatctataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggt

SEQ ID NO: 123 - DNA, 1674 bp, Artificial Sequence
  synthesized, encoding fusion protein comprising: a fragment of TRAIL protein, subunit A of diphteria toxin and sequences of steric linkers.
ggtgcagatgatgttgttgatagcagcaaatcctttgtgatggaaaattttagcagctatcatggcaccaaaccgggttatgttgatagcattcagaaaggcattcagaaaccgaaaagcggcacccagggtaattatgatgatgattggaaaggcttttatagcaccgataataaatatgatgccgcaggctatagcgttgataatgaaaatccgctgtctggtaaagccggtggtgttgttaaagttacctatccgggtctgaccaaagttctggcactgaaagttgataatgccgaaaccattaaaaaagaactgggtctgtctctgaccgaaccgctgatggaacaggttggcaccgaagaatttattaaacgctttggtgatggtgcaagccgtgttgttctgagcctgccgtttgcagaaggtagcagcagcgttgaatatattaataattgggaacaggccaaagcactgagcgttgaactggaaattaattttgaaacccgtggcaaacgtggtcaggatgccatgtatgaatatatggcacaggcatgtgcaggcaatcgtgttcgtcgtagcgttggtagcagcctgagctgtattaatctggattgggatgtgattcgcgataaaaccaaaaccaaaattgaaagcctgaaagaacatggtccgattaaaaataaaatgagcgaaagcccgaataaaaccgtgagcgaagaaaaagcaaaacagtatctggaagaatttcatcagaccgcactggaacatccggaactgagcgaactgaaaaccgttaccggcaccaatccggtttttgccggtgcaaattatgcagcatgggcagttaatgttgcccaggtgattgatagcgaaaccgcagataatctggaaaaaaccaccgcagcactgagcattctgcctggtattggtagcgttatgggtattgcagatggtgcagtgcatcataataccgaagaaattgtggcacagagcattgcactgagcagcctgatggttgcacaggcaattccgctggttggtgaactggttgatatcggctttgcagcctataattttgtggaaagcattatcaacctgtttcaggtggtgcataatagctataatcgtccggcatattctccgggtcataaaacccatggtggtggtggtagcggtggtggtggcagccgtgttgcagcacatattaccggcacccgtggtcgtagcaataccctgagcagcccgaatagcaaaaatgaaaaagccctgggtcgcaaaattaatagctgggaaagcagccgtagcggtcatagctttctgagcaatctgcatctgcgtaatggcgaactggtgattcatgaaaaaggtttttattatatttatagccagacctattttcgctttcaggaagaaattaaagaaaacaccaaaaatgataaacaaatggttcagtacatctataaatataccagctatccggatccgattctgctgatgaaaagcgcacgtaatagctgttggagcaaagatgcagaatatggcctgtatagcatttatcagggtggcatttttgaactgaaagaaaatgatcgcatttttgtgagcgtgaccaatgaacatctgattgatatggatcatgaagccagcttttttggtgcatttctggtgggt

SEQ ID NO: 124 - DNA, 1443 bp, Artificial Sequence
  synthesized,encoding fusion protein comprising: a fragment of TRAIL protein, catalytic domain of diphtheria toxin, sequences of steric linkers, a sequence cleaved by furin and a sequence of transporting domain.
cgtgtggcagcacatattaccggcacccgtggtcgtagcaataccctgagcagcccgaatagcaaaaatgaaaaagcactgggtcgcaaaatcaatagctgggaaagcagccgtagcggtcatagctttctgagcaatctgcatctgcgtaatggtgaactggtgattcatgaaaaaggcttctactatatctatagccagacctatttccgcttccaagaagaaatcaaagaaaataccaaaaacgataaacaaatggtgcagtatatctataaatacaccagctatccggatccgattctgctgatgaaaagcgcacgtaatagctgttggagcaaagatgcagaatatggtctgtatagcatttatcagggtggcatctttgagctgaaagaaaatgatcgcatctttgttagcgtgaccaacgaacatctgatcgatatggatcatgaagccagcttttttggtgcatttctggttggtggtggtggtagcggtggtggtggcagccgtaaaaaacgtccggaaggtggtagcctggcagcactgaccgcacatcaggcatgtcatctgccgctggaaacctttacccgtcatcgtcagcctcgtggttgggaacagctggaacagtgtggttatccggttcagcgtctggttgcactgtatctggcagcacgtctgagctggaatcaggttgatcaggttattcgtaatgcactggcaagtccgggtagtggtggtgatctgggtgaagcaattcgtgaacagccggaacaggcacgtctggcactgaccctggcagcagcagaaagcgaacgttttgttcgtcagggcaccggtaatggtggtggcggtgcagatgatgttgttgatagcagcaaaagttttgtgatggaaaacttcagcagctatcatggcaccaaaccgggttatgttgatagcattcagaaaggtattcagaaaccgaaaagcggcacccagggtaattatgatgatgattggaaaggcttctatagcaccgataacaaatatgatgcagccggttatagcgtggataatgaaaatccgctgagcggtaaagccggtggtgttgttaaagttacctatccgggtctgaccaaagttctggcactgaaagttgataatgccgaaaccatcaaaaaagaactgggtctgagcctgaccgaaccgctgatggaacaggttggcaccgaagaatttatcaaacgttttggtgatggtgcaagccgtgttgttctgagtctgccgtttgcagaaggtagcagcagcgttgaatatatcaataattgggaacaggcaaaagccctgagcgttgaactggaaatcaattttgaaacccgtggtaaacgtggtcaggatgcaatgtatgaatatatggcacaggcatgtgcaggcaatcgtgttcgtcgtaaagaagatctg

SEQ ID NO: 125 - DNA, 1443 bp, Artificial Sequence
  synthesized, encoding fusion protein comprising: a fragment of TRAIL protein, catalytic domain of diphtheria toxin, sequences of steric linkers, sequences cleaved by furin and a sequence of transporting domain.
ggcgcggatgatgtggtggatagcagcaaaagctttgtgatggaaaactttagcagctatcatggcaccaaaccgggctatgtggatagcattcagaaaggcattcagaaaccgaaaagcggcacccagggcaactatgatgatgattggaaaggcttttatagcaccgataacaaatatgatgcggcgggctatagcgtggataacgaaaacccgctgagcggcaaagcgggcggcgtggtgaaagtgacctatccgggcctgaccaaagtgctggcgctgaaagtggataacgcggaaaccattaaaaaagaactgggcctgagcctgaccgaaccgctgatggaacaggtgggcaccgaagaatttattaaacgctttggcgatggcgcgagccgcgtggtgctgagcctgccgtttgcggaaggcagcagcagcgtggaatatattaacaactgggaacaggcgaaagcgctgagcgtggaactggaaattaactttgaaacccgcggcaaacgcggccaggatgcgatgtatgaatatatggcgcaggcgtgcgcgggcaaccgcaaaaaacgcggcggcggcggcagcccggaaggcggcagcctggcggcgctgaccgcgcatcaggcgtgccatctgccgctggaaacctttacccgccatcgccagccgcgcggctgggaacagctggaacagtgcggctatccggtgcagcgcctggtggcgctgtatctggcggcgcgcctgagctggaaccaggtggatcaggtgattcgcaacgcgctggcgagcccgggcagcggcggcgatctgggcgaagcgattcgcgaacagccggaacaggcgcgcctggcgctgaccctggcggcggcggaaagcgaacgctttgtgcgccagggcaccggcaacggccgcaaaaaacgcggcggcggcggcagcggcggcggcggcagccgcgtggcggcgcatattaccggcacccgcggccgcagcaacaccctgagcagcccgaacagcaaaaacgaaaaagcgctgggccgcaaaattaacagctgggaaagcagccgcagcggccatagctttctgagcaacctgcatctgcgcaacggcgaactggtgattcatgaaaaaggcttttattatatttatagccagacctattttcgctttcaggaagaaattaaagaaaacaccaaaaacgataaacagatggtgcagtatatttataaatataccagctatccggatccgattctgctgatgaaaagcgcgcgcaacagctgctggagcaaagatgcggaatatggcctgtatagcatttatcagggcggcatttttgaactgaaagaaaacgatcgcatttttgtgagcgtgaccaacgaacatctgattgatatggatcatgaagcgagcttttttggcgcgtttctggtgggc

SEQ ID NO: 126 - DNA, 1104 bp, Artificial Sequence
  synthesized, encoding fusion protein comprising: a fragment of TRAIL protein, domain A of abrin, and sequences of steric linkers.
gcgcgccatatggaagatcgtccgattaaatttagcaccgaaggtgccacctcccagtcttataaacagtttattgaagcgctgcgtgaacgtctgcgtggcggtctgattcatgatattccggttctgccggatccgacaactctgcaggaacgcaatcgttatattaccgtggaactgagtaattccgatacagaatctattgaagtgggcattgtgacgcatggtatttctttttttcgtagtggcggtaatgataatgaagaaaaagcgcgtaccctgattgttattattcagatggttgctgaagccgctcgctttcgttatatttccaatcgtgtgcgtgtgtctattcagactggtactgcctttcagccggatgcagctatgatttccctggaaaataattgggataatctgtctcgtggtgttcaggaatccgttcaggatacctttccgaatcaggttaccctgaccaatattcgtaatgaaccggttattgttgatagcctgtctcatccgaccgttgctgttctggccctgatgctgtttgtttgcaatcctccgaatggtggtggtggttctggtggcggtggttcagtgcgtgaacgtggtccgcagcgtgttgccgctcatattaccggtactcgtggtcgttctaataccctgtctagcccgaatagtaaaaatgaaaaagccctgggtcgcaaaattaatagttgggaatctagtcgtagtggccattcttttctgagcaatctgcatctgcgtaatggtgaactggtgattcatgaaaaaggtttttattatatctattcccagacttattttcgttttcaggaagaaattaaagaaaacacgaaaaatgataaacagatggtgcagtatatctataaatatacctcttatccggatccgattctgctgatgaaaagtgcccgtaattcttgttggagtaaagatgcggaatatggcctgtattctatttatcagggtggtatttttgaactgaaagaaaatgatcgcatttttgtgtctgtgaccaatgaacatctgattgatatggatcatgaagcatctttttttggtgcctttctggtgggc

SEQ ID NO: 127 - DNA, 1359 bp, Artificial Sequence
  synthesized, encoding fusion protein comprising: a fragment of TRAIL protein, domain A of abrin, sequences of steric linkers, a sequence of integrin ligand and a sequence cleaved by urokinase.
gcacgccacatggaagatcgtccgattaaatttagcaccgaaggtgcaaccagccagagctataaacagtttattgaagcactgcgtgaacgtctgcgtggtggtctgattcatgatattccggttctgccggatccgaccaccctgcaggaacgtaatcgttatattaccgttgaactgagcaatagcgataccgaaagcattgaagttggtattgatgtgaccaatgcctatgttgttgcatatcgtgcaggcacccagagctattttctgcgtgatgcaccgagcagcgcaagcgattacctgtttaccggcaccgatcagcatagcctgccgttttatggcacctatggtgatctggaacgttgggcacatcagagccgtcagcagattccgctgggtctgcaggcactgacccatggtattagcttttttcgtagcggtggcaatgataatgaagaaaaagcacgtaccctgattgtgattattcagatggttgcagaagcagcacgctttcgttatatttcaaatcgtgttcgtgtgagcattcagaccggcaccgcatttcagccggatgcagcaatgattagcctggaaaataattgggataatctgagccgtggtgttcaggaaagcgttcaggatacctttccgaatcaggttaccctgaccaatattcgtaatgaaccggttattgttgatagcctgagccatccgaccgttgcagttctggcactgatgctgtttgtttgtaatcctccgaatgttcgtgaacgtggtccgggtggtggtggttcttgtttttgtgatggtcgctgtgattgtgcacgtaagaaacgcggtggtggtggttctgtgcgtgaacgtggtccgcagcgtgttgccgctcatattaccggtactcgtggtcgttctaataccctgtctagcccgaatagtaaaaatgaaaaagccctgggtcgcaaaattaatagttgggaatctagtcgtagtggccattcttttctgagcaatctgcatctgcgtaatggtgaactggtgattcatgaaaaaggtttttattatatctattcccagacttattttcgttttcaggaagaaattaaagaaaacacgaaaaatgataaacagatggtgcagtatatctataaatatacctcttatccggatccgattctgctgatgaaaagtgcccgtaattcttgttggagtaaagatgcggaatatggcctgtattctatttatcagggtggtatttttgaactgaaagaaaatgatcgcatttttgtgtctgtgaccaatgaacatctgattgatatggatcatgaagcatctttttttggtgcctttctggtgggc

SEQ ID NO: 128 - DNA, 1302 bp, Artificial Sequence
  synthesized,encoding fusion protein comprising: a fragment of TRAIL protein, domain A of abrin, sequences of steric linkers and a sequence cleaved by urokinase.
atggaagatcgtccgattaaatttagcaccgaaggtgccacctcccagtcttataaacagtttattgaagcgctgcgtgaacgtctgcgtggcggtctgattcatgatattccggttctgccggatccgacaactctgcaggaacgcaatcgttatattaccgtggaactgagtaattccgatacagaatctattgaagtgggcattgatgttaccaatgcttatgtggttgcttatcgcgctggcacccagagttattttctgcgtgatgctccgtcatctgccagtgattacctgtttaccggtacggatcagcattccctgccgttttatggtacttatggtgatctggaacgctgggctcatcagtctcgtcagcagattccgctgggtctgcaggctctgacgcatggtatttctttttttcgtagtggcggtaatgataatgaagaaaaagcgcgtaccctgattgttattattcagatggttgctgaagccgctcgctttcgttatatttccaatcgtgtgcgtgtgtctattcagactggtactgcctttcagccggatgcagctatgatttccctggaaaataattgggataatctgtctcgtggtgttcaggaatccgttcaggatacctttccgaatcaggttaccctgaccaatattcgtaatgaaccggttattgttgatagcctgtctcatccgaccgttgctgttctggccctgatgctgtttgtttgcaatcctccgaatggtggtggtggttctggtggcggtggttcacgtaagaaacgcgtgcgtgaacgtggtccgcagcgtgttgccgctcatattaccggtactcgtggtcgttctaataccctgtctagcccgaatagtaaaaatgaaaaagccctgggtcgcaaaattaatagttgggaatctagtcgtagtggccattcttttctgagcaatctgcatctgcgtaatggtgaactggtgattcatgaaaaaggtttttattatatctattcccagacttattttcgttttcaggaagaaattaaagaaaacacgaaaaatgataaacagatggtgcagtatatctataaatatacctcttatccggatccgattctgctgatgaaaagtgcccgtaattcttgttggagtaaagatgcggaatatggcctgtattctatttatcagggtggtatttttgaactgaaagaaaatgatcgcatttttgtgtctgtgaccaatgaacatctgattgatatggatcatgaagcatctttttttggtgcctttctggtgggc

SEQ ID NO: 129 - DNA, 1320 bp, Artificial Sequence
  synthesized,encoding fusion protein comprising: a fragment of TRAIL protein, domain A of abrin, sequences of steric linkers, a sequence cleaved by urokinase and arginine transporting sequence.
gaagatcgtccgattaaatttagcaccgaaggtgccacctcccagtcttataaacagtttattgaagcgctgcgtgaacgtctgcgtggcggtctgattcatgatattccggttctgccggatccgacaactctgcaggaacgcaatcgttatattaccgtggaactgagtaattccgatacagaatctattgaagtgggcattgatgttaccaatgcttatgtggttgcttatcgcgctggcacccagagttattttctgcgtgatgctccgtcatctgccagtgattacctgtttaccggtacggatcagcattccctgccgttttatggtacttatggtgatctggaacgctgggctcatcagtctcgtcagcagattccgctgggtctgcaggctctgacgcatggtatttctttttttcgtagtggcggtaatgataatgaagaaaaagcgcgtaccctgattgttattattcagatggttgctgaagccgctcgctttcgttatatttccaatcgtgtgcgtgtgtctattcagactggtactgcctttcagccggatgcagctatgatttccctggaaaataattgggataatctgtctcgtggtgttcaggaatccgttcaggatacctttccgaatcaggttaccctgaccaatattcgtaatgaaccggttattgttgatagcctgtctcatccgaccgttgctgttctggccctgatgctgtttgtttgcaatcctccgaatcgtcgtcgtcgtcgccgtcgtcgtaagaaacgcggtggtggtggttctggtggcggtggttcagtgcgtgaacgtggtccgcagcgtgttgccgctcatattaccggtactcgtggtcgttctaataccctgtctagcccgaatagtaaaaatgaaaaagccctgggtcgcaaaattaatagttgggaatctagtcgtagtggccattcttttctgagcaatctgcatctgcgtaatggtgaactggtgattcatgaaaaaggtttttattatatctattcccagacttattttcgttttcaggaagaaattaaagaaaacacgaaaaatgataaacagatggtgcagtatatctataaatatacctcttatccggatccgattctgctgatgaaaagtgcccgtaattcttgttggagtaaagatgcggaatatggcctgtattctatttatcagggtggtatttttgaactgaaagaaaatgatcgcatttttgtgtctgtgaccaatgaacatctgattgatatggatcatgaagcatctttttttggtgcctttctggtgggc

SEQ ID NO: 130 - DNA, 1671 bp, Artificial Sequence
  synthesized, encoding fusion protein comprising: a fragment of TRAIL protein, domain A of abrin, sequences of steric linkers, sequences cleaved by urokinase and transporting sequence.
gtgcgtgaacgtggtccgcagcgtgttgccgctcatattaccggtactcgtggtcgttctaataccctgtctagcccgaatagtaaaaatgaaaaagccctgggtcgcaaaattaatagttgggaatctagtcgtagtggccattcttttctgagcaatctgcatctgcgtaatggtgaactggtgattcatgaaaaaggtttttattatatctattcccagacttattttcgttttcaggaagaaattaaagaaaacacgaaaaatgataaacagatggtgcagtatatctataaatatacctcttatccggatccgattctgctgatgaaaagtgcccgtaattcttgttggagtaaagatgcggaatatggcctgtattctatttatcagggtggtatttttgaactgaaagaaaatgatcgcatttttgtgtctgtgaccaatgaacatctgattgatatggatcatgaagcatctttttttggtgcctttctggtgggcggtggtggtggttctggtggcggtggttcacgtaagaaacgcccggaaggcggcagcctggcggcgctgaccgcgcatcaggcgtgccatctgccgctggaaacctttacccgccatcgccagccgcgcggctgggaacagctggaacagtgcggctatccggtgcagcgcctggtggcgctgtatctggcggcgcgcctgagctggaaccaggtggatcaggtgattcgcaacgcgctggcgagcccgggcagcggcggcgatctgggcgaagcgattcgcgaacagccggaacaggcgcgcctggcgctgaccctggcggcggcggaaagcgaacgctttgtgcgccagggcaccggcaacggcggtggcggtggttcacgtaagaaacgcgaagatcgtccgattaaatttagcaccgaaggtgccacctcccagtcttataaacagtttattgaagcgctgcgtgaacgtctgcgtggcggtctgattcatgatattccggttctgccggatccgacaactctgcaggaacgcaatcgttatattaccgtggaactgagtaattccgatacagaatctattgaagtgggcattgatgttaccaatgcttatgtggttgcttatcgcgctggcacccagagttattttctgcgtgatgctccgtcatctgccagtgattacctgtttaccggtacggatcagcattccctgccgttttatggtacttatggtgatctggaacgctgggctcatcagtctcgtcagcagattccgctgggtctgcaggctctgacgcatggtatttctttttttcgtagtggcggtaatgataatgaagaaaaagcgcgtaccctgattgttattattcagatggttgctgaagccgctcgctttcgttatatttccaatcgtgtgcgtgtgtctattcagactggtactgcctttcagccggatgcagctatgatttccctggaaaataattgggataatctgtctcgtggtgttcaggaatccgttcaggatacctttccgaatcaggttaccctgaccaatattcgtaatgaaccggttattgttgatagcctgtctcatccgaccgttgctgttctggccctgatgctgtttgtttgcaatcctccgaatcgtcgtcgtcgtcgccgtcgt

SEQ ID NO: 131 - DNA, 1377 bp, Artificial Sequence
  synthesized, encoding fusion protein comprising: a fragment of TRAIL protein, domain A of abrin, sequences of steric linkers, a sequence cleaved by urokinase and pegylation linker sequence.
gaagatcgtccgattaaatttagcaccgaaggtgcaaccagccagagctataaacagtttattgaagcactgcgtgaacgtctgcgtggtggtctgattcatgatattccggttctgccggatccgaccaccctgcaagaacgtaatcgttatattaccgtggaactgagcaatagcgataccgaaagcattgaagttggtattgatgtgaccaatgcctatgttgttgcatatcgtgcaggcacccagagctattttctgcgtgatgcaccgagcagcgcaagcgattacctgtttaccggcaccgatcagcatagcctgccgttttatggcacctatggtgatctggaacgttgggcacatcagagccgtcagcagattccgctgggtctgcaggcactgacccatggtattagcttttttcgtagcggtggcaatgacaatgaagaaaaagcacgtaccctgattgtgattattcagatggttgcagaagcagcacgctttcgttatatttcaaatcgtgttcgtgtgagcattcagaccggaaccgcatttcagccggatgcagcaatgattagcctggaaaataattgggataatctgagccgtggtgttcaagaaagcgttcaggatacctttccgaatcaggttaccctgaccaatattcgtaatgaaccggttattgttgatagcctgagccatccgaccgttgcagttctggcactgatgctgtttgtttgtaatccgcctaatggtggtggtggtagcggtggtggtggcagccgtaaaaaacgtgcaagcggttgtggtccggaaaccagcgaagaaaccattagcaccgttcaagaaaaacagcagaatattagtccgctggttcgtgaacgtggtccgcagcgtgttgcagcacatattaccggaacccgtggtcgtagcaataccctgagcagcccgaatagcaaaaatgaaaaagcactgggtcgcaaaatcaatagctgggaaagcagccgtagcggtcatagctttctgagcaatctgcatctgcgtaatggtgaactggtgattcatgaaaaaggcttttattatatttatagccagacctattttcgctttcaagaagagattaaagaaaataccaaaaatgataaacaaatggtgcagtatatctataaatataccagctatccggatccgatcctgctgatgaaaagcgcacgtaatagctgttggagcaaagatgcagaatatggtctgtatagcatttatcagggtggcatctttgagctgaaagaaaatgatcgcatctttgttagcgtgaccaacgaacatctgatcgatatggatcatgaagccagcttttttggtgcatttctggtgggt

SEQ ID NO: 132 - DNA, 1329 bp, Artificial Sequence
  synthesized, encoding fusion protein comprising: a fragment of TRAIL protein, domain A of abrin, sequences of steric linkers, a sequence cleaved by urokinase and pegylation linker sequence.
cgtgtggcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgataaacaaatggtgcagtatatctataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggtggtggtggcggtagcgcaagcggttgtggtccggaacgtaaaaaacgcggtggtggtggtagtgcacgccacatggaagatcgtccgattaaatttagcaccgaaggtgcaaccagccagagctataaacagtttattgaagcactgcgtgaacgtctgcgtggtggtctgattcatgatattccggttctgccggatccgaccaccctgcaggaacgtaatcgttatattaccgttgaactgagcaatagcgataccgaaagcattgaagttggtattgatgtgaccaatgcctatgttgttgcatatcgtgcaggcacccagagctattttctgcgtgatgcaccgagcagcgcaagcgattacctgtttaccggcaccgatcagcatagcctgccgttttatggcacctatggtgatctggaacgttgggcacatcagagccgtcagcagattccgctgggtctgcaggcactgacccatggtattagcttttttcgtagcggtggcaatgataatgaagaaaaagcacgtaccctgattgtgattattcagatggttgcagaagcagcacgctttcgttatatttcaaatcgtgttcgtgtgagcattcagaccggcaccgcatttcagccggatgcagcaatgattagcctggaaaataattgggataatctgagccgtggtgttcaggaaagcgttcaggatacctttccgaatcaggttaccctgaccaatattcgtaatgaaccggttattgttgatagcctgagccatccgaccgttgcagttctggcactgatgctgtttgtttgtaatcctccgaatgttcgtgaacgtggtccg

SEQ ID NO: 133 - DNA, 1341 bp, Artificial Sequence
  synthesized, encoding fusion protein comprising: a fragment of TRAIL protein, domain A of abrin, sequences of steric linkers, a sequence cleaved by urokinase, a pegylationlinker sequence and a transporting sequence.
cgtgtggcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgataaacaaatggtgcagtatatctataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggtggtggtggcggtagcgcaagcggttgtggtccggaacgtaaaaaacgcggtggtggtggtagtgcacgccacatggaagatcgtccgattaaatttagcaccgaaggtgcaaccagccagagctataaacagtttattgaagcactgcgtgaacgtctgcgtggtggtctgattcatgatattccggttctgccggatccgaccaccctgcaggaacgtaatcgttatattaccgttgaactgagcaatagcgataccgaaagcattgaagttggtattgatgtgaccaatgcctatgttgttgcatatcgtgcaggcacccagagctattttctgcgtgatgcaccgagcagcgcaagcgattacctgtttaccggcaccgatcagcatagcctgccgttttatggcacctatggtgatctggaacgttgggcacatcagagccgtcagcagattccgctgggtctgcaggcactgacccatggtattagcttttttcgtagcggtggcaatgataatgaagaaaaagcacgtaccctgattgtgattattcagatggttgcagaagcagcacgctttcgttatatttcaaatcgtgttcgtgtgagcattcagaccggcaccgcatttcagccggatgcagcaatgattagcctggaaaataattgggataatctgagccgtggtgttcaggaaagcgttcaggatacctttccgaatcaggttaccctgaccaatattcgtaatgaaccggttattgttgatagcctgagccatccgaccgttgcagttctggcactgatgctgtttgtttgtaatcctccgaatgttcgtgaacgtggtccgaaagaagatctg

SEQ ID NO: 134 - DNA, 1323 bp, Artificial Sequence
  synthesized, encoding fusion protein comprising: a fragment of TRAIL protein, domain A of abrin, sequences of steric linkers, a sequence cleaved by urokinase and a pegylation linker sequence.
gaagatcgtccgattaaatttagcaccgaaggtgcaaccagccagagctataaacagtttattgaagcactgcgtgaacgtctgcgtggtggtctgattcatgatattccggttctgccggatccgaccaccctgcaggaacgtaatcgttatattaccgttgaactgagcaatagcgataccgaaagcattgaagttggtattgatgtgaccaatgcctatgttgttgcatatcgtgcaggcacccagagctattttctgcgtgatgcaccgagcagcgcaagcgattacctgtttaccggcaccgatcagcatagcctgccgttttatggcacctatggtgatctggaacgttgggcacatcagagccgtcagcagattccgctgggtctgcaggcactgacccatggtattagcttttttcgtagcggtggcaatgataatgaagaaaaagcacgtaccctgattgtgattattcagatggttgcagaagcagcacgctttcgttatatttcaaatcgtgttcgtgtgagcattcagaccggcaccgcatttcagccggatgcagcaatgattagcctggaaaataattgggataatctgagccgtggtgttcaggaaagcgttcaggatacctttccgaatcaggttaccctgaccaatattcgtaatgaaccggttattgttgatagcctgagccatccgaccgttgcagttctggcactgatgctgtttgtttgtaatccgccgaatggtggtggtggtagcggtggtggtggcagccgtaaaaaacgtgcaagcggttgtggtccggaatgtgttcgtgaacgtggtccgcagcgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgataaacaaatggtgcagtatatctataaatacaccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggt

SEQ ID NO: 135 - DNA, 1545 bp, Artificial Sequence
  synthesized, encoding fusion protein comprising: a fragment of mutated TRAIL protein, modified P. aeruginosa exotoxin sequence, sequences of steric linkers and transporting sequence.
cgtgttgcagcacatattaccggcacccgtggtcgtagcaataccctgagcagcccgaatagcaaaaatgaaaaagccctgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaacaccaaaaatgataaacaaatggtgcagtatatctataaatataccagctatccgcatccgattctgctgatgaaaagcgcacgtaatagctgttggagcaaagatgcagaatatggcctgtatagcatttatcagggtggcatttttgaactgaaagaaaatgatcgcatttttgtgagcgtgaccaatgaacatctgattgatatggatcatgaagccagcttttttggtgcatttctggttggtggtggtggtagcgcaagcggtggtccggaaggtggtagcctggcagcactgaccgcacatcaggcatgtcatctgccgctggaaacctttacccgtcatcgtcagcctcgtggttgggaacagctggaacagtgtggttatccggttcagcgtctggttgcactgtatctggcagcacgtctgagctggaatcaggttgatcaggttattgcaaatgcactggcatctccgggtagcggtggtgatctgggtgaagcaattcgtgaatctccggaacaggcacgtctggcactgaccctggcagcagcagaaagcgaacgttttgttcgtcagggcaccggtaatgatgaagccggtgcagcaaatggtccggcagatagcggtgatgcactgctggaacgtaattatccgaccggtgcagaatttctgggtgatggcggtgatgttagctttagcacccgtggcacccagaattggaccgttgaacgtctgctgcaggcacatcgtcagctggaagaagccggttacgtttttgtgggttatcatggcacctttctggaagcagcacagagcattgtttttggtggtgttcgtgcacgtagccaggatctggatgcaatttgggcaggcttttatattgccggtgatccggcactggcatatggttatgcacaggatcaggaaccggacgcagccggtcgtattcgtaatggtgcactgctgcgtgtttatgttccgcgtagcagcctgcctggtttttatgcaaccagcctgaccctggctgcaccggaagcagcgggtgaagtggaacgtctgattggtcatccgctgccgctgcgtctggatgccattaccggtccggaagaatctggtggtcgtctggaaaccattctgggttggcctctggcagaacgcaccgttgttattccgagcgcaattccgaccgatccgcgtaatgttggtggcgatctggatccgagcagcattccggatagcgaacaggcaattagcgcactgccggattatgccagccagcctggtaaacctccgaaagatgaactg

SEQ ID NO: 136 - DNA, 1545 bp, Artificial Sequence
  synthesized, encoding fusion protein comprising: a fragment of mutated TRAIL protein, modified P.aeruginosa exotoxin sequence, sequences of steric linkers and transporting sequence.
cgtgttgcagcacatattaccggcacccgtggtcgtagcaataccctgagcagcccgaatagcaaaaatgaaaaagccctgggtcgcaaaattaatagctgggaaagcagccgtagcggtcatagctttctgagcaatctgcatctgcgtaatggtgaactggtgattcatgaaaaaggcttttattatatttatagccagaccaattttaagtttagggaagaaattaaagaaaacaccaaaaatgataaacaaatggtgcagtatatctataaatataccagctatccggatccgattctgctgatgaaaagcgcacgtaatagctgttggagcaaagatgcagaatatggcctgtatagcatttatcagggtggcatttttgaactgaaagaaaatgatcgcatttttgtgagcgtgaccaatgaacgtctgagggatatgcatcatgaagccagcttttttggtgcatttctggttggtggtggtggtagcgcaagcggtggtccggaaggtggtagcctggcagcactgaccgcacatcaggcatgtcatctgccgctggaaacctttacccgtcatcgtcagcctcgtggttgggaacagctggaacagtgtggttatccggttcagcgtctggttgcactgtatctggcagcacgtctgagctggaatcaggttgatcaggttattgcaaatgcactggcatctccgggtagcggtggtgatctgggtgaagcaattcgtgaatctccggaacaggcacgtctggcactgaccctggcagcagcagaaagcgaacgttttgttcgtcagggcaccggtaatgatgaagccggtgcagcaaatggtccggcagatagcggtgatgcactgctggaacgtaattatccgaccggtgcagaatttctgggtgatggcggtgatgttagctttagcacccgtggcacccagaattggaccgttgaacgtctgctgcaggcacatcgtcagctggaagaagccggttacgtttttgtgggttatcatggcacctttctggaagcagcacagagcattgtttttggtggtgttcgtgcacgtagccaggatctggatgcaatttgggcaggcttttatattgccggtgatccggcactggcatatggttatgcacaggatcaggaaccggacgcagccggtcgtattcgtaatggtgcactgctgcgtgtttatgttccgcgtagcagcctgcctggtttttatgcaaccagcctgaccctggctgcaccggaagcagcgggtgaagtggaacgtctgattggtcatccgctgccgctgcgtctggatgccattaccggtccggaagaatctggtggtcgtctggaaaccattctgggttggcctctggcagaacgcaccgttgttattccgagcgcaattccgaccgatccgcgtaatgttggtggcgatctggatccgagcagcattccggatagcgaacaggcaattagcgcactgccggattatgccagccagcctggtaaacctccgaaagatgaactg

SEQ ID NO: 137 - DNA, 1557 bp, Artificial Sequence
  synthesized, encoding fusion protein comprising: a fragment of mutated TRAIL protein, modified P.aeruginosa exotoxin sequence, a sequence of steric linker and a sequence of pegylation linker and transporting sequence.
cgtgtggcggcgcatattaccggcacccgtggccgtagcaacaccctgagcagcccgaacagcaaaaacgaaaaagcgctgggccgtaaaattaacagctgggaaagcagccgtagcggccatagctttctgagcaacctgcatctgcgtaacggcgaactggtgattcatgaaaaaggcttttattatatttatagccagacctattttcgttttcaggaagaaattaaagaaaacaccaaaaacgataaacagatggtgcagtatatttataaatataccagctatccgcatccgattctgctgatgaaaagcgcgcgtaacagctgctggagcaaagatgcggaatatggcctgtatagcatttatcagggcggcatttttgaactgaaagaaaacgatcgtatttttgtgagcgtgaccaacgaacatctgattgatatggatcatgaagcgagcttttttggcgcgtttctggtgggcggcggcggcagcggcgcgagcggctgcggcccggaaccggaaggcggcagcctggcggcgctgaccgcgcatcaggcgtgccatctgccgctggaaacctttacccgtcatcgtcagccgcgtggctgggaacagctggaacagtgcggctatccggtgcagcgtctggtggcgctgtatctggcggcgcgtctgagctggaaccaggtggatcaggtgattgcgaacgcgctggcgagcccgggcagcggcggcgatctgggcgaagcgattcgtgaaagcccggaacaggcgcgtctggcgctgaccctggcggcggcggaaagcgaacgttttgtgcgtcagggcaccggcaacgatgaagcgggcgcggcgaacggcccggcggatagcggcgatgcgctgctggaacgtaactatccgaccggcgcggaatttctgggcgatggcggcgatgtgagctttagcacccgtggcacccagaactggaccgtggaacgtctgctgcaggcgcatcgtcagctggaagaacgtggctatgtgtttgtgggctatcatggcacctttctggaagcggcgcagagcattgtgtttggcggcgtgcgtgcgcgtagccaggatctggatgcgatttgggcgggcttttatattgcgggcgatccggcgctggcgtatggctatgcgcaggatcaggaaccggatgcggcgggccgtattcgtaacggcgcgctgctgcgtgtgtatgtgccgcgtagcagcctgccgggcttttatgcgaccagcctgaccctggcggcgccggaagcggcgggcgaagtggaacgtctgattggccatccgctgccgctgcgtctggatgcgattaccggcccggaagaaagcggcggccgtctggaaaccattctgggctggccgctggcggaacgtaccgtggtgattccgagcgcgattccgaccgatccgcgtaacgtgggcggcgatctggatccgagcagcattccggatagcgaacaggcgattagcgcgctgccggattatgcgagccagccgggcaaaccgccgaaagatgaactg

SEQ ID NO: 138 - DNA, 1545 bp, Artificial Sequence; identical to SEQ ID NO: 106
  synthesized, encoding fusion protein comprising: a fragment of mutated TRAIL protein, modified P.aeruginosa exotoxin sequence, sequences of steric linkers and transporting sequence.

SEQ ID NO: 139 - protein, 108 aa, Pseudomonas aeruginosa
  ref <308>: DATABASE ACCESSION NUMBER: PDB/1IKQ_A
  ref <309>: DATABASE ENTRY DATE: 2012-10-18
  ref <313>: RELEVANT RESIDUES IN SEQ ID NO: (251)..(357)
PEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNG

SEQ ID NO: 140 - protein, 10 aa, Artificial Sequence
  synthesized, a linker comprising a motive binding with integrines.
  ref <301>: AUTHORS: Wang H, Yan Z, Shi J, Han W, Zhang Y.
  ref <302>: TITLE: Expression, purification, and characterization of a neovasculature targeted rmhTNF-alpha in Escherichia coli
  ref <303>: JOURNAL: Protein Expr Purif.
  ref <304>: VOLUME: 45
  ref <305>: ISSUE: 1
  ref <306>: PAGES: 60-5
  ref <307>: DATE: 2005-07-21
CFCDGRCDCA

SEQ ID NO: 141 - protein, 281 aa, Homo sapiens [sequence omitted]

SEQ ID NO: 142 - protein, 161 aa, Artificial Sequence
  synthesized, mutated TRAIL protein sequence.
  ref <302>: TITLE: IMPROVED CYTOKINE DESIGN
  ref <310>: PATENT DOCUMENT NUMBER: WO2009066174
  ref <311>: PATENT FILING DATE: 2008-11-21
  ref <312>: PUBLICATION DATE: 2009-05-28
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPHPILLMKSARNSCWSKDAEYGLYSIYQGGIFELKENDRIFVSVTNEHLIDMDHEASFFGAFLVG

SEQ ID NO: 143 - protein, 161 aa, Artificial Sequence
  synthesized, mutated TRAIL protein sequence.
  ref <301>: AUTHORS: Gasparian ME,
  ref <302>: TITLE: DR4-selective tumor necrosis factor-related apoptosis- inducing ligand(TRAIL) variants obtained by structure-based design
  ref <303>: JOURNAL: J Biol Chem
  ref <304>: VOLUME: 283
  ref <305>: ISSUE: 29
  ref <306>: PAGES: 20560-8
  ref <307>: DATE: 2008-07-18
RVAAHITGTRGRSNTLSSPNSKNEKALGRKINSWESSRSGHSFLSNLHLRNGELVIHEKGFYYIYSQTNFKFREEIKENTKNDKQMVQYIYKYTSYPDPILLMKSARNSCWSKDAEYGLYSIYQGGIFELKENDRIFVSVTNERLRDMHHEASFFGAFLVG

SEQ ID NO: 144 - protein, 433 aa, Artificial Sequence
  synthesized, fusion protein comprising: a fragment of TRAIL protein, a variant of abrin A domain, sequences of steric linkers and cleavage site recognized by furin.
EDRPIKFSTEGATSQSYKQFIEALRERLRGGLIHDIPVLPDPTTLQERNRYITVELSNSDTESIEVGIDATNAYVVAYRAGTQSYFLRDAPSSASDYLFTGTDQHSLPFYGTYADLERWAHQSRQQIPLGLQALTHGISFFRSGGNDNEEKARTLIVIIQMVAEAARFRYISNRVRVSIQTGTAFQPDAAMISLENNWDNLSRGVQESVQDTFPNQVTLTNIRNEPVIVDQLSHPTVAVLALMLFVCNPPNGGGGSGGGGSRKKRVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 145 - protein, 450 aa, Artificial Sequence [sequence omitted]

SEQ ID NO: 146 - protein, 481 aa, Artificial Sequence
  synthesized, fusion protein comprising: a fragment of TRAIL protein, a active domain of diphtheria toxin, sequences of steric linkers, cleavage sites recognized by furin and transporting sequence.
GADDVADSSKSFVMENFSSYHGTKPGYADSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRKKRGGGGSPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNGRKKRGGGGSGGGGSRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 147 - protein, 478 aa, Artificial Sequence
  synthesized, fusion protein comprising: a fragment of TRAIL protein, a mutated active domain of diphtheria toxin, sequences of steric linkers, cleavage sites recognized by furin and transporting sequence.
GADDVSKSFVMENFSSYHGTKPGYADSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRKKRGGGGSPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNGRKKRGGGGSGGGGSRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 148 - protein, 433 aa, Artificial Sequence
  synthesized, fusion protein comprising: a fragment of TRAIL protein, a mutated variant of gelonin, sequences of steric linkers, cleavage site recognized by furin and pegylation linker.
GLDTVSFSTKGATYITYVNFLNELRVKLKPEGNSHGIPLLRKKADDPGKAFVLVALSNDNGQLAEIAIDATSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPKGGGGSRKKRASGCGPEGGGGSRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 149 - protein, 258 aa, Artificial Sequence
  synthesized, fusion protein comprising: a fragment of TRAIL protein, a P1 luffin peptide, sequences of steric linkers, cleavage site recognized by furin and transporting sequence.
TSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGCAAACAACRKKRPRGSPRTEYEACRVRCQVAEHGVERQRRCQQVCEKRLREREGRREVDKDEL

SEQ ID NO: 150 - protein, 253 aa, Artificial Sequence
  synthesized, fusion protein comprising: a fragment of TRAIL protein, a P1 luffin peptide, sequences of steric linkers, cleavage site recognized by furin and a transporting sequence.
PRGSPRTEYEACRVRCQVAEHGVERQRRCQQVCEKRLREREGRREVDKDELRKKRGGGCAAACAACTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 151 - protein, 539 aa, Artificial Sequence
  synthesized, fusion protein comprising: a fragment of TRAIL protein, a mutated variant of trichosantin, sequences of steric linkers, cleavage sites recognized by furin and transporting sequence.
DVSFRLSGATSSSYGVFISNLRKALPNERKLYDIPLLRSSLPGSQRYALIHLTNYADETISVAIDVTNVYIMGYRAGDTSACSNEASATEAAKYVFKDAMRKVTLPYSGNYERLQTAAGKIRENIPLGLPALDSAITTLFYYNANSAASALMVLIQSTSEAARYKFIEQQIGCGVDKTFLPSLAIISLENSWSALSKQIQIASTNNGQFESPVVLINAQNQRVTITNVDAGVVTSNIALLLNRNNMARKKRGGGGSPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNGRKKRGGGGSGGGGSRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 152 - protein, 429 aa, Artificial Sequence
  synthesized, fusion protein comprising: a fragment of TRAIL protein, a mutated variant of trichosantin, sequences of steric linkers, cleavage site recognized by furin and pegylation sequence.
DASFRLSGATSSSYGVFISNLRKALPNERKLYDIPLLRSSLPGSQRYALIHLTNYADETISVAIDATNVYIMGYRAGDTSACSNEASATEAAKYVFKDAMRKVTLPYSGNYERLQTAAGKIRENIPLGLPAGDSAITTLFYYNANSAASALMVLIQSTSEAARYKFIEQQIGCGVDKTFLPSLAIISLENSWSALSKQIQIASTNNGQFESPVVLINAQNQRVTITNVDAGVVTSNIALLLNRNNMAGGGGSRKKRASGCGPEGGGGSRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 153 - protein, 515 aa, Artificial Sequence
  synthesized, fusion protein comprising: a fragment of TRAIL protein, modified P. aeruginosa exotoxin sequence, sequences of steric linkers and transporting sequence.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGSASGGPEGGSLAALTAHQACHLPLETFTRHRQPRGWEQLEQCGYPVQKLVALYLAARLSWNQVDQVIANALASPGSGGDLGEAIRESPEQARLALTLAAAESERFVRQGTGNDEAGAANGPADSGDALLERNYPTGAEFLGDGGDVSFSTRGTQQWTVERLLQAHRQLEEAGYVFVGYHGTFLEAAQSIVFGGVRARSQDLDAIWAGFYIAGDPALAYGYAQDQEPDAAGRIRNGALLRVYVPRSSLPGFYATSLTLAAPEAAGEVERLIGHPLPLRLDAITGPEESGGRLETILGWPLAERTVVIPSAIPTDPKNVGGDLDPSSIPDSEQAISALPDYASQPGKPPKDEL

SEQ ID NO: 154 - protein, 402 aa, Artificial Sequence
  synthesized, fusion protein comprising: a fragment of TRAIL protein, deletion variant of Pseudomonas aeruginosa exotoxin sequence, sequences of steric linkers, cleavage site recognized by furin and a transporting sequence.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGSGGGGRKKRRHRQPRGWEQLPTGAEFLGDGGDVSFSTRGTQNWTVERLLQAHRQLEEAGYVFVGYHGTFLEAAQSIVFGGVRARSQDLDAIWAGFYIAGDPALAYGYAQDQEPDAAGRIRNGALLRVYVPRSSLPGFYATSLTLAAPEAAGEVERLIGHPLPLRLDAITGPEESGGRLETILGWPLAERTVVIPSAIPTDPRNVGGDLDPSSIPDSEQAISALPDYASQPGKPPKEDL

SEQ ID NO: 155 - protein, 403 aa, Artificial Sequence
  synthesized, fusion protein comprising: a fragment of TRAIL protein, deletion variant of Pseudomonas aeruginosa exotoxin sequence, sequences of steric linkers, cleavage site recognized by furin and transporting sequence.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGSGGGGRKKRRHRQPRGWEQLYPTGAEFLGDGGAVSFSTRGTQNWTVERLLQAHRQLEEGGYVFVGYHGTFLEAAQSIVFGGVRARSQDLDAIWAGFYIAGDPALAYGYAQDQEPDAAGRIRNGALLRVYVPRSSLPGFYATSLTLAAPEAAGEVERLIGHPLPLRLDAITGPEEAGGRLETILGWPLAERTVVIPSAIPTDPRNVGGDLDPSSIPDAEAAISALPDYASQPGKPPKEDL

SEQ ID NO: 156 - protein, 470 aa, Artificial Sequence
  synthesized, fusion protein comprising: a fragment of TRAIL protein, deletion variant of P.aeruginosa exotoxin sequence, a sequence of steric linker, a pegylation linker, cleavage sites recognized by furin and a transporting sequence.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGSASGCGPERKKRRHRQPRGWEQLEQSGYPVQRLVALYLAARLSWNQVDQVIRNALASPGSGGDLGEAIREQPEQARLALTLAAAESEYPTGAEFLGDGGAVSFSTRGTQNWTVERLLQAHRQLEEGGYVFVGYHGTFLEAAQSIVFGGVRARSQDLDAIWAGFYIAGDPALAYGYAQDQEPDAAGRIRNGALLRVYVPRSSLPGFYATSLTLAAPEAAGEVERLIGHPLPLRLDAITGPEEAGGRLETILGWPLAERTVVIPSAIPTDPRNVGGDLDPSSIPDAEAAISALPDYASQPGKPPKEDL

SEQ ID NO: 157 - protein, 478 aa, Artificial Sequence [sequence omitted]

SEQ ID NO: 158 - protein, 402 aa, Artificial Sequence
  synthesized, fusion protein comprising: a fragment of TRAIL protein, deletion variant of Pseudomonas aeruginosa exotoxin sequence, sequences of steric linkers, cleavage sites recognized by furin and a transporting sequence.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGSGGGGRKKRRHRQPRGWEQLPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPKEDL

SEQ ID NO: 159 - protein, 467 aa, Artificial Sequence
  synthesized, fusion protein comprising: a fragment of TRAIL protein, deletion variant of Pseudomonas aeruginosa exotoxin sequence, sequences of steric linkers, cleavage site recognized by furin and a transporting sequence.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGSGGGGSRKKRGGGGSGGGGSEQSGYPVQRLVALYLAARLSWNQVDQVIRNALASPGSGGDLGEAIREQPEQARLALTLAAAESEYPTGAEFLGDGGAVSFSTRGTQNWTVERLLQAHRQLEEGGYVFVGYHGTFLEAAQSIVFGGVRARSQDLDAIWAGFYIAGDPALAYGYAQDQEPDAAGRIRNGALLRVYVPRSSLPGFYATSLTLAAPEAAGEVERLIGHPLPLRLDAITGPEEAGGRLETILGWPLAERTVVIPSAIPTDPRNVGGDLDPSSIPDAEAAISALPDYASQPGKPPKEDL

SEQ ID NO: 160 - protein, 474 aa, Artificial Sequence
  synthesized, fusion protein comprising: a fragment of TRAIL protein, deletion variant of Pseudomonas aeruginosa exotoxin sequence, sequences of steric linkers, cleavage site recognized by furin and a transporting sequence.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGSGGGGSRHRQPRGWEQLGGGGSGGGGSEQSGYPVQRLVALYLAARLSWNQVDQVIRNALASPGSGGDLGEAIREQPEQARLALTLAAAESEYPTGAEFLGDGGAVSFSTRGTQNWTVERLLQAHRQLEEGGYVFVGYHGTFLEAAQSIVFGGVRARSQDLDAIWAGFYIAGDPALAYGYAQDQEPDAAGRIRNGALLRVYVPRSSLPGFYATSLTLAAPEAAGEVERLIGHPLPLRLDAITGPEEAGGRLETILGWPLAERTVVIPSAIPTDPRNVGGDLDPSSIPDAEAAISALPDYASQPGKPPKEDL

SEQ ID NO: 161 - protein, 474 aa, Artificial Sequence
  synthesized, fusion protein comprising: a fragment of TRAIL protein, deletion variant of P.aeruginosa exotoxin sequence, sequences of steric linkers, cleavage site recognized by furin and a transporting sequence.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGSGGGGSRHRQPRGWEQLGGGGSGGGGSEQSGYPVQRLVALYLAARLSWNQVDQVIRNALASPGSGGDLGEAIREQPEQARLALTLAAAESEYPTGAEFLGDGGAVSFSTRGTQNWTVERLLQAHRQLEEGGYVFVGYHGTFLEAAQSIVFGGVRARSQDLDAIWAGFYIAGDPALAYGYAQDQEPDAAGRIRNGALLRVYVPRSSLPGFYATSLTLAAPEAAGEVERLIGHPLPLRLDAITGPEEAGGRLETILGWPLAERTVVIPSAIPTDPRNVGGDLDPSSIPDAEAAISALPDYASQPGKPPKDEL

SEQ ID NO: 162 - protein, 474 aa, Artificial Sequence
  synthesized, fusion protein comprising: a fragment of TRAIL protein, deletion variant of Pseudomonas aeruginosa exotoxin sequence, sequences of steric linkers, cleavage site recognized by furin and a transporting sequence.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGSGGGGSRHRQPRGWEQLGGGGSGGGGSEQSGYPVQKLVALYLAARLSWNQVDQVIRNALASPGSGGDLGEAIREQPEQARLALTLAAAESEYPTGAEFLGDGGAVSFSTRGTQNWTVERLLQAHRQLEEGGYVFVGYHGTFLEAAQSIVFGGVRARSQDLDAIWAGFYIAGDPALAYGYAQDQEPDAAGRIRNGALLRVYVPRSSLPGFYATSLTLAAPEAAGEVERLIGHPLPLRLDAITGPEEAGGRLETILGWPLAERTVVIPSAIPTDPKNVGGDLDPSSIPDAEAAISALPDYASQPGKPPKDEL

SEQ ID NO: 163 - protein, 515 aa, Artificial Sequence
  synthesized, fusion protein comprising: a fragment of mutated TRAIL protein, modified Pseudomonas aeruginosa exotoxin sequence, sequences of steric linkers and a transporting sequence.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPHPILLMKSARNSCWSKDAEYGLYSIYQGGIFELKENDRIFVSVTNEHLIDMDHEASFFGAFLVGGGGSASGGPEGGSLAALTAHQACHLPLETFTRHRQPRGWEQLEQCGYPVQKLVALYLAARLSWNQVDQVIANALASPGSGGDLGEAIRESPEQARLALTLAAAESERFVRQGTGNDEAGAANGPADSGDALLERNYPTGAEFLGDGGDVSFSTRGTQQWTVERLLQAHRQLEEAGYVFVGYHGTFLEAAQSIVFGGVRARSQDLDAIWAGFYIAGDPALAYGYAQDQEPDAAGRIRNGALLRVYVPRSSLPGFYATSLTLAAPEAAGEVERLIGHPLPLRLDAITGPEESGGRLETILGWPLAERTVVIPSAIPTDPKNVGGDLDPSSIPDSEQAISALPDYASQPGKPPKDEL

SEQ ID NO: 164 - protein, 475 aa, Artificial Sequence
  synthesized, fusion protein comprising: a fragment of mutated TRAIL protein, mutated deletion variant of P.aeruginosa exotoxin sequence, sequences of steric linkers, cleavage site sequence recognized by furin and a transporting sequence.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPHPILLMKSARNSCWSKDAEYGLYSIYQGGIFELKENDRIFVSVTNEHLIDMDHEASFFGAFLVGGGGGSGGGGSRHRQPRGWEQLGGGGSGGGGSEQSGYPVQRLVALYLAARLSWNQVDQVIRNALASPGSGGDLGEAIREQPEQARLALTLAAAESEYPTGAEFLGDGGAVSFSTRGTQNWTVERLLQAHRQLEEGGYVFVGYHGTFLEAAQSIVFGGVRARSQDLDAIWAGFYIAGDPALAYGYAQDQEPDAAGRIRNGALLRVYVPRSSLPGFYATSLTLAAPEAAGEVERLIGHPLPLRLDAITGPEEAGGRLETILGWPLAERTVVIPSAIPTDPRNVGGDLDPSSIPDAEAAISALPDYASQPGKPPKDEL

SEQ ID NO: 165 - protein, 463 aa, Artificial Sequence
  synthesized, fusion protein comprising: a fragment of mutated TRAIL protein, deletion variant of mutated P.aeruginosa exotoxin sequence, sequences of steric linkers, cleavage site sequence recognized by furin and a transporting sequence.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPHPILLMKSARNSCWSKDAEYGLYSIYQGGIFELKENDRIFVSVTNEHLIDMDHEASFFGAFLVGGGGSGGGSRHRQPRGWEQLEQSGYPVQRLVALYLAARLSWNQVDQVIRNALASPGSGGDLGEAIREQPEQARLALTLAAAESEYPTGAEFLGDGGAVSFSTRGTQNWTVERLLQAHRQLEEGGYVFVGYHGTFLEAAQSIVFGGVRARSQDLDAIWAGFYIAGDPALAYGYAQDQEPDAAGRIRNGALLRVYVPRSSLPGFYATSLTLAAPEAAGEVERLIGHPLPLRLDAITGPEEAGGRLETILGWPLAERTVVIPSAIPTDPRNVGGDLDPSSIPDAEAAISALPDYASQPGKPPKDEL

SEQ ID NO: 166 - protein, 475 aa, Artificial Sequence
  synthesized, fusion protein comprising: a fragment of mutated TRAIL protein, deletion variant of mutated P.aeruginosa exotoxin sequence, sequences of steric linkers, cleavage site sequence recognized by furin and a transporting sequence.
RVAAHITGTRGRSNTLSSPNSKNEKALGRKINSWESSRSGHSFLSNLHLRNGELVIHEKGFYYIYSQTNFKFREEIKENTKNDKQMVQYIYKYTSYPDPILLMKSARNSCWSKDAEYGLYSIYQGGIFELKENDRIFVSVTNERLRDMHHEASFFGAFLVGGGGGSGGGGSRHRQPRGWEQLGGGGSGGGGSEQSGYPVQRLVALYLAARLSWNQVDQVIRNALASPGSGGDLGEAIREQPEQARLALTLAAAESEYPTGAEFLGDGGAVSFSTRGTQNWTVERLLQAHRQLEEGGYVFVGYHGTFLEAAQSIVFGGVRARSQDLDAIWAGFYIAGDPALAYGYAQDQEPDAAGRIRNGALLRVYVPRSSLPGFYATSLTLAAPEAAGEVERLIGHPLPLRLDAITGPEEAGGRLETILGWPLAERTVVIPSAIPTDPRNVGGDLDPSSIPDAEAAISALPDYASQPGKPPKDEL

SEQ ID NO: 167 - protein, 474 aa, Artificial Sequence
  synthesized, fusion protein comprising: a fragment of TRAIL protein, deletion variant of mutated P.aeruginosa exotoxin sequence, sequences of steric linkers, cleavage site sequence recognized by furin and a transporting sequence.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGSGGGGSRHRQPRGWEQLGGGGSGGGGSEQSGYPVQKLVALYLAARLSWNQVDQVIRNALASPGSGGDLGEAIREQPEQARLALTLAAAESEYPTGAEFLGDGGAVSFSTRGTQNWTVERLLQAHRQLEEGGYVFVGYHGTFLEAAQSIVFGGVRARSQDLDAIWAGFYIAGDPALAYGYAQDQEPDAAGRIRNGALLRVYVPRSSLPGFYATSLTLAAPEAAGEVERLIGHPLPLRLDAITGPEEAGGRLETILGWPLAERTVVIPSAIPTDPKNVGGDLDPSSIPDAEAAISALPDYASQPGKPPKEDL

SEQ ID NO: 168 - protein, 232 aa, Artificial Sequence [sequence omitted]

SEQ ID NO: 169 - DNA, 1299 bp, Artificial Sequence
  synthesized, encoding fusion protein comprising: a fragment of mutated TRAIL protein, a variant of abrin A domain, sequences of steric linkers and cleavage site recognized by furin.
gaagatcgtccgatcaaatttagcaccgaaggtgcaaccagccagagctataaacagtttattgaagcactgcgtgaacgtctgcgtggtggtctgattcatgatattccggttctgccggatccgaccaccctgcaagaacgtaatcgttatattaccgttgaactgagcaatagcgataccgaaagcattgaagttggtattgatgcaaccaatgcctatgttgttgcatatcgtgcaggtacacagagctattttctgcgtgatgcaccgagcagcgcaagcgattacctgtttaccggcaccgatcagcatagcctgccgttttatggcacctatgcagatctggaacgttgggcacatcagagccgtcagcagattccgctgggtctgcaggcactgacccatggtattagcttttttcgtagcggtggcaatgataacgaagaaaaagcacgtaccctgattgtgattattcagatggttgcagaagcagcccgttttcgctatatttcaaatcgtgttcgtgttagcattcagaccggtacagcatttcagccggatgcagcaatgattagcctggaaaataactgggataatctgagccgtggtgttcaagaaagcgttcaggatacctttccgaatcaggttaccctgaccaatattcgtaatgaaccggttattgttgatcagctgagccatccgaccgttgcagttctggcactgatgctgtttgtttgtaatccgcctaatggtggtggtggcagcggtggtggcggtagccgtaaaaaacgcgttcgtgaacgtggtccgcagcgtgttgcagcacatattaccggtacacgtggtcgtagcaataccctgagcagcccgaatagcaaaaatgaaaaagccctgggtcgtaaaatcaatagctgggaaagcagccgtagcggtcatagctttctgagcaatctgcatctgcgtaatggtgaactggttattcatgagaaaggcttctactatatctacagccagacctattttcgcttccaagaagagattaaagaaaacaccaaaaacgataaacaaatggtgcagtacatctataaatacaccagctatccggatcctatcctgctgatgaaaagcgcacgtaatagctgttggagcaaagatgcagaatatggcctgtatagcatttatcagggtggcatctttgaactgaaagaaaacgatcgtattttcgtgagcgtgaccaatgaacatctgatcgatatggatcatgaagccagcttttttggtgcatttctggtgggt

SEQ ID NO: 170 - DNA, 1350 bp, Artificial Sequence
  synthesized, encoding fusion protein comprising: a fragment of mutated TRAIL protein, a variant of ricin A domain, sequences of steric linkers, cleavage site recognized by furin, pegylation linker sequence and transporting sequence.
cgtgttgcagcacatattaccggcacccgtggtcgtagcaataccctgagcagcccgaatagcaaaaatgaaaaagcactgggtcgcaaaattaacagctgggaaagcagccgtagcggtcatagctttctgagcaatctgcatctgcgtaatggtgaactggtgattcatgaaaaaggcttctactatatctacagccagacctattttcgcttccaagaagagattaaagaaaacaccaaaaacgataaacaaatggtgcagtacatctataaatacaccagctatccggatccgattctgctgatgaaaagcgcacgtaatagctgttggagcaaagatgcagaatatggcctgtatagcatttatcagggtggcatctttgaactgaaagaaaacgatcgtattttcgtgagcgtgaccaatgaacatctgatcgatatggatcatgaagccagcttttttggtgcatttctggtgggtggtggtggcggtagcgcaagcggttgtggtccggaacgtaaaaaacgtggcggtggtggtagtgaagataataacatttttccgaaacagtacccgatcatcaattttaccaccgcaggcgcaaccgttcagagctataccaactttattcgtgcagttcgtggtcgtctgaccaccggtgcagatgttcgtcatgaaattccggttctgccgaatcgtgttggtctgccgattaatcagcgttttattctggttgaactgagcaaccatgcagaactgagcgttaccctggcaaccaatgcctatgtggttggttatcgtgcaggtaatagcgcctattttttccatccggataatcaagaagatgccgaagcaattacccacctgtttaccgatgttcagaatcgttatacctttgcctttggtggtaattatgatcgtctggaacagctggcaggtagcctgcgtgaaaatattgaactgggtaatggtccgctggaagaagccattagcgcactgtattattacagtaccggtggcacccagctgccgaccctggcacgtagctttattgtttgtattcagatgattagcgaagccgcacgctttcagtatattgaaggtgaaatgcgtacccgcattcgttataatcgtcgtagcgcaccggatccgtcagttattaccctggaaaatagctggggtcgtctgtcaaccgcaattcaagaaagcaatcagggtgcatttgcaagcccgattcagctgcagcgtcgtaatggtagcaaatttagcgtttatgatgtgagcattctgatcccgattattgccctgatggtgtatcgttgtgcaccgcctccgaaagaagatctg

SEQ ID NO: 171 - DNA, 1443 bp, Artificial Sequence
  synthesized,encoding fusion protein comprising: a fragment of mutated TRAIL protein, a active domain of diphtheria toxin, sequences of steric linkers, cleavage sites recognized by furin and transporting sequence.
ggtgcagatgatgttgcagatagcagcaaaagctttgtgatggaaaactttagcagctatcatggcaccaaaccgggttatgccgatagcattcagaaaggtattcagaaaccgaaaagcggcacccagggtaattatgatgatgattggaaaggcttctatagcaccgataacaaatatgatgcagccggttatagcgtggataatgaaaatccgctgagcggtaaagccggtggtgttgttaaagttacctatccgggtctgaccaaagttctggcactgaaagttgataatgccgaaaccatcaaaaaagaactgggtctgagcctgaccgaaccgctgatggaacaggttggcaccgaagaatttatcaaacgttttggtgatggtgcaagccgtgttgtgctgagcctgccgtttgcagaaggtagcagcagcgttgaatatatcaataattgggaacaggcaaaagccctgagcgttgaactggaaatcaattttgaaacccgtggtaaacgtggtcaggatgcaatgtatgaatacatggcacaggcatgtgcaggtaatcgtaaaaaacgcggtggtggtggtagtccggaaggtggtagcctggcagcactgaccgcacatcaggcatgtcatctgccgctggaaacctttacccgtcatcgtcagcctcgtggctgggaacagctggaacagtgtggttatccggttcagcgtctggttgcactgtatctggcagcccgtctgagctggaatcaggttgatcaggttattcgtaatgcactggcaagtccgggtagcggtggtgatctgggtgaagcaattcgtgaacagcctgaacaggcacgtctggccctgaccctggcagccgcagaaagcgaacgttttgttcgtcagggcaccggtaatggtcgcaaaaaacgtggcggtggcggttcagggggtggtggttcacgtgttgcagcacatattaccggcacccgtggtcgtagcaataccctgagcagcccgaatagcaaaaatgaaaaagcgctgggtcgtaaaatcaatagctgggaaagcagccgtagcggtcatagctttctgagcaatctgcatctgcgtaatggtgaactggtgattcatgagaaaggcttttattatatctatagccagacctactttcgcttccaagaagagattaaagaaaacaccaaaaacgataaacaaatggtgcagtacatctataaatacaccagctatccggatccgattctgctgatgaaaagcgcacgtaatagctgttggagcaaagatgcagaatatggcctgtatagcatttatcagggtggcatctttgaactgaaagaaaacgatcgtattttcgtgagcgtgaccaatgaacatctgatcgatatggatcatgaagccagcttttttggtgcatttctggtgggt

SEQ ID NO: 172 - DNA, 1434 bp, Artificial Sequence
  synthesized,encoding fusion protein comprising: a fragment of mutated TRAIL protein, a mutated active domain of diphtheria toxin, sequences of steric linkers, cleavage sites recognized by furin and transporting sequence.
ggtgcagatgatgtgagcaaaagctttgtgatggaaaactttagcagctatcatggcaccaaaccgggttatgcagatagcattcagaaaggtattcagaaaccgaaaagcggcacccagggtaattatgatgatgattggaaaggcttctatagcaccgataacaaatatgatgcagccggttatagcgtggataatgaaaatccgctgagcggtaaagccggtggtgttgttaaagttacctatccgggtctgaccaaagttctggcactgaaagttgataatgccgaaaccatcaaaaaagaactgggtctgagcctgaccgaaccgctgatggaacaggttggcaccgaagaatttatcaaacgttttggtgatggtgcaagccgtgttgtgctgagcctgccgtttgcagaaggtagcagcagcgttgaatatatcaataattgggaacaggcaaaagccctgagcgttgaactggaaatcaattttgaaacccgtggtaaacgtggtcaggatgcaatgtatgaatacatggcacaggcatgtgcaggtaatcgtaaaaaacgcggtggtggtggtagtccggaaggtggtagcctggcagcactgaccgcacatcaggcatgtcatctgccgctggaaacctttacccgtcatcgtcagcctcgtggctgggaacagctggaacagtgtggttatccggttcagcgtctggttgcactgtatctggcagcccgtctgagctggaatcaggttgatcaggttattcgtaatgcactggcaagtccgggtagcggtggtgatctgggtgaagcaattcgtgaacagcctgaacaggcacgtctggccctgaccctggcagccgcagaaagcgaacgttttgttcgtcagggcaccggtaatggtcgcaaaaaacgtggcggtggcggttcagggggtggtggttcacgtgttgcagcacatattaccggcacccgtggtcgtagcaataccctgagcagcccgaatagcaaaaatgaaaaagcgctgggtcgtaaaatcaatagctgggaaagcagccgtagcggtcatagctttctgagcaatctgcatctgcgtaatggtgaactggtgattcatgagaaaggcttttattatatctatagccagacctactttcgcttccaagaagagattaaagaaaacaccaaaaacgataaacaaatggtgcagtacatctataaatacaccagctatccggatccgattctgctgatgaaaagcgcacgtaatagctgttggagcaaagatgcagaatatggcctgtatagcatttatcagggtggcatctttgaactgaaagaaaacgatcgtattttcgtgagcgtgaccaatgaacatctgatcgatatggatcatgaagccagcttttttggtgcatttctggtgggt

SEQ ID NO: 173 - DNA, 1299 bp, Artificial Sequence
  synthesized, encoding fusion protein comprising: a fragment of mutated TRAIL protein, a mutated variant of gelonin, sequences of steric linkers, cleavage site recognized by furin and pegylation linker.
ggtctggataccgttagctttagcaccaaaggtgcaacctatattacctatgtgaactttctgaatgagctgcgcgttaaactgaaaccggaaggtaatagccatggtattccgctgctgcgtaaaaaagcagatgatccgggtaaagcatttgttctggttgcactgagcaatgataatggtcagctggcagaaattgccattgatgcaaccagcgtttatgttgttggttatcaggttcgtaatcgcagctatttcttcaaagatgcaccggatgcagcctatgaaggtctgtttaaaaacaccattaaaacccgtctgcatttcggtggtagctatccgagcctggaaggtgaaaaagcatatcgtgaaaccaccgatctgggtattgaaccgctgcgtattggtatcaaaaaactggatgaaaacgccatcgataactataaaccgaccgaaattgcaagcagcctgctggttgttattcagatggttagcgaagcagcacgctttacctttattgaaaatcagatccgcaacaactttcagcagcgtattcgtccggcaaataataccattagcctggaaaacaaatggggcaaactgagctttcagattcgtaccagcggtgcaaatggtatgtttagtgaagcagttgaactggaacgtgccaatggcaaaaaatactatgttaccgcagttgatcaggtgaaaccgaaaattgcactgctgaaattcgttgacaaagatccgaaaggtggtggtggtagccgtaaaaaacgtgcaagcggttgtggtccggaaggcggtggcggtagtcgtgttgcagcacatattaccggcacccgtggtcgtagcaataccctgagcagcccgaatagcaaaaatgaaaaagccctgggtcgtaaaatcaatagctgggaaagcagccgtagcggtcatagctttctgagcaatctgcatctgcgtaatggtgaactggtgattcatgaaaaaggcttctactatatctacagccagacctattttcgcttccaagaagagattaaagaaaacaccaaaaacgataaacaaatggtgcagtacatctataaatacaccagctatccggatccgattctgctgatgaaaagcgcacgtaatagctgttggagcaaagatgccgaatatggtctgtatagcatttatcagggtggcatcttcgaactgaaagaaaacgatcgtatttttgtgagcgtgaccaacgaacatctgatcgatatggatcatgaagccagcttttttggtgcatttctggttggc

SEQ ID NO: 174 - DNA, 759 bp, Artificial Sequence
  synthesized, encoding fusion protein comprising: a fragment of mutated TRAIL protein, a P1 luffin peptide, sequences of steric linkers, cleavage site recognized by furin and transporting sequence.
cctcgtggtagtccgcgtaccgaatatgaagcatgtcgtgttcgttgtcaggttgcagaacatggtgttgaacgtcagcgtcgttgccagcaggtttgtgaaaaacgtctgcgtgaacgtgaaggtcgtcgtgaagttgataaagatgaactgcgtaaaaaacgtggtggtggttgtgcagcagcatgtgcagcctgtaccagcgaagaaaccattagcaccgttcaagaaaaacagcagaatattagtccgctggttcgtgaacgcggtccgcagcgtgttgcagcacatattaccggcacccgtggtcgtagcaataccctgagcagcccgaatagcaaaaatgaaaaagcactgggtcgcaaaattaacagctgggaaagcagccgtagcggtcatagctttctgagcaatctgcatctgcgtaatggtgaactggtgattcatgaaaaaggcttctactatatctacagccagacctattttcgcttccaagaagagattaaagaaaacaccaaaaacgataaacaaatggtgcagtacatctataaatacaccagctatccggatccgattctgctgatgaaaagcgcacgtaatagctgttggagcaaagatgcagaatatggcctgtatagcatttatcagggtggcatctttgaactgaaagaaaacgatcgtattttcgtgagcgtgaccaatgaacatctgatcgatatggatcatgaagccagcttttttggtgcatttctggtgggt

SEQ ID NO: 175 - DNA, 759 bp, Artificial Sequence; identical to SEQ ID NO: 174
  synthesized, encoding fusion protein comprising: a fragment of mutated TRAIL protein, a P1 luffin peptide, sequences of steric linkers, cleavage site recognized by furin and a transporting sequence.

SEQ ID NO: 176 - DNA, 1617 bp, Artificial Sequence
  synthesized, encoding fusion protein comprising: a fragment of TRAIL protein, a mutated variant of trichosantin, sequences of steric linkers, cleavage sites recognized by furin and transporting sequence.
gatgttagctttcgtctgagcggtgcaaccagcagcagctatggtgtgtttattagcaatctgcgtaaagcactgccgaatgaacgtaaactgtatgatattccgctgctgcgtagcagcctgcctggtagccagcgttatgcactgattcatctgaccaattatgccgatgaaaccattagcgttgcaattgatgttaccaacgtgtatatcatgggttatcgtgccggtgataccagcgcatgtagcaatgaagcaagcgcaaccgaagcagcaaaatatgtttttaaagatgccatgcgcaaagtgaccctgccgtatagcggtaattatgaacgcctgcagaccgcagcaggtaaaattcgtgaaaacattccgctgggtctgcctgcactggatagcgcaattaccaccctgttttattacaatgcaaatagcgcagccagcgcactgatggttctgattcagagcaccagcgaagcagctcgctataaattcattgaacagcagattggttgcggtgtggataaaacctttctgccgagcctggcaattattagcctggaaaatagctggtcagcactgagcaaacaaattcagattgcaagcaccaataacggccagtttgaaagtccggttgttctgattaatgcacagaatcagcgtgtgaccattaccaatgttgatgccggtgttgttaccagtaatattgcactgctgctgaatcgcaataatatggcacgtaaaaaacgcggtggtggtggtagtccggaaggtggtagcctggcagccctgaccgcacatcaggcatgtcatctgccgctggaaacctttacccgtcatcgtcagcctcgtggttgggaacagctggaacagtgtggttatccggttcagcgtctggttgccctgtatctggcagcacgtctgagctggaatcaggttgatcaggttattcgtaatgcactggcaagtccgggtagcggtggtgatctgggtgaagccattcgtgaacagcctgaacaggcacgtctggcactgaccctggcagcagcagaaagcgaacgttttgttcgtcagggcaccggtaacggtcgcaaaaaacgtggcggtggcggttcagggggtggtggttcacgtgttgcagcacatattaccggcacccgtggtcgtagcaataccctgagcagcccgaatagcaaaaatgaaaaagcactgggtcgtaaaatcaacagctgggaaagcagccgtagcggtcatagctttctgagtaatctgcatctgcgcaatggtgaactggtgattcatgaaaaaggcttctactatatctacagccagacctattttcgcttccaagaagagattaaagaaaacaccaaaaacgataaacaaatggtgcagtacatctataaatacaccagctatccggatccgattctgctgatgaaaagcgcacgtaatagctgttggagcaaagatgcagaatatggcctgtatagcatttatcagggtggcatttttgagctgaaagaaaacgatcgtattttcgtgagcgtgaccaatgaacatctgatcgatatggatcatgaagccagcttttttggtgcatttctggtgggt

SEQ ID NO: 177 - DNA, 1287 bp, Artificial Sequence
  synthesized, encoding a fusion protein comprising: a fragment of mutated TRAIL protein, a mutated variant of trichosantin, sequences of steric linkers, cleavage site recognized by furin and pegylation sequence.
gatgcaagctttcgtctgagcggtgcaaccagcagcagctatggtgtgtttattagcaatctgcgtaaagcactgccgaatgaacgtaaactgtatgatattccgctgctgcgtagcagcctgcctggtagccagcgttatgcactgattcatctgaccaattatgccgatgaaaccattagcgttgcaattgatgcaaccaacgtgtatatcatgggttatcgtgccggtgataccagcgcatgtagcaatgaagcaagcgcaaccgaagcagcaaaatatgtttttaaagatgccatgcgcaaagtgaccctgccgtatagcggtaattatgaacgcctgcagaccgcagcaggtaaaattcgtgaaaacattccgctgggtctgcctgcgggtgatagcgcaattaccaccctgttttattacaatgcaaatagcgcagccagcgcactgatggttctgattcagagcaccagcgaagcagctcgctataaattcattgaacagcagattggttgcggtgtggataaaacctttctgccgagcctggcaattattagcctggaaaatagctggtcagcactgagcaaacaaattcagattgcaagcaccaataacggccagtttgaaagtccggttgttctgattaatgcacagaatcagcgtgtgaccattaccaatgttgatgccggtgttgttaccagtaatattgcactgctgctgaatcgcaataatatggcaggcggtggtggtagccgtaaaaaacgtgcaagcggttgtggtccggaaggtggtggtggttcacgtgttgcagcacatattaccggcacccgtggtcgtagcaataccctgagcagcccgaatagcaaaaatgaaaaagcactgggtcgcaaaattaacagctgggaaagcagccgtagcggtcatagctttctgagtaatctgcatctgcgcaatggtgaactggtgattcatgaaaaaggcttctactatatctacagccagacctattttcgcttccaagaagagattaaagaaaacaccaaaaacgataaacaaatggtgcagtacatctataaatacaccagctatccggatccgattctgctgatgaaaagcgcacgtaatagctgttggagcaaagatgcagaatatggcctgtatagcatttatcagggtggcatttttgagctgaaagaaaacgatcgtattttcgtgagcgtgaccaatgaacatctgatcgatatggatcatgaagccagcttttttggtgcatttctggtgggt

SEQ ID NO: 178 - DNA, 1545 bp, Artificial Sequence
  synthesized, encoding fusion protein comprising: a fragment of mutated TRAIL protein, modified P.aeruginosa exotoxin sequence, sequences of steric linkers and transporting sequence.
cgtgttgcagcacatattaccggcacccgtggtcgtagcaataccctgagcagcccgaatagcaaaaatgaaaaagcactgggtcgcaaaattaacagctgggaaagcagccgtagcggtcatagctttctgagcaatctgcatctgcgtaatggtgaactggtgattcatgaaaaaggcttctactatatctacagccagacctattttcgcttccaagaagagattaaagaaaacaccaaaaacgataaacaaatggtgcagtacatctataaatacaccagctatccggatccgattctgctgatgaaaagcgcacgtaatagctgttggagcaaagatgcagaatatggcctgtatagcatttatcagggtggcatctttgaactgaaagaaaacgatcgtattttcgtgagcgtgaccaatgaacatctgatcgatatggatcatgaagccagcttttttggtgcatttctggtgggtggtggtggtagcgcaagcggtggtccggaaggtggtagcctggcagcactgaccgcacatcaggcatgtcatctgccgctggaaacctttacccgtcatcgtcagcctcgtggttgggaacagctggaacagtgtggttatccggttcagaaactggttgcactgtatctggcagcccgtctgagctggaatcaggttgatcaggttattgcaaatgcactggcaagtccgggtagcggtggtgatctgggtgaagcaattcgtgaaagtccggaacaggcacgtctggcactgaccctggcagccgcagaaagcgaacgttttgttcgtcagggcaccggtaatgatgaagccggtgcagcaaatggtccggcagatagcggtgatgcactgctggaacgtaattatccgaccggtgcagaatttctgggtgatggcggtgatgttagctttagtacccgtggcacccagcagtggaccgttgaacgtctgctgcaggcacaccgtcagctggaagaggcaggttatgtttttgttggttatcatggcacctttctggaagcagcacagagcattgtgtttggtggtgttcgtgcacgtagccaggatctggatgcaatttgggcaggtttctatattgccggtgatccggcactggcctatggttatgcacaggatcaagaaccggatgcagcaggtcgtattcgcaatggtgccctgctgcgtgtttatgttccgcgtagcagcctgcctggtttttatgcaaccagcctgacactggctgcacctgaagcagccggtgaagtggaacgtctgattggtcatccgctgccgctgcgtctggatgcgattaccggtcctgaagaaagtggtggtcgtctggaaaccattctgggttggcctctggcagaacgtaccgttgttattccgagcgcaattccgaccgatccgaaaaatgttggtggcgatctggatccgagcagcattccggatagtgaacaggcaattagcgcactgccggattatgccagccagcctggtaaaccgcctaaagatgaactg

SEQ ID NO: 179 - DNA, 1206 bp, Artificial Sequence
  synthesized, encoding fusion protein comprising: a fragment of mutated TRAIL protein, deletion variant of Pseudomonas aeruginosa exotoxin sequence, sequences of steric linkers, cleavage site recognized by furin and a transporting sequence.
cgtgttgcagcacatattaccggcacccgtggtcgtagcaataccctgagcagcccgaatagcaaaaatgaaaaagcactgggtcgcaaaattaacagctgggaaagcagccgtagcggtcatagctttctgagcaatctgcatctgcgtaatggtgaactggtgattcatgaaaaaggcttctactatatctacagccagacctattttcgcttccaagaagagattaaagaaaacaccaaaaacgataaacaaatggtgcagtacatctataaatacaccagctatccggatccgattctgctgatgaaaagcgcacgtaatagctgttggagcaaagatgcagaatatggcctgtatagcatttatcagggtggcatctttgaactgaaagaaaacgatcgtattttcgtgagcgtgaccaatgaacatctgatcgatatggatcatgaagccagcttttttggtgcatttctggtgggtggtggtggtagcggtggtggcggtcgtaaaaaacgtcgtcatcgtcagcctcgtggttgggaacagctgccgaccggtgcagaatttctgggtgatggcggtgatgttagctttagtacccgtggcacccagaattggaccgttgaacgtctgctgcaggcacaccgtcagctggaagaggcaggttatgtttttgttggttatcatggcacctttctggaagcagcacagagcattgtgtttggtggtgttcgtgcacgtagccaggatctggatgcaatttgggcaggtttctatattgccggtgatccggcactggcctatggttatgcacaggatcaagaaccggatgcagcaggtcgtattcgcaatggtgcactgctgcgtgtttatgttccgcgtagcagcctgcctggtttttatgcaaccagcctgaccctggcagcaccggaagcagccggtgaagtggaacgtctgattggtcatccgctgccgctgcgtctggatgccattaccggtccggaagaaagcggtggtcgtctggaaaccattctgggttggcctctggcagaacgtaccgttgttattccgagcgcaattccgaccgatccgcgtaatgttggtggtgatctggatccgagcagcattccggatagtgaacaggcaattagcgcactgccggattatgccagccagcctggtaaaccgcctaaagaagatctg

SEQ ID NO: 180 - DNA, 1209 bp, Artificial Sequence
  synthesized, encoding fusion protein comprising: a fragment of mutated TRAIL protein, deletion variant of Pseudomonas aeruginosa exotoxin sequence, sequences of steric linkers, cleavage site recognized by furin and a transporting sequence.
cagatcttctttaggcggtttaccaggctggctggcataatccggcagtgcgctaattgctgcttcggcatccggaatgctgctcggatccagatcaccaccaacattacgcggatcggtcggaattgcgctcggaataacaacggtacgttctgccagaggccaacccagaatggtttccagacgaccgcctgcttcttccggaccggtaatggcatccagacgcagcggcagcggatgaccaatcagacgttccacttcaccggctgcttccggtgctgccagggtcaggctggttgcataaaaaccaggcaggctgctacgcggaacataaacacgcagcagtgcaccattgcgaatacgacctgctgcatccggttcttgatcctgtgcataaccataggccagtgccggatcaccggcaatatagaaacctgcccaaattgcatccagatcctggctacgtgcacgaacaccaccaaacacaatgctctgtgctgcttccagaaaggtgccatgataaccaacaaaaacataaccaccctcttccagctgacggtgtgcctgcagcagacgttcaacggtccaattctgggtgccacgggtactaaagctaactgcgccaccatcacccagaaattctgcaccggtcggatacagctgttcccaaccacgaggctgacgatgacgacgttttttacgaccgccaccaccgctaccaccaccacccaccagaaatgcaccaaaaaagctggcttcatgatccatatcgatcagatgttcattggtcacgctcacgaaaatacgatcgttttctttcagttcaaagatgccaccctgataaatgctatacaggccatattctgcatctttgctccaacagctattacgtgcgcttttcatcagcagaatcggatccggatagctggtgtatttatagatgtactgcaccatttgtttatcgtttttggtgttttctttaatctcttcttggaagcgaaaataggtctggctgtagatatagtagaagcctttttcatgaatcaccagttcaccattacgcagatgcagattgctcagaaagctatgaccgctacggctgctttcccagctgttaattttgcgacccagtgctttttcatttttgctattcgggctgctcagggtattgctacgaccacgggtgccggtaatatgtgctgcaacacg

SEQ ID NO: 181 - DNA, 1410 bp, Artificial Sequence
  synthesized, encoding fusion protein comprising: a fragment of mutated TRAIL protein, deletion variant of P.aeruginosa exotoxin sequence, a sequence of steric linker, a pegylation linker, cleavage sites recognized by furin and a transporting sequence.
cagatcttctttaggcggtttaccaggctggctggcataatccggcagtgcgctaattgctgcttcggcatccggaatgctgctcggatccagatcgccaccaacattacgcggatcggtcggaattgcgctcggaataacaacggtacgttctgccagaggccaacccagaatggtttccagacgaccgcctgcttcttccggaccggtaatggcatccagacgcagcggcagcggatgaccaatcagacgttccacttcaccggctgcttccggtgcagccagtgtcaggctggttgcataaaaaccaggcaggctgctacgcggaacataaacacgcagcagtgcaccattgcgaatacgacctgctgcatccggttcttgatcctgtgcataaccataggccagtgccggatcaccggcaatatagaaacctgcccaaattgcatccagatcctggctacgtgcacgaacaccaccaaacacaatgctctgtgctgcttccagaaaggtgccatgataaccaacaaaaacataaccaccctcttccagctgacggtgtgcctgcagcagacgttcaacggtccaattctgggtgccacgggtactaaagctaactgcgccaccatcacccagaaattctgcaccggtcggatattcgctttctgctgctgccagggtcagtgccagacgtgcctgttcaggctgttcacgaattgcttcacccagatcaccaccgctacccggacttgccagtgcattacgaataacctgatcaacctgattccagctcagacgtgctgccagatacagtgcaaccagacgctgaaccggataaccgctctgttccagctgttcccaaccacgaggctgacgatgacgacgttttttacgttccggaccacaaccgcttgcgctaccaccaccacccaccagaaatgcaccaaaaaagctggcttcatgatccatatcgatcagatgttcattggtcacgctcacgaaaatacgatcgttttctttcagttcaaagatgccaccctgataaatgctatacaggccatattctgcatctttgctccaacagctattacgtgcgcttttcatcagcagaatcggatccggatagctggtgtatttatagatgtactgcaccatttgtttatcgtttttggtgttttctttaatctcttcttggaagcgaaaataggtctggctgtagatatagtagaagcctttttcatgaatcaccagttcaccattacgcagatgcagattgctcagaaagctatgaccgctacggctgctttcccagctgttaattttgcgacccagtgctttttcatttttgctattcgggctgctcagggtattgctacgaccacgggtgccggtaatatgtgctgcaacacg

SEQ ID NO: 182 - DNA, 1434 bp, Artificial Sequence
  synthesized, encoding fusion protein comprising: a fragment of mutated TRAIL protein, deletion variant of Pseudomonas aeruginosa exotoxin sequence, sequences of steric linkers, cleavage sites recognized by furin and a transporting sequence.
cagatcttctttaggcggtttaccaggctggctggcataatccggcagtgcgctaattgctgcttcggcatccggaatgctgctcggatccagatctccgccaacattacgcggatcggtcggaattgcgctcggaataacaacggtacgttctgccagaggccaacccagaatggtttccagacgaccgcctgcttcttccggaccggtaatggcatccagacgcagcggcagcggatgaccaatcagacgttccacttcaccggctgcttccggtgcagccagtgtcaggctggttgcataaaaaccaggcaggctgctacgcggaacataaacacgcagcagtgcaccattgcgaatacgacctgctgcatccggttcttgatcctgtgcataaccataggccagtgccggatcaccggcaatatagaaacctgcccaaattgcatccagatcctggctacgtgcacgaacaccaccaaacacaatgctctgtgctgcttccagaaaggtgccatgataaccaacaaaaacataaccaccctcttccagctgacggtgtgcctgcagcagacgttcaacggtccaattctgggtgccacgggtactaaagctaactgcgccaccatcacccagaaattctgcaccggtcggatattcgctttctgctgctgccagggtcagtgccagacgtgcctgttccggctgttcacgaattgcttcacccagatcaccaccactacccggacttgccagtgcattacgaataacctgatcaacctgattccagctcagacgtgctgccagatacagtgcaaccagacgctgaaccggataaccgctctgttctgaaccacccccacctgagccaccgccacccagctgctcccaaccacgaggctgacgatgacgacgttttttacgtgaaccgccaccaccgctaccaccaccacccaccagaaatgcaccaaaaaagctggcttcatgatccatatcgatcagatgttcattggtcacgctcacgaaaatacgatcgttttctttcagttcaaagatgccaccctgataaatgctatacaggccatattctgcatctttgctccaacagctattacgtgcgcttttcatcagcagaatcggatccggatagctggtgtatttatagatgtactgcaccatttgtttatcgtttttggtgttttctttaatctcttcttggaagcgaaaataggtctggctgtagatatagtagaagcctttttcatgaatcaccagttcaccattacgcagatgcagattgctcagaaagctatgaccgctacggctgctttcccagctgttaattttgcgacccagtgctttttcatttttgctattcgggctgctcagggtattgctacgaccacgggtgccggtaatatgtgctgcaacacg

SEQ ID NO: 183 - DNA, 1206 bp, Artificial Sequence
  synthesized, encoding fusion protein comprising: a fragment of mutated TRAIL protein, deletion variant of P.aeruginosa exotoxin sequence, sequences of steric linkers, cleavage sites recognized by furin and a transporting sequence.
cagatcttctttaggcggtttaccaggctggcttgcataatccggcagtgcgctaattgcctgttctttatccggaatgctgctcggatccagatcaccaccaacattacgcggatcggtcggaattgcgctcggaataacaacggtacgttctgccagaggccaacccagaatggtttccagacgaccgccttcttcttccggaccggtaatggcatccagacgcagcggcagcggatgaccaatcagacgttccacttcaccggctgcttccggtgctgccagggtcaggctggtacgataaaaaccaggcaggctgctacgcggaacataaacacgcagcagtgcaccattgcgaatgcgaccacgtgcatccggttcttgatcctgtgcataaccataggccagtgccggatcaccggcaatatagaaaccacgccaaattgcatccagatcctggctacgtgcacgaacaccaccaaacacaatgctctgtgctgcttccagaaaggtgccatgataaccaacaaaaacataaccacgttcttccagctgacggtgtgcctgcagcagacgttcaacggtccaattctgggtgccacgggtactaaagctaacatcaccgccatcacccagaaattctgcaccggtcggcagctgttcccaaccacgaggctgacgatgacgacgttttttacgaccgccaccaccgctaccaccaccacccaccagaaatgcaccaaaaaagctggcttcatgatccatatcgatcagatgttcattggtcacgctcacgaaaatacgatcgttttctttcagttcaaagatgccaccctgataaatgctatacaggccatattctgcatctttgctccaacagctattacgtgcgcttttcatcagcagaatcggatccggatagctggtgtatttatagatgtactgcaccatttgtttatcgtttttggtgttttctttaatctcttcttggaagcgaaaataggtctggctgtagatatagtagaagcctttttcatgaatcaccagttcaccattacgcagatgcagattgctcagaaagctatgaccgctacggctgctttcccagctgttaattttgcgacccagtgctttttcatttttgctattcgggctgctcagggtattgctacgaccacgggtgccggtaatatgtgctgcaacacg

SEQ ID NO: 184 - DNA, 1401 bp, Artificial Sequence
  synthesized, encoding fusion protein comprising: a fragment of mutated TRAIL protein, deletion variant of P.aeruginosa exotoxin sequence, sequences of steric linkers, cleavage site recognized by furin and a transporting sequence.
cagatcttctttaggcggtttaccaggctggctggcataatccggcagtgcgctaattgctgcttcggcatccggaatgctgctcggatccagatctccgccaacattacgcggatcggtcggaattgcgctcggaataacaacggtacgttctgccagaggccaacccagaatggtttccagacgaccgcctgcttcttccggaccggtaatggcatccagacgcagcggcagcggatgaccaatcagacgttccacttcaccggctgcttccggtgcagccagtgtcaggctggttgcataaaaaccaggcaggctgctacgcggaacataaacacgcagcagtgcaccattgcgaatacgacctgctgcatccggttcttgatcctgtgcataaccataggccagtgccggatcaccggcaatatagaaacctgcccaaattgcatccagatcctggctacgtgcacgaacaccaccaaacacaatgctctgtgctgcttccagaaaggtgccatgataaccaacaaaaacataaccaccctcttccagctgacgatgtgcctgcagcagacgttcaacggtccaattctgggtgccacgggtactaaagctaactgcgccaccatcacccagaaattctgcaccggtcggatattcgctttctgctgctgccagggtcagtgccagacgtgcctgttccggctgttcacgaattgcttcacccagatcaccaccactacccggacttgccagtgcattacgaataacctgatcaacctgattccagctcagacgtgctgccagatacagtgcaaccagacgctgaaccggataaccgctctgttcgctacctccgccacctgaaccaccgccaccgcgttttttacgtgaaccgccaccaccgctaccaccaccacccaccagaaatgcaccaaaaaagctggcttcatgatccatatcgatcagatgttcattggtcacgctcacgaaaatacgatcgttttctttcagttcaaagatgccaccctgataaatgctatacaggccatattctgcatctttgctccaacagctattacgtgcgcttttcatcagcagaatcggatccggatagctggtgtatttatagatgtactgcaccatttgtttatcgtttttggtgttttctttaatctcttcttggaagcgaaaataggtctggctgtagatatagtagaagcctttttcatgaatcaccagttcaccattacgcagatgcagattgctcagaaagctatgaccgctacggctgctttcccagctgttaattttgcgacccagtgctttttcatttttgctattcgggctgctcagggtattgctacgaccacgggtgccggtaatatgtgctgcaacacg

SEQ ID NO: 185 - DNA, 1422 bp, Artificial Sequence
  synthesized, encoding fusion protein comprising: a fragment of mutated TRAIL protein, deletion variant of P.aeruginosa exotoxin sequence, sequences of steric linkers, cleavage site recognized by furin and a transporting sequence.
cagatcttctttaggcggtttaccaggctggctggcataatccggcagtgcgctaattgctgcttcggcatccggaatgctgctcggatccagatctccgccaacattacgcggatcggtcggaattgcgctcggaataacaacggtacgttctgccagaggccaacccagaatggtttccagacgaccgcctgcttcttccggaccggtaatggcatccagacgcagcggcagcggatgaccaatcagacgttccacttcaccggctgcttccggtgcagccagtgtcaggctggttgcataaaaaccaggcaggctgctacgcggaacataaacacgcagcagtgcaccattgcgaatacgacctgctgcatccggttcttgatcctgtgcataaccataggccagtgccggatcaccggcaatatagaaacctgcccaaattgcatccagatcctggctacgtgcacgaacaccaccaaacacaatgctctgtgctgcttccagaaaggtgccatgataaccaacaaaaacataaccaccctcttccagctgacggtgtgcctgcagcagacgttcaacggtccaattctgggtgccacgggtactaaagctaactgcgccaccatcacccagaaattctgcaccggtcggatattcgctttctgctgctgccagggtcagtgccagacgtgcctgttccggctgttcacgaattgcttcacccagatcaccaccactacccggacttgccagtgcattacgaataacctgatcaacctgattccagctcagacgtgctgccagatacagtgcaaccagacgctgaaccggataaccgctctgttctgaaccacccccacctgagccaccgccacccagctgctcccaaccacgaggctgacgatgacgtgaaccgccaccaccgctaccaccaccacccaccagaaatgcaccaaaaaagctggcttcatgatccatatcgatcagatgttcattggtcacgctcacgaaaatacgatcgttttctttcagttcaaagatgccaccctgataaatgctatacaggccatattctgcatctttgctccaacagctattacgtgcgcttttcatcagcagaatcggatccggatagctggtgtatttatagatgtactgcaccatttgtttatcgtttttggtgttttctttaatctcttcttggaagcgaaaataggtctggctgtagatatagtagaagcctttttcatgaatcaccagttcaccattacgcagatgcagattgctcagaaagctatgaccgctacggctgctttcccagctgttaattttgcgacccagtgctttttcatttttgctattcgggctgctcagggtattgctacgaccacgggtgccggtaatatgtgctgcaacacg

SEQ ID NO: 186 - DNA, 1422 bp, Artificial Sequence
  synthesized, encoding fusion protein comprising: a fragment of mutated TRAIL protein, deletion variant of P.aeruginosa exotoxin sequence, sequences of steric linkers, cleavage site recognized by furin and a transporting sequence.
cgtgttgcagcacatattaccggcacccgtggtcgtagcaataccctgagcagcccgaatagcaaaaatgaaaaagcactgggtcgcaaaattaacagctgggaaagcagccgtagcggtcatagctttctgagcaatctgcatctgcgtaatggtgaactggtgattcatgaaaaaggcttctactatatctacagccagacctattttcgcttccaagaagagattaaagaaaacaccaaaaacgataaacaaatggtgcagtacatctataaatacaccagctatccggatccgattctgctgatgaaaagcgcacgtaatagctgttggagcaaagatgcagaatatggcctgtatagcatttatcagggtggcatctttgaactgaaagaaaacgatcgtattttcgtgagcgtgaccaatgaacatctgatcgatatggatcatgaagccagcttttttggtgcatttctggtgggtggtggtggtagcggtggtggcggttcacgtcatcgtcagcctcgtggttgggagcagctgggtggcggtggctcaggtgggggtggttcagaacagagcggttatccggttcagcgtctggttgcactgtatctggcagcacgtctgagctggaatcaggttgatcaggttattcgtaatgcactggcaagtccgggtagtggtggtgatctgggtgaagcaattcgtgaacagccggaacaggcacgtctggcactgaccctggcagcagcagaaagcgaatatccgaccggtgcagaatttctgggtgatggtggcgcagttagctttagtacccgtggcacccagaattggaccgttgaacgtctgctgcaggcacaccgtcagctggaagagggtggttatgtttttgttggttatcatggcacctttctggaagcagcacagagcattgtgtttggtggtgttcgtgcacgtagccaggatctggatgcaatttgggcaggtttctatattgccggtgatccggcactggcctatggttatgcacaggatcaagaaccggatgcagcaggtcgtattcgcaatggtgcactgctgcgtgtttatgttccgcgtagcagcctgcctggtttttatgcaaccagcctgacactggctgcaccggaagcagccggtgaagtggaacgtctgattggtcatccgctgccgctgcgtctggatgccattaccggtccggaagaagcaggcggtcgtctggaaaccattctgggttggcctctggcagaacgtaccgttgttattccgagcgcaattccgaccgatccgcgtaatgttggcggagatctggatccgagcagcattccggatgccgaagcagcaattagcgcactgccggattatgccagccagcctggtaaaccgcctaaagatgaactg

SEQ ID NO: 187 - DNA, 1422 bp, Artificial Sequence
  synthesized, encoding fusion protein comprising: a fragment of mutated TRAIL protein, deletion variant of P.aeruginosa exotoxin sequence, sequences of steric linkers, cleavage site recognized by furin and a transporting sequence.
cgtgttgcagcacatattaccggcacccgtggtcgtagcaataccctgagcagcccgaatagcaaaaatgaaaaagcactgggtcgcaaaattaacagctgggaaagcagccgtagcggtcatagctttctgagcaatctgcatctgcgtaatggtgaactggtgattcatgaaaaaggcttctactatatctacagccagacctattttcgcttccaagaagagattaaagaaaacaccaaaaacgataaacaaatggtgcagtacatctataaatacaccagctatccggatccgattctgctgatgaaaagcgcacgtaatagctgttggagcaaagatgcagaatatggcctgtatagcatttatcagggtggcatctttgaactgaaagaaaacgatcgtattttcgtgagcgtgaccaatgaacatctgatcgatatggatcatgaagccagcttttttggtgcatttctggtgggtggtggtggtagcggtggtggcggttcacgtcatcgtcagcctcgtggttgggagcagctgggtggcggtggctcaggtgggggtggttcagaacagagcggttatccggttcagaaactggttgcactgtatctggcagcacgtctgagctggaatcaggttgatcaggttattcgtaatgcactggcaagtccgggtagtggtggtgatctgggtgaagcaattcgtgaacagccggaacaggcacgtctggcactgaccctggcagcagcagaaagcgaatatccgaccggtgcagaatttctgggtgatggtggcgcagttagctttagtacccgtggcacccagaattggaccgttgaacgtctgctgcaggcacaccgtcagctggaagagggtggttatgtttttgttggttatcatggcacctttctggaagcagcacagagcattgtgtttggtggtgttcgtgcacgtagccaggatctggatgcaatttgggcaggtttctatattgccggtgatccggcactggcctatggttatgcacaggatcaagaaccggatgcagcaggtcgtattcgcaatggtgcactgctgcgtgtttatgttccgcgtagcagcctgcctggtttttatgcaaccagcctgacactggctgcaccggaagcagccggtgaagtggaacgtctgattggtcatccgctgccgctgcgtctggatgccattaccggtccggaagaagcaggcggtcgtctggaaaccattctgggttggcctctggcagaacgtaccgttgttattccgagcgcaattccgaccgatccgaaaaatgttggcggagatctggatccgagcagcattccggatgccgaagcagcaattagcgcactgccggattatgccagccagcctggtaaaccgcctaaagatgaactg

SEQ ID NO: 188 - DNA, 1545 bp, Artificial Sequence
  synthesized, encoding fusion protein comprising: a fragment of mutated TRAIL protein, modified P.aeruginosa exotoxin sequence, sequences of steric linkers and a transporting sequence.
cgtgttgcagcacatattaccggcacccgtggtcgtagcaataccctgagcagcccgaatagcaaaaatgaaaaagcactgggtcgcaaaattaacagctgggaaagcagccgtagcggtcatagctttctgagcaatctgcatctgcgtaatggtgaactggtgattcatgaaaaaggcttctactatatctacagccagacctattttcgcttccaagaagagattaaagaaaacaccaaaaacgataaacaaatggtgcagtacatctataaatacaccagctatccgcatccgattctgctgatgaaaagcgcacgtaatagctgttggagcaaagatgcagaatatggcctgtatagcatttatcagggtggcatctttgaactgaaagaaaacgatcgtattttcgtgagcgtgaccaatgaacatctgatcgatatggatcatgaagccagcttttttggtgcatttctggtgggtggtggtggtagcgcaagcggtggtccggaaggtggtagcctggcagcactgaccgcacatcaggcatgtcatctgccgctggaaacctttacccgtcatcgtcagcctcgtggttgggaacagctggaacagtgtggttatccggttcagaaactggttgcactgtatctggcagcccgtctgagctggaatcaggttgatcaggttattgcaaatgcactggcaagtccgggtagcggtggtgatctgggtgaagcaattcgtgaaagtccggaacaggcacgtctggcactgaccctggcagccgcagaaagcgaacgttttgttcgtcagggcaccggtaatgatgaagccggtgcagcaaatggtccggcagatagcggtgatgcactgctggaacgtaattatccgaccggtgcagaatttctgggtgatggcggtgatgttagctttagtacccgtggcacccagcagtggaccgttgaacgtctgctgcaggcacaccgtcagctggaagaggcaggttatgtttttgttggttatcatggcacctttctggaagcagcacagagcattgtgtttggtggtgttcgtgcacgtagccaggatctggatgcaatttgggcaggtttctatattgccggtgatccggcactggcctatggttatgcacaggatcaagaaccggatgcagcaggtcgtattcgcaatggtgccctgctgcgtgtttatgttccgcgtagcagcctgcctggtttttatgcaaccagcctgacactggctgcacctgaagcagccggtgaagtggaacgtctgattggtcatccgctgccgctgcgtctggatgcgattaccggtcctgaagaaagtggtggtcgtctggaaaccattctgggttggcctctggcagaacgtaccgttgttattccgagcgcaattccgaccgatccgaaaaatgttggtggcgatctggatccgagcagcattccggatagcgaacaggcaattagcgcactgccggattatgccagccagcctggtaaaccgcctaaagatgaactg

SEQ ID NO: 189 - DNA, 1425 bp, Artificial Sequence
  synthesized, encoding fusion protein comprising: a fragment of mutated TRAIL protein, mutated deletion variant of P.aeruginosa exotoxin sequence, sequences of steric linkers, cleavage site sequence recognized by furin and a transporting sequence.
cgtgttgcagcacatattaccggcacccgtggtcgtagcaataccctgagcagcccgaatagcaaaaatgaaaaagcactgggtcgcaaaattaacagctgggaaagcagccgtagcggtcatagctttctgagcaatctgcatctgcgtaatggtgaactggtgattcatgaaaaaggcttctactatatctacagccagacctattttcgcttccaagaagagattaaagaaaacaccaaaaacgataaacaaatggtgcagtacatctataaatacaccagctatccgcatccgattctgctgatgaaaagcgcacgtaatagctgttggagcaaagatgcagaatatggcctgtatagcatttatcagggtggcatctttgaactgaaagaaaacgatcgtattttcgtgagcgtgaccaatgaacatctgatcgatatggatcatgaagccagcttttttggtgcatttctggtgggtggtggtggcggtagtggcggtggtggtagccgtcatcgtcagcctcgtggttgggagcagctgggaggtggtggttcaggcggtggcggttcagaacagagcggttatccggttcagcgtctggttgcactgtatctggcagcacgtctgagctggaatcaggttgatcaggttattcgtaatgcactggcaagtccgggtagcggtggtgatctgggtgaagcaattcgtgaacagccggaacaggcacgtctggcactgaccctggcagcagcagaaagcgaatatccgaccggtgcagaatttctgggtgatggtggtgcagttagctttagtacccgtggcacccagaattggaccgttgaacgtctgctgcaggcacaccgtcagctggaagagggtggttatgtttttgttggttatcatggcacctttctggaagcagcacagagcattgtgtttggtggtgttcgtgcacgtagccaggatctggatgcaatttgggcaggtttctatattgccggtgatccggcactggcctatggttatgcacaggatcaagaaccggatgcagcaggtcgtattcgcaatggtgcactgctgcgtgtttatgttccgcgtagcagcctgcctggtttttatgcaaccagcctgacactggctgcaccggaagcagccggtgaagtggaacgtctgattggtcatccgctgccgctgcgtctggatgccattaccggtccggaagaagcaggcggtcgtctggaaaccattctgggttggcctctggcagaacgtaccgttgttattccgagcgcaattccgaccgatccgcgtaatgttggtggcgatctggatccgagcagcattccggatgccgaagcagcaattagcgcactgccggattatgccagccagcctggtaaaccgcctaaagatgaactg

SEQ ID NO: 190 - DNA, 1389 bp, Artificial Sequence
  synthesized, encoding fusion protein comprising: a fragment of mutated TRAIL protein, deletion variant of mutated P.aeruginosa exotoxin sequence, sequences of steric linkers, cleavage site sequence recognized by furin and a transporting sequence.
cgtgttgcagcacatattaccggcacccgtggtcgtagcaataccctgagcagcccgaatagcaaaaatgaaaaagcactgggtcgcaaaattaacagctgggaaagcagccgtagcggtcatagctttctgagcaatctgcatctgcgtaatggtgaactggtgattcatgaaaaaggcttctactatatctacagccagacctattttcgcttccaagaagagattaaagaaaacaccaaaaacgataaacaaatggtgcagtacatctataaatacaccagctatccgcatccgattctgctgatgaaaagcgcacgtaatagctgttggagcaaagatgcagaatatggcctgtatagcatttatcagggtggcatctttgaactgaaagaaaacgatcgtattttcgtgagcgtgaccaatgaacatctgatcgatatggatcatgaagccagcttttttggtgcatttctggtgggtggtggtggtagcggtggtggttcacgtcatcgtcagcctcgtggttgggaacagctggaacagagcggttatccggttcagcgtctggttgcactgtatctggcagcacgtctgagctggaatcaggttgatcaggttattcgtaatgcactggcaagtccgggtagtggtggtgatctgggtgaagcaattcgtgaacagcctgaacaggcacgtctggcactgaccctggcagcagcagaaagcgaatatccgaccggtgcagaatttctgggtgatggtggcgcagttagctttagtacccgtggcacccagaattggaccgttgaacgtctgctgcaggcacaccgtcagctggaagagggtggttatgtttttgttggttatcatggcacctttctggaagcagcacagagcattgtgtttggtggtgttcgtgcacgtagccaggatctggatgcaatttgggcaggtttctatattgccggtgatccggcactggcctatggttatgcacaggatcaagaaccggatgcagcaggtcgtattcgcaatggtgcactgctgcgtgtttatgttccgcgtagcagcctgcctggtttttatgcaaccagcctgacactggctgcaccggaagcagccggtgaagtggaacgtctgattggtcatccgctgccgctgcgtctggatgccattaccggtccggaagaagcaggcggtcgtctggaaaccattctgggttggcctctggcagaacgtaccgttgttattccgagcgcaattccgaccgatccgcgtaatgttggcggagatctggatccgagcagcattccggatgccgaagcagcaattagcgcactgccggattatgccagccagcctggtaaaccgcctaaagatgaactg

SEQ ID NO: 191 - DNA, 1425 bp, Artificial Sequence
  synthesized, encoding fusion protein comprising: a fragment of mutated TRAIL protein, deletion variant of mutated P.aeruginosa exotoxin sequence, sequences of steric linkers, cleavage site sequence recognized by furin and a transporting sequence.
cgtgttgcagcacatattaccggcacccgtggtcgtagcaataccctgagcagcccgaatagcaaaaatgaaaaagcactgggtcgcaaaattaacagctgggaaagcagccgtagcggtcatagctttctgagcaatctgcatctgcgtaatggtgaactggtgattcatgaaaaaggcttctattacatctacagccagaccaacttcaaatttcgcgaagagattaaagaaaacaccaaaaacgataaacaaatggtgcagtatatctataaatacaccagctatccggatccgattctgctgatgaaaagcgcacgtaatagctgttggagcaaagatgcagaatatggcctgtatagcatttatcagggtggcatctttgaactgaaagaaaacgatcgtattttcgtgagcgttaccaatgaacgtctgcgtgatatgcatcatgaagcaagcttttttggtgcatttctggtgggtggtggtggcggtagtggcggtggtggtagccgtcatcgtcagcctcgtggttgggagcagctgggaggtggtggttcaggcggtggcggttcagaacagagcggttatccggttcagcgtctggttgcactgtatctggcagcacgtctgagctggaatcaggttgatcaggttattcgtaatgcactggcaagtccgggtagcggtggtgatctgggtgaagcaattcgtgaacagccggaacaggcacgtctggcactgaccctggcagcagcagaaagcgaatatccgaccggtgcagaatttctgggtgatggtggtgcagttagctttagtacccgtggcacccagaattggaccgttgaacgcctgctgcaggcacaccgtcagctggaagagggtggttatgtttttgttggttatcatggcacctttctggaagcagcacagagcattgtgtttggtggtgttcgtgcacgtagccaggatctggatgcaatttgggcaggtttctatattgccggtgatccggcactggcctatggttatgcacaggatcaagaaccggatgcagcaggtcgtattcgcaatggtgcactgctgcgtgtttatgttccgcgtagcagcctgcctggtttttatgcaaccagcctgacactggctgcaccggaagcagccggtgaagtggaacgtctgattggtcatccgctgccgctgcgtctggatgccattaccggtccggaagaagcaggcggtcgtctggaaaccattctgggttggcctctggcagaacgtaccgttgttattccgagcgcaattccgaccgatccgcgtaatgttggtggcgatctggatccgagcagcattccggatgccgaagcagcaattagcgcactgccggattatgccagccagcctggtaaaccgcctaaagatgaactg

SEQ ID NO: 192 - DNA, 1422 bp, Artificial Sequence
  synthesized,encoding fusion protein comprising: a fragment of TRAIL protein, deletion variant of mutated P.aeruginosa exotoxin sequence, sequences of steric linkers, cleavage site sequence recognized by furin and a transporting sequence.
cgtgttgcagcacatattaccggcacccgtggtcgtagcaataccctgagcagcccgaatagcaaaaatgaaaaagcactgggtcgcaaaattaacagctgggaaagcagccgtagcggtcatagctttctgagcaatctgcatctgcgtaatggtgaactggtgattcatgaaaaaggcttctactatatctacagccagacctattttcgcttccaagaagagattaaagaaaacaccaaaaacgataaacaaatggtgcagtacatctataaatacaccagctatccggatccgattctgctgatgaaaagcgcacgtaatagctgttggagcaaagatgcagaatatggcctgtatagcatttatcagggtggcatctttgaactgaaagaaaacgatcgtattttcgtgagcgtgaccaatgaacatctgatcgatatggatcatgaagccagcttttttggtgcatttctggtgggtggtggtggtagcggtggtggcggttcacgtcatcgtcagcctcgtggttgggagcagctgggtggcggtggctcaggtgggggtggttcagaacagagcggttatccggttcagaaactggttgcactgtatctggcagcacgtctgagctggaatcaggttgatcaggttattcgtaatgcactggcaagtccgggtagtggtggtgatctgggtgaagcaattcgtgaacagccggaacaggcacgtctggcactgaccctggcagcagcagaaagcgaatatccgaccggtgcagaatttctgggtgatggtggcgcagttagctttagtacccgtggcacccagaattggaccgttgaacgtctgctgcaggcacaccgtcagctggaagagggtggttatgtttttgttggttatcatggcacctttctggaagcagcacagagcattgtgtttggtggtgttcgtgcacgtagccaggatctggatgcaatttgggcaggtttctatattgccggtgatccggcactggcctatggttatgcacaggatcaagaaccggatgcagcaggtcgtattcgcaatggtgcactgctgcgtgtttatgttccgcgtagcagcctgcctggtttttatgcaaccagcctgacactggctgcaccggaagcagccggtgaagtggaacgtctgattggtcatccgctgccgctgcgtctggatgccattaccggtccggaagaagcaggcggtcgtctggaaaccattctgggttggcctctggcagaacgtaccgttgttattccgagcgcaattccgaccgatccgaaaaatgttggcggagatctggatccgagcagcattccggatgccgaagcagcaattagcgcactgccggattatgccagccagcctggtaaaccgcctaaagaagatctg

SEQ ID NO: 193 - DNA, 696 bp, Artificial Sequence
  synthesized, encoding fusion protein comprising: a fragment of TRAIL protein, Hok protein, sequences of steric linkers and cleavage site sequences recognized by urokinase and metalloprotease.
aaactgcctcgtagcagcctggtttggtgtgttctgattgtttgtctgaccctgctgatttttacctatctgacccgtaaaagcctgtgcgaaattcgttatcgtgatggtcatcgtgaagttgcagcatttatggcctatgaaagcggtaaaggtggtggtggtagccgtgttgttcgtccgctgggtctggcaggcggtggtggcggttcacgtgttgcagcacatattaccggcacccgtggtcgtagcaataccctgagcagcccgaatagcaaaaatgaaaaagcactgggtcgcaaaattaacagctgggaaagcagccgtagcggtcatagctttctgagcaatctgcatctgcgtaatggtgaactggtgattcatgaaaaaggcttctactatatctacagccagacctattttcgcttccaagaagagattaaagaaaacaccaaaaacgataaacaaatggtgcagtacatctataaatacaccagctatccggatccgattctgctgatgaaaagcgcacgtaatagctgttggagcaaagatgcagaatatggcctgtatagcatttatcagggtggcatctttgaactgaaagaaaacgatcgtattttcgtgagcgtgaccaatgaacatctgatcgatatggatcatgaagccagcttttttggtgcatttctggtgggt

SEQ ID NO: 194 - protein, 251 aa, Abrus precatorius
  ref <308>: DATABASE ACCESSION NUMBER: genbank/CAA38655.1
  ref <309>: DATABASE ENTRY DATE: 2005-04-18
  ref <313>: RELEVANT RESIDUES IN SEQ ID NO: (2)..(251)
EDRPIKFSTEGATSQSYKQFIEALRERLRGGLIHDIPVLPDPTTLQERNRYITVELSNSDTESIEVGIDATNAYVVAYRAGTQSYFLRDAPSSASDYLFTGTDQHSLPFYGTYADLERWAHQSRQQIPLGLQALTHGISFFRSGGNDNEEKARTLIVIIQMVAEAARFRYISNRVRVSIQTGTAFQPDAAMISLENNWDNLSRGVQESVQDTFPNQVTLTNIRNEPVIVDQLSHPTVAVLALMLFVCNPPN

SEQ ID NO: 195 - protein, 264 aa, Ricinus communis
  ref <301>: AUTHORS: Baluna et al.
  ref <302>: TITLE: -
  ref <303>: JOURNAL: Proc. Natl. Acad. Sci. USA
  ref <304>: VOLUME: 96
  ref <306>: PAGES: 39573962
  ref <307>: DATE: 1999-03-01
EDNNIFPKQYPIINFTTAGATVQSYTNFIRAVRGRLTTGADVRHEIPVLPNRVGLPINQRFILVELSNHAELSVTLATNAYVVGYRAGNSAYFFHPDNQEDAEAITHLFTDVQNRYTFAFGGNYDRLEQLAGSLRENIELGNGPLEEAISALYYYSTGGTQLPTLARSFIVCIQMISEAARFQYIEGEMRTRIRYNRRSAPDPSVITLENSWGRLSTAIQESNQGAFASPIQLQRRNGSKFSVYDVSILIPIIALMVYRCAPPP

SEQ ID NO: 196 - protein, 189 aa, Corynebacterium diphtheriae
  ref <301>: AUTHORS: Baluna et al.
  ref <302>: TITLE: Evidence for a structural motif in toxins and interleukin-2 that may be responsible for binding to endothelial cells and initiating vascular leak syndrome
  ref <303>: JOURNAL: Proc. Natl. Acad. Sci. USA
  ref <304>: VOLUME: 96
  ref <306>: PAGES: 39573962
  ref <307>: DATE: 1999-03-01
GADDVADSSKSFVMENFSSYHGTKPGYADSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGN

SEQ ID NO: 197 - protein, 186 aa, Corynebacterium diphtheriae
  ref <301>: AUTHORS: Baluna et al.
  ref <302>: TITLE: Evidence for a structural motif in toxins and interleukin-2 that may be responsible for binding to endothelial cells and initiating vascular leak syndrome
  ref <303>: JOURNAL: Proc. Natl. Acad. Sci. USA
  ref <304>: VOLUME: 96
  ref <306>: PAGES: 39573962
  ref <307>: DATE: 1999-03-01
GADDVSKSFVMENFSSYHGTKPGYADSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGN

SEQ ID NO: 198 - protein, 251 aa, Gelonium multiflorum
  ref <301>: AUTHORS: Baluna et al.
  ref <303>: JOURNAL: Proc. Natl. Acad. Sci. USA
  ref <304>: VOLUME: 96
  ref <306>: PAGES: 39573962
  ref <307>: DATE: 1999-03-01
GLDTVSFSTKGATYITYVNFLNELRVKLKPEGNSHGIPLLRKKADDPGKAFVLVALSNDNGQLAEIAIDATSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPK

SEQ ID NO: 199 - protein, 247 aa, Trichosanthes kirilowii
  ref <301>: AUTHORS: An Q, et a
  ref <302>: TITLE: -
  ref <303>: JOURNAL: J Biomed Sci.
  ref <304>: VOLUME: 13
  ref <305>: ISSUE: 5
  ref <306>: PAGES: 637-43
  ref <307>: DATE: 2005-09-01
DVSFRLSGATSSSYGVFISNLRKALPNERKLYDIPLLRSSLPGSQRYALIHLTNYADETISVAIDVTNVYIMGYRAGDTSACSNEASATEAAKYVFKDAMRKVTLPYSGNYERLQTAAGKIRENIPLGLPALDSAITTLFYYNANSAASALMVLIQSTSEAARYKFIEQQIGCGVDKTFLPSLAIISLENSWSALSKQIQIASTNNGQFESPVVLINAQNQRVTITNVDAGVVTSNIALLLNRNNMA

SEQ ID NO: 200 - protein, 247 aa, Artificial Sequence
  synthesized, mutated variant of trichosantin
DASFRLSGATSSSYGVFISNLRKALPNERKLYDIPLLRSSLPGSQRYALIHLTNYADETISVAIDATNVYIMGYRAGDTSACSNEASATEAAKYVFKDAMRKVTLPYSGNYERLQTAAGKIRENIPLGLPAGDSAITTLFYYNANSAASALMVLIQSTSEAARYKFIEQQIGCGVDKTFLPSLAIISLENSWSALSKQIQIASTNNGQFESPVVLINAQNQRVTITNVDAGVVTSNIALLLNRNNMA

SEQ ID NO: 201 - protein, 342 aa, Artificial Sequence
  synthesuzed, mutated variant of P. aeruginosa exotoxin
PEGGSLAALTAHQACHLPLETFTRHRQPRGWEQLEQCGYPVQKLVALYLAARLSWNQVDQVIANALASPGSGGDLGEAIRESPEQARLALTLAAAESERFVRQGTGNDEAGAANGPADSGDALLERNYPTGAEFLGDGGDVSFSTRGTQQWTVERLLQAHRQLEEAGYVFVGYHGTFLEAAQSIVFGGVRARSQDLDAIWAGFYIAGDPALAYGYAQDQEPDAAGRIRNGALLRVYVPRSSLPGFYATSLTLAAPEAAGEVERLIGHPLPLRLDAITGPEESGGRLETILGWPLAERTVVIPSAIPTDPKNVGGDLDPSSIPDSEQAISALPDYASQPGKPP

SEQ ID NO: 202 - protein, 225 aa, Artificial Sequence
  sytnthesized, mutated variant of Pseudomonas aeruginosa exotoxin
RHRQPRGWEQLPTGAEFLGDGGDVSFSTRGTQNWTVERLLQAHRQLEEAGYVFVGYHGTFLEAAQSIVFGGVRARSQDLDAIWAGFYIAGDPALAYGYAQDQEPDAAGRIRNGALLRVYVPRSSLPGFYATSLTLAAPEAAGEVERLIGHPLPLRLDAITGPEESGGRLETILGWPLAERTVVIPSAIPTDPRNVGGDLDPSSIPDSEQAISALPDYASQPGKPP

SEQ ID NO: 203 - protein, 226 aa, Pseudomonas aeruginosa
  ref <301>: AUTHORS: Onda M et al.
  ref <302>: TITLE: -
  ref <303>: JOURNAL: Proc Natl Acad Sci U S A.
  ref <304>: VOLUME: 108
  ref <305>: ISSUE: 14
  ref <306>: PAGES: 5742-7
  ref <307>: DATE: 2011-04-05
RHRQPRGWEQLYPTGAEFLGDGGAVSFSTRGTQNWTVERLLQAHRQLEEGGYVFVGYHGTFLEAAQSIVFGGVRARSQDLDAIWAGFYIAGDPALAYGYAQDQEPDAAGRIRNGALLRVYVPRSSLPGFYATSLTLAAPEAAGEVERLIGHPLPLRLDAITGPEEAGGRLETILGWPLAERTVVIPSAIPTDPRNVGGDLDPSSIPDAEAAISALPDYASQPGKPP

SEQ ID NO: 204 - protein, 279 aa, Artificial Sequence
  sytnthesized, mutated sequence of Pseudomonas aeruginosa exotoxin
EQSGYPVQRLVALYLAARLSWNQVDQVIRNALASPGSGGDLGEAIREQPEQARLALTLAAAESEYPTGAEFLGDGGAVSFSTRGTQNWTVERLLQAHRQLEEGGYVFVGYHGTFLEAAQSIVFGGVRARSQDLDAIWAGFYIAGDPALAYGYAQDQEPDAAGRIRNGALLRVYVPRSSLPGFYATSLTLAAPEAAGEVERLIGHPLPLRLDAITGPEEAGGRLETILGWPLAERTVVIPSAIPTDPRNVGGDLDPSSIPDAEAAISALPDYASQPGKPP

SEQ ID NO: 205 - protein, 279 aa, Artificial Sequence; identical to SEQ ID NO: 204
  sytnthesized, mutated variant of Pseudomonas aeruginosa exotoxin

SEQ ID NO: 206 - protein, 214 aa, Pseudomonas aeruginosa
  ref <301>: AUTHORS: Weldon JE et al.
  ref <303>: JOURNAL: Blood
  ref <304>: VOLUME: 113
  ref <305>: ISSUE: 16
  ref <306>: PAGES: 3792-3800
  ref <307>: DATE: 2009-04-16
PTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPP

SEQ ID NO: 207 - protein, 279 aa, Artificial Sequence
  sytnthesized, mutated variant of Pseudomonas aeruginosa exotoxin
EQSGYPVQKLVALYLAARLSWNQVDQVIRNALASPGSGGDLGEAIREQPEQARLALTLAAAESEYPTGAEFLGDGGAVSFSTRGTQNWTVERLLQAHRQLEEGGYVFVGYHGTFLEAAQSIVFGGVRARSQDLDAIWAGFYIAGDPALAYGYAQDQEPDAAGRIRNGALLRVYVPRSSLPGFYATSLTLAAPEAAGEVERLIGHPLPLRLDAITGPEEAGGRLETILGWPLAERTVVIPSAIPTDPKNVGGDLDPSSIPDAEAAISALPDYASQPGKPP

SEQ ID NO: 208 - protein, 51 aa, Salmonella typhi
  ref <308>: DATABASE ACCESSION NUMBER: GenBank/1209200A
  ref <309>: DATABASE ENTRY DATE: 1996-10-01
  ref <313>: RELEVANT RESIDUES IN SEQ ID NO: (2)..(52)
KLPRSSLVWCVLIVCLTLLIFTYLTRKSLCEIRYRDGHREVAAFMAYESGK

(19) United States
(12) Patent Application Publication
(10) Pub. No.: US 2015/0044162 A1
(43) Pub. Date: Feb. 12, 2015

(54) ANTICANCER FUSION PROTEIN

(71) Applicant: ADAMED SP. Z.O.O., Czosnów k/Warszawy (PL)

(72) Inventors: Jerzy Szczepan Pieczykolan, Radecznica (PL); Sebastian Pawlak, Warszawa (PL); Michal Szymanik, Marki (PL); Anna Maria Pieczykolan, Warszawa (PL); Bartlomiej Maciej Zerek, Dabrowa (PL); Piotr Rózga, Skierniewice (PL); Albert Robert Jaworski, Warszawa (PL); Malgorzata Izabela Teska-Kaminska, Plochocin (PL)

(21) Appl. No.: 14/361,279

(22) PCT Filed: Nov. 28, 2012
(86) PCT No.: PCT/IB2012/056806

§ 371 Date: May 28, 2014

(30) Foreign Application Priority Data
Nov. 28, 2011 (PL) P.397167

(51) Int. Cl. C07K 14/81; C07K 14/525

(52) U.S. Cl. CPC C07K 14/81; C07K 14/525; C07K 2319/50; C07K 2319/55; C07K 2319/00; C07K 2319/04; A61K 38/00
USPC 424/85.1; 530/402

(57) ABSTRACT

A fusion protein comprising domain (a) which is a functional fragment of hTRAIL protein sequence, which fragment begins with an amino acid at a position not lower than hTRAIL95, or a homolog of said functional fragment having at least 70% sequence identity, preferably 85% identity and ending with the amino acid hTRAIL281; and domain (b) which is a sequence of an effector peptide inhibiting protein synthesis, wherein the sequence of domain (b) is attached at the C-terminus or N-terminus of domain (a). The fusion protein can be used for the treatment of cancer diseases.

The invention relates to the field of therapeutic fusion proteins, especially recombinant fusion proteins. More particularly, the invention relates to fusion proteins comprising the fragment of a sequence of the soluble human TRAIL protein and a sequence of a peptide toxin inhibiting protein synthesis, pharmaceutical compositions containing them, their use in therapy, especially as anticancer agents, and to polynucleotide sequences encoding the fusion proteins, expression vectors containing the polynucleotide sequences, and host cells containing these expression vectors.

TRAIL protein, a member of the cytokines family (Tumor Necrosis Factor-Related Apoptosis Inducing Ligand), also known as Apo2L (Apo2-ligand), is a potent activator of apoptosis in tumor cells and in cells infected by viruses. TRAIL is a ligand naturally occurring in the body. TRAIL protein, its amino acid sequence, coding DNA sequences and protein expression systems were disclosed for the first time in EP0835305A1.

TRAIL protein exerts its anticancer activity by binding to pro-apoptotic surface TRAIL receptors 1 and 2 (TRAIL-R1 1R2) and subsequent activation of these receptors. These receptors, also known as DR4 and DR5 (death receptor 4 and death receptor 5), are members of the TNF receptor family and are overexpressed by different types of cancer cells. Activation of these receptors can induce external signaling pathway of suppressor gene p53-independent apoptosis, which by activated caspase-8 leads to the activation of executive caspases and thereby degradation of nucleic acids. Caspase-8 released upon TRAIL activation may also cause the release of truncated Bid protein, which is as translocated to mitochondria, where it stimulates the release of cytochrome c, thus indirectly amplifying the apoptotic signal from death receptors.

TRAIL acts selectively on tumor cells essentially without inducing apoptosis in healthy cells which show resistance to this protein. Therefore, the enormous potential of TRAIL was recognized as an anticancer agent which acts on a wide range of different types of tumor cells, including hematologic malignancies and solid tumors, while sparing normal cells and exerting potentially relatively little side effects.

TRAIL protein is a type II membrane protein having the length of 281 amino acids, and its extracellular region comprising amino acid residues 114-281 upon cleavage by proteases forms soluble sTRAIL molecule of 20 kDa size, which is also biologically active. Both forms, TRAIL and sTRAIL, are capable of triggering apoptosis via interaction with TRAIL receptors present on target cells. Strong antitumor activity and very low systemic toxicity of soluble part of TRAIL molecule was demonstrated using cell lines tests. Also, preliminary human clinical studies with recombinant human soluble TRAIL (rhTRAIL) having amino acid sequence corresponding to amino acids 114-281 of hTRAIL, known under the INN dulanermin, showed its good tolerance and absence of dose limiting toxicity.

Fragments of TRAIL shorter than 114-281 are also able to bind with membrane death receptors and induce apoptosis via these receptors, as recently reported for recombinant circularly permuted mutant of 122-281hTRAIL for example in EP 1 688 498.

Toxic effects of recombinant TRAIL protein on liver cells reported up to now appear to be associated with the presence of modification, i.e. polyhistidine tags, while untagged TRAIL showed no systemic toxicity.

However, in further clinical trials on patients the actual effectiveness of TRAIL as a monotherapy proved to be low. Also problematic was primary or acquired resistance to TRAIL shown by many cancer cells (see for example WO2007/022214). Resistance may be due to various mechanisms and may be specific for a cancer type or patient-dependent (Thorburn A, Behbakht K, Ford H. TRAIL receptor-targeted therapeutics: resistance mechanisms and strategies to avoid them. Drug Resist Updat 2008; 11: 17-24). This resistance limits the usefulness of TRAIL as an anticancer agent. Although the mechanism of resistance to TRAIL has not been fully understood, it is believed that it may manifest itself at different levels of TRAIL-induced apoptosis pathway, ranging from the level of cell surface receptors to the executive caspases within the signaling pathway.

To overcome this low efficiency and the resistance of tumors to TRAIL, various combination therapies with radio- and chemotherapeutic agents were designed, which resulted in synergistic apoptotic effect (WO2009/002947; A. Almasan and A. Ashkenazi, Cytokine Growth Factor Reviews 14 (2003) 337-348; R K Srivastava, Neoplasis, Vol 3, No. 6, 2001, 535-546, Soria J C et al., J. Clin. Oncology, Vol 28, No. 9 (2010), p. 1527-1533). The use of rhTRAIL for cancer treatment in combination with selected conventional chemotherapeutic agents (paclitaxel, carboplatin) and monoclonal anti-VEGF antibodies are described in WO2009/140469. However, such a combination necessarily implies well-known deficiencies of conventional chemotherapy or radiotherapy. Prior art is silent, however, about any data suggesting abolishing of cell resistance to TRAIL obtained by fusing TRAIL protein with other proteins or fragments thereof.

Moreover, the problem connected with TRAIL therapy appeared to be its low stability and rapid elimination from the body after administration.

Anticancer therapies may also be directed to the inhibition of tumor cell protein synthesis. The beneficial effect of inhibiting tumor cell proliferation by inhibiting the intracellular protein synthesis is known. Attempts are being made of clinical use of substances that inhibit or regulate the process of protein synthesis, both as a cancer therapy and complementary cancer therapy.

Substances that inhibit the synthesis of cellular protein are catalytic peptides or protein toxins of bacterial, fungal or plant origin. Single-chain toxins (also known as hemitoxins), possessing a catalytic domain only and lacking a binding domain are as such in their free native form practically non-toxic to cells. Toxins consisting of two or more chains (also known as holotoxins) possess in addition to the catalytic domain also the binding domain, but lacking the cellular selectivity and therefore after systemic administration exhibit undesirable toxicity against healthy tissues and extensive side effects.

To achieve higher specificity, toxins or catalytic domains of protein toxins are conjugated to carriers—ligands selectively binding to the markers present on the tumor cell. The use of a domain or a ligand targeting protein allows specific delivery of the toxic domain of a protein to a cell. Immunotoxins are conjugate or fusion proteins, in which a toxin is linked to a binding ligand, which is an immune system protein, such as antibodies, growth factors, interleukins, and tumor necrosis factor. There are known conjugates of growth factors VEGF, FGF, and PDGF with toxins from the group of ribosome inactivating protein (RIP toxins), conjugates of TNF with RIP toxins, conjugates of IL-2 with Pseudomonas exotoxin, conjugates of IL-13 with Psuedomonas exotoxin as well as used in treatment preparation Ontake® containing conjugate IL2-diphtheria toxin. Other examples are conjugates of toxins such as gelonin and abrin with integrin, fibronectin, I-CAM and granzyme B, as well as conjugate of ebulin with transferrin (Hall, W. A. Targeted toxin therapy for malignant astrocytoma. Neurosurgery 2000, 46, 544-551). In WO2002/069886 and US2003176331 there is mentioned the possibility of conjugation of gelonin RIP toxin with a second polypeptide for targeted delivery of the toxin. Among many possible types of such secondary polypeptides the TRAIL protein is mentioned, however any details concerning the structure and properties of this type of chimeras are disclosed.

In WO2008052322 there is mentioned the possibility of use non-immunoglobulin polypeptides that bind to cell surface structures as carriers of RIP toxins. In WO2008080218 there is noted that a cytokine, including as one of many listed TRAIL, can act as a carrier for modified toxins, the description lacks any information that would be allow to define a therapeutically effective molecule comprising TRAIL and a toxin and its properties.

U.S. Pat. No. 6,627,197 describes a construct comprising a toxin inactivating protein synthesis, a peptide cleavable by HIV protease, a lectin as a element binding to the cell surface, a targeting fragment and the hydrophobic agent, to be applied as an antiviral agent.

In the prior art there is also known the use in chimeric proteins of cleavage sites recognized by specific proteases enabling the release of toxins in the tumor environment and consequently their internalization into the tumor cell. For example, U.S. Pat. No. 7,252,993 discloses chimeric proteins containing a toxic fragment of ricin and targeting peptide—DP178 chemokine, connected via linker recognized by a HIV protease. This description, however, does not provide detailed information on the structure, properties and application of TRAIL-toxin chimeras.

The present invention provides a novel fusion proteins that combine toxic properties of peptide toxins as effector peptides and pro-apoptotic properties and specific targeting to the structures present on cancer cell of TRAIL protein.

Fusion proteins of the invention comprise binding domain derived from TRAIL and peptide toxin domain as an effector peptide having protein synthesis inhibition properties.

Due to the presence of a domain derived from hTRAIL, proteins according to the invention are directed selectively to cancer cells, wherein the elements of the protein exert their effects.

In particular, peptide toxins as the effector peptides inhibit protein synthesis process in the cancer cell. Delivery of the protein of the invention into the tumor environment allows minimization of toxicity and side effects against healthy cells in the body, as well as reduction of the frequency of administration, In addition, targeted therapy with the use of proteins according to the invention allows to avoid the problem of low efficiency of previously known nonspecific therapies based on the protein synthesis inhibition caused by high toxicity and by necessity of administering high doses.

It turned out that in many cases fusion proteins of the invention are more potent than soluble hTRAIL and its variants including the fragment of a sequence. Until now, effector peptides used in the fusion protein of the invention have not been used in medicine as such because of unfavorable kinetics, rapid degradation by nonspecific proteases or accumulation in the body caused by lack of proper sequence of activation of pathways, which is necessary to enable the proper action of the effector peptide at target site. Incorporation of the effector peptides into the fusion protein allows their selective delivery to the site where their action is desirable. Furthermore, the attachment of the effector peptide increases the mass of protein, resulting in prolonged half-life and increased retention of protein in the tumor and its enhanced efficiency. Additionally, in many cases, novel fusion proteins also overcome natural or induced resistance to TRAIL.

### DESCRIPTION OF FIGURES

The invention will now be described in detail with reference to the Figures of the drawing, wherein

FIG. 1 presents tumor volume changes (% of initial stage) in HsdCpb:NMRI-Foxn1 nin mice burdened with colon cancer Colo 205 treated with fusion protein of the invention of Ex. 18^{a}, Ex. 25^{a}, Ex. 37^{a} and Ex. 42^{a} compared to rhTRAIL114-281;

FIG. 2 presents tumor growth inhibition values (% TGI) in HsdCpb:NMRI-Foxn1 nin mice burdened with colon cancer Colo 205 treated with fusion protein of the invention of Ex. 18^{a}, Ex. 25^{a}, Ex. 37^{a} and Ex. 42^{a} compared to rhTRAIL114-281;

FIG. 3 presents tumor volume changes (% of initial stage) in Cby.Cg-foxn1(nu)/J mice burdened with lung cancer A549 treated with fusion protein of the invention of Ex. 18^{a} and Ex. 35^{a} compared to rhTRAIL114-281;

FIG. 4 presents tumor growth inhibition values (% TGI) in Cby.Cg-foxn1(nu)/J mice burdened with lung cancer A549 treated with fusion protein of the invention of Ex. 18^{a} and Ex. 35^{a} compared to rhTRAIL114-281;

FIG. 5 presents tumor volume changes (% of initial stage) in Cby.Cg-foxn1(nu)/J mice burdened with lung cancer A549 treated with fusion protein of the invention of Ex. 18^{a} and Ex. 50^{a} compared to rhTRAIL114-281;

FIG. 6 presents tumor growth inhibition values (% TGI) in Cby.Cg-foxn1(nu)/J mice burdened with lung cancer A549 treated with fusion protein of the invention of Ex. 18^{a} and Ex. 50^{a} compared to rhTRAIL114-281;

FIG. 7 presents tumor volume changes (% of initial stage) inCrl:SHO-Prkdc^{scid}Hr^{hr} burdened with lung cancer A549 treated with fusion protein of the invention of Ex. 2^{a}, Ex. 18^{a} and Ex. 44^{a} compared to rhTRAIL114-281;

FIG. 8 presents tumor growth inhibition values (% TGI) in Crl:SHO-Prkdc^{scid}Hr^{hr} mice burdened with lung cancer A549 treated with fusion protein of the invention of Ex. 2^{a}, Ex. 18^{a} and Ex. 44^{a} compared to rhTRAIL114-281;

FIG. 9 presents tumor volume changes (% of initial stage) in Crl:SHO-Prkdc^{scid}Hr^{hr} mice burdened with lung cancer A549 treated with fusion protein of the invention of Ex. 20^{a}, Ex. 26^{a}, Ex. 43^{a} and Ex. 47^{a} compared to rhTRAIL114-281;

FIG. 10 presents tumor growth inhibition values (% TGI) in Crl:SHO-Prkdc^{scid}Hr^{hr} mice burdened with lung cancer A549 treated with fusion protein of the invention of Ex. 20^{a}, Ex. 26^{a}, Ex. 43^{a} and Ex. 47^{a} compared to rhTRAIL114-281;

FIG. 11 presents tumor volume changes (% of initial stage) in Crl:SHO-Prkdc^{scid}Hr^{hr} mice burdened with pancreas cancer PANC-1 treated with fusion protein of the invention of Ex. 20^{a}, Ex. 51^{a} and Ex. 52^{a} compared to rhTRAIL114-281;

FIG. 12 presents tumor growth inhibition values (% TGI) in Crl:SHO-Prkdc^{scid}Hr^{hr} mice burdened with pancreas cancer PANC-1 treated with fusion protein of the invention of Ex. 20^{a}, Ex. 51^{a} and Ex. 52^{a} compared to rhTRAIL114-281;

FIG. 13 presents tumor volume changes (% of initial stage) in Crl:SHO-Prkdc^{scid}Hr^{hr} mice burdened with pancreas cancer PANC-itreated with fusion protein of the invention of Ex. 18^{a} and Ex. 44^{a} compared to rhTRAIL114-281;

FIG. 14 presents tumor growth inhibition values (% TGI) in Crl:SHO-Prkdc^{scid}Hr^{hr} mice burdened with pancreas cancer PANC-1 treated with fusion protein of the invention of Ex. 18^{a} and Ex. 44^{a} compared to rhTRAIL114-281;

FIG. 15 presents tumor volume changes (% of initial stage) in Cby.Cg-foxn1(nu)/J mice burdened with prostate cancer PC3 treated with fusion protein of the invention of Ex. 18^{a};

FIG. 16 presents tumor growth inhibition values (% TGI) in Cby.Cg-foxn1(nu)/J mice burdened with prostate cancer PC3 treated with fusion protein of the invention of Ex. 18^{a};

FIG. 17 presents tumor volume changes (% of initial stage) in Crl:SHO-Prkdc^{scid}Hr^{hr} mice burdened with liver cancer PCL/PRF/5 treated with fusion protein of the invention of Ex. 51^{a} compared to rhTRAIL114-281;

FIG. 18 presents tumor growth inhibition values (% TGI) in Crl:SHO-Prkdc^{scid}Hr^{hr} mice burdened with liver cancer PCL/PRF/5 treated with fusion protein of the invention of Ex. 51^{a} compared to rhTRAIL114-281;

FIG. 19 presents tumor volume changes (% of initial stage) in Crl:SHO-Prkdc^{scid}Hr^{hr} mice burdened with colon cancer HCT116 treated with fusion proteins of the invention of Ex. 18^{b} and Ex. 2^{b} compared to rhTRAIL114-281;

FIG. 19a presents tumor volume changes (% of initial stage) in Crl:SHO-Prkdc^{scid}Hr^{hr} mice burdened with colon cancer HCT116 treated with fusion protein of the invention of Ex. 18^{b} compared to rhTRAIL114-281;

FIG. 20 presents tumor growth inhibition values (% TGI) in Crl:SHO-Prkdc^{scid}Hr^{hr} mice burdened with colon cancer HCT116 treated with fusion proteins of the invention of Ex. 18^{b} and Ex. 2^{b} compared to rhTRAIL114-281;

FIG. 20a presents tumor growth inhibition values (% TGI) in Crl:SHO-Prkdc^{scid}Hr^{hr} mice burdened with colon cancer HCT116 treated with fusion protein of the invention of Ex. 18^{b} compared to rhTRAIL114-281;

FIG. 21 presents tumor volume changes (% of initial stage) in Crl:SHO-Prkdc^{scid}Hr^{hr} mice burdened with colon cancer SW620 treated with fusion proteins of the invention of Ex. 18^{b}, Ex. 2^{b} and Ex. 54^{b} compared to rhTRAIL114-281;

FIG. 21a presents tumor volume changes (% of initial stage) in Crl:SHO-Prkdc^{scid}Hr^{hr} mice burdened with colon cancer SW620 treated with fusion protein of the invention of Ex. 18^{b} compared to rhTRAIL114-281;

FIG. 22 presents tumor growth inhibition values (% TGI) in Crl:SHO-Prkdc^{scid}Hr^{hr} mice burdened with colon cancer HCT116 treated with fusion proteins of the invention of Ex. 18^{b}, Ex. 2^{b} and Ex. 54^{b} compared to rhTRAIL114-281;

FIG. 22a presents tumor growth inhibition values (% TGI) in Crl:SHO-Prkdc^{scid}Hr^{hr} mice burdened with colon cancer HCT116 treated with fusion protein of the invention of Ex. 18^{b} compared to rhTRAIL114-281;

FIG. 23 presents tumor volume changes (% of initial stage) in Crl:SHO-Prkdc^{scid}Hr^{hr} mice burdened with colon cancer HT-29 treated with fusion proteins of the invention of Ex. 18^{b} and Ex. 51^{b} compared to rhTRAIL114-281;

FIG. 24 presents tumor growth inhibition values (% TGI) in Crl:SHO-Prkdc^{scid}Hr^{hr} mice burdened with colon cancer HT-29 treated with fusion proteins of the invention of Ex. 18^{b} and Ex. 51^{b} compared to rhTRAIL114-281;

FIG. 25 presents tumor volume changes (% of initial stage) in Crl:SHO-Prkdc^{scid}Hr^{hr} mice burdened with liver cancer HepG2 treated with fusion protein of the invention of Ex. 18^{b} compared to rhTRAIL114-281;

FIG. 26 presents tumor growth inhibition values (% TGI) in Crl:SHO-Prkd^{scid}Hr^{hr} mice burdened with liver cancer HepG2 treated with fusion protein of the invention of Ex. 18^{b} compared to rhTRAIL114-281;

FIG. 27 presents tumor volume changes (% of initial stage) in Crl:SHO-Prkdc^{scid}Hr^{hr} mice burdened with lung cancer A549 treated with fusion proteins of the invention of Ex. 18^{b} and Ex. 2^{b} compared to rhTRAIL114-281;

FIG. 28 presents tumor growth inhibition values (% TGI) in Crl:SHO-Prkdc^{scid}Hr^{hr} mice burdened with lung cancer A549 treated with fusion proteins of the invention of Ex. 18^{b} and Ex. 2^{b} compared to rhTRAIL114-281;

FIG. 29 presents tumor volume changes (% of initial stage) in Crl:SHO-Prkdc^{scid}Hr^{hr} mice burdened with uterine sarcoma MES-SA/Dx5 treated with fusion protein of the invention of Ex. 18^{b} compared to rhTRAIL114-281;

FIG. 29 a presents tumor volume changes (% of initial stage) in Crl:SHO-Prkdc^{scid}Hr^{hr} mice burdened with uterine sarcoma MES-SA/Dx5 treated with fusion proteins of the invention of Ex. 18^{b}, Ex. 2^{b} and Ex. 51^{b} compared to rhTRAIL114-281;

FIG. 30 presents tumor growth inhibition values (% TGI) in Crl:SHO-Prkdc^{scid}Hr^{hr} mice burdened with uterine sarcoma MES-SA/Dx5 treated with fusion protein of the invention of Ex. 18^{b} compared to rhTRAIL114-281; and

FIG. 30a presents tumor growth inhibition values (% TGI) in Crl:SHO-Prkdc^{scid}Hr^{hr} mice burdened with uterine sarcoma MES-SA/Dx5 treated with fusion proteins of the invention of Ex. 18^{b}, Ex. 2^{b} and Ex. 51^{b} compared to rhTRAIL114-281.

### DETAILED DESCRIPTION OF THE INVENTION

The invention relates to a fusion protein comprising:
domain (a) which is a functional fragment of the sequence of soluble hTRAIL protein, which fragment begins with an amino acid at a position not lower than hTRAIL95 or a homolog of said functional fragment having at least 70% sequence identity, preferably 85% identity and ending with the amino acid hTRAIL281, and
at least one domain (b) which is the sequence of an effector peptide inhibiting protein synthesis, wherein the sequence of the domain (b) is attached at the C-terminus and/or N-terminus of domain (a), and wherein the fusion protein does not contain a domain binding to the carbohydrate receptors on the cell surface.

The term “the functional soluble fragment of a sequence of soluble hTRAIL” should be understood as denoting any such fragment of soluble hTRAIL, i.e. that is capable of inducing apoptotic signal in mammalian cells upon binding to its receptors on the surface of the cells.

It will be also appreciated by a skilled person that the existence of at least 70% or 85% homology of the TRAIL sequence is known in the art.

It should be understood that domain (b) of the effector peptide in the fusion protein of the invention is neither hTRAIL protein nor a part or fragment of hTRAIL protein.

The term “peptide” in accordance with the invention should be understood as a molecule built from plurality of amino acids linked together by means of a peptide bond. Thus, the term “peptide” according to the invention includes oligopeptides, polypeptides and proteins.

In the present invention the amino acid sequences of peptides will be presented in a conventional manner adopted in the art in the direction from N-terminus (N-end) of the peptide towards its C-terminus (C-end). Any sequence will thus have its N-terminus on the left side and C-terminus on the right side of its linear presentation.

The term TRAIL preceded by a number is used in the present specification to denote an amino acid having this number in the known sequence of hTRAIL.

The fusion protein of the invention incorporates at least one domain (b) of the effector peptide, attached at the C-terminus and/or or at the N-terminus of domain (a).

In a particular embodiment, domain (a) is the fragment of hTRAIL sequence, beginning with an amino acid from the range of hTRAIL95 to hTRAIL121, inclusive, and ending with the amino acid hTRAIL 281.

In particular, domain (a) may be selected from the group consisting of sequences corresponding to hTRAIL95-281, hTRAIL114-281, hTRAIL116-281, hTRAIL119-281, hTRAIL120-281 and hTRAIL121-281. It will be evident to those skilled in the art that hTRAIL95-281, hTRAIL114-281, hTRAIL116-281, hTRAIL119-281, hTRAIL120-281 and hTRAIL121-281 represent a fragment of human TRAIL protein starting with amino acid marked with the number 95, 114, 116, 119, 120 and 121, respectively, and ending with the last amino acid 281, in the known sequence of hTRAIL published in GenBank under Accession No. P50591 and presented in the sequence listing of the present invention as SEQ. No. 141.

In another particular embodiment, domain (a) is a homolog of the functional fragment of soluble hTRAIL protein sequence beginning at amino acid position not lower than hTRAIL95 and ending at amino acid hTRAIL281, the sequence of which is at least in 70%, preferably in 85%, identical to original sequence.

In specific variants of this embodiment domain (a) is a homolog of the fragment selected from the group consisting of sequences corresponding to hTRAIL95-281, hTRAIL114-281, hTRAIL116-281, hTRAIL119-281, hTRAIL120-281 and hTRAIL121-281.

It should be understood that a homolog of the hTRAIL fragment is a variation/modification of the amino acid sequence of this fragment, wherein at least one amino acid is changed, including 1 amino acid, 2 amino acids, 3 amino acids, 4 amino acids, 5 amino acids, 6 amino acids, and not more than 15% of amino acids, and wherein a fragment of the modified sequence has preserved functionality of the hTRAIL sequence, i.e. the ability of binding to cell surface death receptors and inducing apoptosis in mammalian cells. Modification of the amino acid sequence may include, for example, substitution, deletion and/or addition of amino acids.

Preferably, the homolog of hTRAIL fragment having modified sequence shows a modified affinity to the death receptors DR4 (TRAIL-R1) or DR5 (TRAIL-R2) in comparison with the native fragment of hTRAIL.

The term “modified affinity” refers to an increased affinity and/or affinity with altered receptor selectivity.

Preferably, the homolog of the fragment of hTRAIL having modified sequence shows increased affinity to the death receptors DR4 and DR5 compared to native fragment of hTRAIL.

Particularly preferably, the homolog of fragment of hTRAIL having modified sequence shows increased affinity to the death receptor DR5 in comparison with the death receptor DR4, i.e. an increased selectivity DR5/DR4.

Also preferably, the homolog of fragment of hTRAIL having modified sequence shows an increased selectivity towards the death receptors DR4 and/or DR5 in relation to the affinity towards the receptors DR1 (TRAIL-R3) and/or DR2 (TRAIL-R4).

Modifications of hTRAIL resulting in increased affinity and/or selectivity towards the death receptors DR4 and DR5 are known to those skilled in the art, for example from the publication Tur V, van der Sloot A M, Reis C R, Szegezdi E, Cool R H, Samali A, Serrano L, Quax W J. DR4-selective tumor necrosis factor-related apoptosis-inducing ligand (TRAIL) variants obtained by structure-based design. J. Biol. Chem. 2008 Jul. 18; 283(29):20560-8, which describes the D218H mutation having increased selectivity towards DR4, or Gasparian M E, Chernyak B V, Dolgikh D A, Yagolovich A V, Popova E N, Sycheva A M, Moshkovskii S A, Kirpichnikov M P. Generation of new TRAIL mutants DR5-A and DR5-B with improved selectivity to death receptor 5, Apoptosis. 2009 June; 14(6):778-87, which describes the D269H mutation having a reduced affinity towards DR4. hTRAIL mutants resulting in increased affinity towards one receptor selected from the DR4 and DR5 comparing with DR1 and DR2 receptors and increased affinity towards the receptor DR5 comparing with DR4 are also described in WO2009077857 and WO2009066174.

Suitable mutations are one or more mutations in the positions of native hTRAL selected from the group consisting of amino acid 131, 149, 159, 193, 199, 201, 204, 204, 212, 215, 218 and 251, in particular, mutations involving the substitution of an amino acid with a basic amino acid such as lysine, histidine or arginine, or amino acid such as glutamic acid or aspargic acid. Particularly one or more mutations selected from the group consisting of G131R, G131K, R149I, R149M, R149N, R149K, S159R, Q193H, Q193K, N199H, N199R, K201H, K201R, K204E, K204D, K204L, K204Y, K212R, S215E, S215H, S215K, S215D, D218Y, D218H, K251D, K251E and K251Q, as described in WO2009066174, may be specified.

Suitable mutations are also one or more mutations in the positions of native hTRAIL selected from the group consisting of amino acid 195, 269 and 214, particularly mutations involving the substitution of an amino acid with a basic amino acid such as lysine, histidine or arginine. Particularly one or more mutations selected from the group consisting of D269H, E195R, and T214R, as described in WO2009077857, may be specified.

In a particular embodiment, the domain (a) which is a homolog of the fragment of hTRAIL is selected from D218H mutant of the native TRAIL sequence, as described in WO2009066174, or the Y189N-R191K-Q193R-H264R-1266R-D269H mutant of the native TRAIL sequence, as described in Gasparian ME et al. Generation of new TRAIL mutants DR5-A and DR5-B with improved selectivity to death receptor 5, Apoptosis. 2009 June; 14(6): 778-87.

Domain (a), i.e. the fragment of TRAIL, is a domain responsible for binding of the construct of the fusion protein to death receptors on the surface of a cell. Furthermore, domain (a) upon binding will exert its known agonistic activity, i.e. activation of extrinsic pathway of apoptosis.

The fusion protein of the invention does not comprise sequences of domains capable of binding to carbohydrate receptors on the cell surface. Binding to carbohydrate receptors on the cell surface is a non-specific binding.

In particular, the fusion protein of the invention does not comprise sequences of lectin domains (glycoproteins) capable of binding to sugar receptors on the cell surface. By lectin domain capable of binding to carbohydrate receptors on the cell surface should be understood, in particular, both the subunits (chains) A of protein toxins and fragments thereof, as well as lectin proteins occurring alone unaccompanied by domains of a different functionality, including the enzymatic functionality.

In another embodiment, the fusion protein of the invention, except of domain (a), does not include any other domain binding to receptors on the cell surface.

Domain (b) of the fusion protein of the invention is a domain of an effector peptide—a peptide toxin that inhibits protein synthesis process within the cell.

The effector peptide of domain (b) of the fusion protein of the invention may be a toxin inhibiting protein synthesis by inhibition of the stage of translation of the protein synthesis process in the cell.

The effector peptide of domain (b) of the fusion protein of the invention may be a toxin inhibiting protein synthesis by inhibition of transcription and RNA production of the protein synthesis proces in the cell.

In one embodiment the peptide toxin is a peptide inhibiting enzymatically translation of protein at the rybosome level. In this embodiment of the invention, in one of variants the peptide toxin possesses the enzymatic catalytic activity selected from the activity of N-glycosidase, ribonuclease and ADP-ribosyltransferase.

It should be understood, as will be apparent to those skilled in the art, that the peptide toxin, in addition to its main activity as an effector peptide, may possess one or more other activities which may result in the inhibition of protein synthesis in cells, as described for example in W. J. Pneumans et al., The FASEB Journal, 2001, Vol. 15, str. 1493-1506.

Effector peptides with N-glycosidase activity perform modification (depurination) of ribosome by truncation of one specific adenine residue in the subunit 60 of 285 rRNA. This modification is irreversible and prevents the binding of the ribosome with a translational factor EF, thus blocking translation.

Effector peptides having catalytic activity of N-glycosidase can be selected from the group peptide toxins consisting of type 1 ribosome inactivating protein (RIP) (hemitoxins), catalytic subunits (chains) A of type 2 RIP proteins (holotoxins), and their modification with preserved N-glycosidase activity of at least 85% sequence identity with the original sequence.

Type 1 RIP toxins with N-glycosidase activity are single-chain proteins and have a catalytic domain only.

The following known toxins of plant origin may be mentioned as specific effector peptides from the group of single-chain type 1 RIP toxins: gelonin (from Gelonium multiflorum), momordin (protein isolated from plants of the genus Momordica), saporin (from Saponaria Officinalis), dodekandrin (from Phytolacca dodecandra), bouganin (from Bougainvillea spectabilis), PAP protein from pokeweed (Phytolacca Americana), trichosantin (from Trichosanthes kirilowii), trichoanguin (from Trichosanthes anguina), agrostin (from Agrostemma githago), diantrin, luffin P1 (from Luffa cylindrica), momorcharin (from Momordica charantia) and tritin.

Exemplary sequences of the effector peptide in this embodiment are designated as SEQ. No. 55 (bouganin), SEQ. No. 58 (PAP toxin homologue), SEQ. No. 59 (fragment of saporin), SEQ. No. 60 (trichosantin), SEQ. No. 61 (trichoanguin), SEQ. No. 65 (tuffin P1), SEQ. No. 67 (momorcharin), and SEQ. No. 78 (catalytic domain of gelonin).

Further examples of the effector peptide in this embodiment are analogs of gelonin (SEQ. No. 198) and analogs of trichosantin with modified native sequence (SEQ. No. 199 and SEQ. No. 200).

One example of modified trichosantin is SEQ. No. 199, wherein known sequence of trichosantin was modified to lower the immunogenicity of the toxin. Namely, in the known sequence of trichasantin “YFF”81-83 motif was replaced by “ACS”, analogously “KR” 173-174 amino acids were replaced by “CG” residues (the amino acids residues numbers are consistent with the sequence published in GenBank: AAB22585.1) (An Q, Wei S, Mu S, Zhang X, Lei Y. Zhang W, Jia N, Cheng X, Fan A, Li Z, Xu Z. J Biomed Sci.2006 September; 13(5):637-43)).

Further example of modified trichosantin is SEQ. No. 200, wherein known sequence of trichosantin was modified in the following manner. Namely, “YFF” 81-83 motif was replaced by “ACS” to lower the immunogenicity of the toxin, “KR” 173-174 amino acids were replaced by “CG” residues (An Q Wei S, Mu S, Zhang X, Lei Y. Zhang W, Jia N, Cheng X, Fan A, Li Z, Xu Z. J Biomed Sci.2006 September; 13(5):637-43) to reduce the VLS (vascular leak syndrome) problem, the valine residues −2 and 66 were replaced by alanine; and leucine 132 was replaced by gycine (the amino acids residues numbers are consistent with the sequence published in GenBank: AAB22585.1) (Baluna R, Rizo J. Gordon B E, Ghetie V, Vitetta E S. Proc Natl Acad Sci USA. 1999 Mar. 30; 96(7):3957-62)). Gelonin analog with mutation V70A of SEQ. No. 198 is known and described in the literature (Baluna et al. Proc. Natl. Acad. Sci. USA, Vol. 96, pp. 3957-3962. March 199). Trichosantin analog designated as SEQ. No. 199 is known and described in the literature (An Q, et al. J Biomed Sci. 2006 September; 13(5):637-43). Trichosantin analog designated as SEQ. No. 200 is novel and was not described in the literature.

Type 2 RIP toxins with N-glycosidase activity are two-chains proteins and have catalytic domain (subunit A) and lectin binding domain (subunit B) capable of binding to the carbohydrate (sugar) receptors present on the cell surface. According to the invention, catalytic subunits A of type 2 RIP toxins, devoid of lectin binding domain, may be used as effector peptides.

As effector peptides of this type catalytic subunits A of the following plant toxins can be mentioned: ricin (from Ricinnus communis), abrin (from Abbrus precatrius), modeccin (from Adenia digitata), viscumin (a toxin from misletoe Viscum album), volkensin (from Adenia volkensii), ebulin 1 (from Sambucus ebulus), nigrin b (from Sambucus nigra) and bacterial toxin Shiga (from Shigella dysenteriae), or modifications thereof with preserved N-glycosidase activity of at least 85% sequence identity with the original sequence.

Exemplary sequences of effector peptides in this embodiment are designated as SEQ. No. 56 and SEQ. No. 57 (subunit A of ricin); and a variant subunit A of ricin), SEQ. No. 195 (modified subunit A of ricin); SEQ. No. 62 (subunit A of misletoe toxin), SEQ. No. 63 (subunit A of ebulin 1), SEQ. No. 64 (subunit A of nigrin b), SEQ. No. 66 (subunit A of volkensin), SEQ. No. 70 (a wariant of Shiga toxin subunit A), and SEQ. No. 82 (subunit A of abrin); SEQ. No. 194 (modified subunit A of abrin as described in Baluna et al. Proc. Natl. Acad. Sci. USA, Vol. 96, pp. 3957-3962, March 1999 with mutations V71A, G115A and S232Q, the amino acids residues numbers being consistent with the sequence published in GenBank CAA38655.1).

Exemplary sequences of effector peptides in this embodiment are designated as SEQ. No. 56 and SEQ. No. 57 (subunit A of ricin and a variant subunit A of ricin), SEQ. No. 195 (modified subunit A of ricin as described in Baluna et al. Proc. Natl. Acad. Sci. USA, Vol. 96, pp. 3957-3962, March 1999, with deletion 78 LDV 80, the amino acids residues numbers being consistent with the sequence published in GenBank ABG65738.1); SEQ. No. 62 (subunit A of misletoe toxin), SEQ. No. 63 (subunit A of ebulin 1), SEQ. No. 64 (subunit A of nigrin b), SEQ. No. 66 (subunit A of volkensin), SEQ. No. 70 (a variant of Shiga toxin subunit A), and SEQ. No. 82 (subunit A of abrin); SEQ. No. 194 (modified subunit A of abrin as described in Baluna et at. Proc. Natl. Acad. Sci. USA Vol. 96, pp. 395T3962, March 1999; with mutations V71A, G115A and S233Q, the amino acids residues numbers being consistent with the sequence published in GenBank CAA38655.1

Effector peptides with catalytic activity of ribonuclease (also referred to as ribo-toxins) belong to endonucleases and cleave phosphodiester bonds in 285 rRNA, thereby leading to inhibition of the ribosome and stopping translation. As effec for peptides of this group may be mentioned fungal toxins alpha-sacrin, mitogillin, restrictocin from Aspergillus restrictus, and hirsutelin (from Hirsutella thompsonii).

Exemplary sequences of the effector peptide in this embodiment are designated as SEQ. No. 71 (restrictocin) and SEQ. No. 72 (hirsutellin).

Effector peptides with catalytic activity of ADP-ribosyltransferase cause ADP-ribosylation and thus inactivation of the components of protein synthesis machinery, mainly elongation/translation factor EF-2, and inhibition of translation. To this group of effector peptides belong catalytic domains of diphtheria toxin from Corynebacterium diphtheriae, exotoxin A from Pseudomonas aeruginosa, and modifications thereof with preserved ADP-ribosyltransferase activity of at least 85% sequence identity with the original sequence.

Modifications of catalytic domain of Pseudomonas aeruginosa exotoxin A and diphteria toxin may exemplary comprise truncation of the terminal fragment of the peptide, as well as substitutions or deletions in the catalytic domain or fragments thereof. Some of suitable substitutions and deletions are disclosed in Weldon J E et al.. Blood. 2009 Apr. 16; 113(16):3792-800; Onda M et al.. Proc Natl Acad Sci USA. 2011 Apr. 5; 108(14):5742-7.

Exemplary sequences of effector peptides in this embodiment are known Pseudomonas aeruginosa exotoxin catalytic domain A designated as SEQ. No. 69 (native sequence of catalytic domain A), and its mutated analogs designated as SEQ. No. 68; SEQ. No. 83; SEQ. No. 84; SEQ. No. 201; SEQ. No. 202; SEQ. No. 203; SEQ. No. 204; SEQ. No. 205; SEQ. No. 206; and SEQ. No. 207.

Exemplary sequences of effector peptides in this embodiment are known Pseudomonas aeruginosa exotoxin A designated as SEQ. No. 68, and its analogs designated as SEQ. No. 69; SEQ. No. 83; SEQ. No. 84; SEQ. No. 201; SEQ. No. 202; SEQ. No. 203; SEQ. No. 204; SEQ. No. 205; SEQ. No. 206; and SEQ. No. 207. Analogs of Pseudomonas aeruginosa exotoxin A designated as SEQ. No, 69, SEQ. No. 83, SEQ. No. 84, SEQ. No. 203 and SEQ. No. 206 are known and described in the literature.

Analogs of Pseudomonas aeruginosa exotoxin A designated as SEQ. No. 201; SEQ. No. 202; SEQ. No. 204; SEQ. No. 205; and SEQ. No. 207 are novel and are not described in the literature.

Known SEQ. No. 203 is a HA22-LR- 8M variant of Pseudomonas aeruginosa exotoxin A as described in Onda M et al., Proc Natl Acad Sci USA. 2011 Apr. 5; 108(14):5742-7 with 8 mutations reducing immunogenicity.

Known SEQ. No. 206 is a deletion variant HA22 -LR of Pseudomonas aeruginosa exotoxin A as described in Weldon J E et al.. Blood. 2009 Apr. 16; 113(16):3792-800.

Novel SEQ. No. 201 is an analog of Pseudomonas aeruginosa catalytic domain of exotoxin A, wherein three point mutations R318K, N441Q and R601K were introduced in the known sequence to reduce the immunogenicity (the amino acids residues numbers are consistent with the sequence published in GenBank AAB59097.1)

Novel SEQ. No. 202 is a deletion variant A2 -LR of Pseudomonas aeruginosa catalytic domain of exotoxin A as described in Weldon J E et al., Blood. 2009 Apr. 16; 113(16): 3792-800, with introduced further mutations lowering immunogenictity as described in Choe M, Webber K O, Pastan I. Cancer Res. 1994 Jul. 1; 54(13):3460-7 and other mutations as described in WO 2007/016150.

Novel SEQ. No. 204 is a variant of Pseudomonas aeruginosa catalytic domain of exotoxin A, which is a combination of variants HA22 M3 (deletion and mutation C312S) as described in Weldon J E et al.. Blood. 2009 Apr. 16; 113(16):3792-800 and variant HA22 8M with 8 mutations reducing immunogenicity described in Onda Metal. Proc Natl Acad Sci USA. 2011 Apr. 5; 108(14):5742-7).

Novel SEQ. No. 205 is a variant of Pseudomonas aeruginosa catalytic domain of exotoxin A which is a combination of variant HA22 M3 as described in Weldon J E et al.. Blood. 2009 Apr. 16; 113(16):3792-800, i.e. with deletion and mutation C312S, 8 mutations reducing immunogenicity as described in Onda M et al.. Proc Natl Acad Sci USA. 2011 Apr. 5; 108(14):5742-7, with further deletion of a region of cleavage site recognized by furin present in the native Pseudomonas aeruginosa toxin.

Novel SEQ. No. 207 is a variant of Pseudomonas aeruginosa catalytic domain of exotoxin A which is a combination of variant HA22 M3 described in Weldon J E et al.. Blood. 2009 Apr. 16; 113(16):3792-800, i.e. deletion and mutation C312S, variant HA22 8M described in Onda M et al.. Proc Natl Acad Sci USA. 2011 Apr. 5; 108(14):5742-7, i.e. 8 mutations reducing immunogenicity, and with additional mutation R601 K.

Other exemplary sequences of effector peptides in this embodiment are known subunit A of diphteria toxin (catalytic domain) and its known active fragments designated as SEQ. No. 79, SEQ. No. 80, and SEQ. No. 81, SEQ. No. 196 (subunit A of diphteria toxin modified by introducing of two mutations V7A and V27A. Modifications were chosen to eliminate VLS (vascular leak syndrome) due to Baluna R, Rizo J, Gordon 8E, Ghetie V, Vitetta E S. Proc Natl Acad Sci USA. 1999 Mar. 30; 96(7):3957-62) and SEQ. No. 197 (diphteria toxin was modified by introducing of deletion of three amino acids 6VDS9 and mutation V29A. to eliminate VLS (vascular leak syndrome) due to Baluna R, Rizo J, Gordon B E, Ghetie V, Vitetta E S. Proc. Natl. Acad Sci USA. 1999 Mar. 30; 96(7):3957-62).

The effector peptide of domain (b) of the fusion protein of the invention may be a peptide toxin inhibiting protein synthesis belonging to the toxin-antitoxin system, known for example in bacteria. Such toxins may block protein synthesis acting via different mechanisms: binding with a cellular membrane and thus leading to rapid collapse of membrane potential and a concomitant arrest of respiration; inhibition of polymerases (DNA and RNA) by binding to topoisomerase; or acting as endoribonuclease (RNase).

Examples of toxins being constituents of a toxin-antitoxin system with mRNase activity are: StaB protein with RNase activity (Szymanik M., Doctoral thesis. 2006. Warsaw University, Warsaw) designated as SEQ. No. 77; Kid toxin from Salmonella typhi (Bravo A, de Torrontegui G, Diaz R. Identification of components of a new stability system of plasmid R1, ParD, that is close to the origin of replication of this plasmid. Mol Gen Genet. 1987 November; 210(1):101-10), and RelE toxin from Escherichia coli (Gotfredsen M, Gerdes K. The Escherichia coli relBE genes belong to a New toxin-antitoxin gene family. Mol Microbiol. 1998 August; 29(4): 1065-76) designated as SEQ. No. 73 (Kid protein) and SEQ. No. 76 (ReIE protein).

Examples of toxin being constituents of a toxin-antitoxin system inhibiting polymerases by binding to topoisomerases are toxins from CcdB family Escherichia coli proteins and variants thereof with preserved activity of DNA degradation and inhibition of RNA polymerase, eg. CcdBET2 toxin (E. Trovatti et al, Bioorg Med Chem Lett. 2008 Dec. 1; 18(23):6161-4). Exemplary sequences of the effector peptide in this embodiment are designated as SEQ. No. 74 (CcdB protein) and SEQ. No. 75 (CcdB protein variant).

Examples of toxins being constituents of a toxin-antitoxin system binding with a cellular membrane and thus leading to rapid collapse of membrane potential and a concomitant arrest of respiration are small, basic proteins, containing long stretches of hydrophobic residues that insert into the cytoplasmic membraneTisB and Hok. Membrane insertion of Hok or TisB causes loss of electrochemical potential, which account for decrease in intracellular ATP. Thus, both TisB and Hok can kill cells by damaging bacterial membrane (Unoson C, Wagner E G. A small SOS-induced toxin is targeted against the inner membrane in Escherichia coli. Mol Microbiol. 2008 October; 70(1):258-70. Epub 2008 Aug. 29). Exemplary sequence of the effector peptide in this embodiment is designated as SEQ. No. 208).

As mentioned above, some effector peptide are novel and were not described before.

Thus, the invention relates to novel peptides selected from the group consisting of a mutated variant of trichosantin of SEQ. No. 200, a mutated variant of catalytic subunit A of Pseudomonas aeruginosa toxin of SEQ. No. 201, a mutated variant of catalytic subunit A of Pseudomonas aeruginosa toxin of SEQ. No. 202, a mutated variant of catalytic subunit A of Pseudomonas aeruginosa toxin of SEQ. No. 204, a mutated variant of catalytic subunit A of Pseudomonas aeruginosa toxin of SEQ. No. 205, and a mutated variant of catalytic subunit A of Pseudomonas aeruginosa toxin of SEQ. No. 207.

These novel peptides found the utility in particular as effector peptide of domain (b) of the anticancer fusion protein of the invention.

These novel peptides are designed specifically to lower immunogenicity of the parent peptide.

Thus, specific feature of these novel peptides is low immunogenicity.

Advantageous are the peptides selected from the group consisting of a mutated variant of trichosantin of SEQ. No. 200.

Also advantageous are the peptides selected from the group consisting of a mutated variant of catalytic subunit A of Pseudomonas aeruginosa toxin of SEQ. No. 201.

Also advantageous are the peptides selected from the group consisting of a mutated variant of catalytic subunit A of Pseudomonas aeruginosa toxin of SEQ. No. 202.

Also advantageous are the peptides selected from the group consisting of a mutated variant of catalytic subunit A of Pseudomonas aeruginosa toxin of SEQ. No. 204, a mutated variant of catalytic subunit A of Pseudomonas aeruginosa toxin of SEQ. No. 205, and a mutated variant of catalytic subunit A of Pseudomonas aeruginosa toxin of SEQ. No. 207.

Upon binding to TRAIL receptors present on the surface of cancer cells, the fusion protein will exert a double effect. Domain (a), that is a functional fragment of TRAIL or its homolog with preserved functionality, will exert its known agonistic activity, i.e. binding to death receptors on the cell surface and activation of extrinsic pathway of apoptosis. The effector peptide of the domain (b) of the fusion protein will be able to potentially exert its action intracellularly in parallel to the activity of TRAIL domain by inhibition of protein synthesis in tumor cells.

Activation of the effector peptide—functional domain (b) after internalization of the fusion protein into the cell may occur nonspecifically by a cleavage of domain (a) from domain (b) of the fusion protein of the invention by lisosomal enzymes (non-specific proteases).

Preferably however, the fusion protein comprises the domain of a cleavage site recognized by proteases present in the cell environment.

Thus, in a preferred embodiments of the invention, domain (a) and domain (b) are linked by at least one domain (c) comprising the sequence of a cleavage site recognized by proteases present in the cell environment, especially in the tumor cell environment, e.g. such as metalloprotease, urokinase or furin. Sequences recognized by protease may be selected from:
a sequence recognized by metalloprotease MMP Pro Leu Gly Leu Ala Gly Glu Pro/PLGLAGEP, or fragment thereof which with the last amino acid of the sequence to which is attached forms a sequence recognized by metalloprotease MMP,
a sequence recognized by urokinase uPA Arg Val Val Arg/RVVR, or fragment thereof, which with the last amino acid of the sequence to which is attached forms a sequence recognized by urokinase, and combinations thereof, or
a sequence recognized by furin Arg Gln Pro Arg/RQPR, Arg Gln Pro Arg Gly/RQPRG, Arg Lys Lys Arg/RKKR) or others atypical sequences recognized by furin disclosed by M. Gordon et all. In Inf. and Immun, 1995, 63, No. 1, p. 82-87 or native sequence recognized by furin Arg His Arg Gln Pro Arg Gly Trp Glu Gln Leu (RHRQPRGWEQL).

In one of the embodiments of the invention, the protease cleavage site is a combination of the sequence recognized by metalloprotease MMP and/or a sequence recognized by urokinase uPA and/or a sequence recognized by furin located next to each other in any order.

Preferably, in one of the embodiments domain (c) is a sequence recognized by furin selected from Arg Gln Pro Arg/RQPR, Arg Gln Pro Arg Gly/RQPRG, Arg Val Lys Arg/RVKR and Arg Lys Lys Arg/RKKR.

Proteases metalloprotease MMP, urokinase uPA and furin are overexpressed in the tumour environment. The presence of the sequence recognized by the protease enables the cleavage of domain (a) from domain (b), i.e. the release of the functional domain (b) and thus its accelerated activation.

The presence of the protease cleavage site, by allowing quick release of the effector peptide, increases the chances of transporting the peptide to the place of its action as a result of cutting off from the hTRAIL fragment by means of protease overexpressed in the tumor environment before random degradation of the fusion protein by non-specific proteases occurs.

In this regard, preferred effector peptides are diphtheria toxin and Pseudomonas exotoxin, which contain naturally occurring sequences of the cleavage site recognized by furin Arg Val Arg Arg/RVRR (diphteria toxin) and Arg Gin Pro Arg Gly/RQPRG (Pseudomonas exotoxin).

Additionally, a transporting domain (d) may be attached to domain (b) of the effector peptide of the fusion protein of the invention.

Domain (d) may be selected from the group consisting of:
(d1) a domain transporting through the cell membrane derived from Pseudomonas aeruginosa,
(d2) a domain transporting through the membrane targeting to the endoplasmic reticulum, and
(d3) a polyarginine sequence transporting through the cell membrane, consisting of 6, 7, 8, 9, 10 or 11 (Arg/R) residues,
   or fragments thereof, which with the last amino acid of the sequence to which is attached, forms sequences of transporting domains (d1), (d2) or (d3), and
combinations thereof.

The combination of domains (d1) (d2) and (d3) may comprise, in particular, the combination of (d1)/(d2), (d1)/(d3) or (d1)/(d2)/(d3).

Furthermore, the combination of domains (d1), (d2) and (d3) may include domains located next to each other and connected to one end of domain (b) and/or domains linked to different ends of domain (b).

It should be understood that in the case when the fusion protein has both the transporting domain (d) attached to domain (b) and domain (c) of the cleavage site between domains (a) and (b), then domain (c) is located in such a manner that after cleavage of the construct transporting domain (d) remains attached to domain (b). In other words, if the fusion protein contains both the transporting domain (d) and the cleavage site domain (c), then domain (d) is located between domain (b) and domain (c), or is located at the end of domain (b) opposite to the place of attachment of domain (d).

The invention comprises also a variant, in which domain (d), preferably the translocation Pseudomonas aeruginosa domain, is located between two (c) domains, that is the variant wherein after cleavage of the construct transporting domain, preferably the translocation Pseudomonas aeruginosa domain, is not attached neither to to the TRAIL domain nor to the effector peptide domain.

The invention does not comprise such a variant in which domain (d) is located between domain (c) and domain (a), that is the variant wherein after cleavage of the construct transporting domain remains attached to the TRAIL domain.

The transporting domain which is a translocation domain of Pseudomonas aeruginosa toxin or other fragment of a domain transporting through lysosomal membranes derived from Pseudomonas aeruginosa toxin has the ability to translocate across cell membranes and can be used to introduce the effector peptide to the compartments of tumor cells. The sequence of Pseudomonas aeruginosa translocation domain is well known and is designated by SEQ. No. 139.

Preferably, the Pseudomonas aeruginosa translocation domain is located between domains (a) and (b) and additionally separated by (c) domains.

Also preferably, domain (d2) transporting to the endoplasmic reticulum is attached to the C-terminus of the effector peptide and located at the C-terminus of the fusion protein of the invention.

Also preferably, the polyarginine sequence transporting through the cell membrane is attached to the C-terminus of the effector peptide and located between the effector peptide and domain (a); preferably, is additionally separated from (d) domain by means of domain (c).

The sequence (d2) directing to the endoplasmic reticulum may be any signal sequence known in the art directing to the endoplasmic reticulum, such as for example and not limiting Lys Asp Glu Leu/KDEL, His Asp Glu Leu/HDEL, Arg Asp Glu Leu/RDEL, Asp Asp Glu Leu/DDEL, Ala Asp Glu Leu/ADEL, Ser Asp Glu Leu/SDEL, and Lys Glu Asp Leu/KEDL.

Domain (d2) is preferably selected from Lys Asp Glu Leu/KDEL and Lys Glu Asp Leu/KEDL.

Preferably, transporting sequence (d2) is located at the C-terminus of the fusion protein of the invention.

In another embodiment, between domain (a) and domain (b) there is additionally located domain (e) comprising a sequence appropriate for attachment of a PEG molecule to the fusion protein (pegylation linker). Such a linker may be known sequence Ala Ser Gly Cys Gly Pro Glu/ASGCGPE. The pegylation linker may be also selected from the group of the following:
Ala Ala Cys Ala Ala/AACAA,
Ser Gly Gly Cys Gly Gly Ser/SGGCGGS, and
Ser Gly Cys Gly Ser/SGCGS.

Preferably, the sequence of pegylation linker is Ala Ser Gly Cys Gly Pro Glu/ASGCGPE.

Apart from the main functional elements of the fusion protein and the cleavage site domain(s), the fusion proteins of the invention may contain a neutral sequence/sequences of a flexible steric linker. Such steric linkers are well known and described in the literature. Their incorporation into the sequence of the fusion protein is intended to provide the correct folding of proteins produced by the process of its overexpression in the host cells. In particular, steric linker may be a glycine, glycine-serine or glycine-cysteine-alanine linker.

In particular, steric linker may be a combination of glycine and serine residues, such as for example Gly Gly Gly Gly Ser/GGGGS or any fragment thereof acting as steric linker, for example a fragment Gly Gly Gly Ser/GGGS, Gly Gly Gly/GGG or Gly Gly Gly Gly/GGGG. In other embodiment, the steric linker may be any combination of glycine, serine and alanine residues, such as for example Ala Ser Gly Gly/ASGG or any fragment thereof, acting as steric linker, for example AlaSerGly/ASG. It is also possible to use the combination of steric linkers, for example the sequence Gly Gly Gly Ser Gly/GGGGS or any fragment thereof acting as steric linker, for example a fragment Gly Gly Gly/GGG, with another fragment acting as steric linker. In such a case the steric linker may be a combination of glycine, serine and alanine residues, such as for example Gly Gly Gly Ser Ala Ser Gly Gly/GGGSASGG. In still another embodiment, steric linker may be a combination of serine and histidine residues Ser His His Ser/SHHS or Ser His His Ala Ser/SHHAS.

In another embodiment, steric linker may be a combination of alanine and cysteine residues, such as for example CAAACAAC (Cys Ala Ala Ala Cys Ala Ala Cys), CAACAAAC (Cys Ala Ala Cys Ala Ala Ala Cys) or fragments thereof.

In another embodiment ,suitable steric linkers are formed by combination of any types of steric linkers as mentioned above. Examples of such combinations are represented by: Gly Gly Gly Gly Gly Ser Gly Gly Gly Gly Ser (GGGGGSGGGGS), Gly Gly Gly Cys Ala Ala Ala Cys Ala Ala Cys (GGGCAAACAAC), and Gly Gly Gly Gly Ser Gly Gly Gly Gly Cys Ala Ala Ala Ala Ala Cys (GGGGSGGGCAAACAAC).

In one embodiment, the steric linker may be also selected from single amino acid residues, such as single cysteine residue.

In addition, the steric linker may also be useful for activation of functional domain (b), ocurring in a non-specific manner. Activation of domain (b) in a non-specific manner may be performed by cutting off the domain (a) from the domain (b) of the fusion protein according to the invention, due to pH-dependent hydrolysis of the steric linker.

Furthermore, the fusion protein of the invention may comprise a linker containing a motive binding to integrins. Such a linker provides an additional binding to the cell surface and can reduce systemic toxicity.

Integrins are alpha-beta heterodimers present on the surface of many cell types. Ligands for integrins are extracellular matrix adhesive proteins such as fibronectin, collagens, and laminin. In the case of fibronectin and some other ligands, a RGD motive is responsible for interaction with integrins. Peptides containing this motive specifically recognize integrin alpha 5 beta 1 and have inhibiting effect on the invasiveness of tumor cells by limiting their ability to form metastases (Ghelsen et al., (1988) J. Cell Biol. 106, 925-930). Using a method of phage display, from the library of 6-amino acids peptides a sequence comprising the NGR motive was isolated, which binds and recognizes specifically the integrin alpha 5 beta 1 (Koivunen et al., J Biol. Chem. 1993 Sep. 25; 268(27): 20205-10). It was also demonstrated that two motives (NGR and RGD) bind as antagonists to other factors involved in angiogenesis. RGD interacts also with integrins specifically overpresented in the process of neovascularization (Friedlander et al. Definition of two angiogenic pathways by distinct av integrins. Science (Washington D.C.), 270: 1500-1502, 1995), whereas NGR interacts with the aminopeptidase N, a protein also involved in the invasiveness of cancer, particularly strongly exposed in the blood vessels of tumors and other cells subjected to intense angiogenesis (Pasqualini et al., Aminopeptidase N is a receptor for tumor-homing peptides and a target for inhibiting angiogenesis. Cancer Res. 2000 Feb. 1; 60(3):722-7).

Linker from the fusion protein of the invention capable of binding with integrins comprises motive Asn Gly Arg (NGR), Asp Gly Arg (DGR) or Arg Gly Asp (RGD). In a preferred embodiment of the protein of the invention, a linker comprising a motive binding with integrines is designated by SEQ. No. 140.

The SEQ. No. 140 (Cys Phe Cys Asp Gly Arg Cys Asp Cys Ala/CFCDGRCDCA) comprises the motive Asp Gly Arg (DGR) stabilized by cysteine sequences and is known and described in Wang H, Yan Z, Shi J, Han W, Zhang Y Protein Expr Purif. 2006 January; 45(1): 60-5.

Particular embodiments of the fusion protein of the invention are fusion proteins comprising a peptide a peptide acting intracellularly by inhibition of translation process, selected from the group of peptides designated by:

SEQ. No. 55, SEQ. No. 56; SEQ. No. 57, SEQ. No. 58, SEQ. No. 59, SEQ. No. 60, SEQ. No. 61, SEQ. No. 62, SEQ. No. 63, SEQ. No. 64, SEQ. No. 65, SEQ. No. 66, SEQ. No. 67, SEQ. No. 68, SEQ. No. 69, SEQ. No. 70, SEQ. No. 71, SEQ. No. 72, SEQ. No. 73, SEQ. No. 74, SEQ. No. 75, SEQ. No. 76, SEQ. No. 77, SEQ. No. 78, SEQ. No. 79, SEQ. No. 80, SEQ. No. 81, SEQ. No. 82, SEQ. No. 83; SEQ. No. 84 and SEQ. No. 144, SEQ. No. 145; SEQ. No. 146, SEQ. No. 147, SEQ. No. 148, SEQ. No. 149, SEQ. No. 150, SEQ. No. 151, SEQ. No. 152, SEQ. No. 153, SEQ. No. 154, SEQ. No. 155, SEQ. No. 156, SEQ. No. 157, SEQ. No. 158, SEQ. No. 159, SEQ. No. 160, SEQ. No. 161, SEQ. No. 162, SEQ. No. 163, SEQ. No. 164; SEQ. No. 165, SEQ. No. 166; SEQ. No. 167, and SEQ. No. 168.

Anti-cancer activity of TRAIL in the fusion protein according to the invention can potentially be increased by activation of other components—such as for example depurination of adenine in 28S rRNA, ADP-ribosylation of factor EF2, N-glycosylation of adenine in 28SRNA, clevage of 285 RNA, cleavage of mRNA or DNA degradation, resulting in inhibition of protein synthesis and thus blocking reactions of cells at the level of the proteome, reducing the overproduction of proteins that block apoptosis pathway and finally reestablishing apoptosis pathway. Additionally, blocking of cellular protein synthesis process may activate by control points of the cell cycle (such as cyclin-dependent kinases) internally induced apoptosis, synergistic with the signal resulted from the attachment of TRAIL to the functional cell receptors of DR series.

It was found that the fusion proteins of the invention exhibit in many cases more potent activity than soluble TRAIL and its variants including fragments of the sequence. Hitherto, among known effector peptides used in the fusion protein of invention, only diphtheria toxin fused to interleukin-2 (Ontake®) has been used in medicine. Other effector peptides used in the fusion proteins of the invention have not been applied in medicine as such, due to the unfavorable kinetics, rapid degradation by non-specific proteases, and accumulation in the body caused by lack of proper sequence of activation pathways necessary to allow functioning of the effector peptide at the target site. Incorporation of the fusion protein enables their selective delivery to the place where their action is desired.

Moreover, the attachment of the effector peptide increases the weight of protein, which results in prolonged half-life and increased retention of protein in the tumor and in consequence increases its efficiency. Additionally, in many cases, new fusion proteins overcome a natural or induced resistance to TRAIL, probably through destabilization of cellular machinery responsible for protein synthesis. Because cancer cells may acquire resistance to cytotoxic activity of TRAIL, among others by overproduction of proteins blocking the apoptosis pathway (Bcl-2, IAP, XIAP or cFLIP), it appears that blocking the cellular mechanism of protein synthesis can lead to a blockage of cells reaction on the proteome level and thus to unblocking the apoptosis pathway.

A detailed description of the structure of representative fusion proteins mentioned above are shown in the Examples presented below.

In accordance with the present invention, by the fusion protein it is meant a single protein molecule containing two or more proteins or fragments thereof, covalently linked via peptide bond within their respective peptide chains, without additional chemical linkers.

The fusion protein can also be alternatively described as a protein construct or a chimeric protein. According to the present invention, the terms “construct” or “chimeric protein”, if used, should be understood as referring to the fusion protein as defined above.

For a person skilled in the art it will be apparent that the fusion protein thus defined can be synthesized by known methods of chemical synthesis of peptides and proteins.

The fusion protein can be synthesized by methods of chemical peptide synthesis, especially using the techniques of peptide synthesis in solid phase using suitable resins as carriers. Such techniques are conventional and known in the art, and described inter alia in the monographs, such as for example Bodanszky and Bodanszky, The Practice of Peptide Synthesis, 1984, Springer- Verlag, New York, Stewart et al., Solid Phase Peptide Synthesis, 2nd Edition, 1984, Pierce Chemical Company.

The fusion protein can be synthesized by the methods of chemical synthesis of peptides as a continuous protein. Alternatively, the individual fragments (domains) of protein may be synthesized separately and then combined together in one continuous peptide via a peptide bond, by condensation of the amino terminus of one peptide fragment from the carboxyl terminus of the second peptide. Such techniques are conventional and well known.

Preferably, however, the fusion protein of the invention is a recombinant protein, generated by methods of gene expression of a polynucleotide sequence encoding the fusion protein in host cells.

For verification of the structure of the resulting peptide known methods of the analysis of amino acid composition of peptides may be used, such as high resolution mass spectrometry technique to determine the molecular weight of the peptide. To confirm the peptide sequence, protein sequencers can also be used, which sequentially degrade the peptide and identify the sequence of amino acids.

A further aspect of the invention is a polynucleotide sequence, particularly DNA sequence, encoding the fusion protein as defined above.

Preferably, the polynucleotide sequence, particularly DNA, according to the invention, encoding the fusion protein as defined above, is a sequence optimized for expression in E. coli.

Another aspect of the invention is also an expression vector containing the polynucleotide sequence, particularly DNA sequence of the invention as defined above.

Another aspect of the invention is also a host cell comprising an expression vector as defined above.

A preferred host cell for expression of fusion proteins of the invention is an E. coli cell.

Methods for generation of recombinant proteins, including fusion proteins, are well known. In brief, this technique consists in generation of polynucleotide molecule, for example DNA molecule encoding the amino acid sequence of the target protein and directing the expression of the target protein in the host. Then, the target protein encoding polynucleotide molecule is incorporated into an appropriate expression vector, which ensures an efficient expression of the polypeptide. Recombinant expression vector is then introduced into host cells for transfection/transformation, and as a result a transformed host cell is produced. This is followed by a culture of transformed cells to overexpress the target protein, purification of obtained proteins, and optionally cutting off by cleavage the tag sequences used for expression or purification of the protein.

Suitable techniques of expression and purification are described, for example in the monograph Goeddel, Gene Expression Technology, Methods in Enzymology 185, Academic Press, San Diego, Calif. (1990), and A. Staron et al., Advances Mikrobiol., 2008, 47, 2, 1983-1995.

Cosmids, plasmids or modified viruses can be used as expression vectors for the introduction and replication of DNA sequences in host cells. Typically plasmids are used as expression vectors. Suitable plasmids are well known and commercially available.

Expression vector of the invention comprises a polynucleotide molecule encoding the fusion protein of the invention and the necessary regulatory sequences for transcription and translation of the coding sequence incorporated into a suitable host cell. Selection of regulatory sequences is dependent on the type of host cells and can be easily carried out by a person skilled in the art. Examples of such regulatory sequences are transcriptional promoter and enhancer or RNA polymerase binding sequence, ribosome binding sequence, containing the transcription initiation signal, inserted before the coding sequence, and transcription terminator sequence, inserted after the coding sequence. Moreover, depending on the host cell and the vector used, other sequences may be introduced into the expression vector, such as the origin of replication, additional DNA restriction sites, enhancers, and sequences allowing induction of transcription.

The expression vector will also comprise a marker gene sequence, which confers defined phenotype to the transformed cell and enables specific selection of transformed cells. Furthermore, the vector may also contain a second marker sequence which allows to distinguish cells transformed with recombinant plasmid containing inserted coding sequence of the target protein from those which have taken up the plasmid without insert. Most often, typical antibiotic resistance markers are used, however, any other reporter genes known in the field may be used, whose presence in a cell (in vivo) can be easily determined using autoradiography techniques, spectrophotometry or bio- and chemiluminescence. For example, depending on the host cell, reporter genes such as β-galactosidase, β-glucuronidase, luciferase, chloramphenicol acetyltransferase or green fluorescent protein may be used.

Furthermore, the expression vector may contain signal sequence, transporting proteins to the appropriate cellular compartment, e.g. periplasma, where folding is facilitated. Additionally a sequence encoding a label/tag, such as HisTag attached to the N-terminus or GST attached to the C-terminus, may be present, which facilitates subsequent purification of the protein produced using the principle of affinity, via affinity chromatography on a nickel column. Additional sequences that protect the protein against proteolytic degradation in the host cells, as well as sequences that increase its solubility may also be present.

Auxiliary element attached to the sequence of the target protein may block its activity, or be detrimental for another reason, such as for example due to toxicity. Such element must be removed, which may be accomplished by enzymatic or chemical cleavage. In particular, a six-histidine tag HisTag or other markers of this type attached to allow protein purification by affinity chromatography should be removed, because of its described effect on the liver toxicity of soluble TRAIL protein. Heterologous expression systems based on various well-known host cells may be used, including prokaryotic cells: bacterial, such as Escherichia coli or Bacillus subtilis, yeasts such as Saccharomyces cervisiae or Pichia pastoris, and eukaryotic cell lines (insect, mammalian, plant).

Preferably, due to the ease of culturing and genetic manipulation, and a large amount of obtained product, the E. coli expression system is used. Accordingly, the polynucleotide sequence containing the target sequence encoding the fusion protein of the invention will be optimized for expression in E. coli, i.e. it will contain in the coding sequence codons optimal for expression in E. coli, selected from the possible sequence variants known in the state of art. Furthermore, the expression vector will contain the above described elements suitable for E. coli attached to the coding sequence.

Accordingly, in a preferred embodiment of the invention a polynucleotide sequence comprising a sequence encoding a fusion protein of the invention, optimized for expression in E. coli is selected from the group of polynucleotide sequences consisting of:

SEQ. No. 85; SEQ. No. 86; SEQ. No. 87; SEQ. No. 88; SEQ. No. 89; SEQ. No. 90; SEQ. No. 91; SEQ. No. 92; SEQ. No. 93; SEQ. No. 94; SEQ. No. 95; SEQ. No. 96; SEQ. No. 97; SEQ. No. 98; SEQ. No. 99; SEQ. No. 100; SEQ. No. 101; SEQ. No. 102; SEQ. No. 103; SEQ. No. 104; SEQ. No. 105; SEQ. No. 106; SEQ. No. 107; SEQ. No. 108; SEQ. No. 109; SEQ. No. 110, SEQ. No. 111; SEQ. No. 111; SEQ. No. 113; SEQ. No. 114; SEQ. No. 115; SEQ. No. 116; SEQ. No. 117; SEQ. No. 118; SEQ. No. 119; SEQ. No. 120; SEQ. No. 121; SEQ. No. 122; SEQ. No. 123; SEQ. No. 124; SEQ. No. 125; SEQ. No. 126; SEQ. No. 127; SEQ. No. 128; SEQ. No. 129; SEQ. No. 130; SEQ. No. 131; SEQ. No. 132; SEQ. No. 133; SEQ. No. 134; SEQ. No. 135; SEQ. No. 136; SEQ. No. 137; SEQ. No. 138, SEQ. No. 169; SEQ. No. 170; SEQ. No. 171; SEQ. No. 172; SEQ. No. 173; SEQ. No. 174; SEQ. No. 175; SEQ. No. 176; SEQ. No. 177; SEQ. No. 178; SEQ. No. 179; SEQ. No. 180; SEQ. No. 181; SEQ. No, 182; SEQ. No. 183; SEQ. No. 184; SEQ. No. 185; SEQ. No. 186; SEQ. No. 187; SEQ. No. 188; SEQ. No. 189; SEQ. No. 190; SEQ. No. 191; SEQ. No. 192 and SEQ. No. 193;

which encode fusion proteins having amino acid sequences corresponding to amino acid sequences selected from the group consisting of amino acid sequences, respectively:

SEQ. No. 1; SEQ. No. 2; SEQ. No. 3; SEQ. No. 4; SEQ. No. 5; SEQ. No. 6; SEQ. No. 7; SEQ. No. 8; SEQ. No. 9; SEQ. No. 10; SEQ. No. 11; SEQ. No. 12; SEQ. No. 13; SEQ. No. 14; SEQ. No. 15; SEQ. No. 16; SEQ. No. 17; SEQ. No. 18; SEQ. No. 19; SEQ. No. 20; SEQ. No. 21; SEQ. No. 22; SEQ. No. 23; SEQ. No. 24; SEQ. No. 25; SEQ. No. 26, SEQ. No. 27; SEQ. No. 28; SEQ. No. 29; SEQ. No. 30; SEQ. No. 31; SEQ. No. 32; SEQ. No. 33; SEQ. No. 34; SEQ. No. 35; SEQ. No. 36; SEQ. No. 37; SEQ. No. 38; SEQ. No. 39; SEQ. No. 40; SEQ. No. 41; SEQ. No. 42; SEQ. No. 43; SEQ. No. 44; SEQ. No. 45; SEQ. No. 46; SEQ. No. 47; SEQ. No. 48; SEQ. No. 49; SEQ. No. 50; SEQ. No. 51; SEQ. No. 52; SEQ. No. 53, SEQ. No. 54144; SEQ. No. 145; SEQ. No. 146; SEQ. No. 147; SEQ. No. 148; SEQ. No. 149; SEQ. No. 150; SEQ. No. 151; SEQ. No. 152; SEQ. No. 153; SEQ. No. 154; SEQ. No. 155; SEQ. No. 156; SEQ. No. 157; SEQ. No. 158; SEQ. No. 159; SEQ. No. 160; SEQ. No. 161; SEQ. No. 162; SEQ. No. 163; SEQ. No. 164; SEQ. No. 165; SEQ. No. 166; SEQ. No. 167 and SEQ. No. 168.

In a preferred embodiment, the invention provides also an expression vector suitable for transformation of E. coli, comprising the polynucleotide sequence selected from the group of polynucleotide sequences SEQ. No. 85 to SEQ. No. 138 and from SEQ. No. 169 to SEQ. No. 193 indicated above, as well as E. coli cell transformed with such an expression vector.

Transformation, i.e. introduction of a DNA sequence into bacterial host cells, particularly E. coli, is usually performed on the competent cells, prepared to take up the DNA for example by treatment with calcium ions at low temperature (4° C.), and then subjecting to the heat-shock (at 37-42° C.) or by electroporation.

Such techniques are well known and are usually determined by the manufacturer of the expression system or are described in the literature and manuals for laboratory work, such as Maniatis et al., Molecular Cloning. Cold Spring Harbor, N.Y., 1982).

The procedure of overexpression of fusion proteins of the invention in E. coli expression system will be further described below.

The invention also provides a pharmaceutical composition containing the fusion protein of the invention as defined above as an active ingredient and a suitable pharmaceutically acceptable carrier, diluent and conventional auxiliary components. The pharmaceutical composition will contain an effective amount of the fusion protein of the invention and pharmaceutically acceptable auxiliary components dissolved or dispersed in a carrier or diluent, and preferably will be in the form of a pharmaceutical composition formulated in a unit dosage form or formulation containing a plurality of doses. Pharmaceutical forms and methods of their formulation as well as other components, carriers and diluents are known to the skilled person and described in the literature. For example, they are described in the monograph Remington's Pharmaceutical Sciences, ed. 20, 2000, Mack Publishing Company, Easton, USA.

The terms “pharmaceutically acceptable carrier, diluent, and auxiliary ingredient” comprise any solvents, dispersion media, surfactants, antioxidants, stabilizers, preservatives (e.g. antibacterial agents, antifungal agents), isotonizing agents, known in the art. The pharmaceutical composition of the invention may contain various types of carriers, diluents and excipients, depending on the chosen route of administration and desired dosage form, such as liquid, solid and aerosol forms for oral, parenteral, inhaled, topical, and whether that selected form must be sterile for administration route such as by injection. The preferred route of administration of the pharmaceutical composition according to the invention is parenteral, including injection routes such as intravenous, intramuscular, subcutaneous, intraperitoneal, intratumoral, or by single or continuous intravenous infusions.

In one embodiment, the pharmaceutical composition of the invention may be administered by injection directly to the tumor. In another embodiment, the pharmaceutical composition of the invention may be administered intravenously. In yet another embodiment, the pharmaceutical composition of the invention can be administered subcutaneously or intraperitoneally. A pharmaceutical composition for parenteral administration may be a solution or dispersion in a pharmaceutically acceptable aqueous or non-aqueous medium, buffered to an appropriate pH and isoosmotic with body fluids, if necessary, and may also contain antioxidants, buffers, bacteriostatic agents and soluble substances, which make the composition compatible with the tissues or blood of recipient. Other components, which may included in the composition, are for example water, alcohols such as ethanol, polyols such as glycerol, propylene glycol, liquid polyethylene glycol, lipids such as triglycerides, vegetable oils, liposomes. Proper fluidity and the particles size of the substance may be provided by coating substances, such as lecithin, and surfactants, such as hydroxypropyl-celulose, polysorbates, and the like.

Suitable isotonizing agents for liquid parenteral compositions are, for example, sugars such as glucose, and sodium chloride, and combinations thereof.

Alternatively, the pharmaceutical composition for administration by injection or infusion may be in a powder form, such as a lyophilized powder for reconstitution immediately prior to use in a suitable carrier such as, for example, sterile pyrogen-free water.

The pharmaceutical composition of the invention for parenteral administration may also have the form of nasal administration, including solutions, sprays or aerosols. Preferably, the form for intranasal administration will be an aqueous solution and will be isotonic or buffered o maintain the pH from about 5.5 to about 6.5, so as to maintain a character similar to nasal secretions. Moreover, it will contain preservatives or stabilizers, such as in the well-known intranasal preparations.

The composition may contain various antioxidants which delay oxidation of one or more components. Furthermore, in order to prevent the action of microorganisms, the composition may contain various antibacterial and anti fungal agents, including, for example, and not limited to, parabens, chlorobutanol, himerosal, sorbic acid, and similar known substances of this type.

In general, the pharmaceutical composition of the invention can include, for example at least about 0.01 wt % of active ingredient. More particularly, the composition may contain the active ingredient in the amount from 1% to 75% by weight of the composition unit, or for example from 25% to 60% by weight, but not limited to the indicated values. The actual amount of the dose of the composition according to the present invention administered to patients, including man, will be determined by physical and physiological factors, such as body weight, severity of the condition, type of disease being treated, previous or concomitant therapeutic interventions, the patient and the route of administration. A suitable unit dose, the total dose and the concentration of active ingredient in the composition is to be determined by the treating physician.

The composition may for example be administered at a dose of about 1 microgram/kg of body weight to about 1000 mg/kg of body weight of the patient, for example in the range of 5 mg/kg of body weight to 100 mg/kg of body weight or in the range of 5 mg/kg of body weight to 500 mg/kg of body weight. The fusion protein and the compositions containing it exhibit anticancer or antitumor and can be used for the treatment of cancer diseases. The invention also provides the use of the fusion protein of the invention as defined above for treating cancer diseases in mammals, including humans. The invention also provides a method of treating neoplastic/cancer diseases in mammals, including humans, comprising administering to a subject in need of such treatment an anit-neoplastic/anticancer effective amount of the fusion protein of the invention as defined above, optionally in the form of appropriate pharmaceutical composition.

The fusion protein of the invention can be used for the treatment of hematologic malignancies, such as leukaemia, granulomatosis, myeloma and other hematologic malignancies. The fusion protein can also be used for the treatment of solid tumors, such as breast cancer, lung cancer, including non-small cell lung cancer, colon cancer, pancreatic cancer, ovarian cancer, bladder cancer, prostate cancer, kidney cancer, brain cancer, and the like. Appropriate route of administration of the fusion protein in the treatment of cancer will be in particular parenteral route, which consists in administering the fusion protein of the invention in the form of injections or infusions, in the composition and form appropriate for this administration route. The invention will be described in more detail in the following general procedures and examples of specific fusion proteins.

General Procedure for Overexpression of the Fusion Protein

Preparation of a Plasmid

Amino acid sequence of a target fusion protein was used as a template to generate a DNA sequence encoding it, comprising codons optimized for expression in Escherichia coli. Such a procedure allows to increase the efficiency of further step of target protein synthesis in Escherichia coli . Resulting nucleotide sequence was then automatically synthesized. Additionally, the cleavage sites of restriction enzymes Ndel (at the 5′-end of leading strand) and Xhol (at the 3′-end of leading strand) were added to the resulting gene encoding the target protein. These were used to clone the gene into the vector pET28a (Novagen). They may be also be used for cloning the gene encoding the protein other vectors. Target protein expressed from this construct can be optionally equipped at the N-terminus with a polyhistidine tag (six histidines), preceded by a site recognized by thrombin, which subsequently serves to its purification via affinity chromatography. Some targets were expressed without any tag, in particular without histidine tag, and those were subsequently purified on SP Sepharose. The correctness of the resulting construct was confirmed firstly by restriction analysis of isolated plasmids using the enzymes Ndel and Xhol, followed by automatic sequencing of the entire reading frame of the target protein. The primers used for sequencing were complementary to the sequences of T7 promoter (5′-TAATACGACTCACTATAGG-3′) and T7 terminator (5°-GCTAGTTATTGCTCAGCGG-3′) present in the vector. Resulting plasmid was used for overexpression of the target fusion protein in a commercial E. coli strain, which was transformed according to the manufacturers recommendations. Colonies obtained on the selection medium (LB agar, kanamycin 50 μg/ml, 1% glucose) were used for preparing an overnight culture in LB liquid medium supplemented with kanamycin (50 μg/ml) and 1% glucose. After about 15h of growth in shaking incubator, the cultures were used to inoculate the appropriate culture.

Overexpression and Purification of Fusion Proteins—General Procedure A

LB medium with kanamycin (30 μg/ml) and 100 μM zinc sulfate was inoculated with overnight culture. The culture was incubated at 37° C. until the optical density (OD) at 600 nm reached 0.60-0.80. Then IPTG was added to the final concentration in the range of 0.25-1 mM. After incubation (3.5-20 h) with shaking at 25° C. the culture was centrifuged for 25 min at 6,000 g. Bacterial pellets were resuspended in a buffer containing 50 mM KH₂PO₄, 0.5 M NaCl, 10 mM imidazole, pH 7.4. The suspension was sonicated on ice for 8 minutes (40% amplitude, 15-second pulse, 10 s interval). The resulting extract was clarified by centrifugation for 40 minutes at 20000 g, 4° C. Ni-Sepharose (GE Healthcare) resin was pre-treated by equilibration with buffer, which was used for preparation of the bacterial cells extract. The resin was then incubated is overnight at 4° C. with the supernatant obtained after centrifugation of the extract. Then it was loaded into chromatography column and washed with 15 to 50 volumes of buffer 50 mM KH₂PO₄, 0.5 M NaCl, 20 mM imidazole, pH 7.4. The obtained protein was eluted from the column using imidazole gradient in 50 mM KH₂PO₄ buffer with 0.5 M NaCl, pH 7.4. Obtained fractions were analyzed by SDS-PAGE. Appropriate fractions were combined and dialyzed overnight at 4° C. against 50 mM Tris buffer, pH 7.2, 150 mM NaCl, 500 mM L-arginine, 0.1 mM ZnSO₄, 0.01% Tween 20, and at the same time Histag, if present, was cleaved with thrombin (1:50). After the cleavage, thrombin was separated from the target fusion protein expressed with His tag by purification using Benzamidine Sepharose™ resin. Purification of target fusion proteins expressed without Histag was performed on SP Sepharose. The purity of the product was analyzed by SDS-PAGE electrophoresis (Maniatis et al, Molecular Cloning. Cold Spring Harbor, N.Y., 1982).

Overexpression and Purification of Fusion Proteins—General Procedure B

LB medium with kanamycin (30 μg/ml) and 100 μM zinc sulfate was inoculated with overnight culture. Cultures were incubated at 37° C. until optical density (OD) at 600 nm reached 0.60-0.80. Then IPTG was added to the final concentration in the range 0.5-1 mM. After 20 h incubation with shaking at 25° C. the culture was centrifuged for 25 min at 6000 g. Bacterial cells after overexpression were disrupted in a French Press in a buffer containing 50 mM KH₂PO₄, 0.5 M NaCl, 10 mM imidazole, 5 mM beta-mercaptoethanol, 0.5 mM PMSF (phenylmethylsulphonyl fluoride), pH 7.8. Resulting extract was clarified by centrifugation for 50 minutes at 8000 g. The Ni-Sepharose resin was incubated overnight with the obtained supernatant. Then the resin with bound protein was packed into the chromatography column. To wash-out the fractions containing non-binding proteins, the column was washed with 15 to 50 volumes of buffer 50 mM KH₂PO₄, 0.5 M NaCl, 10 mM imidazole, 5 mM beta-mercaptoethanol, 0.5 mM PMSF (phenylmethylsulphonyl fluoride), pH 7.8. Then, to wash-out the majority of proteins binding specifically with the bed, the column was washed with a buffer containing 50 mM KH₂PO₄, 0.5 M NaCl, 500 mM imidazole, 10% glycerol, 0.5 mM PMSF, pH 7.5. Obtained fractions were analyzed by SDS-PAGE (Maniatis et al, Molecular Cloning. Cold Spring Harbor, N.Y., 1982). The fractions containing the target protein were combined and, if the protein was expressed with histidine tag, cleaved with thrombin (1U per 4 mg of protein, 8 h at 16° C.) to remove polyhistidine tag. Then the fractions were dialyzed against formulation buffer (500 mM L-arginine, 50 mM Tris, 2.5 mM ZnSO₄, pH 7.4).

In this description Examples of proteins originally expressed with histidine tag that was subsequently removed are designated with superscript a) next to the Example number. Proteins that were originally expressed without histidine tag are designated with superscript b) next to the Example number.

Characterization of Fusion Proteins by 2-D Electrophoresis

In order to further characterize obtained proteins and to select precisely chromatographic conditions, isoelectric points of the proteins were determined. For this purpose, two-dimensional electrophoresis (2-D) method was used, in two stages according to the following schedule.

Step 1. Isoelectrofocusing of Proteins in a pH Gradient and Denaturing Conditions.

Protein preparations at concentrations of 1-2 mg/ml were precipitated by mixing in a 1:1 ratio with a precipitation solution containing 10% trichloroacetic acid and 0.07% beta-mercaptoethanol in acetone. The mixture was incubated for 30 min at −20° C. and then centrifuged for 25 min at 15,000 g and 4° C. The supernatant was removed and the pellet was washed twice with cold acetone with 0.07% beta-mercaptoethanol. Then the residues of acetone were evaporated until no detectable odour. The protein pellet was suspended in 250 ml of rehydration buffer 8M urea, 1% CHAPS, 15 mM DTT, 0.5% ampholyte (GE Healthcare) with a profile of pH 3-11 or 6-11, depending on the strip subsequently used. The protein solution was placed in a ceramic chamber for isoelectrofocusing, followed by 13 cm DryStrip (GE Healthcare) with appropriate pH profile (3-11 or 6-11). The whole was covered with a layer of mineral oil. The chambers were placed in the Ettan IPGphor III apparatus, where isoelectrofocusing was conducted according to the following program assigned to the dimensions of the strip and the pH profile:

16 h dehydration at 20° C.

Focusing in the electric field at a fixed pH gradient

| Time | Voltage | |
|---|---|---|
| 1 h | 500 | V |
| 1 h | gradient 500-1000 | V |
| 2 h 30 min | gradient 1000-8000 | V |
| 30 min | 8000 | V |

Then, the strip containing the focused proteins was washed for 1 min in deionised water, stained with Coomassie Brilliant and then decolorized and archived as an image to mark the location of proteins. Discoloured strip was equilibrated 2×15 min with a buffer of the following composition: 50 mM Tris-HCl pH 8.8, 6M urea, 1% DTT, 2% SDS, 30% glycerol.

Step 2. Separation in a Second Direction by SDS-PAGE.

The strip was placed over the 12.5% polyacrylamide gel containing a single well per standard size and then separation was performed in an apparatus for SDS-PAGE, at a voltage of 200V for 3 hours. The gel was stained with Coomassie Brilliant then archived with the applied scale. Proteins were identified by determining its weight on the basis of the standard of size, and its IPI was read for the scale of 6-11 on the basis of the curves provided by the manufacturer (GE Healthcare) (ratio of pH to % of length of the strip from the end marked as anode) or a scale of 3-11 on the basis of the curve determined experimentally by means of isoelectrofocusing calibration kit (GE Healthcare).

### EXAMPLES

The representative examples of the fusion proteins of the invention are shown in the following Examples.

The following designations of the amino acids sequences components are used:
LINKER1: steric linker sequence (Gly Gly Gly Gly Ser/GGGGS)
LINKER2: steric linker sequence (Gly Gly Gly Gly/GGGG)
LINKER3: steric linker sequence (Ala Ser Gly Gly/ASGG)
LINKER4: steric linker sequence (Gly Gly Gly Ser/GGGS)
LINKERS: steric linker sequence (Ser His Ala Ser/SHAS)
FURIN: sequence cleaved by furin (Arg Lys Lys Arg/RKKR)
UROKIN: sequence cleaved by urokinase (Arg Val Val Arg/RWR)
PEG: pegylation linker sequence (Ala Ser Gly Cys Gly Pro Glu/ASGCGPE)
TRANS1: transporting sequence (Lys Asp Glu Leu/KDEL)
TRANS2: transporting sequence (Arg Arg Arg Arg Arg Arg Arg Arg/RRRRRRRR)
TRANS3: (Lys Glu Asp Leu /KEDL)
LINKER6: (Cys Ala Ala Ala Cys AlaAla Cys/CAAACAAC)
LINKER7: (Gly Gly Gly/GGG)
MMP: (Pro Leu Gly Leu Ala Gly/PLGLAG)
FURIN.NAT: (Arg His Arg Gln Pro Arg Gly Trp Glu Gln Leu/RHRQPRGWEQL)

### Example 1

### Fusion Protein of SEQ. No. 1

The protein of SEQ. No. 1 is a fusion protein having the length of 430 amino acids and the mass of 48.3 kDa, wherein domain (a) is formed by a sequence of TRAIL121-281, and domain (b) of effector peptide is a 248-amino acids boguanin domain A (SEQ. No. 55), and is attached at the N-terminus of domain (a).

Additionally, between domain (a) and domain(b) there are sequentially incorporated steric linker sequence (GGGGS), sequence cleaved by furin (RKKR), pegylation linker sequence (ASGCGPE) and steric linker sequence (GGGGS).

Thus, the structure of the fusion protein of the invention is as follows:
(SEQ. No. 55)-LINKER1-FURIN-PEG-LINKER1-(TRAIL121-281)

The amino acid sequence and the DNA encoding sequence comprising codons optimized for expression in E. coli are, respectively, SEQ. No. 1 and SEQ. No. 85, as shown in the attached Sequence Listing.

The amino acid sequence SEQ. No. 1 of the structure described above was used as a template to generate its coding DNA sequence SEQ. No. 85. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

Protein was expressed with histidine tag.

### Example 2

### The Fusion Protein of SEQ. No. 2

The protein of SEQ. No. 2 is a fusion protein having the length of 267 amino acids and the mass of 50.8 kDa, wherein domain (a) is TRAIL121-281, and domain (b) of the effector peptide is y 267-amino acids domain of ricin A (SEQ. No. 56), and is attached at the C-terminus of domain (a).

Additionally, domain (a) is separated from domain (b) by steric linker sequence (GGGGS), pegylation sequence (ASGCGPE) and a sequence of cleavage site recognized by furin (RKKR). Additionally, at the C-terminus of domain (b) is attached a transporting sequence KDEL, directing the effector peptide to the endoplasmic reticulum, forming C-terminal fragment of entire construct.

Thus, the structure of the fusion protein of the invention is as follows:
(TRAIL 121-281)-LINKER1-PEG-FURIN-LINKER1-(SEQ. No. 56)-TRANS1

The amino acid sequence and the DNA encoding sequence comprising codons optimized for expression in E. coli are, respectively, SEQ. No. 2 and SEQ. No. 86, as shown in the attached Sequence Listing.

The amino acid sequence SEQ. No. 2 of the structure described above was used as a template to generate its coding DNA sequence SEQ. No. 86. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

Protein was expressed both with histidine tag (Ex. 2^{a}) and without histidine tag (Ex. 2^{b}).

### Example 3

### The Fusion Protein of SEQ. No. 3

The protein of SEQ. No. 3 is a fusion protein having the length of 378 amino acids and the mass of 42 kDa, wherein domain (a) is TRAIL121-281, and domain (b) of the effector peptide is 267-amino acids variant of ricin A domain (SEQ. No. 57), and is attached at the C-terminus of domain (a).

Additionally, domain (a) is separated from domain (b) by sequentially the sequence of steric linker (GGGGS), pegylation sequence (ASGCGPE), the sequence of cleavage site recognized by furin (RKKR) and the sequence of steric linker (GGGGS). Additionally, to the C-terminus of domain (b) there is attached a transporting sequence KDEL, directing the effector peptide to the endoplasmic reticulum, forming C-terminal fragment of entire construct.

Thus, the structure of the fusion protein of the invention is as follows:
(TRAIL 121-281)-LINKER1-PEG-FURIN-LINKER1-(SEQ. No. 57)-TRANS1

The amino acid sequence and the DNA encoding sequence comprising codons optimized for expression in E. coli are, respectively, SEQ. No. 3 and SEQ. No. 87, as shown in the attached Sequence Listing.

The amino acid sequence SEQ. No. 3 of the structure described above was used as a template to generate its coding DNA sequence SEQ. No. 87. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

Protein was expressed with histidine tag.

### Example 4

### The Fusion Protein of SEQ. No. 4

The protein of SEQ. No. 4 is a fusion protein having the length of 473 amino acids and the mass of 53,2 kDa, wherein domain (a) is TRAIL121-281, and domain (b) of the effector peptide is a 290-amino acids homolog of PAP toxin (SEQ. No. 58), and is attached at the C-terminus of domain (a).

Additionally, domain (a) is separated from domain (b) by sequentially steric linker sequence (GGGGS), pegylation sequence (ASGCGPE) and steric linker sequence (GGGGS). Additionally, to the C-terminus of domain (b) there is attached transporting sequence (KDEL), directing the effector peptide to the endoplasmic reticulum, forming C-terminal fragment of entire construct.

Thus, the structure of the fusion protein of the invention is as follows:
(TRAIL 121-281)-LINKER1-PEG-LINKER1 -(SEQ. No. 58)-TRANS1

The amino acid sequence and the DNA encoding sequence comprising codons optimized for expression in E. coli are, respectively, SEQ. No. 4 and SEQ. No. 88, as shown in the attached Sequence Listing.

The amino acid sequence SEQ. No. 4 of the structure described above was used as a template to generate its coding DNA sequence SEQ. No. 88. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

Protein was expressed with histidine tag.

### Example 5

### The Fusion Protein of SEQ. No. 5

The protein of SEQ. No. 5 is a fusion protein having the length of 430 amino acids and the mass of 48.3 kDa, wherein domain (a) is TRAIL121-281, and domain (b) of the effector peptide is a 252-amino acids fragment of saporin (SEQ. No. 59), and is attached at the C-terminus of domain (a).

Additionally, domain (a) is separated from domain (b) by sequentially steric linker sequence (GGGGS), pegylation sequence (ASGCGPE) and steric linker sequence (GGGGS).

Thus, the structure of the fusion protein of the invention is as follows:
(TRAIL121-281)-LINKER1-PEG-LINKER1-(SEQ. No. 59)

The amino acid sequence and the DNA encoding sequence comprising codons optimized for expression in E. coli are, respectively, SEQ. No. 5 and SEQ. No. 89 as shown in the attached Sequence Listing.

The amino acid sequence SEQ. No. 5 of the structure described above was used as a template to generate its coding DNA sequence SEQ. No. 89. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

Protein was expressed with histidine tag.

### Example 6

### The Fusion Protein of SEQ. No. 6

The protein of SEQ. No. 6 is a fusion protein having the length of 442 amino acids and the mass of 49.7 kDa, wherein domain (a) is TRAIL121-281, and domain (b) of the effector peptide is 252-amino acids fragment of saporin (SEQ. No. 59), and is attached at the C-terminus of domain (a).

Additionally, between domains (a) and (b) are incorporated sequentially pegylation linker sequence (ASGCGPE), two sequences of steric linker (GGGGS) and a sequence cleaved by furin (RKKR).

Thus, the structure of the fusion protein of the invention is as follows:
(TRAIL121-281)-PEG-LINKER1-LINKER1-FURIN-(SEQ. No. 59)

The amino acid sequence and the DNA encoding sequence comprising codons optimized for expression in E. coli are, respectively, SEQ. No. 6 and SEQ. No. 90 as shown in the attached Sequence Listing.

The amino acid sequence SEQ. No. 6 of the structure described above was used as a template to generate its coding DNA sequence SEQ. No. 90. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

Protein was expressed with histidine tag.

### Example 7

### The Fusion Protein of SEQ. No, 7

The protein of SEQ. No. 7 is a fusion protein having the length of 429 amino acids and the mass of 47.5 kDa, wherein domain (a) is TRAIL121-281, and domain (b) of the effector peptide is 247-amino acids peptide trichosantin (SEQ. No. 60), and is attached at the N-terminus of domain (a).

Additionally, between domains (b) and (a) are incorporated sequentially steric linker sequence (GGGGS), sequence cleaved by furin (RKKR), pegylation linker sequence (ASGCGPE) and steric linker sequence (GGGGS).

Thus, the structure of the fusion protein of the invention is as follows:
(SEQ. No. 60)-LINKER1-FURIN-PEG-LINKER1-(TRAIL121-281)

The amino acid sequence and the DNA encoding sequence comprising codons optimized for expression in E. coli are, respectively, SEQ. No. 7 and SEQ. No. 91 as shown in the attached Sequence Listing.

The amino acid sequence SEQ. No. 7 of the structure described above was used as a template to generate its coding DNA sequence SEQ. No. 91. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

Protein was expressed with histidine tag.

### Example 8

### The Fusion Protein of SEQ. No. 8

The protein of SEQ. No. 8 is a fusion protein having the length of 427 amino acids and the mass of 47.5 kDa, wherein domain (a) is TRAIL 121-281, and domain (b) of the effector peptide is 247-amino acids peptide trichoanguin (SEQ. No. 61), and is attached at the N-terminus of domain (a).

Additionally, between domains (b) and (a) there are sequentially incorporated steric linker sequence (GGGGS), sequence cleaved by furin (RKKR), pegylation linker sequence (ASGCGPE) and steric linker sequence (GGGGS).

Thus, the structure of the fusion protein of the invention is as follows:
(SEQ. No. 61)-LINKER1-FURIN-PEG-LINKER1-(TRAIL121-281)

The amino acid sequence and the DNA encoding sequence comprising codons optimized for expression in E. coli are, respectively, SEQ. No. 8 and SEQ. No. 92 as shown in the attached Sequence Listing.

The amino acid sequence SEQ. No. 8 of the structure described above was used as a template to generate its coding DNA sequence SEQ. No. 92. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

Protein was expressed with histidine tag.

### Example 9

### The Fusion Protein of SEQ. No. 9

The protein of SEQ. No. 9 is a fusion protein having the length of 427 amino acids and the mass of 47.7 kDa, wherein domain (a) is TRAIL 121-281 sequence, and domain (b) of the effector peptide is 249-amino acids chain of mistletoe lectin A (SEQ. No. 62), and is attached at the N-terminus of domain (a).

Additionally, between domains (b) and (a) there are sequentially incorporated steric linker sequence (GGGGS), pegylation linker sequence (ASGCGPE) and steric linker sequence (GGGGS).

Thus, the structure of the fusion protein of the invention is as follows:
(SEQ. No. 62)-LINKER1-PEG-LINKER1-(TRAIL121-281)

The amino acid sequence and the DNA encoding sequence comprising codons optimized for expression in E. coli are, respectively, SEQ. No. 9 and SEQ. No. 93 as shown in the attached Sequence Listing.

The amino acid sequence SEQ. No. 9 of the structure described above was used as a template to generate its coding DNA sequence SEQ. No. 93. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

Protein was expressed with histidine tag.

### Example 10

### The Fusion Protein of SEQ. No. 10

The protein of SEQ. No. 10 is a fusion protein having the length of 462 amino acids and the mass of 51.9 kDa, wherein domain (a) is TRAIL114-281, and domain (b) of the effector peptide is 273-amino acids subunit A of ebulin (SEQ. No. 63), and is attached at the N-terminus of domain (a).

Additionally, between domains (b) and (a) there are sequentially incorporated steric linker sequence (GGGGS), pegylation linker sequence (ASGCGPE), sequence cleaved by furin (RKKR) and steric linker sequence (GGGG).

Thus, the structure of the fusion protein of the invention is as follows:
(SEQ. No. 63)-LINKER1-PEG-FURIN-LINK2-(TRAIL114-281)

The amino acid sequence and the DNA encoding sequence comprising codons optimized for expression in E. coli are, respectively, SEQ. No. 10 and SEQ. No. 94 as shown in the attached Sequence Listing.

The amino acid sequence SEQ. No. 10 of the structure described above was used as a template to generate its coding DNA sequence SEQ. No. 94. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

Protein was expressed with histidine tag.

### Example 11

### The Fusion Protein of SEQ. No. 11

The protein of SEQ. No. 11 is a fusion protein having the length of 454 amino acids and the mass of 50.7 kDa, wherein domain (a) is TRAIL121-281 sequence, and domain (b) of the effector peptide is 272-amino acids subunit A of nigrin (SEQ. No. 64), and is attached at the N-terminus of domain (a).

Additionally, between domains (b) and (a) there are sequentially incorporated steric linker sequence (GGGGS), sequence cleaved by furin (RKKR), pegylation linker sequence (ASGCGPE) and steric linker sequence (GGGGS),

Thus, the structure of the fusion protein of the invention is as follows:
(SEQ. No. 64)-LINKER1-FURIN-PEG-LINKER1-(TRAIL121-281)

The amino acid sequence and the DNA encoding sequence comprising codons optimized for expression in E. coli are, respectively, SEQ. No. 11 and SEQ. No. 95 as shown in the attached Sequence Listing.

The amino acid sequence SEQ. No. 11 of the structure described above was used as a template to generate its coding DNA sequence SEQ. No. 95. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

Protein was expressed with histidine tag.

### Example 12

### The Fusion Protein of SEQ. No. 12

The protein of SEQ. No. 12 is a fusion protein having the length of 221 amino acids and the mass of 25.7 kDa, wherein domain (a) is TRAIL 121-281 sequence, and domain (b) of the effector peptide is 47-amino acids luffin P1 peptide (SEQ. No. 65), and is attached at the C-terminus of domain (a).

Additionally, between domains (a) and (b) there are sequentially incorporated steric linker sequence (GGGGS) and sequence cleaved by furin (RKKR).

Thus, the structure of the fusion protein of the invention is as follows:
(TRAIL 121-281)-LINKER1-FURIN-(SEQ. No. 65)

The amino acid sequence and the DNA encoding sequence comprising codons optimized for expression in E. coli are, respectively, SEQ. No. 12 and SEQ. No. 96 as shown in the attached Sequence Listing.

The amino acid sequence SEQ. No. 12 of the structure described above was used as a template to generate its coding DNA sequence SEQ. No. 96. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

Protein was expressed with histidine tag.

### Example 13

### The Fusion Protein of SEQ. No. 13

The protein of SEQ. No. 13 is a fusion protein having the length of 221 amino acids and the mass of 26 kDa, wherein domain (a) is TRAIL 121-281 sequence, and domain (b) of the effector peptide is 47-amino acids luffin P1 peptide (SEQ. No. 65), and is attached at the C-terminus of domain (a).

Additionally, between domains (a) and (b) there are sequentially incorporated sequences of steric linkers (ASGG) and (GGGS), pegylation linker sequence (ASGCGPE), sequence cleaved by furin (RKKR) and steric linker sequence (ASGG).

Thus, the structure of the fusion protein of the invention is as follows:
(TRAIL121-281)-LINKER4-PEG-FURIN-LINKER3-(SEQ. No. 65)

The amino acid sequence and the DNA encoding sequence comprising codons optimized for expression in E. coli are, respectively, SEQ. No. 13 and SEQ. No. 97 as shown in the attached Sequence Listing.

The amino acid sequence SEQ. No. 13 of the structure described above was used as a template to generate its coding DNA sequence SEQ. No. 97. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

Protein was expressed with histidine tag.

### Example 14

### The Fusion Protein of SEQ. No. 14

The protein of SEQ. No. 14 is a fusion protein having the length of 254 amino acids and the mass of 29.2 kDa, wherein domain (a) is a sequence TRAIL 95-281, and domain (b) of the effector peptide is 47-amino acids luffin P1 peptide (SEQ. No. 65), and is attached at the C-terminus of domain (a).

Additionally, between domains (a) and (b) there are sequentially incorporated steric linker sequence (GGGGS), pegylation linker sequence (ASGCGPE) and sequence cleaved by furin (RKKR). Additionally, to the C-terminus of domain (b) is attached a transporting sequence KDEL, directing the effector peptide to the endoplasmic reticulum, forming C-terminal fragment of entire construct.

Thus, the structure of the fusion protein of the invention is as follows:
(TRAIL 95-281)-LINKER1-PEG-FURIN-(SEQ. No. 65)-TRANS1

The amino acid sequence and the DNA encoding sequence comprising codons optimized for expression in E. coli are, respectively, SEQ. No. 14 and SEQ. No. 98 as shown in the attached Sequence Listing.

The amino acid sequence SEQ. No. 14 of the structure described above was used as a template to generate its coding DNA sequence SEQ. No. 98. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

Protein was expressed both with histidine tag (Ex. 14^{a}) and without histidine tag (Ex. 14^{b}).

### Example 15

### The Fusion Protein of SEQ. No. 15

The protein of SEQ. No. 15 is a fusion protein having the length of 438 amino acids and the mass of 49 kDa, wherein domain (a) is TRAIL 121-281 sequence, and domain (b) of the effector peptide is a 244-amino acids subunit A of volkensin (SEQ. No. 66), and is attached at the N-terminus of domain (a).

Additionally, between domains (b) and (a) there are sequentially incorporated steric linker sequence (GGGGS), sequence cleaved by furin (RKKR), pegylation linker sequence (ASGCGPE) and steric linker sequence (GGGGS).

Thus, the structure of the fusion protein of the invention is as follows:
(SEQ. No. 66)-LINKER1-FURIN-PEG-LINKER1-(TRAIL121-281)

The amino acid sequence and the DNA encoding sequence comprising codons optimized for expression in E. coli are, respectively, SEQ. No. 15 and SEQ. No. 99 as shown in the attached Sequence Listing.

The amino acid sequence SEQ. No. 15 of the structure described above was used as a template to generate its coding DNA sequence SEQ. No. 99. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

Protein was expressed both with histidine tag (Ex. 15^{a}) and without histidine tag (Ex. 15^{b}).

### Example 16

### The Fusion Protein of SEQ. No. 16

The protein of SEQ. No. 16 is a fusion protein having the length of 431 amino acids and the mass of 48.3 kDa, wherein domain (a) is TRAIL 121-281 sequence, and domain (b) of the effector peptide is a 244-amino acids subunit A of volkensin (SEQ. No. 66), and is attached at the C-terminus of domain (a).

Additionally, between domains (a) and (b) there are sequentially incorporated steric linker sequence (GGGGS), pegylation linker sequence (ASGCGPE) and steric linker sequence (GGGGS).

Additionally, to the C-terminus of domain (b) there is attached transporting sequence KDEL, directing the effector peptide to the endoplasmic reticulum, forming C-terminal fragment of entire construct.

Thus, the structure of the fusion protein of the invention is as follows:
(TRAIL 121-281)-LINKER1-PEG-LINKER1-(SEQ. No. 66)-TRANS1

The amino acid sequence SEQ. No. 16 of the structure described above was used as a template to generate its coding DNA sequence SEQ. No. 100. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

Protein was expressed with histidine tag.

### Example 17

### The Fusion Protein of SEQ. No. 17

The protein of SEQ. No. 17 is a fusion protein having the length of 428 amino acids and the mass of 47.8 kDa, wherein domain (a) is TRAIL 121-281 sequence, and domain (b) of the effector peptide is 246-amino acids subunit A of volkensin (SEQ. No. 67), and is attached at the N-terminus of domain (a).

Additionally, between domains (b) and (a) there are sequentially incorporated steric linker sequence (GGGGS), sequence cleaved by furin (RKKR), pegylation linker sequence (ASGCGPE) and steric linker sequence (GGGGS).

Thus, the structure of the fusion protein of the invention is as follows:
(SEQ. No. 67)-LINKER1-FURIN-PEG-LINKER1-(TRAIL121-281)

The amino acid sequence and the DNA encoding sequence comprising codons optimized for expression in E. coli are, respectively, SEQ. No. 17 and SEQ. No. 101 as shown in the attached Sequence Listing.

The amino acid sequence SEQ. No. 17 of the structure described above was used as a template to generate its coding DNA sequence SEQ. No. 101. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

Protein was expressed with histidine tag.

### Example 18

### The Fusion Protein of SEQ. No. 18

The protein of SEQ. No. 18 is a fusion protein having the length of 515 amino acids and the mass of 55.9 kDa, wherein domain (a) is TRAIL 121-281 sequence, and domain (b) of the effector peptide is 342-amino acids homolog of a fragment of modified sequence of Pseudomonas aeruginosa exotoxin (SEQ. No. 68), and is attached at the C-terminus of domain (a).

Additionally, between domains (a) and (b) there are sequentially incorporated steric linker sequence (GGGS) and steric linker sequence (ASGG). Additionally, to the C-terminus of domain (b) there is attached a transporting sequence (KDEL), directing the effector peptide to the endoplasmic reticulum, forming C-terminal fragment of entire construct.

Thus, the structure of the fusion protein of the invention is as follows:
(TRAIL121-281)-LINKER4-LINKER3-(SEQ. No. 68)-TRANS1

The amino acid sequence and the DNA encoding sequence comprising codons optimized for expression in E. coli are, respectively, SEQ. No. 18 and SEQ. No. 102 as shown in the attached Sequence Listing.

The amino acid sequence SEQ. No. 18 of the structure described above was used as a template to generate its coding DNA sequence SEQ. No. 102. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. Protein was expressed both with histidine tag (Ex. 18^{a}) and without histidine tag (Ex. 18^{b}).

### Example 19

### The Fusion Protein of SEQ. No. 19

The protein of SEQ. No. 19 is a fusion protein having the length of 526 amino acids and the mass of 57.1 kDa, wherein domain (a) is sequence TRAIL 119-281, and domain (b) of the effector peptide is 342-amino acids homolog of the fragment of modified Pseudomonas aeruginosa exotoxin sequence (SEQ. No. 68), and is attached at the C-terminus of domain (a).

Additionally, between domains (a) and (b) there are sequentially incorporated steric linker sequence (GGGS), pegylation linker sequence (ASGCGPE), sequence cleaved by furin (RKKR) and steric linker sequence (ASGG). Additionally, to the C-terminus of domain (b) is attached transporting sequence (KDEL), directing the effector peptide to the endoplasmic reticulum, forming C-terminal fragment of entire fusion protein.

Thus, the structure of the fusion protein of the invention is as follows:
(TRAIL119-281)-LINKER4-PEG-FURIN-LINKER3-(SEQ. No. 68)-TRANS1

The amino acid sequence and the DNA encoding sequence comprising codons optimized for expression in E. coli are, respectively, SEQ. No. 19 and SEQ. No. 103 as shown in the attached Sequence Listing.

The amino acid sequence SEQ. No. 19 of the structure described above was used as a template to generate its coding DNA sequence SEQ. No. 103. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

Protein was expressed with histidine tag.

### Example 20

### The Fusion Protein of SEQ. No. 20

The protein of SEQ. No. 20 is a fusion protein having the length of 526 amino acids and the mass of 57.2 kDa, wherein domain (a) is TRAIL 121-281 sequence, and domain (b) of the effector peptide is 354-amino acids homolog of the fragment of modified Pseudomonas aeruginosa exotoxin sequence (SEQ. No. 84), and is attached at the C-terminus of domain (a).

Additionally, between domains (a) and (b) there are sequentially incorporated steric linker sequence (GGGS), pegylation linker sequence (ASGCGPE), sequence cleaved by furin (RKKR) and steric linker sequence (ASGG).

Additionally, to the C-terminus of domain (b) there is attached transporting sequence KDEL, directing the effector peptide to the endoplasmic reticulum, forming C-terminal fragment of entire fusion protein.

Thus, the structure of the fusion protein of the invention is as follows:

(TRAIL121-281)-LINKER4-PEG-FURIN-LINKER3-(SEQ. No. 84)-TRANS1

The amino acid sequence and the DNA encoding sequence comprising codons optimized for expression in E. coli are, respectively, SEQ. No. 20 and SEQ. No. 104 as shown in the attached Sequence Listing.

The amino acid sequence SEQ. No. 20 of the structure described above was used as a template to generate its coding DNA sequence SEQ. No. 104. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

Protein was expressed both with histidine tag (Ex. 20^{a}) and without histidine tag (Ex. 20^{b}).

### Example 21

### The Fusion Protein of SEQ. No. 21

The protein of SEQ. No. 21 is a fusion protein having the length of 534 amino acids and the mass of 58.5 kDa, wherein domain (a) is TRAIL 121-281 sequence, and domain (b) of the effector peptide is 354-amino acids homolog of the fragment of modified Pseudomonas aeruginosa exotoxin sequence (SEQ. No. 69), and is attached at the C-terminus of domain (a).

Additionally, between domains (a) and (b) there are sequentially incorporated steric linker sequence (GGGS), pegylation linker sequence (ASGCGPE), sequence cleaved by furin (RKKR) and steric linker sequence (ASGG).

Thus, the structure of the fusion protein of the invention is as follows:
(TRAIL121-281)-LINKER4-PEG-FURIN-LINKER3-(SEQ. No. 69)

The amino acid sequence and the DNA encoding sequence comprising codons optimized for expression in E. coli are, respectively, SEQ. No. 21 and SEQ. No. 105 as shown in the attached Sequence Listing.

The amino acid sequence SEQ. No. 21 of the structure described above was used as a template to generate its coding DNA sequence SEQ. No. 105. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

Protein was expressed with histidine tag.

### Example 22

### The Fusion Protein of SEQ. No. 22

The protein of SEQ. No. 22 is a fusion protein having the length of 534 amino acids and the mass of 56.1 kDa, wherein domain (a) is TRAIL 121-281 sequence, and domain (b) of the effector peptide is 342-amino acids fragment of modified Pseudomonas aeruginosa exotoxin sequence (SEQ. No. 83), and is attached at the C-terminus of domain (a). Additionally, between domains (a) and (b) a steric linker sequence (GGGS) is incorporated. Thus, the structure of the fusion protein of the invention is as follows:
(TRAIL121-281)-LINKER4-(SEQ. No. 83)

The amino acid sequence and the DNA encoding sequence comprising codons optimized for expression in E. coli are, respectively, SEQ. No. 22 and SEQ. No. 106 as shown in the attached Sequence Listing.

The amino acid sequence SEQ. No. 22 of the structure described above was used as a template to generate its coding DNA sequence SEQ. No. 106. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strains from Novagen. The protein was separated by electrophoresis in accordance with the general procedure described above.

Protein was expressed with histidine tag.

### Example 23

### The Fusion Protein of SEQ. No. 23

The protein of SEQ. No. 23 is a fusion protein having the length of 526 amino acids and the mass of 57.2 kDa, wherein domain (a) is TRAIL 119-281, and domain (b) of the effector peptide is 342-amino acids fragment of modified Pseudomonas aeruginosa exotoxin sequence (SEQ. No. 83), and is attached at the C-terminus of domain (a).

Additionally, between domains (a) and (b) there are sequentially incorporated steric linker sequence (GGGS), pegylation linker sequence (ASGCGPE), sequence cleaved by furin (RKKR) and steric linker sequence (ASGG).

Additionally, to the C-terminus of domain (b) there is attached transporting sequence KDEL, directing the effector peptide to the endoplasmic reticulum, forming C-terminal fragment of entire fusion protein.

Thus, the structure of the fusion protein of the invention is as follows:
(TRAIL119-281)-LINKER4-PEG-FURIN-LINKER3-(SEQ. No. 83)-TRANS1

The amino acid sequence and the DNA encoding sequence comprising codons optimized for expression in E. coli are, respectively, SEQ. No. 23 and SEQ. No. 107 as shown in the attached Sequence Listing.

The amino acid sequence SEQ. No. 23 of the structure described above was used as a template to generate its coding DNA sequence SEQ. No. 107. A plasmid containing the coding sequence of DNA was generated and overexpression of the fusion protein was carried out in accordance with the general procedures described above. Overexpression was performed according to the general procedure B, using E. coli BL21 (DE3) or E. coli Tuner (DE3) strain from Novagen. The protein was separated by electrophoresis in accordance with the general procedure described above.

Protein was expressed with histidine tag.

### Example 24

### The Fusion Protein of SEQ. No. 24

The protein of SEQ. No. 24 is a fusion protein having the length of 526 amino acids and the mass of 57.2 kDa, wherein domain (a) is TRAIL 121-281 sequence, and domain (b) of the effector peptide is 342-amino acids fragment of modified Pseudomonas aeruginosa exotoxin sequence (SEQ. No. 83), and is attached at the C-terminus of domain (a).

Additionally, between domains (a) and (b) there are sequentially incorporated steric linker sequence (GGGS), pegylation linker sequence (ASGCGPE), sequence cleaved by furin (RKKR) and steric linker sequence (ASGG).

Additionally, to the C-terminus of domain (b) there is attached transporting sequence KDEL, directing the effector peptide to the endoplasmic reticulum, forming C-terminal fragment of entire fusion protein.

Thus, the structure of the fusion protein of the invention is as follows:
(TRAIL119-281)-LINKER4-PEG-FURIN-LINKER3-(SEQ. No. 83)-TRANS1

The amino acid sequence and the DNA encoding sequence comprising codons optimized for expression in E. coli are, respectively, SEQ. No. 24 and SEQ. No. 108 as shown in the attached Sequence Listing.

The amino acid sequence SEQ. No. 24 of the structure described above was used as a template to generate its coding DNA sequence SEQ. No. 108. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

Protein was expressed with histidine tag.

### Example 25

### The Fusion Protein of SEQ. No. 25

The protein of SEQ. No. 25 is a fusion protein having the length of 423 amino acids and the mass of 47.3 kDa, wherein domain (a) is TRAIL 114-281, and domain (b) of the effector peptide is 239-amino acids variant of Shiga toxin stx (SEQ. No. 70), and is attached at the N-terminus of domain (a).

Additionally, between domains (b) and (a) there are sequentially incorporated steric linker sequence (SHHAS), sequence cleaved by furin (RKKR) and steric linker sequence (GGGGS).

Thus, the structure of the fusion protein of the invention is as follows:
(SEQ. No. 70)-LINKER5-FURIN-LINKER1-(TRAIL114-281)

The amino acid sequence and the DNA encoding sequence comprising codons optimized for expression in E. coli are, respectively, SEQ. No. 25 and SEQ. No. 109 as shown in the attached Sequence Listing.

The amino acid sequence SEQ. No. 25 of the structure described above was used as a template to generate its coding DNA sequence SEQ. No. 109. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

Protein was expressed with histidine tag.

### Example 26

### The Fusion Protein of SEQ. No. 26

The protein of SEQ. No. 26 is a fusion protein having the length of 432 amino acids and the mass of 47.9 kDa, wherein domain (a) is TRAIL 120-281, and domain (b) of the effector peptide is 239-amino acids variant of Shiga toxin stx (SEQ. No. 70), and is attached at the C-terminus of domain (a).

Additionally, between domains (a) and (b) there are sequentially incorporated steric linker sequence (GGGS), pegylation sequence (ASGCGPE), sequence cleaved by furin (RKKR) and steric linker sequence (GGGS).

Additionally, to the C-terminus of domain (b) there is attached transporting sequence KDEL, directing the effector peptide to the endoplasmic reticulum, forming C-terminal fragment of entire fusion protein.

Thus, the structure of the fusion protein of the invention is as follows:
(TRAIL 120-281)-LINKER4-PEG-FURIN-LINKER4-(SEQ. No. 70)-TRANS1.

The amino acid sequence and the DNA encoding sequence comprising codons optimized for expression in E. coli are, respectively, SEQ. No. 26 and SEQ. No. 110 as shown in the attached Sequence Listing.

The amino acid sequence SEQ. No. 26 of the structure described above was used as a template to generate its coding DNA sequence SEQ. No. 110. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

Protein was expressed both with histidine tag (Ex. 26^{a}) and without histidine tag (Ex. 26^{b}).

### Example 27

### The Fusion Protein of SEQ. No. 27

The protein of SEQ. No. 27 is a fusion protein having the length of 526 amino acids and the mass of 38 kDa, wherein domain (a) is TRAIL 114-281, and domain (b) of the effector peptide is 149-amino acids restrictocin peptide (SEQ. No. 71), and is attached at the N-terminus of domain (a). Additionally, between domains (b) and (a) there are sequentially incorporated two sequences of steric linker (GGGGS), sequence cleaved by furin (RKKR) and pegylation linker sequence (ASGCGPE).

Thus, the structure of the fusion protein of the invention is as follows:
(SEQ. No. 71)-LINKER1-LINKER1-FURIN-PEG-(TRAIL114-281)

The amino acid sequence and the DNA encoding sequence comprising codons optimized for expression in E. coli are, respectively, SEQ. No. 27 and SEQ. No. 111 as shown in the attached Sequence Listing.

The amino acid sequence SEQ. No. 27 of the structure described above was used as a template to generate its coding DNA sequence SEQ. No. 111. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strains from Novagen. The protein was separated by electrophoresis in accordance with the general procedure described above.

Protein was expressed both with histidine tag (Ex. 27^{a}) and without histidine tag (Ex. 27^{b}).

### Example 28

### The Fusion Protein of SEQ. No. 28

The protein of SEQ. No. 28 is a fusion protein having the length of 335 amino acids and the mass of 37.7 kDa, wherein domain (a) is TRAIL 121-281 sequence, and domain (b) of the effector peptide is 149-amino acids restrictocin peptide (SEQ. No. 71), and is attached at the C-terminus of domain (a). Additionally, between domains (a) and (b) there are sequentially incorporated steric linker sequence (GGGGS), pegylation linker sequence (ASGCGPE), sequence cleaved by Turin (RKKR) and steric linker sequence (GGGGS). Additionally, to the C-terminus of domain (b) there is attached transporting sequence KEDL, directing the effector peptide to the endoplasmic reticulum, forming C-terminal fragment of entire fusion protein.

Thus, the structure of the fusion protein of the invention is as follows:
(TRAIL121-281)-LINKER1-PEG-FURIN-LINKER1-(SEQ. No. 71)-TR2

The amino acid sequence and the DNA encoding sequence comprising codons optimized for expression in E. coli are, respectively, SEQ. No. 28 and SEQ. No. 112 as shown in the attached Sequence Listing.

The amino acid sequence SEQ. No. 28 of the structure described above was used as a template to generate its coding DNA sequence SEQ. No. 112. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

Protein was expressed both with histidine tag (Ex. 28^{a}) and without histidine tag (Ex. 28^{b}).

### Example 29

### The Fusion Protein of SEQ. No. 29

The protein of SEQ. No. 29 is a fusion protein having the length of 319 amino acids and the mass of 35.7 kDa, wherein domain (a) is TRAIL 114-281, and domain (b) of the effector peptide is 130-amino acids hirsutellin peptide (SEQ. No. 72), and is attached at the N-terminus of domain (a).

Additionally, between domains (b) and (a) there are sequentially incorporated two sequences of steric linkers (GGGGS), sequence cleaved by furin (RKKR) and pegylation linker sequence (ASGCGPE).

Thus, the structure of the fusion protein of the invention is as follows:
(SEQ. No. 72)-LINKER1-LINKER1-FURIN-PEG-(TRAIL114-281)

The amino acid sequence and the DNA encoding sequence comprising codons optimized for expression in E. coli are, respectively, SEQ. No. 29 and SEQ. No. 113 as shown in the attached Sequence Listing.

The amino acid sequence SEQ. No. 29 of the structure described above was used as a template to generate its coding DNA sequence SEQ. No. 113. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

Protein was expressed both with histidine tag (Ex. 29^{a}) and without histidine tag (Ex. 29^{b}).

### Example 30

### The Fusion Protein of SEQ. No. 30

The protein of SEQ. No. 30 is a fusion protein having the length of 290 amino acids and the mass of 32.3 kDa, wherein domain (a) is TRAIL 121-281 sequence, and domain (b) of the effector peptide is 109-amino acids Kid protein (SEQ. No. 73), and is attached at the C-terminus of domain (a).

Additionally, between domains (a) and (b) there are sequentially incorporated steric linker sequence (GGGGS), pegylation linker sequence (ASGCGPE) and sequence cleaved by furin (RKKR).

Additionally, to the C-terminus of domain (b) there is attached transporting sequence KDEL, directing the effector peptide to the endoplasmic reticulum, forming C-terminal fragment of entire fusion protein.

Thus, the structure of the fusion protein of the invention is as follows:
(TRAIL121-281)-LINKER1-PEG-FURIN-(SEQ. No. 73)-TRANS1

The amino acid sequence and the DNA encoding sequence comprising codons optimized for expression in E. coli are, respectively, SEQ. No. 30 and SEQ. No. 114 as shown in the attached Sequence Listing.

The amino acid sequence SEQ. No. 30 of the structure described above was used as a template to generate its coding DNA sequence SEQ. No. 114. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

Protein was expressed with histidine tag.

### Example 31

### The Fusion Protein of SEQ. No. 31

The protein of SEQ. No. 31 is a fusion protein having the length of 277 amino acids and the mass of 31.7 kDa, wherein domain (a) is TRAIL 121-281 sequence, and domain (b) of the effector peptide is 100-amino acids CcdB protein (SEQ. No. 74), and is attached at the C-terminus of domain (a). Additionally, between domains (a) and (b) there are sequentially incorporated steric linker sequence (GGGGS), pegylation linker sequence (ASGCGPE) and sequence cleaved by furin (RKKR).

Thus, the structure of the fusion protein of the invention is as follows:
(TRAIL121-281)-LINKER1-PEG-FURIN- (SEQ. No.74)

The amino acid sequence and the DNA encoding sequence comprising codons optimized for expression in E. coli are, respectively, SEQ. No. 31 and SEQ. No. 115 as shown in the attached Sequence Listing.

The amino acid sequence SEQ. No. 31 of the structure described above was used as a template to generate its coding DNA sequence SEQ. No. 115. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above.

Protein was expressed with histidine tag.

### Example 32

### The Fusion Protein of SEQ. No. 32

The protein of SEQ. No. 32 is a fusion protein having the length of 228 amino acids and the mass of 25.7 kDa, wherein domain (a) is TRAIL 121-281, and domain (b) of the effector peptide is 47-amino acids variant of CcdB protein (SEQ. No. 75), and is attached at the C-terminus of domain (a).

Additionally, between domains (a) and (b) there are sequentially incorporated steric linker sequence (GGGGS), pegylation linker sequence (ASGCGPE) and sequence cleaved by furin (RKKR).

Additionally, to the C-terminus of domain (b) there is attached transporting sequence KDEL, directing the effector peptide to the endoplasmic reticulum, forming C-terminal fragment of entire fusion protein.

Thus, the structure of the fusion protein of the invention is as follows:
(TRAIL121-281)-LINKER1-PEG-FURIN-(SEQ. No. 75)-TRANS1

The amino acid sequence and the DNA encoding sequence comprising codons optimized for expression in E. coli are, respectively, SEQ. No. 32 and SEQ. No. 116 as shown in the attached Sequence Listing.

The amino acid sequence SEQ. No. 32 of the structure described above was used as a template to generate its coding DNA sequence SEQ. No. 116. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above. Protein was expressed both with histidine tag (Ex. 32^{a}) and without histidine tag (Ex. 32^{b}).

### Example 33

### The Fusion Protein of SEQ. No. 33

The protein of SEQ. No. 33 is a fusion protein having the length of 275 amino acids and the mass of 31.7 kDa, wherein domain (a) is TRAIL 121-281, and domain (b) of the effector peptide is 94-amino acids ReLE protein (SEQ. No. 76), and is attached at the C-terminus of domain (a).

Additionally, between domains (a) and (b) there are sequentially incorporated steric linker sequence (GGGGS), pegylation linker sequence (ASGCGPE) and sequence cleaved by furin (RKKR).

Additionally, to the C-terminus of domain (b) there is attached transporting sequence KDEL, directing the effector peptide to the endoplasmic reticulum, forming C-terminal fragment of entire fusion protein.

Thus, the structure of the fusion protein of the invention is as follows:
(TRAIL121-281)-LINKER1-PEG-FURIN-(SEQ. No. 76)-TRANS1

The amino acid sequence and the DNA encoding sequence comprising codons optimized for expression in E. coli are, respectively, SEQ. No. 33 and SEQ. No. 117 as shown in the attached Sequence Listing.

The amino acid sequence SEQ. No. 33 of the structure described above was used as a template to generate its coding DNA sequence SEQ. No. 117. A plasmid containing the coding sequence of DNA was generated and overexpression of the fusion protein was carried out in accordance with the general procedures described above. Overexpression was performed according to the general procedure A, using strain E. coli Tuner (DE3) from Novagen. The protein was separated by electrophoresis in accordance with the general procedure described above.

Protein was expressed with histidine tag.

### Example 34

### The Fusion Protein of SEQ. No. 34

The protein of SEQ. No. 34 is a fusion protein having the length of 271 amino acids and the mass of 30.7 kDa, wherein domain (a) is TRAIL 121-281, and domain (b) of the effector peptide is 90-amino acids StaB protein (SEQ. No. 77), and is attached at the C-terminus of domain (a).

Additionally, between domains (a) and (b) there are sequentially incorporated steric linker sequence (GGGGS), pegylation linker sequence (ASGCGPE) and sequence cleaved by furin (RKKR).

Additionally, to the C-terminus of domain (b) there is attached transporting sequence KDEL, directing the effector peptide to the endoplasmic reticulum, forming C-terminal fragment of entire fusion protein.

Thus, the structure of the fusion protein of the invention is as follows:
(TRAIL121-281)-LINKER1-PEG-FURIN-(SEQ. No. 77)-TRANS1

The amino acid sequence and the DNA encoding sequence comprising codons optimized for expression in E. coli are, respectively, SEQ. No. 34 and SEQ. No. 118 as shown in the attached Sequence Listing.

The amino acid sequence SEQ. No. 34 of the structure described above was used as a template to generate its coding DNA sequence SEQ. No. 118. A plasmid containing the coding sequence of DNA was generated and overexpression of the fusion protein was carried out in accordance with the general procedures described above. Overexpression was performed according to the general procedure A, using strains E. coli Tuner (DE3) from Novagen. The protein was separated by electrophoresis in accordance with the general procedure described above.

Protein was expressed with histidine tag.

### Example 35

### The Fusion Protein of SEQ. No. 35

The protein of SEQ. No. 35 is a fusion protein having the length of 429 amino acids and the mass of 48.2 kDa, wherein domain (a) is TRAIL 114-281, and domain (b) of the effector peptide is 251-amino acids gelonin peptide (SEQ. No. 78), and is attached at the N-terminus of domain (a). Additionally, between domains (b) and (a) there are sequentially incorporated two sequences of steric linker (GGGGS).

Thus, the structure of the fusion protein of the invention is as follows:
(SEQ. No. 78)-LINKER1-LINKER1-(TRAIL 114-281)

The amino acid sequence and the DNA encoding sequence comprising codons optimized for expression in E. coli are, respectively, SEQ. No. 35 and SEQ. No. 119 as shown in the attached Sequence Listing.

The amino acid sequence SEQ. No. 35 of the structure described above was used as a template to generate its coding DNA sequence SEQ. No. 119. A plasmid containing the coding sequence of DNA was generated and overexpression of the fusion protein was carried out in accordance with the general procedures described above. Overexpression was performed according to the general procedure A, using strains E. coli Tuner (DE3) from Novagen. The protein was separated by electrophoresis in accordance with the general procedure described above.

Protein was expressed with histidine tag.

### Example 36

### The Fusion Protein of SEQ. No. 36

The protein of SEQ. No. 36 is a fusion protein having the length of 434 amino acids and the mass of 48.6 kDa, wherein domain (a) is TRAIL 120-281, and domain (b) of the effector peptide is 251-amino acids gelonin peptide (SEQ. No. 78), and is attached at the N-terminus of domain (a).

Additionally, between domains (b) and (a) there are sequentially incorporated steric linker sequence (GGGGS), sequence cleaved by furin (RKKR), pegylation linker sequence (ASGCGPE) and steric linker sequence (GGGGS).

Thus, the structure of the fusion protein of the invention is as follows:
(SEQ. No. 78)-LINKER1-FURIN-PEG-LINKER1-(TRAIL120-281)

The amino acid sequence and the DNA encoding sequence comprising codons optimized for expression in E. coli are, respectively, SEQ. No. 36 and SEQ. No. 120 as shown in the attached Sequence Listing.

The amino acid sequence SEQ. No. 36 of the structure described above was used as a template to generate its coding DNA sequence SEQ. No. 120. A plasmid so containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above.

Protein was expressed with histidine tag.

### Example 37

### The Fusion Protein of SEQ. No. 37

The protein of SEQ. No. 37 is a fusion protein having the length of 427 amino acids and the mass of 48 kDa, wherein domain (a) is TRAIL 121-281 sequence, and domain (b) of the effector peptide is 251-amino acids gelonin peptide (SEQ. No. 78), and is attached at the C-terminus of domain (a).

Additionally, between domains (a) and (b) there are sequentially incorporated pegylation linker sequence (ASGCGPE) and steric linker sequence (GGGGS).

Additionally, to the C-terminus of domain (b) there is attached transporting sequence KDEL, directing the effector peptide to the endoplasmic reticulum, forming C-terminal fragment of entire fusion protein.

Thus, the structure of the fusion protein of the invention is as follows:
(TRAIL121-281)-PEG-LINKER1-(SEQ. No. 78)-TRANS1

The amino acid sequence and the DNA encoding sequence comprising codons optimized for expression in E. coli are, respectively, SEQ. No. 37 and SEQ. No. 121 as shown in the attached Sequence Listing.

The amino acid sequence SEQ. No. 37 of the structure described above was used as a template to generate its coding DNA sequence SEQ. No. 121. A plasmid containing the coding sequence of DNA was generated and overexpression of the fusion protein was carried out in accordance with the general procedures described above. Overexpression was performed according to the general procedure A, using strains E. coli Tuner (DE3) from Novagen. The protein was separated by electrophoresis in accordance with the general procedure described above.

Protein was expressed with histidine tag.

### Example 38

### The Fusion Protein of SEQ. No. 38

The protein of SEQ. No. 38 is a fusion protein having the length of 433 amino acids and the mass of 48.5 kDa, wherein domain (a) is TRAIL 121-281, and domain (b) of the effector peptide is 251-amino acids gelonin peptide (SEQ. No. 78), and is attached at the N-terminus of domain (a).

Additionally, between domains (b) and (a) there are sequentially incorporated steric linker sequence (GGGGS), sequence cleaved by furin (RKKR), pegylation linker sequence (ASGCGPE) and steric linker sequence (GGGGS).

Thus, the structure of the fusion protein of the invention is as follows:
(SEQ. No. 78)-LINKER1-FURIN-PEG-LINKER1-(TRAIL121-281)

The amino acid sequence and the DNA encoding sequence comprising codons optimized for expression in E. coli are, respectively, SEQ. No. 38 and SEQ. No. 122 as shown in the attached Sequence Listing.

The amino acid sequence SEQ. No. 38 of the structure described above was used as a template to generate its coding DNA sequence SEQ. No. 122. A plasmid containing the coding sequence of DNA was generated and overexpression of the fusion protein was carried out in accordance with the general procedures described above. Overexpression was performed according to the general procedure A, using strains E. coli Tuner (DE3) from Novagen. The protein was separated by electrophoresis in accordance with the general procedure described above.

Protein was expressed with histidine tag.

### Example 39

### The Fusion Protein of SEQ. No. 39

The protein of SEQ. No. 39 is a fusion protein having the length of 558 amino acids and the mass of 61.4 kDa, wherein domain (a) is TRAIL 121-281, and domain (b) of the effector peptide is 387-amino acids subunit A of diphteria toxin (SEQ. No. 79), and is attached at the N-terminus of domain (a). Additionally, between domains (b) and (a) there are sequentially incorporated two sequences of steric linker (GGGGS).

Thus, the structure of the fusion protein of the invention is as follows:
(SEQ. No. 79)-LINKER1-LINKER1-(TRAIL121-281)

The amino acid sequence and the DNA encoding sequence comprising codons optimized for expression in E. coli are, respectively, SEQ. No. 39 and SEQ. No. 123 as shown in the attached Sequence Listing.

The amino acid sequence SEQ. No. 39 of the structure described above was used as a template to generate its coding DNA sequence SEQ. No. 123. A plasmid containing the coding sequence of DNA was generated and overexpression of the fusion protein was carried out in accordance with the general procedures described above. Overexpression was performed according to the general procedure A, using strains E. coli Tuner (DE3) from Novagen. The protein was separated by electrophoresis in accordance with the general procedure described above.

Protein was expressed with histidine tag.

### Example 40

### The Fusion Protein of SEQ. No. 40

The protein of SEQ. No. 40 is a fusion protein having the length of 481 amino acids and the mass of 53.2 kDa, wherein domain (a) is TRAIL 121-281, and domain (b) of the effector peptide is 193-amino acids catalytic domain of diphtheria toxin (SEQ. No. 80), and is attached at the C-terminus of domain (a). Additionally, between domains (a) and (b) there are sequentially incorporated steric linker sequence (GGGGS), sequence cleaved by furin (RKKR), sequence of transporting domain derived from Pseudomonas toxin (SEQ. No. 139), and steric linker sequence (GGGGS).

Thus, the structure of the fusion protein of the invention is as follows:
(TRAIL121-281)-LINKER1-FURIN-(SEQ. No. 139)-LINKER1-(SEQ. No. 80)

The amino acid sequence and the DNA encoding sequence comprising codons optimized for expression in E. coli are, respectively, SEQ. No. 40 and SEQ. No. 124 as shown in the attached Sequence Listing.

The amino acid sequence SEQ. No. 40 of the structure described above was used as a template to generate its coding DNA sequence SEQ. No. 124. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above.

Protein was expressed both with histidine tag (Ex. 40^{a}) and without histidine tag (Ex. 40^{b}).

### Example 41

### The Fusion Protein of SEQ. No. 41

The protein of SEQ. No. 41 is a fusion protein having the length of 481 amino acids and the mass of 53.2 kDa, wherein domain (a) is TRAIL 121-281, and domain (b) of the effector peptide is 189-amino acids catalytic domain of diphteria toxin (SEQ. No. 81), and is attached at the N-terminus of domain (a). Additionally, between domains (b) and (a) there are sequentially incorporated sequence cleaved by furin (RKKR), steric linker sequence (GGGGS), sequence of transporting domain derived from Pseudomonas toxin (SEQ. No. 139), sequence cleaved by furin (RKKR), and two sequences of steric linker (GGGGS).

Thus, the structure of the fusion protein of the invention is as follows:
(SEQ. No. 81)-FURIN-LINKER1-(SEQ. No. 139)-FURIN-LINKER1-LINKER1-(TRAIL121-281)

The amino acid sequence and the DNA encoding sequence comprising codons optimized for expression in E. coli are, respectively, SEQ. No. 41 and SEQ. No. 125 as shown in the attached Sequence Listing.

The amino acid sequence SEQ. No. 41 of the structure described above was used as a template to generate its coding DNA sequence SEQ. No. 125. A plasmid containing the coding sequence of DNA was generated and overexpression of the fusion protein was carried out in accordance with the general procedures described above. Overexpression was performed according to the general procedure A, using strains E. coli Tuner (DE3) from Novagen. The protein was separated by electrophoresis in accordance with the general procedure described above.

Protein was expressed with histidine tag.

### Example 42

### The Fusion Protein of SEQ. No. 42

The protein of SEQ. No. 42 is a fusion protein having the length of 432 amino acids and the mass of 48.7 kDa, wherein domain (a) is TRAIL 114-281, and domain (b) of the effector peptide is 251-amino acids domain A of abrin (SEQ. No. 82), and is attached at the N-terminus of domain (a). Additionally, between domains (b) and (a) there are sequentially incorporated two sequences of steric linker (GGGGS).

Thus, the structure of the fusion protein of the invention is as follows:
(SEQ. No. 82)-LINKER1-LINKER1-(TRAIL114-281)

The amino acid sequence and the DNA encoding sequence comprising codons optimized for expression in E. coli are, respectively, SEQ. No. 42 and SEQ. No. 126 as shown in the attached Sequence Listing.

The amino acid sequence SEQ. No. 42 of the structure described above was used as a template to generate its coding DNA sequence SEQ. No. 126. A plasmid containing the coding sequence of DNA was generated and overexpression of the fusion protein was carried out in accordance with the general procedures described above. Overexpression was performed according to the general procedure A, using strains E. coli Tuner (DE3) from Novagen. The protein was separated by electrophoresis in accordance with the general procedure described above.

Protein was expressed both with histidine tag (Ex. 42^{a}) and without histidine tag (Ex. 42^{b}).

### Example 43

### The Fusion Protein of SEQ. No. 43

The protein of SEQ. No. 43 is a fusion protein having the length of 443 amino acids and the mass of 49.7 kDa, wherein domain (a) is TRAIL 114-281, and domain (b) of the effector peptide is 251-amino acids domain A of abrin (SEQ. No. 82), and is attached at the N-terminus of domain (a). Additionally, between domains (b) and (a) there are sequentially incorporated steric linker sequence (GGGGS), sequence of integrin ligand (SEQ. No. 140), sequence cleaved by urokinase (RWR), and steric linker sequence (GGGGS)

Thus, the structure of the fusion protein of the invention is as follows:
(SEQ. No. 82)-LINKER1-(SEQ. No. 140)-UROKIN-LINKER1-(TRAIL114-281)

The amino acid sequence and the DNA encoding sequence comprising codons optimized for expression in E. coli are, respectively, SEQ. No. 43 and SEQ. No. 127 as shown in the attached Sequence Listing.

The amino acid sequence SEQ. No. 43 of the structure described above was used as a template to generate its coding DNA sequence SEQ. No. 127. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above.

Protein was expressed both with histidine tag (Ex. 43^{a}) and without histidine tag (Ex. 43^{b}).

### Example 44

### The Fusion Protein of SEQ. No. 44

The protein of SEQ. No. 44 is a fusion protein having the length of 433 amino acids and the mass of 48.7 kDa, wherein domain (a) is TRAIL 114-281, and domain (b) of the effector peptide is 251-amino acids domain A of abrin (SEQ. No. 82), and is attached at the N-terminus of domain (a). Additionally, between domains (b) and (a) there are sequentially incorporated two sequences of steric linker (GGGGS) and sequence cleaved by urokinase (RVVR).

Thus, the structure of the fusion protein of the invention is as follows:
(SEQ. No. 82)-LINKER1-LINKER1-UROKIN-(TRAIL114-281)

The amino acid sequence and the DNA encoding sequence comprising codons optimized for expression in E. coli are, respectively, SEQ. No. 44 and SEQ. No. 128 as shown in the attached Sequence Listing.

The amino acid sequence SEQ. No. 44 of the structure described above was used as a template to generate its coding DNA sequence SEQ. No. 128. A plasmid containing the coding sequence of DNA was generated and overexpression of the fusion protein was carried out in accordance with the general procedures described above. Overexpression was performed according to the general procedure A, using strains E. coli Tuner (DE3) from Novagen. The protein was separated by electrophoresis in accordance with the general procedure described above.

Protein was expressed both with histidine tag (Ex. 44^{a}) and without histidine tag (Ex. 44^{b}).

### Example 45

### The Fusion Protein of SEQ. No. 45

The protein of SEQ. No. 45 is a fusion protein having the length of 441 amino acids and the mass of 50 kDa, wherein domain (a) is TRAIL 114-281, and domain (b) of the effector peptide is 251-amino acids domain A of abrin (SEQ. No. 82), and is attached at the N-terminus of domain (a). Additionally, between domains (b) and (a) there are sequentially incorporated transporting sequence consisting of 8 arginine residues (RRRRRRRR), sequence cleaved by urokinase (RVVR), and sequentially two sequences of steric linker (GGGGS).

Thus, the structure of the fusion protein of the invention is as follows:
(SEQ. No. 82)-TRANS2-UROKIN-LINKER1-LINKER1-(TRAIL114-281)

The amino acid sequence and the DNA encoding sequence comprising codons optimized for expression in E. coli are, respectively, SEQ. No. 45 and SEQ. No. 129 as shown in the attached Sequence Listing.

The amino acid sequence SEQ. No. 45 of the structure described above was used as a template to generate its coding DNA sequence SEQ. No. 129. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above.

Protein was expressed with histidine tag.

### Example 46

### The Fusion Protein of SEQ. No. 46

The protein of SEQ. No. 46 is a fusion protein having the length of 550 amino acids and the mass of 61.3 kDa, wherein domain (a) is TRAIL 114-281, and domain (b) of the effector peptide is 251-amino acids domain A of abrin (SEQ. No. 82), and is attached at the C-terminus of domain (a). Additionally, between domains (a) and (b) there are sequentially incorporated steric linker sequence (GGGGS), sequence cleaved by urokinase (RVVR), transporting domain sequence derived from Pseudomonas (SEQ. No. 139), steric linker sequence (GGGGS), and sequence cleaved by urokinase (RVVR).

Thus, the structure of the fusion protein of the invention is as follows:
(TRAIL114-281)-LINKER1-UROKIN-(SEQ. No. 139)-LINKER1-UROKIN-(SEQ. No. 82)

The amino acid sequence and the DNA encoding sequence comprising codons optimized for expression in E. coli are, respectively, SEQ. No. 46 and SEQ. No. 130 as shown in the attached Sequence Listing.

The amino acid sequence SEQ. No. 46 of the structure described above was used as a template to generate its coding DNA sequence SEQ. No. 130. A plasmid containing the coding sequence of DNA was generated and overexpression of the fusion protein was carried out in accordance with the general procedures described above. Overexpression was performed according to the general procedure A, using strains E. coli Tuner (DE3) from Novagen. The protein was separated by electrophoresis in accordance with the general procedure described above. Protein was expressed both with histidine tag (Ex. 46^{a}) and without histidine tag (Ex. 46^{b}).

### Example 47

### The Fusion Protein of SEQ. No. 47

The protein of SEQ. No. 47 is a fusion protein having the length of 459 amino acids and the mass of 51.5 kDa, wherein domain (a) is TRAIL 95-281, and domain (b) of the effector peptide is 251-amino acids domain A of abrin (SEQ. No. 82), and is attached at the N-terminus of domain (a). Additionally, between domains (b) and (a) there are sequentially incorporated two sequences of steric linker (GGGGS), sequence cleaved by urokinase (RVVR), and pegylation linker sequence (ASGCGPE).

Thus, the structure of the fusion protein of the invention is as follows:
(SEQ. No. 82)-LINKER1-LINKER1-UROKIN-PEG-(TRAIL95-281)

The amino acid sequence and the DNA encoding sequence comprising codons optimized for expression in E. coli are, respectively, SEQ. No. 47 and SEQ. No. 131, as shown in the attached Sequence Listing.

The amino acid sequence SEQ. No. 47 of the structure described above was used as a template to generate its coding DNA sequence SEQ. No. 131. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above.

Protein was expressed both with histidine tag (Ex. 47^{a}) and without histidine tag (Ex. 47^{b}).

### Example 48

### The Fusion Protein of SEQ. No. 48

The protein of SEQ. No. 48 is a fusion protein having the length of 443 amino acids and the mass of 49.7 kDa, wherein domain (a) is TRAIL 121-281 sequence, and domain (b) of the effector peptide is 251-amino acids domain A of abrin (SEQ. No. 82), and is attached at the C-terminus of domain (a). Additionally, between domains (a) and (b) there are sequentially incorporated steric linker sequence (GGGGS), pegylation linker sequence (ASGCGPE), sequence cleaved by urokinase (RVVR) and steric linker sequence (GGGGS).

Thus, the structure of the fusion protein of the invention is as follows:
(TRAIL121-281)-LINKER1-PEG-UROKIN-LINKER1-(SEQ. No. 82)

The amino acid sequence and the DNA encoding sequence comprising codons optimized for expression in E. coli are, respectively, SEQ. No. 48 and SEQ. No. 132, as shown in the attached Sequence Listing.

The amino acid sequence SEQ. No. 48 of the structure described above was used as a template to generate its coding DNA sequence SEQ. No. 132. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

Protein was expressed with histidine tag.

### Example 49

### The Fusion Protein of SEQ. No. 49

The protein of SEQ. No. 49 is a fusion protein having the length of 447 amino acids and the mass of 50.2 kDa, wherein domain (a) is TRAIL 121-281, and domain (b) of the effector peptide is 251-amino acids domain A of abrin (SEQ. No. 82), and is attached at the C-terminus of domain (a). Additionally, between domains (a) and (b) there are sequentially incorporated steric linker sequence (GGGGS), pegylation linker sequence (ASGCGPE), sequence cleaved by urokinase (RVVR), and steric linker sequence (GGGGS). Additionally, on the C-terminus of domain (b) there is transporting sequence KDEL, directing the effector peptide the endoplasmic reticulum, forming C-terminal fragment of entire fusion protein.

Thus, the structure of the fusion protein of the invention is as follows:
(TRAIL 121-281)-LINKER1-PEG-UROKIN-LINKER1-(SEQ. No. 82)-TRANS1

The amino acid sequence and the DNA encoding sequence comprising codons optimized for expression in E. coli are, respectively, SEQ. No. 49 and SEQ. No. 133, as shown in the attached Sequence Listing.

The amino acid sequence SEQ. No. 49 of the structure described above was used as a template to generate its coding DNA sequence SEQ. No. 133. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

Protein was expressed both with histidine tag (Ex. 49^{a}) and without histidine tag (Ex. 49^{b}).

### Example 50

### The Fusion Protein of SEQ. No. 50

The protein of SEQ. No. 50 is a fusion protein having the length of 441 amino acids and the mass of 49.4 kDa, wherein domain (a) is TRAIL 114-281, and domain (b) of the effector peptide is 251-amino acids domain A of abrin (SEQ. No. 82), and is attached at the N-terminus of domain (a). Additionally, between domains (a) and (b) there are sequentially incorporated two sequences of steric linker (GGGGS), sequence cleaved by urokinase (RVVR), and pegylation linker sequence (ASGCGPE).

Thus, the structure of the fusion protein of the invention is as follows:
(SEQ. No. 82)-LINKER1-LINKER1-UROKIN-PEG-(TRAIL114-281)

The amino acid sequence and the DNA encoding sequence comprising codons optimized for expression in E. coli are, respectively, SEQ. No. 50 and SEQ. No. 134, as shown in the attached Sequence Listing.

The amino acid sequence SEQ. No. 50 of the structure described above was used as a template to generate its coding DNA sequence SEQ. No. 134. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

Protein was expressed both with histidine tag (Ex. 50^{a}) and without histidine tag (Ex. 50^{b}).

### Example 51

### The Fusion Protein of SEQ. No. 51

The protein of SEQ. No. 51 is a fusion protein having the length of 515 amino acids and the mass of 55.9 kDa, wherein domain (a) is TRAIL121-281 containing D218H mutation (SEQ. No. 142), and domain (b) of the effector peptide is a 342-amino acids homolog of the fragment of modified Pseudomonas aeruginosa exotoxin sequence (SEQ. No. 68), and is attached at the C-terminus of domain (a). Additionally, between domains (a) and (b) there are sequentially incorporated steric linker sequences (GGGS) and (ASGG). Additionally, to the C-terminus of domain (b) there is attached transporting sequence KDEL, directing the effector peptide to endoplasmic reticulum, forming C-terminal fragment of entire fusion protein.

Thus, the structure of the fusion protein of the invention is as follows:
(SEQ. No. 142)-LINKER4-LINKER3-(SEQ. No. 68)-TRANS1

The amino acid sequence and the DNA encoding sequence comprising codons optimized for expression in E. coli are, respectively, SEQ. No. 51 and SEQ. No. 135 as shown in the attached Sequence Listing.

The amino acid sequence SEQ. No. 51 of the structure described above was used as a template to generate its coding DNA sequence SEQ. No. 135. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above.

Protein was expressed both with histidine tag (Ex. 51^{a}) and without histidine tag (Ex. 51^{b}).

### Example 52

### The Fusion Protein of SEQ. No. 52

The protein of SEQ. No. 52 is a fusion protein having the length of 515 amino acids and the mass of 55.9 kDa, wherein domain (a) is TRAIL121-281 containing mutations Y189N/R191K/Q193R/H264R/1266R/D269H (SEQ. No. 143), and domain (b) of the effector peptide is a 342-amino acids homolog of the fragment of modified Pseudomonas aeruginosa exotoxin sequence (SEQ. No. 68), and is attached at the C-terminus of domain (a). Additionally, between domains (a) and (b) there are sequentially incorporated steric linker sequences (GGGS) and (ASGG). Additionally, to the C-terminus of domain (b) there is attached transporting sequence KDEL, directing the effector peptide to endoplasmic reticulum, forming C-terminal fragment of entire fusion protein.

Thus, the structure of the fusion protein of the invention is as follows:
(SEQ. No. 143)-LINKER4-LINKER3-(SEQ. No. 68)-TRANS1

The amino acid sequence and the DNA encoding sequence comprising codons optimized for expression in E. coli are, respectively, SEQ. No. 52 and SEQ. No. 136 as shown in the attached Sequence Listing.

The amino acid sequence SEQ. No. 52 of the structure described above was used as a template to generate its coding DNA sequence SEQ. No. 136. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

Protein was expressed with histidine tag.

### Example 53

### The Fusion Protein of SEQ. No. 53

The protein of SEQ. No. 53 is a fusion protein having the length of 515 amino acids and the mass of 55.9 kDa, wherein domain (a) is TRAIL121-281 containing mutation D218H (SEQ. No, 142), and domain (b) of the effector peptide is a 342-amino acids homolog of the fragment of modified Pseudomonas aeruginosa exotoxin sequence (SEQ. No. 83), and is attached at the C-terminus of domain (a). Additionally, between domains (a) and (b) there are sequentially incorporated steric linker sequences (GGGS) and pegylation linker sequence (ASGCGPE). Additionally, to the C-terminus of domain (b) there is attached transporting sequence KDEL, directing the effector peptide to endoplasmic reticulum, forming C-terminal fragment of entire fusion protein.

Thus, the structure of the fusion protein of the invention is as follows:
(SEQ. No. 142)-LINKER4-PEG-(SEQ. No. 83)-TRANS1

The amino acid sequence and the DNA encoding sequence comprising codons optimized for expression in E. coli are, respectively, SEQ. No. 53 and SEQ. No. 137 as shown in the attached Sequence Listing.

The amino acid sequence SEQ. No. 53 of the structure described above was used as a template to generate its coding DNA sequence SEQ. No. 137. A plasmid containing the coding sequence of DNA was generated and overexpression of the fusion protein was carried out in accordance with the general procedures described above. Overexpression was performed according to the general procedure A, using strain E. coli Tuner (DE3) from Novagen. The protein was separated by electrophoresis in accordance with the general procedure described above.

Protein was expressed with histidine tag.

### Example 54

### The Fusion Protein of SEQ. No. 54

The protein of SEQ. No. 54 is a fusion protein having the length of 515 amino acids and the mass of 55.9 kDa, wherein domain (a) is TRAIL121-281 containing mutations Y189N/R191K/Q193R/H264R/1266R/D269H (SEQ. No. 143), and domain (b) of the effector peptide is a 342-amino acids homolog of the fragment of modified Pseudomonas aeruginosa exotoxin sequence (SEQ. No. 83), and is attached at the C-terminus of domain (a). Additionally, between domains (a) and (b) there are sequentially incorporated steric linker sequences (GGGS) and (ASGG). Additionally, to the C-terminus of domain (b) there is attached transporting sequence KDEL, directing the effector peptide to endoplasmic reticulum, forming C-terminal fragment of entire fusion protein.

Thus, the structure of the fusion protein of the invention is as follows:
(SEQ. No. 143)-LINKER4-LINKER3-(SEQ. No. 83)-TRANS1

The amino acid sequence and the DNA encoding sequence comprising codons optimized for expression in E. coli are, respectively, SEQ. No. 54 and SEQ. No. 138 as shown in the attached Sequence Listing.

The amino acid sequence SEQ. No. 54 of the structure described above was used as a template to generate its coding DNA sequence SEQ. No. 138. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above.

Protein was expressed both with histidine tag (Ex. 54^{a}) and without histidine tag (Ex. 54^{b}).

### Example 55

### The Fusion Protein of SEQ. No. 144

The protein of SEQ. No. 144 is a fusion protein having the length of 433 amino acids and the mass of 48.8 kDa, wherein domain (a) is TRAIL114-281, and domain (b) of the effector peptide is attached at the N-terminus of domain (a) and is a 251-amino acids variant of abrin A domain (SEQ. No. 194). Additionally, between domains (b) and (a) there are sequentially incorporated two sequences of the steric linker (GGGGS), and cleavage site recognized by furin (RKKR). Thus, the structure of the fusion protein of the invention is as follows:
(SEQ. No. 194)-LINKER1-LINKER1-FURIN-(TRAIL114-281)

The amino acid sequence and the DNA encoding sequence comprising codons optimized for expression in E. coli are, respectively, SEQ. No. 144 and SEQ. No. 169 as shown in the attached Sequence Listing.

The amino acid sequence SEQ. No. 144 of the structure described above was used as a template to generate its coding DNA sequence SEQ. No. 169. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above.

Protein was expressed without histidine tag.

### Example 56

### The Fusion Protein of SEQ. No. 145

The protein of SEQ. No. 145 is a fusion protein having the length of 450 amino acids and the mass of 50.5 kDa, wherein domain (a) is TRAIL121-281, and domain (b) of the effector peptide is attached at the C-terminus of domain (a) and is a 264-amino acids deletional variant of ricin A domain (SEQ. No. 195).

Additionally, between domains (a) and (b) there are sequentially incorporated steric linker sequence (GGGGS), pegylation linker sequence (ASGCGPE), sequence recognized by furin and steric linker sequence (GGGGS). Additionally, to the C-terminus of domain (b) there is attached transporting sequence KEDL, directing the effector peptide to endoplasmic reticulum, forming C-terminal fragment of entire fusion protein.

Thus, the structure of the fusion protein of the invention is as follows:
(TRAIL121-281)-LINKER1-PEG-FURIN-LINKER1-(SEQ. No. 195)-TRANS3

The amino acid sequence and the DNA encoding sequence comprising codons optimized for expression in E. coli are, respectively, SEQ. No. 145 and SEQ. No. 170 as shown in the attached Sequence Listing.

The amino acid sequence SEQ. No. 145 of the structure described above was used as a template to generate its coding DNA sequence SEQ. No. 170. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above.

Protein was expressed without histidine tag.

### Example 57

### The Fusion Protein of SEQ. No. 146

The protein of SEQ. No. 146 is a fusion protein having the length of 481 amino acids and the mass of 53 kDa, wherein domain (a) is TRAIL121-281, and domain (b) of the effector peptide is attached at the N-terminus of domain (a) and is a 189-amino acids mutated active domain of diphtheria toxin (SEQ. No. 196).

Additionally, between domains (b) and (a) there are sequentially incorporated cleavage site sequence recognized by furin (RKKR), sequence of steric linker (GGGGS), sequence of transporting domain derived from Pseudomonas toxin (SEQ. No. 139), another cleavage site sequence recognized by furin (RKKR) followed by two sequences of steric linker (GGGGS).

Thus, the structure of the fusion protein of the invention is as follows:
(SEQ. No. 196)-FURIN-LINKER1-SEQ. No. 139-FURIN-LINKER1-LINKER1-(TRAIL121-281)

The amino acid sequence and the DNA encoding sequence comprising codons optimized for expression in E. coli are, respectively, SEQ. No. 146 and SEQ. No. 171 as shown in the attached Sequence Listing.

The amino acid sequence SEQ. No. 146 of the structure described above was used as a template to generate its coding DNA sequence SEQ. No. 171. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above

Protein was expressed without histidine tag.

### Example 58

### The Fusion Protein of SEQ. No. 147

The protein of SEQ. No. 147 is a fusion protein having the length of 478 amino acids and the mass of 52.7 kDa, wherein domain (a) is TRAIL121-281, and domain (b) of the effector peptide is attached at the N-terminus of domain (a) and is a 186-amino acids mutated active domain of diphtheria toxin (SEQ. No. 197).

Additionally, between domains (b) and (a) there are sequentially incorporated cleavage site sequence recognized by furin (RKKR), sequence of steric linker (GGGGS), sequence of transporting domain derived from Pseudomonas toxin (SEQ. No. 139), another cleavage site sequence recognized by furin (RKKR) followed by two sequences of steric linker (GGGGS).

Thus, the structure of the fusion protein of the invention is as follows:

(SEQ.No.197)-FURIN-LINKER1-SEQ.No.139-FURIN-LINKER1-LINKER1-(TRAIL121-281)

The amino acid sequence and the DNA encoding sequence comprising codons optimized for expression in E. coli are, respectively, SEQ. No. 147 and SEQ. No. 172 as shown in the attached Sequence Listing.

The amino acid sequence SEQ. No. 147 of the structure described above was used as a template to generate its coding DNA sequence SEQ. No. 172. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above

Protein was expressed without histidine tag.

### Example 59

### The Fusion Protein of SEQ. No. 148

The protein of SEQ. No. 148 is a fusion protein having the length of 433 amino acids and the mass of 48.5 kDa, wherein domain (a) is TRAIL121-281, and domain (b) of the effector peptide is attached at the N-terminus of domain (a) and is a 251-amino acids mutated variant of gelonin (SEQ. No. 198).

Additionally, between domains (b) and (a) there are sequentially incorporated sequence of steric linker (GGGGS), cleavage site sequence recognized by furin (RKKR), pegylation linker (ASGCGPE) and sequence of steric linker (GGGGS).

Thus, the structure of the fusion protein of the invention is as follows:
(SEQ. No 198)- LINKER1-FURIN-PEG-LINKER1-(TRAIL121-281)

The amino acid sequence and the DNA encoding sequence comprising codons optimized for expression in E. coli are, respectively, SEQ. No. 148 and SEQ. No. 173 as shown in the attached Sequence Listing.

The amino acid sequence SEQ. No. 148 of the structure described above was used as a template to generate its coding DNA sequence SEQ. No. 173. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above

Protein was expressed both with histidine tag (Ex. 59^{a}) and without histidine tag (Ex. 59^{b}).

### Example 60

### The Fusion Protein of SEQ. No. 149

The protein of SEQ. No. 149 is a fusion protein having the length of 258 amino acids and the mass of 29.5 kDa, wherein domain (a) is TRAIL95-281, and domain (b) of the effector peptide is attached at the C-terminus of domain (a) and is a 47-amino acids P1 luffin peptide (SEQ. No. 65).

Additionally, between domains (a) and (b) there are sequentially incorporated three sequences of steric linkers (GGGGS), (GGG) and (CAAACAAC) followed by sequence of cleavage site recognized by furin (RKKR). Additionally, to the C-terminus of domain (b) there is attached transporting sequence KDEL, directing the effector peptide to endoplasmic reticulum, forming C-terminal fragment of entire fusion protein.

Thus, the structure of the fusion protein of the invention is as follows:
(TRAIL95-281)-LINKER1-LINKER7-LINKER6-FURIN-(SEQ.No. 65)-TRANS1

The amino acid sequence and the DNA encoding sequence comprising codons optimized for expression in E. coli are, respectively, SEQ. No. 149 and SEQ. No. 174 as shown in the attached Sequence Listing.

The amino acid sequence SEQ. No. 149 of the structure described above was used as a template to generate its coding DNA sequence SEQ. No. 174. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above

Protein was expressed without histidine tag.

### Example 61

### The Fusion Protein of SEQ. No. 150

The protein of SEQ. No. 150 is a fusion protein having the length of 253 amino acids and the mass of 29.2 kDa, wherein domain (a) is TRAIL95-281, and domain (b) of the effector peptide is attached at the N-terminus of domain (a) and is a 47-amino acids P1 luffin peptide (SEQ. No. 65).

Additionally, between domains (b) and (a) there are sequentially incorporated sequence of cleavage site recognized by furin (RKKR) and sequences of steric linkers (GGG) and (CAAACAAC). Additionally, to the C-terminus of domain (b) there is attached transporting sequence KDEL, directing the effector peptide to endoplasmic reticulum, forming C-terminal fragment of entire fusion protein.

Thus, the structure of the fusion protein of the invention is as follows:
(SEQ.No. 65)-TRANS1-FURIN-LINKER7-LINKER6-(TRAIL95-281)

The amino acid sequence and the DNA encoding sequence comprising codons optimized for expression in E. coli are, respectively, SEQ. No. 150 and SEQ. No. 175 as shown in the attached Sequence Listing.

The amino acid sequence SEQ. No. 150 of the structure described above was used as a template to generate its coding DNA sequence SEQ. No. 175. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above.

Protein was expressed without histidine tag.

### Example 62

### The Fusion Protein of SEQ. No. 151

The protein of SEQ. No. 151 is a fusion protein having the length of 539 amino acids and the mass of 59.3 kDa, wherein domain (a) is TRAIL121-281, and domain (b) of the effector peptide is attached at the N-terminus of domain (a) and is a 247-amino acids mutated variant of trichosantin (SEQ. No. 199).

Additionally, between domains (b) and (a) there are sequentially incorporated sequence of cleavage site recognized by furin (RKKR) and sequence of steric Linker (GGGGS) followed by sequence of transporting domain derived from Pseudomonas toxin (SEQ. No. 139), another cleavage site recognized by furin (RKKR) and two sequences of steric linkers (GGGGS).

Thus, the structure of the fusion protein of the invention is as follows:
(SEQ. No. 199)-FURIN-LINKER1-SEQ. No. 139-FURIN-LINKER1-LINKER1-(TRAIL121-281)

The amino acid sequence and the DNA encoding sequence comprising codons optimized for expression in E. coli are, respectively, SEQ. No. 151 and SEQ. No. 176 as shown in the attached Sequence Listing.

The amino acid sequence SEQ. No. 151 of the structure described above was used as a template to generate its coding DNA sequence SEQ. No. 176. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above.

Protein was expressed without histidine tag.

### Example 63

### The Fusion Protein of SEQ. No. 152

The protein of SEQ. No. 152 is a fusion protein having the length of 429 amino acids and the mass of 47.2 kDa, wherein domain (a) is TRAIL121-281, and domain (b) of the effector peptide is attached at the N-terminus of domain (a) and is a 247-amino acids mutated variant of trichosantin (SEQ. No. 200).

Additionally, between domains (b) and (a) there are sequentially incorporated sequence of steric linker (GGGGS) and sequence of cleavage site recognized by furin (RKKR) followed by pegylation sequence (ASGCGPE) and sequence of steric linker (GGGGS).

Thus, the structure of the fusion protein of the invention is as follows:
(SEQ. No. 200)-LINKER1-FURIN-PEG-LINKER1-(TRAIL121-281)

The amino acid sequence and the DNA encoding sequence comprising codons optimized for expression in E. coli are, respectively, SEQ. No. 152 and SEQ. No. 177 as shown in the attached Sequence Listing.

The amino acid sequence SEQ. No. 152 of the structure described above was used as a template to generate its coding DNA sequence SEQ. No. 177. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above.

Protein was expressed without histidine tag.

### Example 64

### The fusion protein of SEQ. No. 153

The protein of SEQ. No. 153 is a fusion protein having the length of 515 amino acids and the mass of 55.9 kDa, wherein domain (a) is TRAIL121-281, and domain (b) of the effector peptide is 342-amino acids modified Pseudomonas aeruginosa exotoxin sequence with point mutations R318K, N441Q and R601K (SEQ. No. 201), and is attached at the C-terminus of domain (a).

Additionally, between domains (a) and (b) there are sequentially incorporated two sequences of steric linkers (GGGS) and (ASGG). Additionally, to the C-terminus of domain (b) there is attached transporting sequence KDEL, directing the effector peptide to endoplasmic reticulum, forming C-terminal fragment of entire fusion protein.

Thus, the structure of the fusion protein of the invention is as follows:
(TRAIL121-281)-LINKER4-LINKER3-SEQ.No. 201-(TRANS1)

The amino acid sequence and the DNA encoding sequence comprising codons optimized for expression in E. coli are, respectively, SEQ. No. 153 and SEQ. No. 178 as shown in the attached Sequence Listing.

The amino acid sequence SEQ. No. 153 of the structure described above was used as a template to generate its coding DNA sequence SEQ. No. 178. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above.

Protein was expressed without histidine tag.

### Example 65

### The Fusion Protein of SEQ. No. 154

The protein of SEQ. No. 154 is a fusion protein having the length of 402 amino acids and the mass of 43.3 kDa, wherein domain (a) is TRAIL121-281, and domain (b) of the effector peptide is a 225-amino acids deletion variant of Pseudomonas aeruginosa exotoxin sequence (SEQ. No. 202), and is attached at the C-terminus of domain (a).

Additionally, between domains (a) and (b) there are sequentially incorporated two sequences of steric linkers (GGGS) and (GGGG) and sequence of cleavage site recognized by furin (RKKR). Additionally, to the C-terminus of domain (b) there is attached transporting sequence KEDL, directing the effector peptide to endoplasmic reticulum, forming C-terminal fragment of entire fusion protein.

Thus, the structure of the fusion protein of the invention is as follows:
(TRAIL121-281)-LINKER4-LINKER2-FURIN-(SEQ. No. 202)-TRANS3

The amino acid sequence and the DNA encoding sequence comprising codons optimized for expression in E. coli are, respectively, SEQ. No. 154 and SEQ. No. 179 as shown in the attached Sequence Listing.

The amino acid sequence SEQ. No. 154 of the structure described above was used as a template to generate its coding DNA sequence SEQ. No. 179. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above.

Protein was expressed both with histidine tag (Ex. 65^{a}) and without histidine tag (Ex. 65^{b}).

### Example 66

### The Fusion Protein of SEQ. No. 155

The protein of SEQ. No. 155 is a fusion protein having the length of 403 amino acids and the mass of 44.3 kDa, wherein domain (a) is TRAIL121-281, and domain (b) of the effector peptide is a 226-amino acids deletion variant of Pseudomonas aeruginosa exotoxin sequence with several point mutations (SEQ. No. 203), and is attached at the C-terminus of domain (a).

Additionally, between domains (a) and (b) there are sequentially incorporated two sequences of steric linkers (GGGGS) and (GGGG) and sequence of cleavage site recognized by furin (RKKR). Additionally, to the C-terminus of domain (b) there is attached transporting sequence KEDL, directing the effector peptide to endoplasmic reticulum, forming C-terminal fragment of entire fusion protein.

Thus, the structure of the fusion protein of the invention is as follows:
TRAIL121-281-LINKER1-LINKER2-FURIN-SEQ. No. 203-TRANS3

The amino acid sequence and the DNA encoding sequence comprising codons optimized for expression in E. coli are, respectively, SEQ. No. 155 and SEQ. No. 180 as shown in the attached Sequence Listing.

The amino acid sequence SEQ. No. 155 of the structure described above was used as a template to generate its coding DNA sequence SEQ. No. 180. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above.

Protein was expressed both with histidine tag (Ex. 66^{a}) and without histidine tag (Ex. 66^{b}).

### Example 67

### The Fusion Protein of SEQ. No. 156

The protein of SEQ. No. 156 is a fusion protein having the length of 470 amino acids and the mass of 51.5 kDa, wherein domain (a) is TRAIL121-281, and domain (b) of the effector peptide is a 279-amino acids deletion variant of Pseudomonas aeruginosa exotoxin sequence with several point mutations (SEQ. No. 204), and attached at the C-terminus of domain (a).

Additionally, between domains (a) and (b) there are sequentially incorporated a sequence of steric linker (GGGGS) and pegylation linker (ASGCGPE) followed by a sequence recognized by furin (RKKR) and native sequence of cleavage site recognized by furin (RHRQPRGWEQL). Additionally, to the C-terminus of domain (b) there is attached transporting sequence KEDL, directing the effector peptide to endoplasmic reticulum, forming C-terminal fragment of entire fusion protein.

Thus, the structure of the fusion protein of the invention is as follows:
(TRAIL121-281)-LINKER1-PEG-FURIN-FURIN.NAT-(SEQ. No. 204)-TRANS3

The amino acid sequence and the DNA encoding sequence comprising codons optimized for expression in E. coli are, respectively, SEQ. No. 156 and SEQ. No. 181 as shown in the attached Sequence Listing.

The amino acid sequence SEQ. No. 156 of the structure described above was used as a template to generate its coding DNA sequence SEQ. No. 181. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above.

Protein was expressed both with histidine tag (Ex. 67^{a}) and without histidine tag (Ex. 67^{b}).

### Example 68

### The Fusion Protein of SEQ. No. 157

The protein of SEQ. No. 157 is a fusion protein having the length of 478 amino acids and the mass of 51.8 kDa, wherein domain (a) is TRAIL121-281, and domain (b) of the effector peptide is a 279-amino acids deletion variant of Pseudomonas aeruginosa exotoxin sequence with several point mutations (SEQ. No. 205), and is attached at the C-terminus of domain (a).

Additionally, between domains (a) and (b) there are sequentially incorporated repeated sequence of steric linker (GGGGS) followed by cleavage site recognized by furin (RKKR), native sequence of cleavage site recognized by furin (RHRQPRGWEQL) and repeated sequence of steric linker (GGGGS). Additionally, to the C-terminus of domain (b) there is attached transporting sequence KEDL, directing the effector peptide to endoplasmic reticulum, forming C-terminal fragment of entire fusion protein.

Thus, the structure of the fusion protein of the invention is as follows:
(TRAIL121-281)-LINKER1-LINKER1-FURIN-FURIN.NAT-LINKER1-LINKER1-(SEQ.No.205)-TRANS3

The amino acid sequence and the DNA encoding sequence comprising codons optimized for expression in E. coli are, respectively, SEQ. No. 157 and SEQ. No. 182 as shown in the attached Sequence Listing.

The amino acid sequence SEQ. No. 157 of the structure described above was used as a template to generate its coding DNA sequence SEQ. No. 182. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above.

Protein was expressed both with histidine tag (Ex. 68^{b}) and without histidine tag (Ex. 68^{b}).

### Example 69

### The Fusion Protein of SEQ. No. 158

The protein of SEQ. No. 158 is a fusion protein having the length of 402 amino acids and the mass of 44.7 kDa, wherein domain (a) is TRAIL121-281, and domain (b) of the effector peptide is a 214-amino acids mutated deletion variant of Pseudomonas aeruginosa exotoxin sequence (SEQ. No. 206), and is attached at the C-terminus of domain (a).

Additionally, between domains (a) and (b) there are sequentially incorporated a sequence of steric linker (GGGGS), followed by sequence of steric linker (GGGG), cleavage site recognized by furin (RKKR) and native sequence of cleavage site recognized by furin (RHRQPRGWEQL) Additionally, to the C-terminus of domain (b) there is attached transporting sequence KEDL, directing the effector peptide to endoplasmic reticulum, forming C-terminal fragment of entire fusion protein.

Thus, the structure of the fusion protein of the invention is as follows:
(TRAIL121 -281)-LINKER1-LINKER2-FURIN-FURIN.NAT-(SEQ. No. 206)-TRANS3

The amino acid sequence and the DNA encoding sequence comprising codons optimized for expression in E. coli are, respectively, SEQ. No. 158 and SEQ. No. 183 as shown in the attached Sequence Listing.

The amino acid sequence SEQ. No. 158 of the structure described above was used as a template to generate its coding DNA sequence SEQ. No. 183. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above.

Protein was expressed without histidine tag.

### Example 70

### The Fusion Protein of SEQ. No. 159

The protein of SEQ. No. 159 is a fusion protein having the length of 467 amino acids and the mass of 50.4 kDa, wherein domain (a) is TRAIL121-281, and domain (b) of the effector peptide is a 279-amino acids mutated deletion variant of Pseudomonas aeruginosa exotoxin sequence with several point mutations (SEQ. No. 205), and is attached at the C-terminus of domain (a).

Additionally, between domains (a) and (b) there are sequentially incorporated repeated sequence of steric linker (GGGGS) followed by cleavage site recognized by furin (RKKR) and another repeated sequence of steric linker (GGGGS). Additionally, to the C-terminus of domain (b) there is attached transporting sequence KEDL, directing the effector peptide to endoplasmic reticulum, forming C-terminal fragment of entire fusion protein.

Thus, the structure of the fusion protein of the invention is as follows:
(TRAIL121-281)-LINKER1-LINKER1-FURIN- LINKER1-LINKER1-(SEQ. No. 205)-TRANS3

The amino acid sequence and the DNA encoding sequence comprising codons optimized for expression in E. coli are, respectively, SEQ. No. 159 and SEQ. No. 184 as shown in the attached Sequence Listing.

The amino acid sequence SEQ. No. 159 of the structure described above was used as a template to generate its coding DNA sequence SEQ. No. 184. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above.

Protein was expressed without histidine tag.

### Example 71

### The Fusion Protein of SEQ. No. 160

The protein of SEQ. No. 160 is a fusion protein having the length of 474 amino acids and the mass of 51.3 kDa, wherein domain (a) is TRAIL121-281, and domain (b) of the effector peptide is a 279-amino acids mutated deletion variant of Pseudomonas oeruginosa exotoxin sequence with several point mutations (SEQ. No. 205), and is attached at the C-terminus of domain (a).

Additionally, between domains (a) and (b) there are sequentially incorporated repeated sequence of steric linker (GGGGS) followed by native cleavage site sequence recognized by furin (RHRQPRGWEQL) and another repeated sequence of steric linker (GGGGS). Additionally, to the C-terminus of domain (b) there is attached transporting sequence KEDL, directing the effector peptide to endoplasmic reticulum, forming C-terminal fragment of entire fusion protein.

Thus, the structure of the fusion protein of the invention is as follows:
TRAIL121-281-LINKER1-LINKER1-FURIN.NAT-LINKER1-LINKER1-SEQ.No.205-TRANS3

The amino acid sequence and the DNA encoding sequence comprising codons optimized for expression in E. coli are, respectively, SEQ. No. 160 and SEQ. No. 185 as shown in the attached Sequence Listing.

The amino acid sequence SEQ. No. 160 of the structure described above was used as a template to generate its coding DNA sequence SEQ. No. 185. A plasmid containing the coding sequence of DNA was generated and overexpression of the fusion protein was carried out in accordance with the general procedures described above. Overexpression was performed according to the general procedure B, using E. coli BLZ1 (DE3) or Tuner (DE3) strain from Novagen. The protein was separated by electrophoresis in accordance with the general procedure described above.

Protein was expressed both with histidine tag (Ex. 71^{a}) and without histidine tag (Ex. 71^{b}).

### Example 72

### The Fusion Protein of SEQ. No. 161

The protein of SEQ. No. 161 is a fusion protein having the length of 474 amino acids and the mass of 51.3 kDa, wherein domain (a) is TRAIL121-281, and domain (b) of the effector peptide is a 279-amino acids mutated deletion variant of Pseudomonas aeruginosa exotoxin sequence with several point mutations (SEQ. No. 205), and is attached at the C-terminus of domain (a).

Additionally, between domains (a) and (b) there are sequentially incorporated repeated sequence of steric linker (GGGGS) followed by native cleavage site sequence recognized by furin (RHRQPRGWEQL) and another repeated sequence of steric linker (GGGGS). Additionally, to the C-terminus of domain (b) there is attached transporting sequence KDEL, directing the effector peptide to endoplasmic reticulum, forming C-terminal fragment of entire fusion protein.

Thus, the structure of the fusion protein of the invention is as follows:
(TRAIL121-281)-LINKER1-LINKER1-FURIN.NAT-LINKER1-LINKER1-(SEQ.No.205)-TRANS1

The amino acid sequence and the DNA encoding sequence comprising codons optimized for expression in E. coli are, respectively, SEQ. No. 161 and SEQ. No. 186 as shown in the attached Sequence Listing.

The amino acid sequence SEQ. No. 161 of the structure described above was used as a template to generate its coding DNA sequence SEQ. No. 186. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above.

Protein was expressed without histidine tag.

### Example 73

### The fusion protein of SEQ. No. 162

The protein of SEQ. No. 162 is a fusion protein having the length of 474 amino acids and the mass of 51.2 kDa, wherein domain (a) is TRAIL121-281, and domain (b) of the effector peptide is a 279-amino acids deletion variant of Pseudomonas aeruginosa exotoxin sequence with mutations (SEQ. No. 207), and is attached at the C-terminus of domain (a).

Additionally, between domains (a) and (b) there are sequentially incorporated repeated sequence of steric linker (GGGGS) followed by native cleavage site sequence recognized by furin (RHRQPRGWEQL) and another repeated sequence of steric linker (GGGGS). Additionally, to the C-terminus of domain (b) there is attached transporting sequence KDEL, directing the effector peptide to endoplasmic reticulum, forming C-terminal fragment of entire fusion protein.

Thus, the structure of the fusion protein of the invention is as follows:
(TRAM 21-281)-LINKER1-LINKER1-FURIN.NAT-LINKER1-LINKER1-(SEQ.No.207)-TRANS1

The amino acid sequence and the DNA encoding sequence comprising codons optimized for expression in E. coli are, respectively, SEQ. No. 162 and SEQ. No. 187 as shown in the attached Sequence Listing.

The amino acid sequence SEQ. No. 162 of the structure described above was used as a template to generate its coding DNA sequence SEQ. No. 187. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above. Protein was expressed without histidine tag.

### Example 74

### The Fusion Protein of SEQ. No. 163

The protein of SEQ. No. 163 is a fusion protein having the length of 515 amino acids and the mass of 55.9 kDa, wherein domain (a) is TRAIL121-281 containing mutation D218H (SEQ. No. 142), and domain (b) of the effector peptide is a 342-amino acids modified Pseudomonas aeruginosa exotoxin sequence with three point mutations R318K, N441Q and R601K (SEQ. No. 201), and is attached at the C-terminus of domain (a). Additionally, between domains (a) and (b) there are sequentially incorporated steric linker sequences (GGGS) and (ASGG). Additionally, to the C-terminus of domain (b) there is attached transporting sequence KDEL, directing the effector peptide to endoplasmic reticulum, forming C-terminal fragment of entire fusion protein.

Thus, the structure of the fusion protein of the invention is as follows:
(SEQ. No. 142)-LINKER4-LINKER3-(SEQ. No. 201)-TRANS1

The amino acid sequence and the DNA encoding sequence comprising codons optimized for expression in E. coli are, respectively, SEQ. No. 163 and SEQ. No. 188 as shown in the attached Sequence Listing.

The amino acid sequence SEQ. No. 163 of the structure described above was used as a template to generate its coding DNA sequence SEQ. No. 188. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above.

Protein was expressed without histidine tag.

### Example 75

### The Fusion Protein of SEQ. No. 164

The protein of SEQ. No. 164 is a fusion protein having the length of 475 amino acids and the mass of 51.4 kDa, wherein domain (a) is TRAIL121-281 containing mutation D218H (SEQ. No. 142), and domain (b) of the effector peptide is a 279-amino acids mutated deletion variant of Pseudomonas aeruginosa exotoxin sequence with several point mutations (SEQ. No. 205), and is attached at the C-terminus of domain (a). Additionally, between domains (a) and (b) there are sequentially incorporated repeated sequence of steric linker (GGGGS), followed by native cleavage site sequence recognized by furin (RHRQPRGWEQL) and another repeated sequence of steric linker (GGGGS). Additionally, to the C-terminus of domain (b) there is attached transporting sequence KDEL, directing the effector peptide to endoplasmic reticulum, forming C-terminal fragment of entire fusion protein.

Thus, the structure of the fusion protein of the invention is as follows:
(SEQ.No.142)-LINKER1-LINKER1-FURIN.NAT-LINKER1-LINKER1-(SEQ.No.205)-TRANS1

The amino acid sequence and the DNA encoding sequence comprising codons optimized for expression in E. coli are, respectively, SEQ. No. 164 and SEQ. No. 189 as shown in the attached Sequence Listing.

The amino acid sequence SEQ. No. 164 of the structure described above was used as a template to generate its coding DNA sequence SEQ. No. 189. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above.

Protein was expressed without histidine tag.

### Example 76

### The Fusion Protein of SEQ. No. 165

The protein of SEQ. No. 165 is a fusion protein having the length of 463 amino acids and the mass of 50.6 kDa, wherein domain (a) is TRAIL121-281 containing mutation D218H (SEQ. No. 142), and domain (b) of the effector peptide is a 279-amino acids deletion variant of Pseudomonas aeruginosa exotoxin sequence with several point mutations (SEQ. No. 204), and is attached at the C-terminus of domain (a). Additionally, between domains (a) and (b) there are sequentially incorporated two sequences of steric linker (GGGS) followed by a native sequence of cleavage site recognized by furin (RHRQPRGWEQL).

Additionally, to the C-terminus of domain (b) there is attached transporting sequence KDEL, directing the effector peptide to endoplasmic reticulum, forming C-terminal fragment of entire fusion protein.

Thus, the structure of the fusion protein of the invention is as follows:
(SEQ. No. 142)- LINKER4-LINKER4-FURIN.NAT-(SEQ. No. 204)-TRANS1

The amino acid sequence and the DNA encoding sequence comprising codons optimized for expression in E. coli are, respectively, SEQ. No. 165 and SEQ. No. 190 as shown in the attached Sequence Listing.

The amino acid sequence SEQ. No. 165 of the structure described above was used as a template to generate its coding DNA sequence SEQ. No. 190. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above.

Protein was expressed without histidine tag.

### Example 77

### The Fusion Protein of SEQ. No. 166

The protein of SEQ. No. 166 is a fusion protein having the length of 475 amino acids and the mass of 51.4 kDa, wherein domain (a) is TRAIL121-281 containing mutations Y189N/R191K/Q193R/H264R/I266R/D269H (SEQ. No. 143), and domain (b) of the effector peptide is a 279-amino acids mutated deletion variant of Pseudomonas aeruginosa exotoxin sequence with several point mutations (SEQ. No. 205), and is attached at the C-terminus of domain (a). Additionally, between domains (a) and (b) there are sequentially incorporated two sequences of steric linker (GGGGS) followed by a native sequence of cleavage site recognized by furin (RHRQPRGWEQL) and two sequences of steric linker (GGGGS).

Additionally, to the C-terminus of domain (b) there is attached transporting sequence KDEL, directing the effector peptide to endoplasmic reticulum, forming C-terminal fragment of entire fusion protein.

Thus, the structure of the fusion protein of the invention is as follows:
(SEQ. No. 143)-LINKER1-LINKER1-FURIN.NAT-LINKER1-LINKER1-(SEQ. No. 205)-TRANS1

The amino acid sequence and the DNA encoding sequence comprising codons optimized for expression in E. coli are, respectively, SEQ. No. 166 and SEQ. No. 191 as shown in the attached Sequence Listing.

The amino acid sequence SEQ. No. 166 of the structure described above was used as a template to generate its coding DNA sequence SEQ. No. 191. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above.

Protein was expressed without histidine tag.

### Example 78

### The Fusion Protein of SEQ. No. 167

The protein of SEQ. No. 167 is a fusion protein having the length of 474 amino acids and the mass of 51.24 kDa, wherein domain (a) is TRAIL121-281, and domain (b) of the effector peptide is a 279-amino acids deletion variant of Pseudomonas aeruginosa exotoxin A sequence with mutations (SEQ. No. 207), and is attached at the C-terminus of domain (a). Additionally, between domains (a) and (b) there are sequentially incorporated two sequences of steric linker (GGGGS) followed by a native sequence of cleavage site recognized by furin (RHRQPRGWEQL) and two sequences of steric linker (GGGGS).

Additionally, to the C-terminus of domain (b) there is attached transporting sequence KEDL, directing the effector peptide to endoplasmic reticulum, forming C-terminal fragment of entire fusion protein.

Thus, the structure of the fusion protein of the invention is as follows:
(TRAIL121-281)-LINKER) -LINKER1-FURIN.NAT-LINKER1-LINKER1-(SEQ. No. 207)-TRANS3

The amino acid sequence and the DNA encoding sequence comprising codons optimized for expression in E. coli are, respectively, SEQ. No. 167 and SEQ. No. 192 as shown in the attached Sequence Listing.

The amino acid sequence SEQ. No. 167 of the structure described above was used as a template to generate its coding DNA sequence SEQ. No. 192. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above.

Protein was expressed both with histidine tag (Ex. 78^{a}) and without histidine tag (Ex. 78^{b}).

### Example 79

### The Fusion Protein of SEQ. No. 168

The protein of SEQ. No. 168 is a fusion protein having the length of 232 amino acids and the mass of 26.2 kDa, wherein domain (a) is TRAIL121-281, and domain (b) of the effector peptide is 51 amino acids Hok protein sequence (SEQ. No. 208), and is attached at the C-terminus of domain (a). Additionally, between domains (b) and (a) there are sequentially incorporated a sequence of steric linker (GGGGS) followed by sequences of cleavage site recognized by urokinase (RWR) and metalloprotease MMP (PLGLAG) and a sequence of steric linker (GGGGS).

Thus, the structure of the fusion protein of the invention is as follows:
(SEQ. No. 208)-LINKER1-UROKIN-MMP-LINKER1-(TRAIL121-281)

The amino acid sequence and the DNA encoding sequence comprising codons optimized for expression in E. coli are, respectively, SEQ. No. 168 and SEQ. No. 193 as shown in the attached Sequence Listing.

The amino acid sequence SEQ. No. 168 of the structure described above was used as a template to generate its coding DNA sequence SEQ. No. 193. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above.

### Example 80

### Examination of Anti-Tumor Activity of the Fusion Proteins

Examination of anti-tumor activity of the fusion proteins was carried out in vitro in a cytotoxicity assay on tumor cell lines and in vivo in mice. For comparison purposes, rhTRAIL114-281 protein and placebo were used.

1. Measurement of Circular Dichroism: Determination of Secondary Structures Composition of the Obtained Proteins

Quality of the preparations of fusion proteins in terms of their structures was determined by circular dichroism for the fusion proteins of Ex. 2^{a}, Ex. 11^{a}, Ex. 12^{a}, Ex. 13^{a}, Ex. 14^{a}, Ex. 15^{a}, Ex. 18^{a}, Ex. 20^{a}, Ex. 26^{a}, Ex. 29^{a}, Ex. 42^{a}, Ex. 43^{a}, Ex. 44^{a}, Ex. 50^{a}, Ex. 51^{a}, and Ex. 52^{a}. Circular dichroism is used for determination of secondary structures and conformation of proteins. Co method uses optical activity of the protein structures, manifested in rotating the plane of polarization of light and the appearance of elliptical polarization. CD spectrum of proteins in far ultraviolet (UV) provides precise data on the conformation of the main polypeptide chain.

Samples of the protein to be analysed, after formulation into a buffer consisting of 50 mM Tris-HCl pH 8.0, 100 mM NaCl, 10% glycerol, 0.1 mM ZnCl₂, 80 mM saccharose, 5 mM DTT, were dialysed in dialysis bags (Sigma-Aldrich) with cut-off 12 kDa. Dialysis was performed against 100 fold excess (v/v) of buffer with respect to protein preparations, with stirring for several hours at 4° C. After dialysis was completed, each preparation was centrifuged (25 000 rpm., 10 min., 4′C) and supernatants were collected.

Protein Concentration in the Samples thus Obtained was Determined by Bradford Method.

Measurement of circular dichroism for proteins in the concentration range of 0.1-2.7 mg/ml was performed on Jasco J-710 spectropolarimeter, in a quartz cuvette with optical way 0.2 mm or 1 mm. The measurement was performed under the flow of nitrogen at 7 l/min, which allowed to perform the measurement in the wavelength range from 195 to 250 nm. Parameters of the measurement: spectral resolution of—1 nm; half width of the light beam 1 nm; sensitivity 20 mdeg, the averaging time for one wavelength—8 s, scan speed 10 nm/min.

Obtained spectra were analyzed numerically in the range of 193-250 nm using CDPro software. Points for which the voltage at the photomultiplier exceeded 700 V were omitted, due to too low signal to noise ratio in this wavelength range.

The data obtained served for calculations of particular secondary structures content in the analyzed proteins with use of CDPro software (Table 1).

TABLE 1
Content of secondary structures in the analyzed proteins.

| | NRMSD | | β- | | |
|---|---|---|---|---|---|
| Protein | (Exp-Cal) | α-helix | sheet | Schift | Disorder |
| rhTRAIL 114-281 | 0.389 | 4.9% | 33.7% | 23.1% | 38.3% |
| hrTRAIL* | | 1.94% | 50.97% | 7.74% | 39.35% |
| Ex. 2^{a} | 0.454 | 22.8% | 30.4% | 24.3% | 22.5% |
| Ex. 11^{a} | 0.016 | 58.7% | 6.7% | 11.0% | 23.6% |
| Ex. 12^{a} | 0.061 | 6.6% | 35.7% | 27.5% | 30.2% |
| Ex. 13^{a} | 0.258 | 3.6% | 41.3% | 21.2% | 33.8% |
| Ex. 14^{a} | 0.184 | 4.3% | 39.4% | 21.7% | 34.6% |
| Ex. 18^{a} | 0.011 | 72.5% | 3.1% | 2.2% | 22.2% |
| Ex. 15^{a} | 0.032 | 20.9% | 20.7% | 29.6% | 28.9% |
| Ex. 20^{a} | 0.042 | 25.5% | 20.3% | 31.6% | 22.7% |
| Ex. 42^{a} | 0.045 | 24.9% | 20.9% | 32.2% | 21.9% |
| Ex. 26^{a} | 0.129 | 5.2% | 38.7% | 22.1% | 34.1% |
| Ex. 29^{a} | 0.149 | 3.7% | 42.0% | 21.1% | 33.2% |
| Ex. 43^{a} | 0.035 | 34.7% | 16.0% | 20.5% | 28.9% |
| Ex. 44^{a} | 0.052 | 26.3% | 21.3% | 31.7% | 20.8% |
| Ex. 50^{a} | 0.036 | 22.8% | 19.2% | 34.1% | 23.9% |
| Ex. 51^{a} | 0.212 | 16.6% | 32.2% | 23.0% | 28.2% |
| Ex. 52^{a} | 0.039 | 17.5% | 27.7% | 22.1% | 32.8% |
| **Pseudomonas | | 51% | 13% | | |
| exotoxin | | | | | |
| **Shiga toxin | | 43% | 22% | | |
| **abrin | | 46% | 20% | | |
| **ricin | | 48% | 20% | | |

*value obtained on the basis of crystalline structure 1D4V
**values obtained on the basis of crystalline structures 1IKQ, 1R4Q, 1ABR, 3PX8

The control molecule (rhTRAIL114-281) shows CD spectrum characteristic for the proteins with predominantly type β-sheet structures (sharply outlined ellipticity minimum at the wavelength of 220 nm). This confirms the calculation of secondary structure components, suggesting a marginal number of α-helix elements.

The obtained result is also consistent with the data from the crystal structure of hTRAIL protein, and characteristic for fusion proteins of the invention (Ex. 12^{a}, Ex. 13^{a}, Ex. 14^{a} and Ex. 29^{a}), wherein beta elements constitute 32-44% of their structure. For all Examples, dichroism spectra are characterized by one minimum at wavelength 220 nm. Since small peptides attached to TRAIL constitute a small portion of the protein and do not need to create a defined secondary structure, analyzed proteins should not differ significantly from the starting protein.

In the case of constructs of Ex. 2^{a}, Ex. 11^{a}, Ex. 15^{a}, Ex. 20^{a}, Ex. 26^{a}, Ex. 42^{a}, Ex. 43^{a}, Ex. 44^{a}, Ex. 50^{a}, Ex. 51^{a} and Ex. 52^{a}, mixed content of secondary structures alpha/beta was observed, which is consistent with expectations based on the known crystal structure of the effector peptides domains. The content of alpha structures at the level of 50% in the case of these bulky domains has a significant impact on the structure of the fusion protein.

Only the protein of Ex. 18^{a} has over 70% of alpha-helix content and low content of beta structures.

2.Tests on Cell Lines In Vitro

Cell Lines

The cell lines were obtained from ATCC and CLS, and then propagated and deposited in the Laboratory of Biology Adamed's Cell Line Bank. During the experiment, cells were routinely checked for the presence of Mycoplasma by PCR technique using the kit Venor®GeM Mycoplasma PCR Detection Kit (Minerva Biolabs, Berlin, Germany). The cultures were maintained at standard conditions: 37° C., 5% CO₂ (in case of DMEM—10% CO₂), and 85% relative humidity. Particular cell lines were cultured in appropriate media as recommended by ATCC.

TABLE 2
Adherent cell lines

| | | | number of |
|---|---|---|---|
| | | | cells per |
| | | | well |
| Cell line | Cancer type | Medium | (thousands) |
| Colo 205 | human colorectal | RPMI + 10% FBS + penicillin + | 5 |
| ATCC | cancer | streptomycin | |
| #CCL-222 | | | |
| HT-29 | human colorectal | McCoy's + 10% FBS + penicillin + | 5 |
| ATCC | cancer | streptomycin | |
| # CCL-2 | | | |
| DU-145 | human prostate | RPMI + 10% FBS + penicillin + | 3 |
| ATCC | cancer | streptomycin | |
| # HTB-81 | | | |
| PC-3 | human prostate | RPMI + 10% FBS + penicillin + | 4 |
| ATCC | cancer | streptomycin | |
| # CRL-1435 | | | |
| MCF-7 | human breast cancer | MEM + 10% FBS + penicillin + | 4.5 |
| ATCC | | streptomycin | |
| #HTB-22 | | | |
| MDA-MB-231 | human breast cancer | DMEM + 10% FBS + penicillin + | 4.5 |
| ATCC | | streptomycin | |
| # HTB-26 | | | |
| MDA-MB-435s | human breast cancer | DMEM + 10% FBS + penicillin + | 4 |
| ATCC# HTB-129 | | streptomycin | |
| UM-UC-3 | human bladder | MEM + 10% FBS + penicillin + | 3.5 |
| ATCC | cancer | streptomycin | |
| # CLR-1749 | | | |
| SW780 | human bladder | DMEM + 10% FBS + penicillin + | 3 |
| ATCC | cancer | streptomycin | |
| #CRL-2169 | | | |
| SW620 | human colorectal | DMEM + 10% FBS + penicillin + | 5 |
| ATCC | cancer | streptomycin | |
| #CCL-227 | | | |
| BxPC-3 | human pancreatic | RPMI + 10% FBS + penicillin + | 4.5 |
| ATCC | cancer | streptomycin | |
| #CRL-1687 | | | |
| SK-OV-3 | human ovarian | McCoy's + 10% FBS + penicillin + | 4 |
| ATCC | cancer | streptomycin | |
| # HTB-77 | | | |
| NIH: OVCAR-3 | human ovarian | RPMI + 20% FBS + 0.01 mg/ml | 7 |
| ATCC | cancer | insulina + penicillin + | |
| #HTB-161 | | streptomycin | |
| HepG2 | human liver | MEM + 10% FBS + penicillin + | 7 |
| ATCC | hepatoma | streptomycin | |
| # HB-8065 | | | |
| 293 | Human embrional | MEM + 10% FBS + penicillin + | 4 |
| ATCC | kidney cells | streptomycin | |
| # CLR-1573 | | | |
| ACHN | human kidney cancer | MEM + 10% FBS + penicillin + | 4 |
| ATCC | | streptomycin | |
| #CCL-222 | | | |
| CAKI 1 | human kidney cancer | McCoy's + 10% FBS + penicillin + | 3.5 |
| ATCC | | streptomycin | |
| #HTB-46 | | | |
| CAKI 2 | human kidney cancer | McCoy's + 10% FBS + penicillin + | 3.5 |
| ATCC | | streptomycin | |
| # HTB-47 | | | |
| NCI-H69AR | human small cell | RPMI + 10% FBS + penicillin + | 10 |
| ATCC | lung cancer | streptomycin | |
| #CRL-11351 | | | |
| HT144 | human melanoma | McCoy's + 10% FBS + penicillin + | 7 |
| ATCC | cells | streptomycin | |
| # HTB-63 | | | |
| NCI-H460 | human lung cancer | RPMI + 10% FBS + penicillin + | 2.5 |
| ATCC | | streptomycin | |
| #HTB-177 | | | |
| A549 | human lung cancer | RPMI + 10% FBS + penicillin + | 2.5 |
| ATCC | | streptomycin | |
| # CCL-185 | | | |
| MES-SA | human uterine | McCoy's + 10% FBS + penicillin + | 3.5 |
| ATCC | sarcoma | streptomycin | |
| # CRL-1976 | | | |
| MES-SA/Dx5 | multidrug resistant | McCoy's + 10% FBS + penicillin + | 4 |
| ATCC | human uterine | streptomycin | |
| #CRL-1977 | sarcoma | | |
| MES-SA/Mx2 | human uterine | Waymouth's MB 752/1 + | 4 |
| ATCC | sarcoma | McCoy's (1:1) + | |
| #CRL-2274 | | 10% FBS + penicillin + | |
| | | streptomycin | |
| SK-MES-1 ATCC | human lung cancer | MEM + 10% FBS + penicillin + | 5 |
| # HTB-58 | | streptomycin | |
| HCT-116 ATCC | human colorectal | McCoy's + 10% FBS + penicillin + | 3 |
| # CCL-247 | cancer | streptomycin | |
| MCF10A ATCC | mammary epithelial | DMEM: F12 + 5% horse plasma + | 5 |
| # CRL-10317 | cells | 0.5 μg/ml hydrocortisone + 10 μg/ml | |
| | | insuline + 20 ng/ml | |
| | | growth factor EGF | |
| Panc-1 CLS | human pancreatic | DMEM + 10% FBS + penicillin + | 5 |
| 330228 | cancer | streptomycin | |
| Panc03.27 | human pancreatic | RPMI + 10% FBS + penicillin + | 5 |
| ATCC | cancer | streptomycin | |
| # CRL-2549 | | | |
| PLC/PRF/5 CLS | human liver | DMEM + 10% FBS + penicillin + | 5 |
| 330315 | hepatoma | streptomycin | |
| LNCaP | human prostate | RPMI + 10% FBS + penicillin + | 4.5 |
| ATCC | cancer | streptomycin | |
| # CRL-1740 | | | |
| SK-Hep-1 | human liver | RPMI + 10% FBS + penicillin + | 10 |
| CLS300334 | hepatoma | streptomycin | |
| A498 | human kidney cancer | MEM + 10% FBS + penicillin + | 3 |
| CLS 300113 | | streptomycin | |
| HT1080 ATCC | Human fibrosarcoma | MEM + 10% FBS + penicillin + | 3 |
| #CCL-121 | | streptomycin | |
| HUV-EC-C | human umbilical | M199 + 20% FBS + penicylina + | 8.5 |
| ATCC | vein endothelial | 0.05 mg/ml ECGS + 0.1 mg/ml | |
| # CRL-1730 | cells | heparyny + penicylina + | |
| | | streptomycyna | |

TABLE 3
Nonadherent cells:

| | | | number of |
|---|---|---|---|
| | | | cells per |
| | | | well |
| Cell line | Cancer type | Medium | (thousands) |
| NCI-H69 | human | RPMI + 10% FBS + | 22 |
| ATCC # HTB-119 | small cell | penicillin + | |
| | lung cancer | streptomycin | |
| Jurkat A3 | human | RPMI + 10% FBS + | 10 |
| ATCC #CRL-2570 | leukaemia | penicillin + | |
| | | streptomycin | |
| HL60 | human | RPMI + 20% FBS + | 10 |
| ATCC # CCL-240 | leukaemia | penicillin + | |
| | | streptomycin | |
| CCRF-CEM | human | RPMI + 20% FBS + | 10 |
| ATCC # CCL-119 | leukaemia | penicillin + | |
| | | streptomycin | |

MTT Cytotoxicity Test

MTT assay is a colorimetric assay used to measure proliferation, viability and cytotoxicity of cells. It consists in decomposition of a yellow tetrazolium salt MTT (4,5-dimethyl-2-thiazolyl)-2,5-diphenyltetrazolium bromide) to the water-insoluble purple dye formazan by mitochondrial enzyme succinate-tetrazolium reductase 1. MTT reduction occurs only in living cells. Data analysis consists in determining IC₅₀ concentration of the protein (in ng/ml), at which the 50% reduction in the number of cells occurs in the population treated compared to control cells. Results were analyzed using GraphPad Prism 5.0 software. The test was performed according to the literature descriptions (Celis, J E, (1998). Cell Biology, a Laboratory Handbook, second edition, Academic Press, San Diego; Yang, Y., Koh, L W, Tsai, J H., (2004); Involvement of viral and chemical factors with oral cancer in Taiwan, Jpn J Clin Oncol, 34 (4), 176-183).

Cell culture medium was diluted to a defined density (10⁴-10⁵ cells per 100 μl). Then 100 μl of appropriately diluted cell suspension was applied to a 96-well plate in triplicates. Thus prepared cells were incubated for 24 h at 37° C. in 5% or 10% CO₂, depending on the medium used, and then to the cells (in 100 μl of medium) further 100 μl of the medium containing various concentrations of tested proteins were added. After incubation of the cells with tested proteins over the period of next 72 hours, which is equivalent to 3-4 times of cell division, the medium with the test protein was added with 20 ml of MTT working solution [5 mg/ml], and incubation was continued for 3 h at 37° C. in 5% CO₂. Then the medium with MTT solution was removed, and formazan crystals were dissolved by adding 100 μl of DMSO. After stirring, the absorbance was measured at 570 nm (reference filter 690 nm).

EZ4U Cytotoxicity Test

EZ4U (Biomedica) test was used for testing cytotoxic activity of the proteins in nonadherent cell lines. The test is a modification of the MTT method, wherein formazan formed in the reduction of tetrazolium salt is water-soluble. Cell viability study was carried out after continuous 72-hour incubation of the cells with protein (seven concentrations of protein, each in triplicates). On this basis IC₅₀ values were determined (as an average of two independent experiments) using the GraphPad Prism 5 software. Control cells were incubated with the solvent only.

The results of in vitro cytotoxicity tests are summarized as IC₅₀ values (ng/ml), which corresponds to the protein concentration at which the cytotoxic effect of fusion proteins is observed at the level of 50% with respect to control cells treated only with solvent. Each experiment represents the average value of at least two independent experiments performed in triplicates. As a criterion of lack of activity of protein preparations the IC₅₀ limit of 2000 ng/ml was adopted. Fusion proteins with an IC₅₀ value above 2000 were considered inactive.

Cells selected for this test included tumor cell lines that are naturally resistant TRAIL protein (the criterion of natural resistance to TRAIL: IC₅₀ for TRAIL protein>2000), as well as tumor cell lines sensitive to TRAIL protein and resistant to doxorubicin line MES-SA/DX5 as a cancer line resistant to conventional anticancer medicaments.

Undifferentiated HUVEC cell line was used as a healthy control cell line for assessment of the effect/toxicity of the fusion proteins in non-cancer cells.

The results obtained confirm the possibility of overcoming the resistance of the cell lines to TRAIL by administration of certain fusion proteins of the invention to cells naturally resistant to TRAIL. When fusion proteins of the invention were administered to the cells sensitive to TRAIL, in some cases a clear and strong potentiation of the potency of action was observed, which was manifested in reduced IC₅₀ values of the fusion protein compared with IC₅₀ for the TRAIL alone. Furthermore, cytotoxic activity of the fusion protein of the invention in the cells resistant to classical anti-cancer medicament doxorubicin was obtained, and in some cases it was stronger than activity of TRAIL alone.

The IC₅₀ values above 2000 obtained for the non-cancer cell lines show the absence of toxic effects associated with the use of proteins of the invention for healthy cells, which indicates potential low systemic toxicity of the protein.

Determination of Cytotoxic Activity of Selected Protein Preparations Against Extended Panel of Tumor Cell Lines

Table 4 presents the results of the tests of cytotoxic activity in vitro for selected fusion proteins of the invention against a broad panel of tumor cells from different organs, corresponding to the broad range of most common cancers.

The experimental results are presented as a mean value±standard deviation (SD). All calculations and graphs were prepared using the GraphPad Prism 5.0 software.

Obtained IC₅₀ values confirm high cytotoxic activity of fusion proteins and thus their potential utility in the treatment of cancer.

TABLE 4
Cytotoxic activity of the fusion proteins of the invention

| | Continuous incubation of preparations with cells over 72 h (test MTT, ng/ml) | | | | | | | | | | | |
|---|---|---|---|---|---|---|---|---|---|---|---|---|
| | A549 | | MCF10A | | HCT116 | | MES-SA | | MES-SA/Dx5 | | SK-MES-1 | |
| Protein | IC₅₀ | ±SD | IC₅₀ | ±SD | IC₅₀ | ±SD | IC₅₀ | ±SD | IC₅₀ | ±SD | IC₅₀ | ±SD |
| TRAIL 95-281 | 10000 | | | | | | | | | | | |
| Ex. 42^{a} | 1976 | | 1106 | | 36.24 | | 27.7 | | 2.627 | | 26 | |
| Ex. 43^{a} | 996 | | 2329 | | 11.75 | | 21.36 | | 2.073 | | 9.492 | |
| Ex. 44^{a} | 5.35 | 2.75 | 8.99 | 0.22 | 9.55 | 8.13 | 0.65 | 0.12 | 0.19 | 0.08 | 0.4 | 0.24 |
| Ex. 45^{a} | | | 64.3 | 7.98 | | | 41.92 | 8.78 | 41.99 | 8.23 | 54.31 | 1.55 |
| Ex. 47^{a} | 31.53 | 7.81 | 683 | 202.2 | 2.73 | 0.71 | 23.84 | | 0.64 | 0.14 | 6.69 | 0.37 |
| Ex. 49^{a} | 50.64 | 1.82 | 70.59 | 1.86 | 3.2 | 1.21 | 3.67 | 0.16 | 0.76 | 0.03 | 3.39 | 0.13 |
| Ex. 50^{a} | 57.56 | 14.94 | 104.57 | 33.1 | 2.63 | 1.24 | 3.06 | 1.24 | 0.57 | 0.16 | 3.27 | 0.31 |
| Ex. 11^{a} | 390.5 | 14.85 | 404.9 | 93.6 | 23 | 6.65 | 53.95 | 25.67 | 1.18 | | 19.19 | 3.22 |
| Ex. 12^{a} | 25.33 | 3.36 | 20.82 | 1.09 | 14.95 | 6.01 | 0.95 | 0.36 | 0.11 | | 0.26 | 0.04 |
| Ex. 13^{a} | 352.7 | 113.7 | 350.95 | 96.24 | 9.45 | 0.45 | 2.51 | 1.2 | 1.47 | 0.16 | 0.77 | 0.02 |
| Ex. 14^{a} | | | 5350 | 694.4 | 59.91 | 30.46 | 16.06 | 1.92 | 15.15 | 1.49 | 50.49 | 5.25 |

| | Continuous incubation of preparations with cells over 72 h (test MTT, ng/ml) | | | | | | | | | | | |
|---|---|---|---|---|---|---|---|---|---|---|---|---|
| | A549 | | MCF10A | | HCT116 | | MES-SA | | MES-SA/Dx5 | | SK-MES-1 | |
| Protein | IC₅₀ | ±SD | IC₅₀ | ±SD | IC₅₀ | ±SD | IC₅₀ | ±SD | IC₅₀ | ±SD | IC₅₀ | ±SD |
| Ex. 10^{a} | 294.2 | 45.68 | 122.6 | 8.98 | 12.47 | 7.62 | 3.58 | 0.99 | 8.43 | | 2.53 | 0.6 |
| Ex. 18^{a} | 1.44 | 0.07 | 202.9 | 5.44 | 3.61 | 1.09 | 329.6 | | 15.95 | 1.87 | 6.39 | 0.63 |
| Ex. 35^{a} | 759.7 | 224.2 | 1001.6 | 6.22 | 7.87 | 3.16 | 7.67 | 2.48 | 6.95 | 1.68 | 3.36 | 0.19 |
| Ex. 37^{a} | 226 | | 55.9 | | 29.6 | | 21.84 | | 2.65 | | 6.12 | |
| Ex. 27^{a} | 1090.9 | 179.8 | | | 199.3 | 64.63 | 209.6 | 23.19 | 187.1 | 2.97 | 52.64 | 24.43 |
| Ex. 28^{a} | 302.8 | 12.6 | 512.2 | 17.25 | 35.46 | 18.73 | 14.63 | 5.69 | 18.19 | 11.5 | 8.64 | 1.79 |
| Ex. 2^{a} | 31.31 | 0.7 | 516 | 77.21 | 9.07 | 7.03 | 29.82 | 11.11 | 1.95 | 0.24 | 8.38 | 1.99 |
| Ex. 3^{a} | 989.25 | 472 | 773.9 | | 12.67 | 10.28 | 13.12 | 2.51 | 3.95 | 1.01 | 3.71 | 0.07 |
| Ex. 5^{a} | 1160 | | 10000 | | 1.26 | | 39.23 | | 1.84 | | 4.95 | |
| Ex. 6^{a} | 93.84 | 25.7 | 253 | 116.11 | 2.51 | | | | 0.51 | 0.29 | 1.27 | 0.1 |
| Ex. 25^{a} | 207.15 | 32.17 | 345.8 | 47.8 | 13.7 | 5.88 | 8.27 | 0.13 | 8.8 | 0.18 | 6.31 | 0.3 |
| Ex. 26^{a} | 35.47 | 3.72 | 7.6 | 1.74 | 2.61 | 2.55 | 0.6 | 0.16 | 0.24 | 0.02 | 0.27 | 0.03 |

| | Continuous incubation of preparations with cells over 72 h (test MTT, ng/ml) | | | | | | | | | | | |
|---|---|---|---|---|---|---|---|---|---|---|---|---|
| | A549 | | HCT116 | | MCF10A | | MES-SA | | MES-SA/Dx5 | | SK-MES-1 | |
| protein | IC₅₀ | ±SD | IC₅₀ | ±SD | IC₅₀ | ±SD | IC₅₀ | ±SD | IC₅₀ | ±SD | IC₅₀ | ±SD |
| Ex. 7^{a} | 230.36 | 185.0 | 43.19 | 14.06 | 346.65 | 10.96 | 32.64 | 2.86 | 27.04 | 6.18 | 9.81 | 0.14 |
| Ex. 16^{a} | | | 239.6 | 85.42 | 3705.5 | 1307.4 | 311.25 | 15.91 | 61.85 | 24.63 | 30.03 | 7.07 |
| Ex. 41^{a} | 236.2 | | 127.3 | 85.42 | | | | | | | 4.572 | |
| Ex. 40^{a} | 2457 | | 2457 | | 192.7 | | | | | | 7.07 | |
| Ex. 29^{a} | 278.8 | | 60.37 | | 179 | | 34.22 | | 34.22 | | 50.93 | |

| | Continuous incubation of preparations with cells over 72 h (test MTT, ng/ml)) | | | | | | | | | | | |
|---|---|---|---|---|---|---|---|---|---|---|---|---|
| | Colo 205 | | DU 145 | | MCF 7 | | MDA-MB-231 | | PC 3 | | SW620 | |
| Protein | IC₅₀ | ±SD | IC₅₀ | ±SD | IC₅₀ | ±SD | IC₅₀ | ±SD | IC₅₀ | ±SD | IC₅₀ | ±SD |
| Ex. 43^{a} | 2.76 | 0.25 | 105.35 | 12.24 | 4093.5 | 1440.4 | 66.57 | 0.07 | 2553.5 | 1438.96 | 7648.5 | 1642.61 |
| Ex. 49^{a} | 2.49 | 0.44 | 20.54 | 13.39 | 240.5 | 126.57 | 62.88 | 6.19 | 160.1 | 19.66 | 225.55 | 11.95 |
| Ex. 50^{a} | 2.67 | 1.48 | 4.38 | | 369.9 | 1.27 | 111.3 | 6.36 | 40.07 | 0.76 | 115.95 | 7 |
| Ex. 12^{a} | 0.93 | 0.76 | | | 2317.5 | 94.05 | 6.93 | 2.91 | 1641 | 199.4 | 228.5 | 126.57 |
| Ex. 10^{a} | 1.13 | 0.8 | 17.85 | 11.1 | 3442 | 1496.2 | 17.56 | 2.04 | 1157.5 | 130.81 | 3311.5 | 342.95 |
| Ex. 18^{a} | 1.03 | 0.01 | 18.74 | 0.61 | 51.89 | 31.28 | 251 | 54.86 | 106.1 | 32.19 | 26.37 | 0.1 |
| Ex. 5^{a} | 0.45 | 0.01 | 59.76 | 15.2 | 207.4 | 128.13 | 108.95 | 1.34 | 15.36 | 0.49 | 60.42 | 1.3 |
| Ex. 25^{a} | 6.57 | 0.22 | 31.65 | 6.51 | 520.85 | 159.59 | 92.03 | 34.62 | 115.64 | 28.38 | | |
| Ex. 16^{a} | 13.35 | 0.64 | 261.5 | 43.13 | 3310.5 | 581.95 | 209.6 | 9.19 | 2026.5 | 37.48 | | |
| Ex. 12^{a} | | | | | | | | | | | 228.5 | 126.57 |

| | Continuous incubation of preparations with cells over 72 h (test MTT, ng/ml) | | | | | | | | | | | |
|---|---|---|---|---|---|---|---|---|---|---|---|---|
| | SW780 | | UM-UC-3 | | 293 | | ACHN | | SK-OV-3 | | BxPC3 | |
| Protein | IC₅₀ | ±SD | IC₅₀ | ±SD | IC₅₀ | ±SD | IC₅₀ | ±SD | IC₅₀ | ±SD | IC₅₀ | ±SD |
| Ex. 43^{a} | 3.68 | 1.02 | 8.51 | 0.42 | 1530 | 439.8 | 38.88 | 6.26 | 4184 | 60.81 | 11.95 | 2.71 |
| Ex. 49^{a} | 3.96 | 0.6 | 7.6 | 0.31 | 11.73 | 0.07 | 29.6 | 2.69 | 700.95 | 104.58 | 11.04 | 0.37 |
| Ex. 50^{a} | 8.29 | 3.37 | 6.5 | 1.83 | 11.34 | 4.47 | 30.29 | 1.71 | 262 | 69.3 | 9.02 | 1.36 |
| Ex. 12^{a} | 1.29 | 0.28 | 2.69 | 0.98 | 151.3 | 56.14 | 9.86 | 0.21 | | | 0.95 | 0.34 |
| Ex. 10^{a} | 1.69 | 0.45 | 2.17 | 1.05 | 1790.5 | 81.32 | 13.76 | 1.77 | 264 | 159.81 | 2.46 | 1.35 |
| Ex. 18^{a} | 2.22 | 0.96 | 89.21 | 7.43 | | | | | 114.4 | 0.14 | 32.07 | 3.97 |
| Ex. 5^{a} | 1.16 | 0.26 | 1.35 | 0.48 | 0.93 | 0.62 | 46.09 | 0.16 | 2887.5 | 265.17 | 9.26 | 4.04 |
| Ex. 25^{a} | 7.89 | 2.21 | 36.49 | 12.52 | | | | | 113.02 | 32.22 | 8.68 | 2.79 |
| Ex. 16^{a} | 29.97 | 0.76 | 36.47 | 4.06 | | | 336.35 | 57.49 | 3586 | 585.48 | 43.24 | 6.39 |

| | Continuous incubation of preparations with cells over 72 h (test MTT, ng/ml) | | | | | | | | | | | |
|---|---|---|---|---|---|---|---|---|---|---|---|---|
| | HT29 | | HepG2 | | NCI-H460 | | OV-CAR-3 | | JURKAT A3 | | PLC/PRF/5 | |
| Protein | IC₅₀ | ±SD | IC₅₀ | ±SD | IC₅₀ | ±SD | IC₅₀ | ±SD | IC₅₀ | ±SD | IC₅₀ | ±SD |
| Ex. 43^{a} | 2827.5 | 169 | 3042 | 39.6 | 11.74 | 0.93 | 4.95 | 3.27 | 3.63 | 0.38 | | |
| Ex. 44^{a} | 5028 | | 3321.5 | 842.16 | 1.65 | 0.86 | | | 0.28 | 0.02 | 23.2 | 13.72 |
| Ex. 47^{a} | 47.18 | 2.86 | 1571 | 650.54 | 4.63 | 0.97 | | | | | 23.2 | 13.72 |
| Ex. 49^{a} | 630.8 | 16.26 | 144.5 | 0.71 | 4.53 | 0.79 | 2.66 | 0.75 | 4.64 | 1.44 | | |
| Ex. 50^{a} | 289.1 | 4.38 | 211 | 42.43 | 4.34 | 0.48 | 2.34 | 0.09 | 3.66 | 1.44 | | |
| Ex. 11^{a} | 1439.5 | 236 | | | 22.75 | 7 | | | | | 638.5 | 170.41 |
| Ex. 12^{a} | 498 | 59.4 | 210.25 | 32.88 | 1.47 | 0.16 | 1.06 | 0.06 | 0.5 | 0.21 | 1282 | |
| Ex. 13^{a} | | | 8190 | 2560 | 9079 | 1302 | | | | | 3545 | |
| Ex. 10^{a} | 2862.5 | 1243.8 | 279.6 | 54.38 | 1.82 | 0.01 | 0.81 | 0.25 | 3.6 | 2 | | |
| Ex. 18^{a} | 6.13 | 0.2 | 2.86 | 0.24 | 7.51 | 0.24 | 43.5 | 30.1 | 104.81 | 44.82 | 2 | 0.91 |
| Ex. 2^{a} | 59.23 | 9.66 | 39.1 | | 4.59 | 0.41 | | | | | 15.22 | |
| Ex. 5^{a} | | | 1156 | 308.3 | 2.09 | 0.41 | 2.74 | 0.45 | 141.75 | 23.41 | | |
| Ex. 25^{a} | | | 87.2 | 6.39 | | | 3.37 | 2.04 | | | | |

| | Continuous incubation of preparations with cells over 72 h (test MTT, ng/ml) | | | | | | | | | | | |
|---|---|---|---|---|---|---|---|---|---|---|---|---|
| | CAKI 2 | | H69AR | | HT 144 | | LNCaP | | HL60 | | PANC-1 | |
| Protein | IC₅₀ | ±SD | IC₅₀ | ±SD | IC₅₀ | ±SD | IC₅₀ | ±SD | IC₅₀ | I ±SD | IC₅₀ | ±SD |
| Ex. 43^{a} | 4200 | 1665.94 | | | 8.76 | 0.8 | 4449.5 | 2462.9 | | | | |
| Ex. 44^{a} | | | | | | | | | 292.7 | 30.12 | 9.4 | 2.31 |
| Ex. 47^{a} | | | | | | | | | | | 14.95 | 2.48 |
| Ex. 49^{a} | 658 | 367.7 | 3100.5 | 878.9 | 8.1 | 1.05 | 4.06 | 1.77 | | | | |
| Ex. 50^{a} | 82 | 7.35 | 1586.5 | 458.9 | 6.63 | 0.28 | 2.57 | 0.35 | | | | |
| Ex. 1^{a} | | | | | | | | | | | 315.9 | 33.8 |
| Ex. 12^{a} | 28.52 | 6.2 | 463.35 | 10.39 | 0.64 | 0.01 | 58.78 | 40.19 | 434 | 155 | 1143 | |
| Ex. 13^{a} | | | | | | | | | | | 125.1 | 27.15 |
| Ex. 10^{a} | 15.53 | 0.95 | 4500 | | 0.97 | 0.01 | 948 | 333.75 | | | | |
| Ex. 18^{a} | | | | | | | | | | | 8.9 | 1 |
| Ex. 2^{a} | | | | | | | | | | | 18.51 | 3.23 |
| Ex. 5^{a} | | | 160 | 7.07 | 0.59 | 0.12 | 3.28 | 3.88 | | | | |

| | Continuous incubation of preparations with cells over 72 h (test MTT, ng/ml) | | | | | | | | | | | |
|---|---|---|---|---|---|---|---|---|---|---|---|---|
| | SK-MES-1 | | SW620 | | HT 144 | | HepG2 | | NCI-H460 | | JURKAT A3 | |
| Protein | IC₅₀ | ±SD | IC₅₀ | ±SD | IC₅₀ | ±SD | IC₅₀ | ±SD | IC₅₀ | ±SD | IC₅₀ | ±SD |
| Ex. 7^{a} | 9.81 | 0.14 | | | | | | | | | | |
| Ex. 16^{a} | 30.03 | 7.07 | | | 47.12 | 2.07 | | | 41.9 | 0.83 | 23.51 | 5.93 |
| Ex. 41^{a} | 4.572 | | | | | | | | | | | |
| Ex. 40^{a} | 7.07 | | | | | | | | | | | |
| Ex. 29^{a} | 50.93 | | | | | | | | | | | |
| Ex. 44^{a} | | | 369 | | | | | | | | | |
| Ex. 47^{a} | | | 14.92 | 2.52 | | | | | | | | |
| Ex. 49^{a} | | | | | | | | | | | | |
| Ex. 37^{a} | | | | | | | | | 26 | | | |
| Ex. 11^{a} | | | 287.6 | 160.37 | | | | | | | | |
| Ex. 2^{a} | | | 583.2 | | | | | | | | | |
| Ex. 25^{a} | | | | | | | 87.2 | 6.93 | | | | |

| | Continuous incubation of preparations with cells over 72 h (test MTT, ng/ml)) | | | | | | | | | | | |
|---|---|---|---|---|---|---|---|---|---|---|---|---|
| | A549 | | HCT116 | | MCF10A | | MES-SA | | MES-SA/Dx5 | | SK-MES-1 | |
| Protein | IC₅₀ | ±SD | IC₅₀ | ±SD | IC₅₀ | ±SD | IC₅₀ | ±SD | IC₅₀ | ±SD | IC₅₀ | ±SD |
| TRAIL 95-281 | >2000 | | >2000 | | >2000 | | >2000 | | 27.59 | 13.34 | 100.71 | 26.43 |
| Ex. 27^{b} | 1090 | 179 | 199.3 | 64.63 | 891.65 | 344.15 | 209.6 | 23.19 | 187.1 | 2.97 | 50.85 | 8.7 |
| Ex. 28^{b} | 302.8 | 12.59 | 35.46 | 18.73 | 512.2 | 17.25 | 14.63 | 5.69 | 18.19 | 11.5 | 8.64 | 1.79 |
| Ex. 26^{b} | — | — | 2.04 | 0.38 | — | — | — | — | — | — | — | — |
| Ex. 18^{b} | — | — | — | — | — | — | 475.2 | 75.7 | 42.0 | 7.4 | — | |
| Ex. 29^{b} | 278.8 | | 60.37 | | 179.0 | | 34.22 | | 34.22 | | 50.93 | |
| Ex. 40^{b} | >2000 | | 476.7 | 42.99 | — | — | — | — | — | — | 203.35 | 15.06 |
| Ex. 32^{b} | 131.1 | 8.34 | 9.5 | 1.7 | 88.09 | 4.41 | 13.3 | 0.04 | 0.917 | 0.07 | 1.49 | 0.523 |
| Ex. 42^{b} | 58.66 | 49.46 | 9.21 | 3.0 | 432.75 | 50.28 | 15.58 | 2.23 | 1.61 | 0.66 | 7.03 | 3.31 |
| Ex. 43^{b} | 1102 | 150.6 | 12.08 | 0.46 | 326.0 | 48.08 | 19.03 | 3.3 | 2.01 | 0.09 | 8.15 | 1.9 |

| | Continuous incubation of preparations with cells over 72 h (test MTT, ng/ml)) | | | | | | | | | | | |
|---|---|---|---|---|---|---|---|---|---|---|---|---|
| | A549 | | HCT116 | | MCF10A | | MES-SA | | MES-SA/Dx5 | | SK-MES-1 | |
| Protein | IC₅₀ | ±SD | IC₅₀ | ±SD | IC₅₀ | ±SD | IC₅₀ | ±SD | IC₅₀ | ±SD | IC₅₀ | ±SD |
| Ex. 44^{b} | 5.35 | 2.75 | 1.62 | 0.07 | 1159.5 | 26.16 | 0.65 | 0.12 | 0.19 | 0.08 | 0.4 | 0.24 |
| Ex. 46^{b} | 90.29 | 13.62 | 48.96 | 6.75 | 452.5 | 21.5 | 45.25 | 14.11 | 12.73 | 4.45 | 14.08 | 1.51 |
| Ex. 47^{b} | 31.53 | 7.81 | 2.73 | 0.71 | 683.0 | 202.23 | 1.76 | 1.28 | 0.64 | 0.14 | 6.69 | 0.37 |
| Ex. 49^{b} | 50.64 | 1.82 | 3.2 | 1.21 | 70.59 | 1.86 | 3.67 | 0.16 | 0.76 | 0.03 | 3.39 | 0.13 |
| Ex. 50^{b} | 57.56 | 14.94 | 2.63 | 1.24 | 104.57 | 33.14 | 3.06 | 1.24 | 0.57 | 0.16 | 3.27 | 0.31 |
| Ex. 59^{b} | 800.0 | | 332.0 | | 88.47 | | 94.01 | | 18.32 | | 59.6 | |
| Ex. 78^{b} | >2000 | | 143.0 | | — | — | 36.95 | | — | — | 75.02 | |
| Ex. 67^{b} | 1118 | | 550 | | 1934 | | 1288 | | — | — | — | — |
| Ex. 71^{b} | 13.31 | 5.83 | 6.49 | 2.01 | 37.83 | 17.15 | 31.46 | 14.66 | 3.22 | 0.80 | 737.9 | 318.8 |
| Ex. 68^{b} | 433 | | 228 | | 500 | | 320 | | 61.6 | | 29.7 | |

| | Continuous incubation of preparations with cells over 72 h (test MTT, ng/ml)) | | | | | | | | | | | |
|---|---|---|---|---|---|---|---|---|---|---|---|---|
| | A549 | | HCT116 | | MCF10A | | MES-SA | | MES-SA/Dx5 | | SK-MES-1 | |
| Protein | IC₅₀ | ±SD | IC₅₀ | ±SD | IC₅₀ | ±SD | IC₅₀ | ±SD | IC₅₀ | ±SD | IC₅₀ | ±SD |
| Ex. 66^{b} | 41.7 | | 56.5 | | 398 | | 639 | | 29.1 | | 6.0 | |
| Ex. 65^{b} | 5.4 | | 3.9 | | 99.3 | | 361 | | 4.3 | | 3.8 | |
| Ex. 15^{b} | 55.4 | 20.6 | 34.6 | 4.7 | 287 | 161 | 159 | 58 | 105 | 7 | 41.5 | 1.5 |
| Ex. 20^{b} | 0.393 | 0.12 | 1.30 | 0.46 | 346 | 17 | 61.7 | 11.2 | 2.32 | 0.02 | 4 | 0.16 |
| Ex. 2^{b} | — | — | 5.86 | 0.54 | 318 | 104 | 11.38 | 0.41 | — | — | 7.86 | 0.62 |
| Ex. 14^{b} | — | — | 43.8 | 7.7 | — | — | — | — | — | — | — | — |

| | Continuous incubation of preparations with cells over 72 h (test MTT, ng/ml)) | | | | | | | | | | | |
|---|---|---|---|---|---|---|---|---|---|---|---|---|
| | MES-SA/MX2 | | PANC03.27 | | A498 | | SK-Hep-1 | | MDA-MB-435s | | Caki-1 | |
| Protein | IC₅₀ | ±SD | IC₅₀ | ±SD | IC₅₀ | ±SD | IC₅₀ | ±SD | IC₅₀ | ±SD | IC₅₀ | ±SD |
| TRAIL 95-281 | 38.95 | 6.14 | 315 | | 1611.0 | 102.53 | >2000 | | >2000 | | 13.42 | 2.16 |
| Ex. 32^{b} | 1.05 | 0.5 | 87.98 | 27.04 | 15.49 | 2.52 | 332.1 | 31.96 | 19.65 | 0.26 | 42.,58 | 2.57 |
| Ex. 2^{b} | 0.55 | 0.43 | 46.49 | 1.12 | 3.4 | 0.67 | 33.2 | 9.1 | 9.2 | 1.81 | 9.31 | 0.93 |
| Ex. 18^{b} | 52.7 | 18.3 | 170.7 | 80.5 | 37.84 | 4.38 | — | — | 41.01 | 12.49 | 36.6 | 5.38 |

| | Continuous incubation of preparations with cells over 72 h (test MTT, ng/ml)) | | | | | | | | | | | |
|---|---|---|---|---|---|---|---|---|---|---|---|---|
| | HT-29 | | SW620 | | BxPC-3 | | Colo 205 | | SK-OV-3 | | MDA-MB-231 | |
| Protein | IC₅₀ | ±SD | IC₅₀ | ±SD | IC₅₀ | ±SD | IC₅₀ | ±SD | IC₅₀ | ±SD | IC₅₀ | ±SD |
| TRAIL 95-281 | >2000 | | >2000 | | 60.61 | 22.78 | 59.02 | 21.16 | >2000 | | >2000 | |
| Ex. 32^{b} | 1252 | 385.0 | 175.8 | 25.4 | 9.88 | 1.21 | 10.85 | 2.08 | 1093.0 | 210.0 | 30.47 | 10.74 |
| Ex. 43^{b} | >2000 | | 8.51 | 0.42 | 12.0 | 2.7 | 2.76 | 0.25 | >2000 | | 66.57 | 0.07 |
| Ex. 44^{b} | 4104 | 655.9 | 369.8 | 0 | 0.268 | 0.004 | 0.64 | 0.23 | 7.07 | 0.93 | 10.6 | 6.9 |
| Ex. 47^{b} | 47.18 | 2.86 | 14.92 | 2.52 | — | — | — | — | — | — | — | — |
| Ex. 49^{b} | 630.8 | 16.26 | 225.6 | 12.0 | 11.04 | 0.37 | 2.49 | 0.44 | 700.95 | 104.6 | 62.88 | 6.19 |
| Ex. 50^{b} | 289.1 | 4.38 | 116.0 | 7.0 | 9.02 | 1.36 | 2.67 | 1.48 | 262.0 | 69.3 | 111.3 | 6.36 |
| Ex. 2^{b} | — | — | — | — | 9.46 | 2.38 | 4.12 | 0.13 | 1060.0 | 275.0 | 35.13 | 12.18 |

| | Continuous incubation of preparations with cells over 72 h (test MTT, ng/ml)) | | | | | | | | | | | |
|---|---|---|---|---|---|---|---|---|---|---|---|---|
| | HepG2 | | MCF-7 | | ACHN | | Caki-2 | | OV-CAR-3 | | HT-144 | |
| Protein | IC₅₀ | ±SD | IC₅₀ | ±SD | IC₅₀ | ±SD | IC₅₀ | ±SD | IC₅₀ | ±SD | IC₅₀ | ±SD |
| TRAIL 95-281 | >2000 | | >2000 | | >2000 | | >2000 | | 963.0 | 144.25 | 1134 | 375.0 |
| Ex. 32^{b} | 228.1 | 85.3 | 1140 | 64.35 | 70.52 | 24.06 | 33.82 | 4.38 | 1.5 | 0.73 | 24.82 | 8.96 |
| Ex. 43^{b} | >2000 | | >2000 | | 38.88 | 6.26 | >2000 | | 4.95 | 3.27 | 8.76 | 0.8 |
| Ex. 44^{b} | 9.0 | 0.32 | >2000 | | — | — | — | — | 0.14 | 0.01 | — | — |
| Ex. 47^{b} | 1571 | 650.5 | — | — | — | — | — | — | — | — | — | — |
| Ex. 49^{b} | 144.5 | 0.71 | 240.5 | 126.6 | 29.6 | 2.69 | 658.0 | 367.7 | 2.66 | 0.75 | 8.1 | 1.05 |
| Ex. 50^{b} | 211.0 | 42.43 | 369.9 | 1.27 | 30.29 | 1.71 | 82.0 | 7.35 | 2.34 | 0.09 | 6.63 | 0.28 |
| Ex. 2^{b} | 43.11 | 11.75 | 104.8 | 17.2 | 36.46 | 9.39 | 28.6 | 1.9 | 3.32 | 0.36 | 12.8 | 2.1 |
| Ex. 18^{b} | — | — | 12.69 | 1.74 | 34.39 | 11.84 | 9.2 | 4.2 | 67.08 | 4.4 | 502.7 | 127.5 |

| | Continuous incubation of preparations with cells over 72 h (test MTT, ng/ml)) | | | | | | | | | | | |
|---|---|---|---|---|---|---|---|---|---|---|---|---|
| | SW780 | | DU 145 | | Jurkat-A3 | | CCRF-CEM | | PC-3 | | UM-UC-3 | |
| Protein | IC₅₀ | ±SD | IC₅₀ | ±SD | IC₅₀ | ±SD | IC₅₀ | ±SD | IC₅₀ | ±SD | IC₅₀ | ±SD |
| TRAIL 95-281 | 120.0 | 42.43 | >2000 | | >2000 | | >2000 | | >2000 | | >2000 | |
| Ex. 43^{b} | 3.68 | 1.02 | 105.3 | 12.24 | — | — | — | — | >2000 | | >2000 | |
| Ex. 44^{b} | 0.4 | 0.13 | 13.42 | 4.26 | 0.28 | 0.02 | 369.8 | | 206.7 | 97.6 | 1.26 | 0.06 |
| Ex. 49^{b} | 3.96 | 0.6 | 20.54 | 13.39 | 4.64 | 1.44 | >2000 | | 160.1 | 19.66 | 7.6 | 0.31 |
| Ex. 50^{b} | 8.29 | 3.37 | 4.38 | 0 | 3.66 | 1.44 | >2000 | | 40.07 | 0.76 | 6.5 | 1.83 |

| | Continuous incubation of preparations with cells over 72 h (test MTT, ng/ml)) | | | | | | | | | | | |
|---|---|---|---|---|---|---|---|---|---|---|---|---|
| | LNCaP | | 293 | | H69AR | | NCI-H69 | | | | | |
| Protein | IC₅₀ | ±SD | IC₅₀ | ±SD | IC₅₀ | ±SD | IC₅₀ | ±SD | IC₅₀ | ±SD | IC₅₀ | ±SD |
| TRAIL 95-281 | >2000 | | >2000 | | >2000 | | >2000 | | | | | |
| Ex.43^{b} | >2000 | | 1530 | 439.8 | >2000 | | >2000 | | | | | |
| Ex. 49^{b} | 4.06 | 1.77 | 11.73 | 0.07 | >2000 | | 614.5 | 88.39 | | | | |
| Ex. 50^{b} | 2.57 | 0.35 | 11.34 | 4.47 | 1586.5 | 458.91 | >2000 | | | | | |

| | Continuous incubation of preparations with cells over 72 h (test MTT, ng/ml)) | | | | | | | | | | | |
|---|---|---|---|---|---|---|---|---|---|---|---|---|
| | NCI-H460 | | PANC-1 | | PLC/PRF/5 | | HT-1080 | | HL-60 | | HUV-EC-C | |
| Protein | IC₅₀ | ±SD | IC₅₀ | ±SD | IC₅₀ | ±SD | IC₅₀ | ±SD | IC₅₀ | ±SD | IC₅₀ | ±SD |
| TRAIL 95-281 | 438.2 | 77.2 | >2000 | | >2000 | | >2000 | | >2000 | | >2000 | |
| Ex. 32^{b} | 14.89 | 0.51 | 43.25 | 6.22 | 114.77 | 59.72 | 1277 | 333.0 | — | — | >2000 | |
| Ex. 43^{b} | 11.74 | 0.93 | 0.93 | | | | — | — | — | — | — | — |
| Ex. 44^{b} | 1.65 | 0.86 | 9.4 | 2.31 | 27.46 | 8.68 | — | — | 292.7 | 30.12 | — | — |
| Ex. 47^{b} | 4.63 | 0.97 | 14.95 | 2.48 | — | — | — | — | — | — | — | — |
| Ex. 49^{b} | 4.53 | 0.79 | — | — | — | — | — | — | — | — | — | — |
| Ex. 50^{b} | 4.34 | 0.48 | — | — | — | — | — | — | >2000 | | — | — |
| Ex. 14^{b} | 50.5 | 5.3 | — | — | — | — | — | — | — | — | — | — |
| Ex. 2^{b} | | | — | — | 21.2 | 2.8 | 869.0 | 1.98 | — | — | >2000 | |

### 3. Antitumor Effectiveness of Fusion Proteins In Vivo on Xenografts

Antitumor activity of protein preparations was tested in a mouse model of human colon cancer Colo 205 and HCT-116, SW620, human lung cancer A549, human prostate cancer PC-3, human pancreas cancer Panc-1, human liver cancer PCL/PRF/5, HT-29, HepG2, and human uterine sarcoma MES-SA.Dx5.

Cells

The cells of human colon cancer Colo 205 were maintained in RPMI1640 medium (HyClone, Logan, Utah, USA) (optionally mixed in the ratio of 1:1 with Opto-MEM (Invitrogen, Cat. No. 22600-134)) supplemented with 10% fetal calf serum and 2 mM glutamine. On the day, of mice grafting, the cells were detached from the support by washing the cells with trypsin (Invitrogen), then the cells were centrifuged at 1300 rpm, 4′C, 8 min., suspended in HBSS buffer (Hanks medium).

The cells of human lung cancer A549 were maintained in RPMI1640 medium (HyClone, Logan, Utah, USA) supplemented with 10% fetal calf serum and 2 mM glutamine. On the day of mice grafting, the cells were detached from the support by washing the cells with trypsin (Invitrogen), then the cells were centrifuged at 1300 rpm, 4° C., 8 min., suspended in HBSS buffer (Hanks medium).

The cells of human prostate cancer PC3 were maintained in RPMI1640 medium (HyClone, Logan, Utah, USA) supplemented with 10% fetal calf serum and 2 mM glutamine. On the day of mice grafting, the cells were detached from the support by washing the cells with trypsin (Invitrogen), then the cells were centrifuged at 1300 rpm, 4° C., 8 min., suspended in HBSS buffer (Hanks medium).

The cells of human pancreas cancer PANC-1 were maintained in DMEM medium (HyClone, Logan, Utah, USA) supplemented with 10% fetal calf serum and 2 mM glutamine. On the day of mice grafting, the cells were detached from the support by washing the cells with trypsin (Invitrogen), then the cells were centrifuged at 1300 rpm, 4° C., 8 min., suspended in HBSS buffer (Hanks medium).

The cells of human liver cancer /PRF/5 (CLS) and human colon cancer SW-620 were maintained in DMEM medium (HyClone, Logan, Utah, USA) supplemented with 10% fetal calf serum and 2 mM glutamine. On the day of mice grafting, the cells were detached from the support by washing the cells with trypsin (Invitrogen), then the cells were centrifuged at 1300 rpm, 4° C., 8 min., suspended in HBSS buffer (Hanks medium).

The cells of human colon cancer HCT-116 and HT-29 were maintained in McCoy's medium (HyClone, Logan, Utah, USA) supplemented with 10% fetal calf serum and 2 mM glutamine. On the day of mice grafting, the cells were detached from the support by washing the cells with trypsin (Invitrogen), then the cells were centrifuged at 1300 rpm, 4° C., 8 min., suspended in HBSS buffer (Hanks medium).

The cells of human liver cancer HepG2 were maintained in MEM medium (HyClone, Logan, Utah, USA) supplemented with 10% fetal calf serum and 2 mM glutamine. On the day of mice grafting, the cells were detached from the support by washing the cells with trypsin (Invitrogen), then the cells were centrifuged at 1300 rpm, 4° C., 8 min., suspended in HBSS buffer (Hanks medium).

The cells of multidrug resistant human uterine sarcoma MES-SA.Dx5 were maintained in McCoy's medium (HyClone, Logan, Utah, USA) supplemented with 10% fetal calf serum and 2 mM glutamine, and 1 μM doxorubicin hydrochloride (Sigma, Cat. No. D1515-10MG). Three days before the cells implantation, the cells were cultured in medium without doxorubicin. On the day of mice grafting, the cells were detached from the support by washing the cells with trypsin (Invitrogen), then the cells were centrifuged at 1300 rpm, 4° C., 8 min., suspended in HBSS buffer (Hanks medium).

Mice

Examination of antitumor activity of proteins of the invention was conducted on 7-9 week-old CD-nude (Crl:CD1-Foxn1^{nu} 1) mice obtained from Centrum Medy-cyny Doświadczalnej in Bialystok, 7-8 week-old Hsd:Athymic-Nude-Foxn1^{nu} (female) obtained from Harlan UK, 8-10 week-old HsdCpb:NMRI-Foxn1^{nu} mice obtained from Harlan UK, 8-10 week-old female Cby.Cg-foxn1(nu)/J mice obtained from Centrum Medycyny Dowiadczalnej in Bialystok and 4-5 week old female Crl:SHO-Prkdc^{scid}Hr^{hr} mice obtained from Charles River Germany. Mice were kept under specific pathogen-free conditions with free access to food and demineralised water (ad libitum). All experiments on animals were carried in accordance with the guidelines: “Interdisciplinary Principles and Guidelines for the Use of Animals in Research, Marketing and Education” issued by the New York Academy of Sciences' Ad Hoc Committee on Animal Research and were approved by the IV Local Ethics Committee on Animal Experimentation in Warsaw (No. 71/2009).

The Course and Evaluation of the Experiments

Tumour size was measured using electronic calliper, tumour volume was calculated using the formula: (a²×b)/2, where a=shorter diagonal of the tumour (mm) and b =longer diagonal of the tumour (mm). Inhibition of tumour growth was calculated using the formula:

TGI [%](Tumour growth inhibition)=(WT/WC)×100−100%

wherein WT is the average tumour volume in the treatment group, and WC is the average tumour volume in the control group.

The experimental results are presented as a mean value ±standard deviation (SD). All calculations and graphs were prepared using the program GraphPad Prism 5.0.

### Human Colon Cancer Model

A. Colo205

On day 0 mice were grafted subcutaneously (sc) in the right side with 5×10⁶ of Colo205 cells suspended in 0.15 ml RPMI1640 medium by means of a syringe with a 0.5×25 mm needle (Bogmark). On the 10th day of experiment mice were randomized to obtain the average size of tumours in the group of −100 mm³ and assigned to treatment groups. The treatment groups were administered with the preparations of fusion proteins of the invention of Ex. 18^{a} (3 mg/kg), Ex. 25^{a} (3 mg/kg), Ex. 37^{a} (5 mg/kg), and Ex. 42^{a} (10 mg/kg), rhTRAIL114-281 (10 mg/kg) as a comparison and water for injections as a control. The preparations were administered intravenously (i.v.) 6 times once daily every second day. On the 27th day of experiment mice were sacrificed through disruption of the spinal cord.

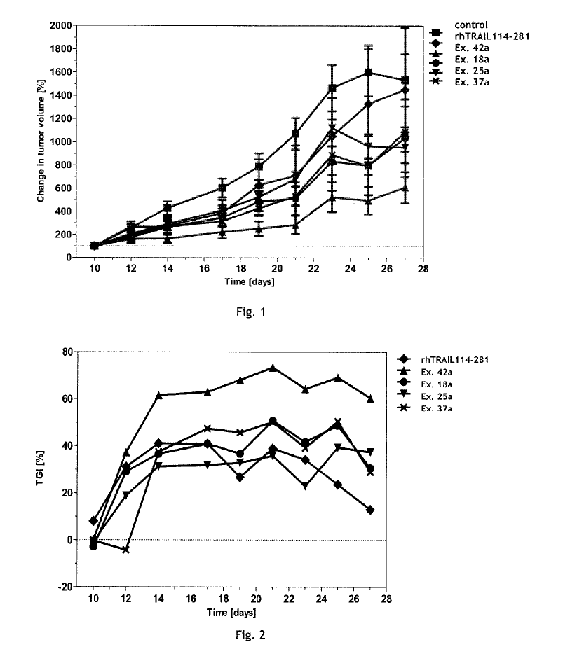

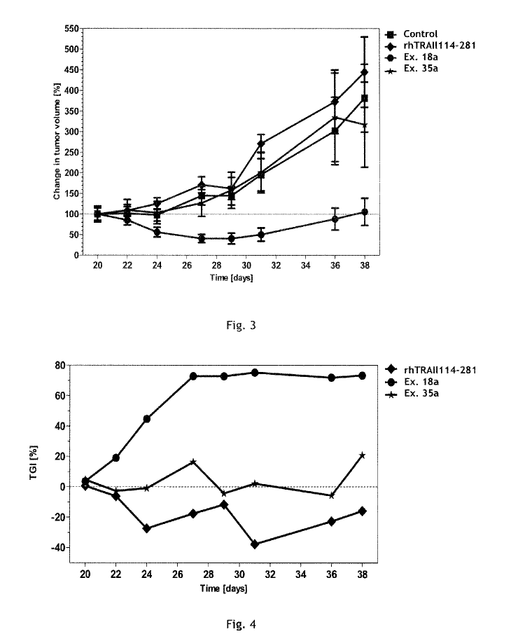

The experimental results are shown on FIG. 1 and FIG. 2, as a diagram of changes of the tumor volume (FIG. 1) and tumor growth inhibition (% TGI) as the percentage of control (FIG. 2).

The experimental results presented in FIG. 1 and FIG. 2 show that administration of the fusion proteins of the invention of Ex. 18^{a}, Ex. 25^{a}, Ex. 37^{a} and Ex. 42^{a} caused tumor Colo 205 growth inhibition, with TGI 30.5%, 37%, 29% and 60.2%, respectively, relative to the control on 27^{th} day of the experiment. For rhTRAIL114-281 used as the comparative reference, a slight inhibitory effect on tumor cell growth was obtained relative to the control, with TGI at the level of 12%. Thus, fusion proteins of the invention exert much stronger effect compared to TRAIL alone.

The tested fusion proteins did not cause significant side effects manifested by a decrease in body weight of mice (i.e. less than 10% of the baseline body weight).

This shows low systemic toxicity of the protein.

B. HCT-116

On day 0 mice Crl:SHO-Prkdc^{scid}Hr^{hr} were grafted subcutaneously (s.c.) in the right side with 5×10⁶ of HCT116 cells suspended in 0.1 ml 3:1 mixture of HBSS buffer:Matrigel using syringe with a 0.5×25 mm needle (Bogmark). When tumors reached the size of 71-432 mm³ (day 13), mice were randomized to obtain the average size of tumors in the group of −180 mm³ and assigned to treatment groups. The treatment groups were administered with the preparations of fusion proteins of the invention of Ex. 18^{b} (3 mg/kg), Ex. 2^{b} (5 mg/kg) and rhTRAIL114-281 (65 mg/kg) as a comparison against formulation buffer (50 mM Trizma Base, 200 mM NaCl, 5 mM glutathione, 0.1 mM ZnCl₂, 10% glycerol, 80 mM saccharose, pH 8.0) as a control. rhTRAIL114-281 and Ex. 2^{b} were administered intravenously (i.v.) six times every second day, Ex. 18^{b} was administered intravenously (i. v.) in 13, 15, 21, 24th day of the experiment. The control group received formulation buffer. On 24th day of the experiment mice were sacrificed by disruption of the spinal cord.

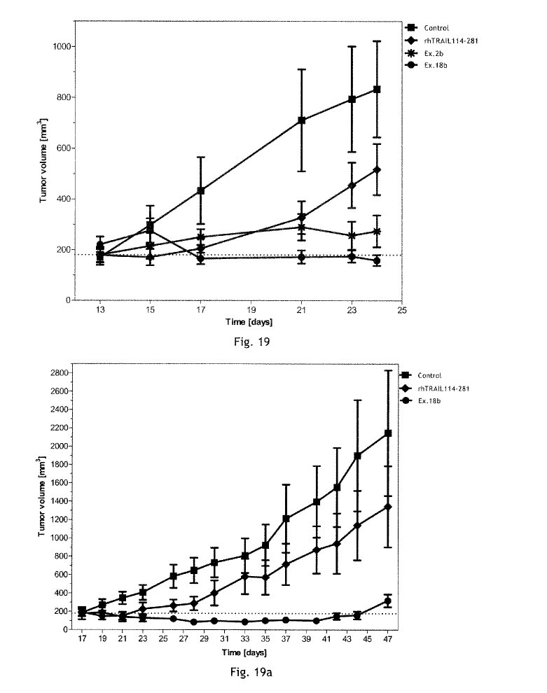

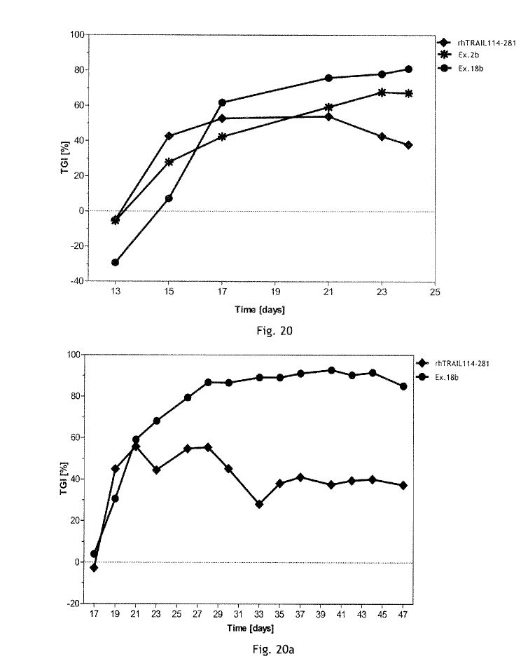

The results of experiments are shown in FIG. 19 as a diagram of changes of the tumor volume and in FIG. 20 which shows tumor growth inhibition (% TGI) as the percentage of control.

The results of experiments presented in FIGS. 1 and 2 show that administration of the fusion protein of the invention of Ex. 18^{b} and Ex. 2^{b} caused HCT116 tumor growth inhibition, respectively with TGI 81% and 67% relative to the control on 24^{th} day of the experiment. For rhTRAIL114-281 used as the comparative reference, a slight inhibitory effect on tumor cell growth was obtained relative to the control, with TGI at the level of 38%. Thus, fusion proteins of the invention exert much stronger effect compared to TRAIL alone.

B1. HCT116

On day 0 mice Crl:SHO-Prkdc^{scid}Hr^{hr} were grafted subcutaneously (s.c.) in the right side with 5×10⁶ of HCT116 cells suspended in 0.1 ml 3:1 mixture of HBSS buffer:Matrigel using syringe with a 0.5×25 mm needle (Bogmark). When tumors reached the size of 63-370 mm³ (day 17), mice were randomized to obtain the average size of tumors in the group of −190 mm³ and assigned to treatment groups. The treatment groups were administered with the preparations of fusion protein of the invention of Ex. 18^{b} (3 mg/kg) and rhTRAIL114-281 (70 mg/kg) as a comparison against formulation buffer (50 mM Trizma Base, 200 mM NaCl, 5 mM glutathione, 0.1 mM ZnCl₂, 10% glycerol, 80 mM saccharose, pH 8.0) as a control. rhTRAIL114-281 was administered intravenously (i. v.) six times every second day and Ex. 18^{b} was administered intravenously (i.v.) six times every fourth day. The control group received formulation buffer. On 47^{th} day of the experiment mice were sacrificed by disruption of the spinal cord.

The results of experiments are shown in FIG. 19a as a diagram of changes of the tumor volume and in FIG. 20a which shows tumor growth inhibition (% TGI) as the percentage of control.

The results of experiments presented in FIGS. 19a and 20a show that administration of the fusion protein of the invention of Ex.18^{b} caused HCT116 tumor growth inhibition with TGI 85% relative to the control on 47^{th} day of the experiment. For rhTRAIL114-281 used as the comparative reference, a slight inhibitory effect on tumor cell growth was obtained relative to the control, with TGI at the level of 37%. Thus, fusion proteins of the invention exert much stronger effect compared to TRAIL alone.

C. SW620 TAZD

On day 0 mice Crl:SHO-Prkdc^{scid}Hr^{hr} were grafted subcutaneously (s.c.) in the right side with 5×10⁶ of SW620 cells suspended in 0.1 ml 3:1 mixture of HBSS buffer:Matrigel using syringe with a 0.5×25 mm needle (Bogmark). When tumors reached the size of 92-348 mm³ (day 13), mice were randomized to obtain the average size of tumors in the group of ˜207 mm³ and assigned to treatment groups. The treatment groups were administered with the preparations of fusion proteins of the invention of Ex. 2^{b} (5 mg/kg), Ex. 18^{b} (3 mg/kg) and Ex. 51^{b} (5 mg/kg) and rhTRAIL114-281 (50 mg/kg) as a comparison against formulation buffer (50 mM Trizma Base, 200 mM NaCl, 5 mM glutathione, 0.1 mM ZnCl₂, 10% glycerol, 80 mM saccharose, pH 8.0) as a control. The preparations were administered intravenously (i. v.) six times every second day, The control group received formulation buffer [f25].

On 26^{th} day of the experiment mice were sacrificed by disruption of the spinal cord.

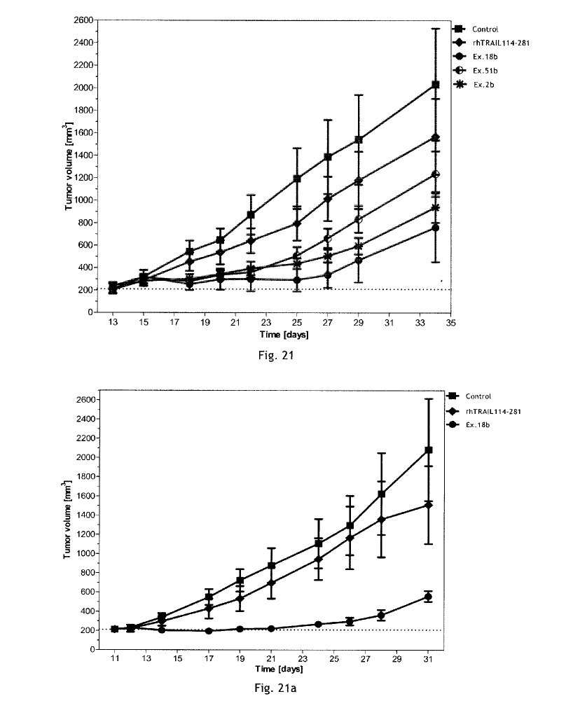

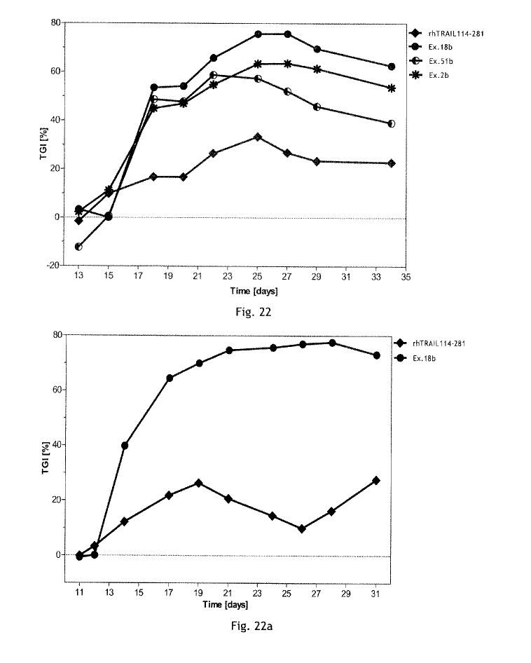

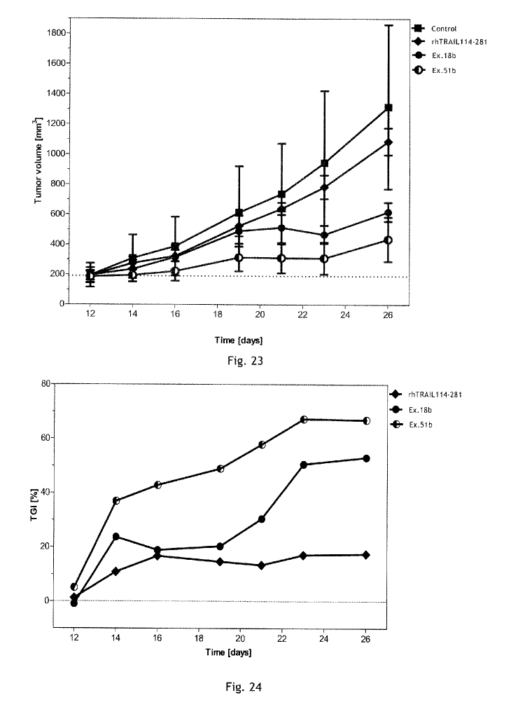

The results of experiments are shown in FIG. 21 as a diagram of changes of the tumor volume and in FIG. 22 which shows tumor growth inhibition (% TGI) as the percentage of control.

The results of experiments presented in FIGS. 21 and 22 show that administration of the fusion protein of the invention of Ex. 18^{b}, Ex. 51^{b}, and Ex. 2^{b} caused SW620 tumor growth inhibition, respectively with TGI 62.6%, 39% and 54% relative to the control on 34^{th} day of the experiment. For rhTRAIL114-281 used as the comparative reference, a slight inhibitory effect on tumor cell growth was obtained relative to the control, with TGI at the level of 23%. Thus, fusion proteins of the invention exert much stronger effect compared to TRAIL alone.

C1 SW620

On day 0 mice Crl:SHO-Prkdc^{scid}Hr^{hr} were grafted subcutaneously (s.c.) in the right side with 5×10⁶ of SW620 cells suspended in 0.1 ml 3:1 mixture of HBSS buffer:Matrigel using syringe with a 0.5×25 mm needle (Bogmark). When tumors reached the size of 126-300 mm³ (day 11), mice were randomized to obtain the average size of tumors in the group of −210 mm³ and assigned to treatment groups. The treatment groups were administered with the preparations of fusion proteins of the invention of Ex. 18^{b} (5 mg/kg), and rhTRAIL114-281 (50 mg/kg) as a comparison against formulation buffer (50 mM Trizma Base, 200 mM NaCl, 5 mM glutathione, 0.1 mM ZnCl₂, 10% glycerol, 80 mM saccharose, pH 8.0) as a control. The preparations were administered intravenously (i.v.) five times every third day. The control group received formulation buffer [f25].

On 31^{th} day of the experiment mice were sacrificed by disruption of the spinal cord.

The results of experiments are shown in FIG. 21a as a diagram of changes of the tumor volume and in FIG. 22a which shows tumor growth inhibition (% TGI) as the percentage of control.

The results of experiments presented in FIGS. 21a and 22a show that administration of the fusion protein of the invention of Ex. 18^{b} caused SW620 tumor growth inhibition with TGI 73% relative to the control on 31th day of the experiment. For rhTRAIL114-281 used as the comparative reference, a slight inhibitory effect on tumor cell growth was obtained relative to the control, with TGI at the level of 27.6%. Thus, fusion proteins of the invention exert much stronger effect compared to TRAIL alone.

D. HT-29

On day 0 mice Crl:SHO-Prkdc^{scid}Hr^{hr} were grafted subcutaneously (s.c.) in the right side with 5×10⁶ of HT-29 cells suspended in 0.1 ml 3:1 mixture of HBSS buffer:Matrigel using syringe with a 0.5×25 mm needle (Bogmark). When tumors reached the size of 80-348 mm³ (day 12), mice were randomized to obtain the average size of tumors in the group of—188 mm³ and assigned to treatment groups. The treatment groups were administered with the preparations of fusion proteins of the invention of Ex. 18^{b} (4 doses 3 mg/kg, remaining 2 doses 6 mg/kg), Ex. 51^{b} (5 mg/kg) and rhTRAIL114-281 (50 mg/kg) as a comparison against formulation buffer [f25]. The preparations were administered intravenously (i.v.) six times every second day. The control group received formulation buffer (50 mM Trizma Base, 200 mM NaCl, 5 mM glutathione, 0.1 mM ZnCl₂, 10% glycerol, 80 mM saccharose, pH 8.0) as a control. On 26^{th} day of the experiment mice were sacrificed by disruption of the spinal cord.

The experimental results are shown in FIG. 23 as a diagram of changes of the tumor volume and in FIG. 24 which shows tumor growth inhibition (% TGI) as the percentage of control.

The results of experiments presented in FIGS. 23 and 24 show that administration of the fusion proteins of the invention of Ex. 18^{b} and Ex. 51^{b} caused HT-29 tumor growth inhibition, respectively with TGI 53% and 67% relative to the control on 26^{th} day of the experiment. For rhTRAIL114-281 used as the comparative reference, a slight inhibitory effect on tumor cell growth was obtained relative to the control, with TGI at the level of 17.5%. Thus, fusion proteins of the invention exert much stronger effect compared to TRAIL alone.

Lung Cancer Model

A. On day 0 Cby.Cg-foxn1(^{nu})/J mice were grafted subcutaneously (sc) in the right side with 5×10⁶ of A549 cells suspended in 0.15 ml HBSS medium by means of a syringe with a 0.5 ×25 mm needle (Bogmark). On the 20th day of experiment mice were randomized to obtain the average size of tumours in the group of −45 mm³ and assigned to treatment groups. The treatment groups were administered with the preparations of fusion proteins of the invention of Ex. 18^{a} (5 mg/kg) and Ex. 35^{a} (5 mg/kg), rhTRAIL114-281 (15 mg/kg) as a comparison and water for injections as a control. The preparations were administered intravenously (i.v.) as follows: administration (day 1), one day pause, everyday administration on days 3rd, 4th, 5th, one day pause, administration (day 7th), one day pause, administration (day 9th). On the 38th day of experiment mice were sacrificed through disruption of the spinal cord.

The experimental results are shown on FIG. 3 and FIG. 4, as a diagram of changes of the tumor volume (FIG. 3) and tumor growth inhibition (% TGI) as the percentage of control (FIG. 4).

The results of experiments presented in FIG. 3 and FIG. 4 show that administration of the fusion proteins of the invention of Ex. 18^{a} and Ex. 35^{a} caused tumor A549 growth inhibition, with TGI 73.3% and 20.7%, respectively, relative to the control on 38th day of the experiment. For rhTRAIL114-281 used as the comparative reference, a slight inhibitory effect on tumor cell growth was obtained relative to the control, with TGI at the level of 16%. Thus, fusion proteins of the invention exert much stronger effect compared to TRAIL alone.

The tested fusion proteins did not cause significant side effects manifested by a decrease in body weight of mice (i.e. less than 10% of the baseline body weight). This shows low systemic toxicity of the protein.

B. On day 0 Cby.Cg-foxn1(^{nu})/J mice were grafted subcutaneously (sc) in the right side with 5×10⁶ of A549 cells suspended in 0.10 ml mixture of HBSS medium and Matrigel (4:1) by means of a syringe with a 0.5×25 mm needle (Bogmark). On the 19th day of experiment mice were randomized to obtain the average size of tumours in the group of −75 mm³ and assigned to treatment groups. The treatment groups were administered with the preparations of fusion proteins of the invention of Ex. 18^{a} (5 mg/kg) and Ex. 50^{a} (20 mg/kg), rhTRAIL114-281 (15 mg/kg) as a comparison and water for injections as a control. The preparations were administered intravenously (i.v.) six times every second day. On the 35th day of experiment mice were sacrificed through disruption of the spinal cord.

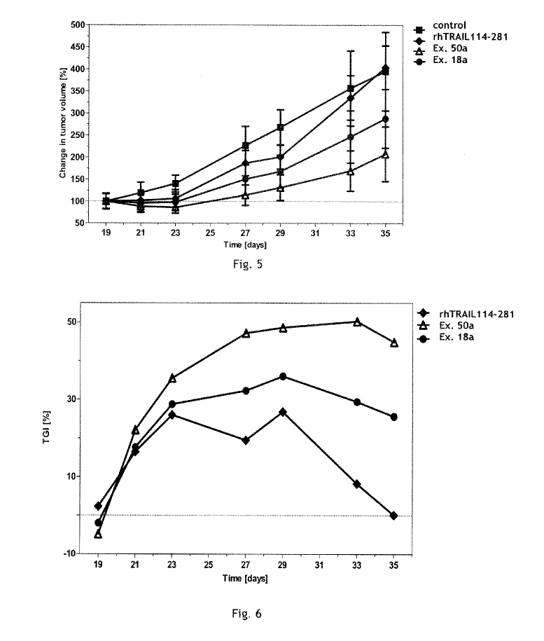

The experimental results are shown on FIG. 5 and FIG. 6, as a diagram of changes of the tumor volume (FIG. 5) and tumor growth inhibition (% TGI) as the percentage of control (FIG. 6).

The results of experiments show that administration of the fusion proteins of the invention of Ex. 18^{a} and Ex. 50^{a} caused tumor A549 growth inhibition, with TGI 26% and 45%, respectively, relative to the control on 35^{th} day of the experiment. For rhTRAIL114-281 used as the comparative reference, no inhibitory effect on tumor cell growth was obtained relative to the control, with TGI at the level of 0%. Thus, fusion proteins of the invention exert much stronger effect compared to TRAIL a(one.

The tested fusion proteins did not cause significant side effects manifested by a decrease in body weight of mice (i.e. less than 10% of the baseline body weight). This shows low systemic toxicity of the protein.

C. On day 0 mice were grafted subcutaneously (sc) in the right side with 5×10⁶ of A549 cells suspended in 0.10 ml mixture of HBSS medium and Matrigel (3:1) by means of a syringe with a 0.5 ×25 mm needle (Bogmark). On the 17th day of experiment mice were randomized to obtain the average size of tumours in the group of −100-120 mm³ and assigned to treatment groups. The treatment groups were administered with the preparations of fusion proteins of the invention of Ex. 2^{a} (5 mg/kg), Ex. 18^{a} (3 mg/kg) and Ex. 44^{a} (20 mg/kg), rhTRAIL114-281 (20 mg/kg) as a comparison and formulation buffer (19 mM NaH₂PO₄, 81 mM Na₂HPO₄, 50 mM NaCl, 5 mM glutation, 0.1 mM ZnCl₂, 10% glycerol, pH 7.4) as a control. The preparations were administered intravenously (i.v.) six times every second day. On the 34th day of experiment mice were sacrificed through disruption of the spinal cord.

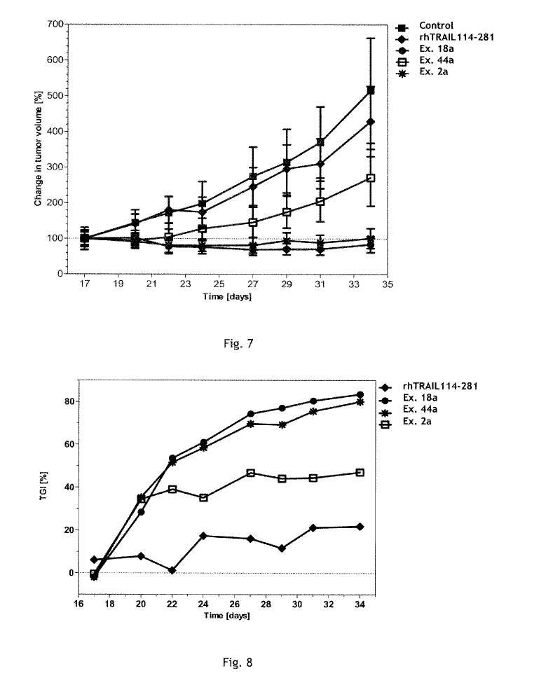

The experimental results are shown on FIG. 7 and FIG. 8, as a diagram of changes of the tumor volume (FIG. 7) and tumor growth inhibition (% TGI) as the percentage of control (FIG. 8).

The results of experiments show that administration of the fusion proteins of the invention of Ex. 2^{a}, Ex. 18^{a} and of Ex. 44^{a} caused tumor A549 growth inhibition, with TGI 83.5%, 80% and 47%, respectively, relative to the control on 34^{th} day of the experiment. For rhTRAIL114-281 used as the comparative reference, a slight inhibitory effect on tumor cell growth was obtained relative to the control, with TGI at the level of 21.8%. Thus, fusion proteins of the invention exert much stronger effect compared to TRAIL alone.

The tested fusion proteins did not cause significant side effects manifested by a decrease in body weight of mice (i.e. less than 10% of the baseline body weight). This shows low systemic toxicity of the protein.

D. On day 0 mice were grafted subcutaneously (Sc) in the right side with 7×10⁶ of A549 cells suspended in 0.10 ml mixture of HBSS medium and Matrigel (3:1) by means of a syringe with a 0.5×25 mm needle (Bogmark). On the 21th day of experiment mice were randomized to obtain the average size of tumours in the group of −160-180 mm³ and assigned to treatment groups. The treatment groups were administered with the preparations of fusion proteins of the invention of Ex. 20^{a} (15 mg/kg), Ex. 26^{a} (6 mg/kg), Ex. 43^{a} (10 mg/kg) and Ex. 47^{a} (5 mg/kg), rhTRAIL114-281 (40 mg/kg) as a comparison and formulation buffer (5 mM NaH₂PO₄, 95 mM Na2HPO₄, 200 mM NaCl, 5 mM glutation, 0.1 mM ZnCl₂, 10% glycerol, 80 mM saccharose, pH 7.4) as a control. The preparations were administered intravenously (i. v.) six times every second day. On the 35th day of experiment mice were sacrificed through disruption of the spinal cord.

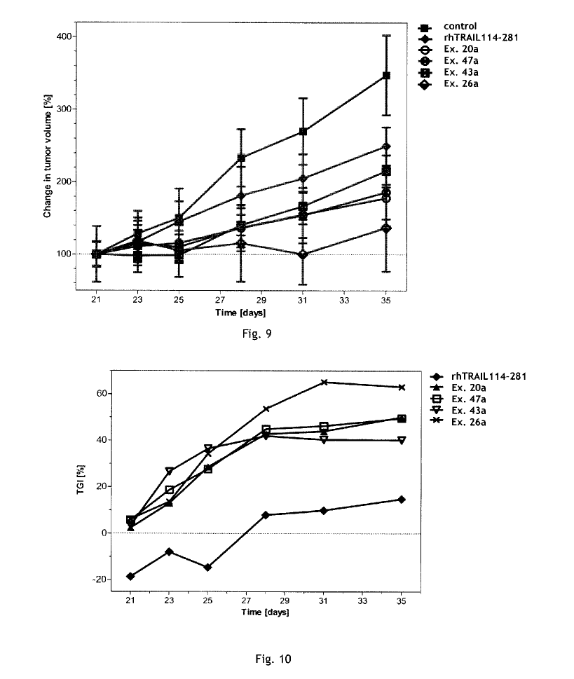

The experimental results are shown on FIG. 9 and FIG. 10, as a diagram of changes of the tumor volume (FIG. 9) and tumor growth inhibition (% TGI) as the percentage of control (FIG. 10).

The results of experiments show that administration of the fusion proteins of the invention of Ex. 20^{a}, Ex. 26^{a}, Ex. 43^{a} and Ex. 47^{a} caused tumor A549 growth inhibition, with TGI 49.5%, 64%, 40.2% and 49.5%, respectively, relative to the control on 35^{th} day of the experiment. For rhTRAIL114-281 used as the comparative reference, a slight inhibitory effect on tumor cell growth was obtained relative to the control, with TGI at the level of 15%. Thus, fusion proteins of the invention exert much stronger effect compared to TRAIL alone.

The tested fusion proteins did not cause significant side effects manifested by a decrease in body weight of mice (i.e. less than 10% of the baseline body weight).

This shows low systemic toxicity of the protein.

E. A549-regrowth of tumor

On day 0 mice Crl:SHO-Prkdc^{scid}Hr^{hr} were grafted subcutaneously (s.c.) in the right side with 7×10⁶ of A549 cells suspended in 0.1 ml 3:1 mixture of HBSS buffer:Matrigel using syringe with a 0.5×25 mm needle (Bogmark). When tumors reached the size of 85-302 mm³ (day 17), mice were randomized to obtain the average size of tumors in the group of −177 mm³ and assigned to treatment groups. The treatment groups were administered with the preparations of fusion proteins of the invention of Ex. 2^{b} (5 mg/kg), Ex. 18^{b} (3 mg/kg) and rhTRAIL114-281 (90 mg/kg) as a comparison against formulation buffer (50 mM Trizma Base, 200 mM NaCl, 5 mM glutathione, 0.1 mM ZnCl₂, 10% glycerol, 80 mM saccharose, pH 8.0) as a control. rhTRAIL114-281 was administered intravenously (i.v.) twelve times every second day, Ex. 2^{b} was administered intravenously (i.v.) seven times every second day and Ex. 18^{b} was administered intravenously (i. v.) on 17, 20, 25, and 29th day of the experiment. The control group received formulation buffer. In 45^{th} day of the experiment mice were sacrificed by disruption of the spinal cord.

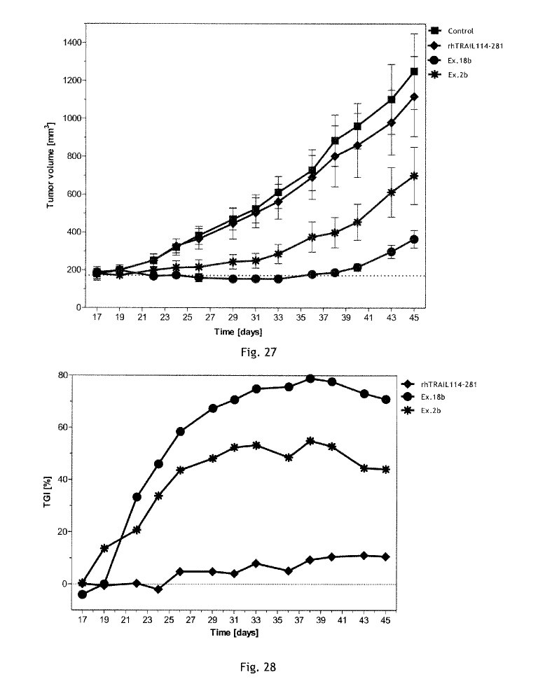

The experimental results are shown in FIG. 27 as a diagram of changes of the tumor volume and in FIG. 28 which shows tumor growth inhibition (% TGI) as the percentage of control.

The results of experiments presented in FIGS. 27 and 28 show that administration of the fusion protein of the invention of Ex. 18^{b} and Ex. 2^{b} caused A549 tumor growth inhibition with TGI 71% and 44%, respectively, relative to the control on 45^{th} day of the experiment. For rhTRAIL114-281 used as the comparative reference, a slight inhibitory effect on tumor cell growth was obtained relative to the control, with TGI at the level of 10.6%. Thus, fusion proteins of the invention exert much stronger effect compared to TRAIL alone.

Pancreas Cancer Model

On day 0 mice were grafted subcutaneously (sc) in the right side with 7×10⁶ of PANC-1 cells suspended in 0.10 ml mixture of HBSS medium and Matrigel (3:1) by means of a syringe with a 0.5 ×25 mm needle (Bogmark). On the 27th day of experiment mice were randomized to obtain the average size of tumours in the group of ˜95 mm³ and assigned to treatment groups. The treatment groups were administered with the preparations of fusion proteins of the invention of Ex. 20^{a} (5 mg/kg), Ex. 51^{a} (10 mg/kg) and Ex. 52^{a} (10 mg/kg), rhTRAIL114-281 (20 mg/kg) as a comparison and formulation buffer (5 mM NaH₂PO₄, 95 mM Na₂HPO₄, 200 mM NaCl, 5 mM glutation, 0.1 mM ZnCl₂, 10% glycerol, 80 mM saccharose, pH 7.4) as a control. The preparations were administered intravenously (i. v.) six times every second day. On the 40th day of experiment mice were sacrificed through disruption of the spinal cord.

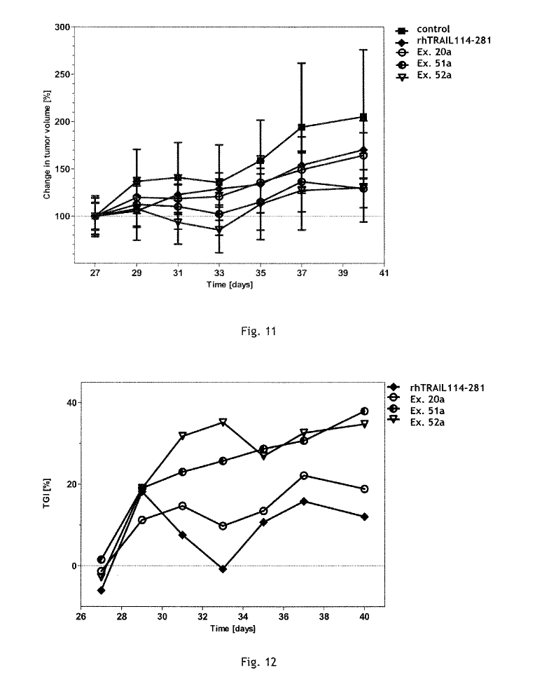

The experimental results are shown on FIG. 11 and FIG. 12, as a diagram of changes of the tumor volume (FIG. 11) and tumor growth inhibition (% TGI) as the percentage of control (FIG. 12).

The results of experiments show that administration of the fusion proteins of the invention of Ex. 20^{a}, Ex. 51^{a} and Ex. 52^{a} a caused tumor PANC-1 growth inhibition, with TGI 19%, 38 and 34%, respectively, relative to the control on 40th day of the experiment. For rhTRAIL114-281 used as the comparative reference, a slight inhibitory effect on tumor cell growth was obtained relative to the control, with TGI at the level of 12%. Thus, fusion proteins of the invention exert much stronger effect compared to TRAIL alone.

The tested fusion proteins did not cause significant side effects manifested by a decrease in body weight of mice (i.e. less than 10% of the baseline body weight). This shows low systemic toxicity of the protein.

B. On day 0 mice were grafted subcutaneously (sc) in the right side with 5×10⁶ of PANC-1 cells suspended in 0.10 ml mixture of HBSS medium and Matrigel (3:1) by means of a syringe with a 0.5×25 mm needle (Bogmark). On the 31st day of experiment mice were randomized to obtain the average size of tumours in the group of ˜110 mm³ and assigned to treatment groups. The treatment groups were administered with the preparations of fusion proteins of the invention of Ex. 18^{a} (3 mg/kg) and Ex. 44^{a} (20 mg/kg), rhTRAIL114-281 (20 mg/kg) as a comparison and formulation buffer ((19 mM NaH₂PO₄, 81 mM Na₂HPO₄, 50 mM NaCl, 5 mM glutation, 0.1 mM ZnCl₂, 10% glycerol, pH 7.4) as a control. The preparations were administered intravenously (i. v.) six times every second day. On the 42nd day of experiment mice were sacrificed through disruption of the spinal cord.

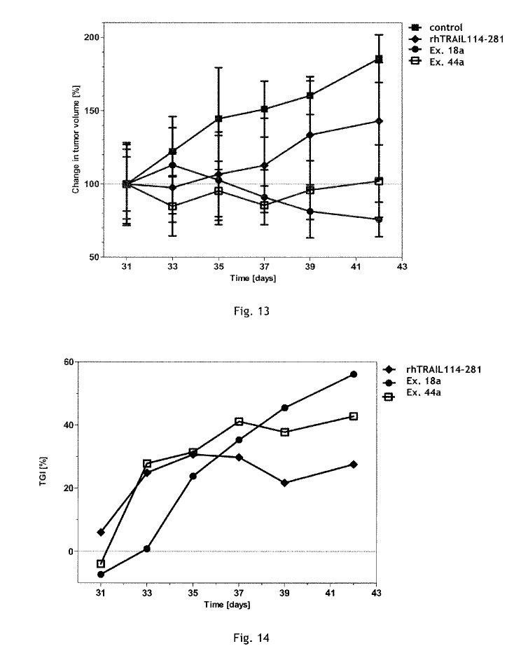

The experimental results are shown on FIG. 13 and FIG. 14, as a diagram of changes of the tumor volume (FIG. 13) and tumor growth inhibition (% TGI) as the percentage of control (FIG. 14).

The results of experiments show that administration of the fusion proteins of the invention of Ex. 18^{a} and Ex. 44^{a} caused tumor PANC-1 growth inhibition, with TGI 56% and 43%, respectively, relative to the control on 42^{nd} day of the experiment. For rhTRAIL114-281 used as the comparative reference, a slight inhibitory effect on tumor cell growth was obtained relative to the control, with TGI at the level of 27.5%. Thus, fusion proteins of the invention exert much stronger effect compared to TRAIL alone.

The tested fusion proteins did not cause significant side effects manifested by a decrease in body weight of mice (i.e. less than 10% of the baseline body weight). This shows low systemic toxicity of the protein.

Prostate Cancer Model

On day 0 mice were grafted subcutaneously (se) in the right side with 5×10⁶ of PC3 cells suspended in 0.20 ml mixture of HBSS medium and Matrigel (9:1) by means of a syringe with a 0.5 ×25 mm needle (Bogmark). On the 29th day of experiment mice were randomized to obtain the average size of tumours in the group of ˜90 mm³ and assigned to treatment groups. The treatment groups were administered with the preparations of fusion proteins of the invention of Ex. 18^{a} (5 mg/kg) and water for injection as a control. The preparations were administered intravenously (i. v.) six times every second day. On the 60th day of experiment mice were sacrificed through disruption of the spinal cord.

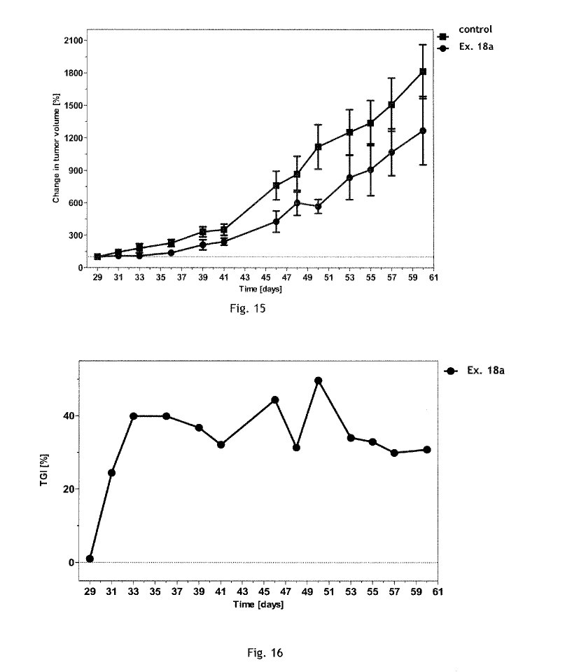

The experimental results are shown on FIG. 15 and FIG. 16, as a diagram of changes of the tumor volume (FIG. 15) and tumor growth inhibition (% TGI) as the percentage of control and (FIG. 16).

The results of experiments show that administration of the fusion protein of the invention of Ex. 18^{a} caused tumor PC3 growth inhibition, with TGI 30.8% relative to the control on 60th day of the experiment.

The tested fusion proteins did not cause significant side effects manifested by a decrease in body weight of mice (i.e. less than 10% of the baseline body weight).

This shows low systemic toxicity of the protein.

Liver Cancer Model

A. PCL/PRF/5

On day 0 mice Crl:SHO-Prkdc^{scid}Hr^{hr} were grafted subcutaneously (sc) in the right side with 7×10⁶ of PCL/PRF/5 cells suspended in 0.10 ml mixture of HBSS medium and Matrigel (3:1) by means of a syringe with a 0.5×25 mm needle (Bogmark). On the 31st day of experiment mice were randomized to obtain the average size of tumours in the group of ˜200 mm³ and assigned to treatment groups. The treatment groups were administered with the preparations of fusion proteins of the invention of Ex. 51^{a} (10 mg/kg) and rhTRAIL114-281 (30 mg/kg) as a comparison and formulation buffer (5 mM NaHzPO₄, 95 mM Na₂HPO₄, 200 mM NaCl, 5 mM glutation, 0.1 mM ZnCl₂, 10% glycerol, 80 mM saccharose, pH 7.4) as a control. The preparations were administered intravenously (i.v.) six times every second day. On the 49th day of experiment mice were sacrificed through disruption of the spinal cord.

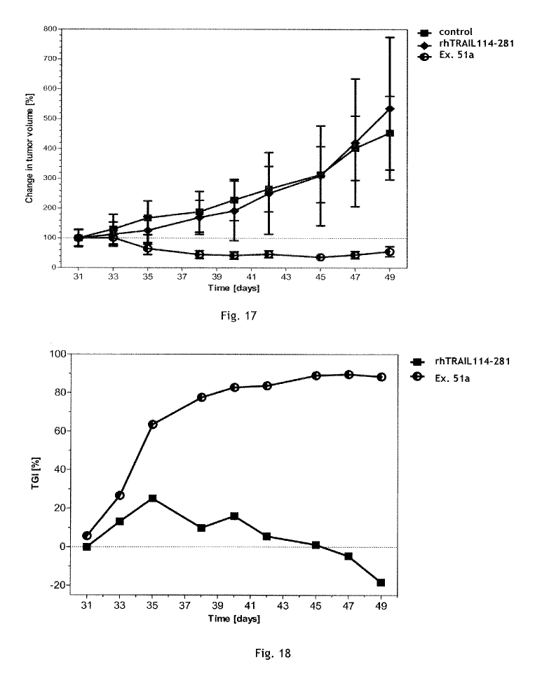

The experimental results are shown on FIG. 17 and FIG. 18, as a diagram of changes of the tumor volume (FIG. 17) and tumor growth inhibition (% TGI) as the percentage of control and (FIG. 18).

The results of experiments show that administration of the fusion protein of the invention of Ex. 51^{a} caused tumor PCL/PRF/5 growth inhibition, with TGI 88.5% relative to the control on 49^{th} day of the experiment. For rhTRAIL114-281 used as a comparative reference, a slight inhibitory effect on tumor cell growth was obtained relative to the control, with TGI at the level of 18%. Thus, fusion proteins of the invention exert much stronger effect compared to TRAIL alone.

B. HepG2

On day 0 mice Crl:SHO-Prkdc^{scid}Hr^{hr} were grafted subcutaneously (s.c.) in the right side with 7×10⁶ of HepG2 cells suspended in 0.1 ml 3:1 mixture of HBSS buffer:Matrigel using syringe with a 0.5×25 mm needle (Bogmark). When tumors reached the size of 64-530 mm³ (day 25), mice were randomized to obtain the average size of tumors in the group of −228 mm³ and assigned to treatment groups. The treatment groups were administered with the preparations of fusion protein of the invention of Ex.18^{a} (5 mg/kg supplemented with 10 mg/kg HSA) and rhTRAIL114-281 (50 mg/kg) as a comparison against formulation buffer (50 mM Trizma Base, 200 mM NaCl, 5 mM glutathione, 0.1 mM ZnCl₂, 10% glycerol, 80 mM saccharose, pH 8.0) as a control and reference compound 5FU (20 mg/kg). rhTRAIL114-281 was administered intravenously (i.v.) six times every second day, Ex.18^{b} was administered intravenously (i.v.) on 25, 27, 29, 37, and 42th day of the experiment. 5FU (20 mg/kg) was administered intraperitoneally (i.p.) six times every second day. The control group received formulation buffer. On 49^{th} day of the experiment mice were sacrificed by disruption of the spinal cord.

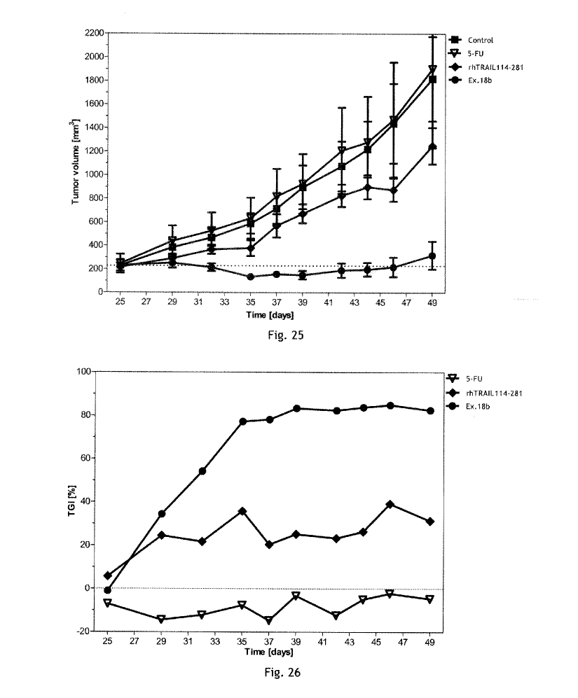

The results of experiments are shown in FIG. 25 as a diagram of changes of the tumor volume and in FIG. 26 which shows tumor growth inhibition (% TGI) as the percentage of control.

The results of experiments presented in FIGS. 25 and 26 show that administration of the fusion protein of the invention of Ex. 18^{b} caused HepG2 tumor growth inhibition with TGI 82.5% relative to the control on 49th day of the experiment. For rhTRAIL114-281 and 5FU used as a comparative reference, a slight inhibitory effect on tumor cell growth was obtained relative to the control, with TGI at the level of 31% and -4.7%, respectively. Thus, fusion proteins of the invention exert much stronger effect compared to TRAIL alone and standard chemotherapy.

The tested fusion proteins did not cause significant side effects manifested by a decrease in body weight of mice (i.e. less than 10% of the baseline body weight). This shows low systemic toxicity of the protein.

Multidrug Resistant Uterine Sarcoma Model

MES-SA. Dx5

On day 0 mice Crl:SHO-Prkdc^{scid}Hr^{hr} were grafted subcutaneously (s.c.) in the right side with 7×10⁶ of MES-SA.Dx5 cells suspended in 0.1 ml 3:1 mixture of HBSS buffer:Matrigel using syringe with a 0.5×25 mm needle (Bogmark). When tumors reached the size of 64-323 mm³ (day 13), mice were randomized to obtain the average size of tumors in the group of ˜180 mm³ and assigned to treatment groups. The treatment groups were administered with the preparations of fusion protein of the invention of Ex. 18^{b} (5 mg/kg) and rhTRAIL114-281 (50 mg/kg) as a comparison against formulation buffer (50 mM Trizma Base, 200 mM NaCl, 5 mM glutathione, 0.1 mM ZnCl₂, 10% glycerol, 80 mM saccharose, pH 8.0) as a control and reference compound CPT-11 (camptothecin, Pfeizer) (30 mg/kg), rhTRAIL114-281 and Ex. 18^{b} were administered intravenously (i.v.) six times every second day. CPT-11 was administered intraperitoneally (i.p.) six times every second day. The control group received formulation buffer. On 34th day of the experiment mice were sacrificed by disruption of the spinal cord.

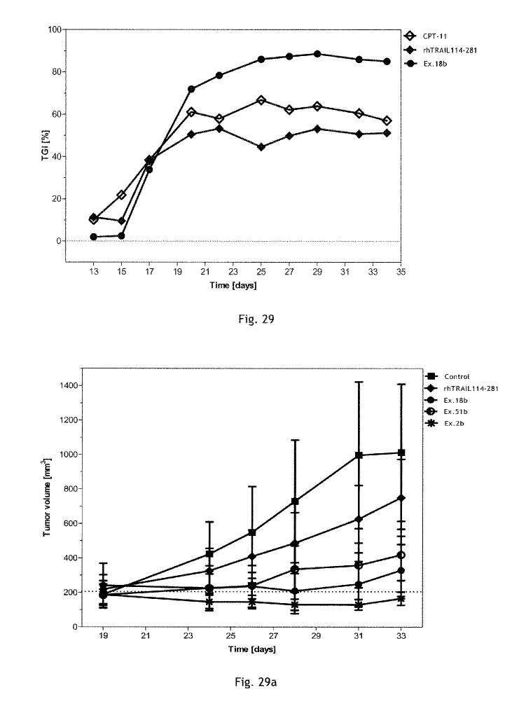

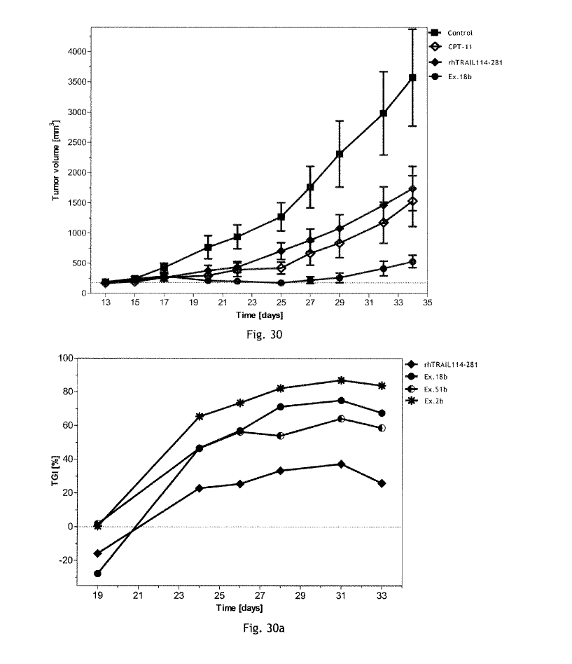

The results of experiments are shown in FIG. 29 as a diagram of changes of the tumor volume and in FIG. 30 which shows tumor growth inhibition (% TGI) as the percentage of control.

The results of experiments presented in FIGS. 29 and 30 show that administration of the fusion protein of the invention of Ex. 18^{b} caused MES-SA/Dx5 tumor growth inhibition with TGI 85% relative to the control on 34th day of the experiment. For rhTRAIL114-281 and CPT-11 used as the comparative reference, a slight inhibitory effect on tumor cell growth was obtained relative to the control, with TGI at the level of 51% and 57%, respectively. Thus, fusion proteins of the invention exert much stronger effect compared to TRAIL alone and standard chemotherapy.

MES-SA. Dx5

On day 0 mice Crl:SHO-Prkdc^{scid}Hr^{hr} were grafted subcutaneously (s.c.) in the right side with 7×10⁶ of MES-SA.Dx5 cells suspended in 0.1 ml 3:1 mixture of HBSS buffer:Matrigel using syringe with a 0.5×25 mm needle (Bogmark). When tumors reached the size of 26-611 mm³ (day 19), mice were randomized to obtain the average size of tumors in the group of ˜180 mm³ and assigned to treatment groups. The treatment groups were administered with the preparations of fusion protein of the invention of Ex. 2^{b} (3 mg/kg), Ex. 18^{b} (3 mg/kg), Ex. 51^{b} (7.5 mg/kg) and rhTRAIL114-281 (60 mg/kg) as a comparison against formulation buffer (50 mM Trizma Base, 200 mM NaCl, 5 mM glutathione, 0.1 mM ZnCl₂, 10% glycerol, 80 mM saccharose, pH 8.0). rhTRAIL114-281, Ex. 2^{b} and Ex. 51^{b} were administered intravenously (i.v.) six times every second day. Ex. 18b was administered intravenously (i.v.) four times every second day. The control group received formulation buffer.

On the 33th day of the experiment mice were sacrificed by disruption of the spinal cord.

The experimental results are shown in FIG. 29a as a diagram of changes of the tumor volume and in FIG. 30a which shows tumor growth inhibition (% TGI) as the percentage of control.

The results of experiments presented in the graphs in FIGS. 29a and 30a show that administration of the fusion proteins of the invention of Ex. 2^{b}, Ex. 18^{b} and Ex. 51^{b} caused MES-SA/Dx5 tumor growth inhibition with TGI 84%, 67.5% and 58.6%, respectively, relative to the control on 33th day of the experiment. For rhTRAIL114-281 used as the comparative reference, a slight inhibitory effect on tumor cell growth was obtained relative to the control, with TGI at the level of 25.8%. Thus, fusion proteins of the invention exert much stronger effect compared to TRAIL alone.

1. A fusion protein comprising:
domain (a) which is a functional fragment of the sequence of soluble hTRAIL protein, which fragment begins with an amino acid at a position not lower than hTRAIL95 or a homolog of said functional fragment having at least 70% sequence identity, preferably 85% identity and ends with the amino acid hTRAIL281, and
at least one domain (b) which is the sequence of an effector peptide inhibiting protein synthesis, wherein the sequence of the domain (b) is attached at the C-terminus and/or N-terminus of domain (a),
and wherein the fusion protein does not contain a domain binding to carbohydrate receptors on the cell surface.

2. The fusion protein according to claim 1, wherein domain (a) comprises a fragment of soluble hTRAIL protein sequence which begins with an amino acid in the range from hTRAIL95 to hTRAIL121, inclusive, and ends with the amino acid 281.

3. The fusion protein according to claim 1, wherein domain (a) is selected from the group consisting of hTRAIL95-281, hTRAIL114-281, hTRAIL116-281, hTRAIL119-281, hTRAIL12D-281, and hTRAIL121-281.

4. The fusion protein according to claim 1, wherein domain (a) is selected from the group consisting of domains set forth as SEQ. No. 142 and SEQ. No. 143.

5. (canceled)

6. The fusion protein according to claim 1, wherein the effector peptide of domain (b) is a peptide which inhibits enzymatically protein translation on the level of ribosome.

7. The fusion protein according to claim 6, wherein the effector peptide is a peptide with enzymatic activity of N-glycosidase selected from the group consisting of protein toxins inactivating ribosomes RIP type 1 and catalytic subunits A of protein toxins inactivating ribosomes RIP type 2 or modifications thereof with preserved N-glycosidase activity of at least 85% sequence identity with the original sequence.

8.-9. (canceled)

10. The fusion protein according to claim 7, in which the effector peptide is selected from the group consisting of peptides set forth as SEQ. No, 55, SEQ. No. 56, SEQ. No. 57, SEQ. No. 58, SEQ. No. 59, SEQ. No. 60, SEQ. No. 61, SEQ. No. 62, SEQ. No. 63, SEQ. No. 64, SEQ. No. 65, SEQ. No. 66, SEQ, No. 67, SEQ. No. 70, SEQ. No. 78, SEQ. No. 82, SEQ. No. 194, SEQ. No. 195, SEQ. No. 198, SEQ. No. 199 and SEQ. No, 200.

11. The fusion protein according to claim 6, in which the effector peptide is a peptide with ribonuclease enzymatic activity.

12. (canceled)

13. The fusion protein according to claim 11, in which the effector peptide is selected from the group consisting of SEQ. No. 71 and SEQ. No. 72.

14. The fusion protein according to claim 6, in which the effector peptide with enzymatic activity of ADP-ribosyltransferase.

15. (canceled)

16. The fusion protein according to claim 14, in which the effector peptide is selected from the group consisting of SEQ. No, 79, SEQ. No, 80, SEQ. No. 81, SEQ. No, 83, SEQ. No. 84, SEQ. No. 196, SEQ. No. 197, SEQ. No. 201, SEQ. No. 202, SEQ. No. 203, SEQ. No, 204, SEQ. No. 205, SEQ. No. 206 and SEQ. No. 207.

17. The fusion protein according to claim 1, in which the effector peptide of domain (b) is a toxin inhibiting protein synthesis which belongs to a toxin-antitoxin system, and is selected from the group consisting of CcdB protein set forth as SEQ. No. 74 CcdB protein set forth as SEQ. No. 75, Kid protein set forth as SEQ. No. 73, RelE protein set forth as SEQ. No, 76 StaB protein set forth as SEQ. No. 77 and Hok protein set forth as SEQ. No. 208, and modifications thereof with preserved topoisomerase activity, mRNAse activity or binding with a cellular membrane activity of at least 85% sequence identity with the original sequence.

18.-19. (canceled)

20. The fusion protein according to any of the claim 1, which between domain (a) and domain (b) or between domains (b) contains domain (c) containing protease cleavage site recognized by protease present in the tumor environment.

21. (canceled)

22. The fusion protein according to claim 1, in which effector peptide of domain (b) is additionally connected with transporting domain (d), selected from the group consisting of:
(d1) a domain transporting through a cell membrane derived from Pseudomonas set forth as SEQ. No. 139;
(d2) a domain transporting through a membrane directing to endoplasmic reticulum selected from Lys Asp Glu Leu/KDEL, His Asp Glu Leu/HDEL, Arg Asp Glu Leu/RDEL, Asp Asp Glu Leu/DDEL, Ala Asp Glu Leu/ADEL, Ser Asp Glu Leu/SDEL, and Glu Asp Leu/KEDL;
(d3) polyarginine sequence transporting through a cell membrane, consisting of 6, 7, 8, 9, 10 or 11 Arg residues, and combinations thereof, wherein transporting domain (d) is located on C-terminus and/or N-terminus of effector peptide domain (b).

23.-26. (canceled)

27. The fusion protein according to of claim 20, which between domains (a), (b) and/or (c) contains domain (e) which is a linker for attachment of PEG molecule, selected from Ala Ser Gly Cys Gly Pro Glu/ASGCGPE, Ala Ala Cys Ala Ala/AACAA, Ser Gly Gly Cys Gly Gly Ser/SGGCGGS or Ser Gly Cys Gly Ser /SGCGS.

28. The fusion protein according to claim 20, which between domain (b) and domain (c) additionally contains a motive binding with integrins selected from the group consisting of Asn Gly Arg/NGR, Asp Gly Arg/DGR and Arg Gly Asp/RGD.

29. The fusion protein according to claim 1, having the amino acid sequence selected from the group consisting of SEQ. No. 1; SEQ. No. 2; SEQ. No, 3; SEQ. No. 4; SEQ. No. 5; SEQ. No. 6; SEQ. No. 7; SEQ. No. 8; SEQ. No. 9; SEQ. NO. 10; SEQ. No. 11; SEQ. No. 12; SEQ. No. 13; SEQ. No. 14; SEQ. No. 15; SEQ. No. 16; SEQ. No. 17; SEQ. No. 18; SEQ. No. 19; SEQ. No. 20; SEQ. No. 21; SEQ. No. 22; SEQ. No. 23; SEQ. No. 24; SEQ. No. 25; SEQ. No. 26, SEQ. No. 27; SEQ. No. 28; SEQ. No. 29; SEQ. No. 30; SEQ. No. 31; SEQ. No. 32; SEQ. No. 33; SEQ. No. 34; SEQ. No. 35; SEQ. No, 36; SEQ. No. 37; SEQ. No. 38; SEQ. No. 39; SEQ. No. 40; SEQ. No. 41; SEQ. No. 42; SEQ. No. 43; SEQ. No. 44; SEQ. No. 45; SEQ. No. 46; SEQ. No. 47; SEQ. No. 48; SEQ. No. 49; SEQ. No. 50; SEQ. No. 51; SEQ. No. 52; SEQ. No. 53. SEQ. No. 54; SEQ. No. 144, SEQ. No, 145; SEQ. No. 146, SEQ. No. 147, SEQ. No. 148, SEQ. No, 149, SEQ. No, 150, SEQ. No. 151, SEQ. No. 152, SEQ. No. 153, SEQ. No, 154, SEQ. No. 155, SEQ. No. 156, SEQ. No, 157, SEQ. No, 158, SEQ. No, 159, SEQ. No, 160, SEQ. No. 161, SEQ. No. 162, SEQ. No. 163, SEQ. No, 164; SEQ. No, 165, SEQ. No, 166; SEQ. No. 167, and SEQ. No, 168.

30.-36. (canceled)

37. A pharmaceutical composition comprising as an active ingredient the fusion protein as defined in claim 1, in combination with a pharmaceutically acceptable carrier.

38.-39. (canceled)

40. A method of treating cancer diseases in mammal, including human, which comprises administration to a subject in a need thereof an anti-neoplastic-effective amount of the fusion protein as defined in claim 1, or the pharmaceutical composition as defined in claim 37 or 38.

41. (canceled)
